(19) United States
(12) Patent Application Publication
(10) Pub. No.: US 2025/0066325 A1
(43) Pub. Date: Feb. 27, 2025

(54) DIRECTED DEGRON MOLECULES AND APPLICATIONS THEREOF

(71) Applicants: The Brigham and Women's Hospital, Inc., Boston, MA (US); The Broad Institute, Inc., Cambridge, MA (US)

(72) Inventors: Amit Choudhary, Boston, MA (US); Tuan Nguyen, Boston, MA (US); Sreekanth Vedagopuram, Boston, MA (US); Veronika Shoba, Cambridge, MA (US); Arghya Deb, Cambridge, MA (US); Praveen Tiwari, Boston, MA (US); Praveen Kokkonda, Boston, MA (US); Jaron Mercer, Cambridge, MA (US)

(21) Appl. No.: 18/706,896

(22) PCT Filed: Nov. 4, 2022
(86) PCT No.: PCT/US2022/049024

§ 371 Date: May 2, 2024

Related U.S. Application Data
(60) Provisional application No. 63/275,861, filed on Nov. 4, 2021.

(51) Int. Cl. C07D 401/14; A61K 31/454; A61K 31/4545; A61K 31/496; A61K 31/497; A61K 31/517; A61K 31/5377; A61K 47/55; C07D 401/04; C07D 487/10; C07D 491/107

(52) U.S. Cl. CPC C07D 401/14; A61K 31/454; A61K 31/4545; A61K 31/496; A61K 31/497; A61K 31/517; A61K 31/5377; A61K 47/55; C07D 401/04; C07D 487/10; C07D 491/107

(57) ABSTRACT

This invention is related to molecules and methods of use of said molecules or compounds comprising said molecules. A molecule may be a pomalidomide or a thalidomide analogue. A method of inducing degradation of a target protein comprising one or more zinc finger polypeptides in a cell may comprise exposing a cell transfected with the target protein with a molecule as described herein, a pharmaceutically acceptable salt thereof, or any combination thereof. A method of inducing degradation of a target protein comprising one or more FK506 binding protein (FKBP) domains or degradation of a target amine in a cell may comprise exposing a cell transfected with the target protein or comprising the target amine with a composition comprising a molecule as described herein, a pharmaceutically acceptable salt thereof, or any combination of compositions comprising molecules as described herein and pharmaceutically acceptable salts thereof. A method may improve on-target effects and reduce off-target effects.

### CROSS-REFERENCE TO RELATED APPLICATIONS

This application claims the benefit of and priority to U.S. Provisional Patent Application No. 63/275,861, filed on Nov. 4, 2021, the contents of which is incorporated by reference herein in its entirety.

### TECHNICAL FIELD

The subject matter disclosed herein is generally directed to molecules utilized to induce modifications in target protease substrates.

### BACKGROUND

Imide-based molecular glues (e.g., Pomalidomide) induce proximity between a ubiquitin ligase, such as cereblon (CRBN), and proteins with Zn-finger (ZF) motifs to trigger ubiquitination and degradation of the latter. Typically, pomalidomide is appended to target protein binders to generate Proteolysis Targeting Chimeras (PROTACs) that induce proximity-mediated target protein degradation. However, these pomalidomide-based PROTACs can also recruit other proteins with ZF motifs that serve key biological functions in normal development and disease progression. For example, tissue-specific deletion of pomalidomide-degradable ZF protein ZFP91 in regulatory T cells (Tregs) leads to Treg dysfunction and increases the severity of inflammation-driven colorectal cancer. Furthermore, there are numerous other proteins with important roles in cellular function, such as transcription factors, that also harbor ZF domains. The off-target degradation of these key ZF-containing proteins may have long-term implications such as the development of new cancers, dysregulation of lymphocyte development, and teratogenic effects. The ability of pomalidomide to degrade other proteins in a PROTAC-independent manner raises concerns about the dangers of off-target ubiquitination and degradation of these compounds, several of which are already in clinical trials. Thus, there is an urgent need to develop new molecular glues to control off-target degradation for use in such PROTACs.

Citation or identification of any document in this application is not an admission that such a document is available as prior art to the present disclosure.

### SUMMARY

In one aspect, the present invention provides for a molecule according to the formula

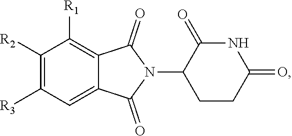

wherein R₁ is selected from —H, —R₄, —NHC(O)R₅, —NR₆R₇, —NHR₈, and —NHS(O₂)R₉; wherein R₂ is selected from —H, —R₄, —NH₂, —NHC(O)R₅, —NR₆R₇, —NHR₈, and —NHS(O₂)R₉; wherein R₃ is selected from —H, —R₄, and —NR₆R₇; wherein R₄-R₉ are independently selected from one or more nitrile, nitro, ether, alcohol, thiol, sulfone, sulfonate, halogen, carbonyl, acyl, ketone, carboxylate ester, amide, enone, anhydride, imide, alkyl, alkenyl, alkynyl, saturated cyclic hydrocarbon, unsaturated cyclic hydrocarbon, heteroalkyl, heterocyclic ring, aryl ring, and heteroaryl ring groups, and one or more fused rings thereof, more preferably selected from alkyl, amide, heteroalkyl, cycloalkyl, heterocyclic, aryl and heteroaryl groups; and wherein: when R₂ and R₃ are —H, R₁ is selected from —R₄, —NHC(O)R₅, —NR₆R₇, —NHR₈, and —NHS(O₂)R₉; and when R₁ and R₃ are —H, R₂ is selected from —R₄, —NH₂, —NHC(O)R₅, —NR₆R₇, —NHR₈, and —NHS(O₂)R₉.

In one example embodiment, R₂ and R₃ are —H, and R₁ is selected from —R₄, —NHC(O)R₅, and —NR₆R₇.

In one example embodiment, R₁ is according to —R₄, and —R₄ is selected from halogen, aryl, heteroaryl, and alkynyl groups. In one example embodiment, the halogen group is a bromine or a fluorine group. In one example embodiment, the aryl group is a phenyl group and the heteroaryl group is a pyridinyl group. In one example embodiment, the heteroaryl group is selected from indolyl, pyridinyl, isoxazolyl, and thiophene groups. In one example embodiment, the indolyl group is a 1-methyl-indolyl

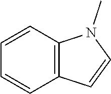

group, the isoxazolyl group is a 3,5-dimethyl-isoxazolyl

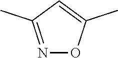

group, or the thiophene group is a benzothiophene group

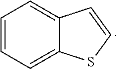

In one example embodiment, the alkynyl group is a 2-phenyl-acetylenyl group.

In one example embodiment, R₁ is —NHC(O)R₅, and R₅ is selected from alkyl, cycloalkyl, heterocyclic, heteroaryl, and aryl groups. In one example embodiment, R₅ is selected from methyl, phenyl, cyclopropyl, cyclobutyl, cyclopentyl, isoxazolyl, pyridinyl, and pyrazinyl groups.

In one example embodiment, R₁ is according to —NR₆R₇, and N, R₆, and R₇ taken together form a heterocyclic amine group. In one example embodiment, the heterocyclic amine group is selected from morpholinyl, azetidinyl, pyrrolidinyl, piperidinyl, piperazinyl, and diazaspiro groups. In one example embodiment, the pyrrolidinyl group is an unsubstituted pyrrolidinyl group or a 3, 3′-difluoro-pyrrolidinyl group. In one example embodiment, the piperazinyl group is a 4-acetyl-1-piperazinyl, a 4-Boc-1-piperazinyl, or a 4-methyl-1-piperazinyl group. In one example embodiment, the diazaspiro group is a 2,6-diazaspiro[3.3]heptan

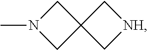

a 2-oxa-6-azaspiro[3.3]heptane

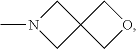

a 2-Boc-2,6-diazaspiro[3.3]heptane

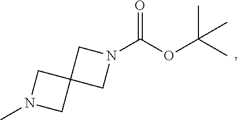

or a 3-Boc-3,9-diazaspiro[5.5]undecane

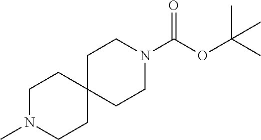 [SMILES: CN1CCC2(CC1)CCN(C(=O)OC(C)(C)C)CC2]

group.

In one example embodiment, R₁ is according to —NR₆R₇ or —NHR₈, R₆ and R₇ are independently selected from alkyl and cycloalkyl groups, and R₈ is a cycloalkyl group. In one example embodiment, —NR₆R₇ is a methylcyclohexyl amine group, and R₈ is a cyclohexyl group or a morpholinyl group.

In one example embodiment, R₁ and R₃ are —H, and R₂ is selected from —R₄, —NH₂, —NHC(O)R₅, —NR₆R₇, —NHR₈, and —NHS(O₂)R₉. In one example embodiment, R₂ is according to —R₄, and —R₄ is selected from halogen, nitro, heteroaryl, aryl, and alkynyl groups. In one example embodiment, the halogen group is a fluorine or bromine group. In one example embodiment, the heteroaryl group is selected from indolyl, pyridinyl, isoxazolyl, and thiophene groups. In one example embodiment, the indolyl group is a 1-methyl-indolyl

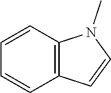

group, the isoxazolyl group is a 3,5-dimethyl-isoxazolyl

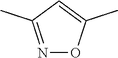

group, and the thiophene group is a benzothiophene group

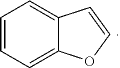

In one example embodiment, the aryl group is selected from phenyl. In one example embodiment, the alkynyl group is a 2-phenyl-acetylenyl group.

In one example embodiment, R₂ is according to —NHC(O)R₅, and R₅ is selected from alkyl, cycloalkyl, heterocyclic, heteroaryl, and aryl groups. In one example embodiment, R₅ is a methyl, a phenyl, a cyclopropyl, a cyclobutyl, a cyclopentyl, an isoxazolyl, a pyridinyl, or a pyrazinyl group.

In one example embodiment, R₂ is according to —NR₆R₇, and N, R₆, and R₇ taken together form a heterocyclic amine group. In one example embodiment, the heterocyclic amine group is selected from morpholinyl, azetidinyl, pyrrolidinyl, piperidinyl, piperazinyl, and diazaspiro groups. In one example embodiment, the pyrrolidinyl group is an unsubstituted pyrrolidinyl group or a 3, 3′-difluoro-pyrrolidinyl group. In one example embodiment, the piperazinyl group is a 4-acetyl-1-piperazinyl, a 4-Boc-1-piperazinyl, or a 4-methyl-1-piperazinyl group. In one example embodiment, the diazaspiro group is a 2,6-diazaspiro[3.3]heptane

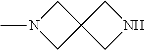

group, a 2-oxa-6-azaspiro[3.3]heptane

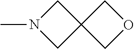

group, a 2-Boc-2,6-diazaspiro[3.3]heptane

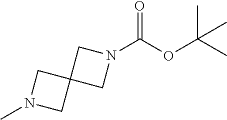

group, or a 3-Boc-3,9-diazaspiro[5.5]undecane

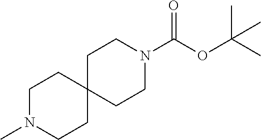 [SMILES: CN1CCC2(CC1)CCN(C(=O)OC(C)(C)C)CC2]

group.

In one example embodiment, R₂ is according to —NR₆R₇ or —NHR₈, R₆ and R₇ are independently selected from alkyl and cycloalkyl groups, and R₈ is selected from cycloalkyl and heterocyclic groups. In one example embodiment, wherein —NR₆R₇ is a methylcyclohexyl amine group, and R₈ is a cyclohexyl group or a morpholinyl group.

In one example embodiment, wherein R₂ is according to —NHS(O₂)R₉, and R₉ is an aryl group.

In one example embodiment, wherein R₁ is —H and R₂ and R₃ are according to the same —R₄ or —NR₆R₇, and N, R₆, and R₇ taken together form a heterocyclic amine group. In one example embodiment, R₂ and R₃ are according to the same —R₄, and —R₄, is a halogen. In one example embodiment, the halogen is a fluorine group. In one example embodiment, R₂ and R₃ are according to the same —NR₆R₇, and —NR₆R₇ is a morpholinyl group.

In one example embodiment, R₁ is —H, R₃ is according to —R₄, R₂ is according to —NR₆R₇, and N, R₆, and R₇ taken together form a heterocyclic amine group. In one example embodiment, —R₄ is a halogen and the heterocyclic amine group is selected from morpholinyl, piperazinyl, and diazaspiro groups. In one example embodiment, the halogen is a fluorine group. In one example embodiment, the piperazinyl group is a 4-acetyl-1-piperazinyl, a 4-Boc-1-piperazinyl, or a 4-methyl-1-piperazinyl group. In one example embodiment, the diazaspiro group is a 2-oxa-6-azaspiro[3.3]heptane

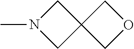

group, a 2-Boc-2,6-diazaspiro[3.3]heptane

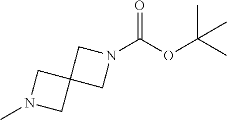

group, or a 3-Boc-3,9-diazaspiro[5.5]undecane

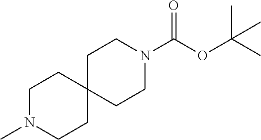 [SMILES: CN1CCC2(CC1)CCN(C(=O)OC(C)(C)C)CC2]

group. In one example embodiment, —R₄ is an aryl group and the heterocyclic amine group is a morpholinyl group. In one example embodiment, the aryl group is a phenyl group.

In one example embodiment, the molecule is selected from

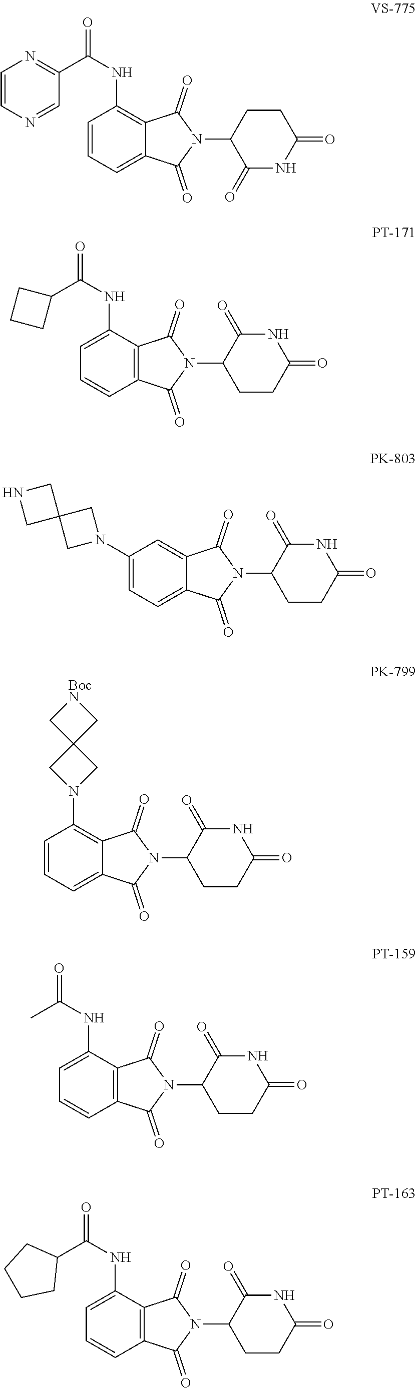 [SMILES: O=C1CCC(N2C(=O)c3cccc(NC(=O)c4cnccn4)c3C2=O)C(=O)N1 ; O=C1CCC(N2C(=O)c3cccc(NC(=O)C4CCC4)c3C2=O)C(=O)N1 ; O=C1CCC(N2C(=O)c3ccc(N4CC5(CNC5)C4)cc3C2=O)C(=O)N1 ; CC(C)(C)OC(=O)N1CC2(C1)CN(c1cccc3c1C(=O)N(C1CCC(=O)NC1=O)C3=O)C2 ; CC(=O)Nc1cccc2c1C(=O)N(C1CCC(=O)NC1=O)C2=O ; O=C1CCC(N2C(=O)c3cccc(NC(=O)C4CCCC4)c3C2=O)C(=O)N1]

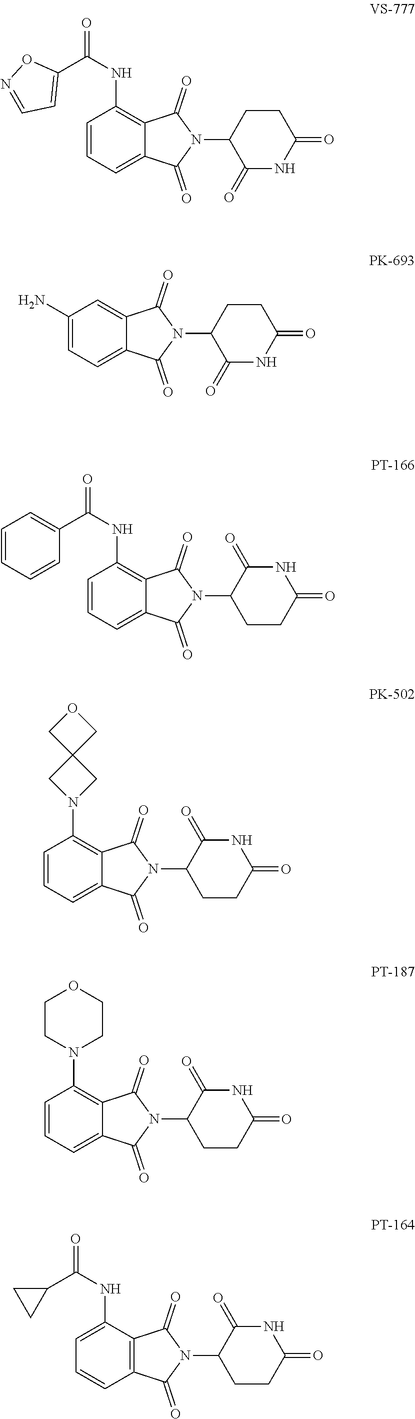 [SMILES: O=C1CCC(N2C(=O)c3cccc(NC(=O)c4ccno4)c3C2=O)C(=O)N1 ; Nc1ccc2c(c1)C(=O)N(C1CCC(=O)NC1=O)C2=O ; O=C1CCC(N2C(=O)c3cccc(NC(=O)c4ccccc4)c3C2=O)C(=O)N1 ; O=C1CCC(N2C(=O)c3cccc(N4CC5(COC5)C4)c3C2=O)C(=O)N1 ; O=C1CCC(N2C(=O)c3cccc(N4CCOCC4)c3C2=O)C(=O)N1 ; O=C1CCC(N2C(=O)c3cccc(NC(=O)C4CC4)c3C2=O)C(=O)N1]

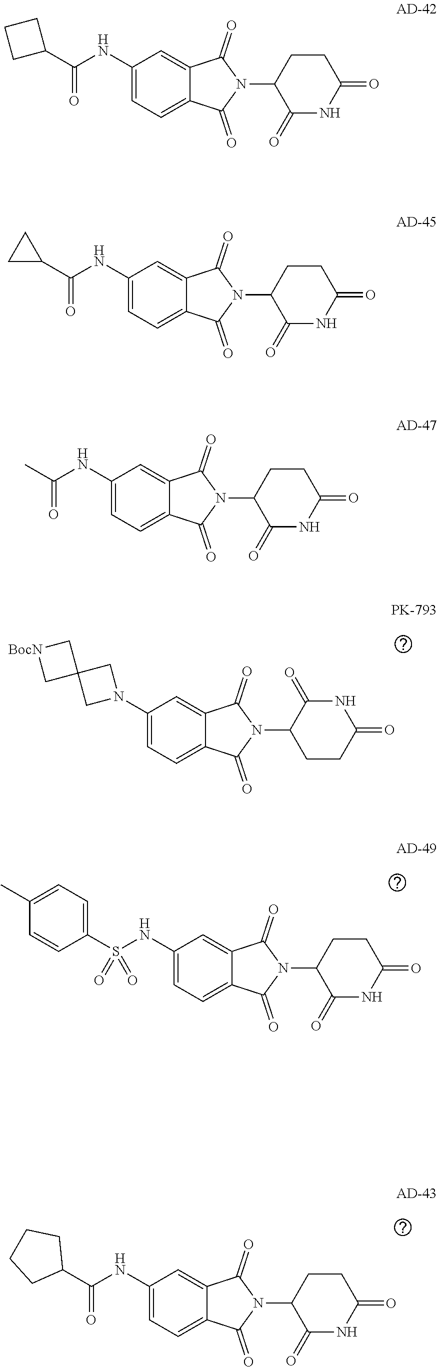 [SMILES: O=C1CCC(N2C(=O)c3ccc(NC(=O)C4CCC4)cc3C2=O)C(=O)N1 ; O=C1CCC(N2C(=O)c3ccc(NC(=O)C4CC4)cc3C2=O)C(=O)N1 ; CC(=O)Nc1ccc2c(c1)C(=O)N(C1CCC(=O)NC1=O)C2=O ; CC(C)(C)OC(=O)N1CC2(C1)CN(c1ccc3c(c1)C(=O)N(C1CCC(=O)NC1=O)C3=O)C2 ; Cc1ccc(S(=O)(=O)Nc2ccc3c(c2)C(=O)N(C2CCC(=O)NC2=O)C3=O)cc1 ; O=C1CCC(N2C(=O)c3ccc(NC(=O)C4CCCC4)cc3C2=O)C(=O)N1]

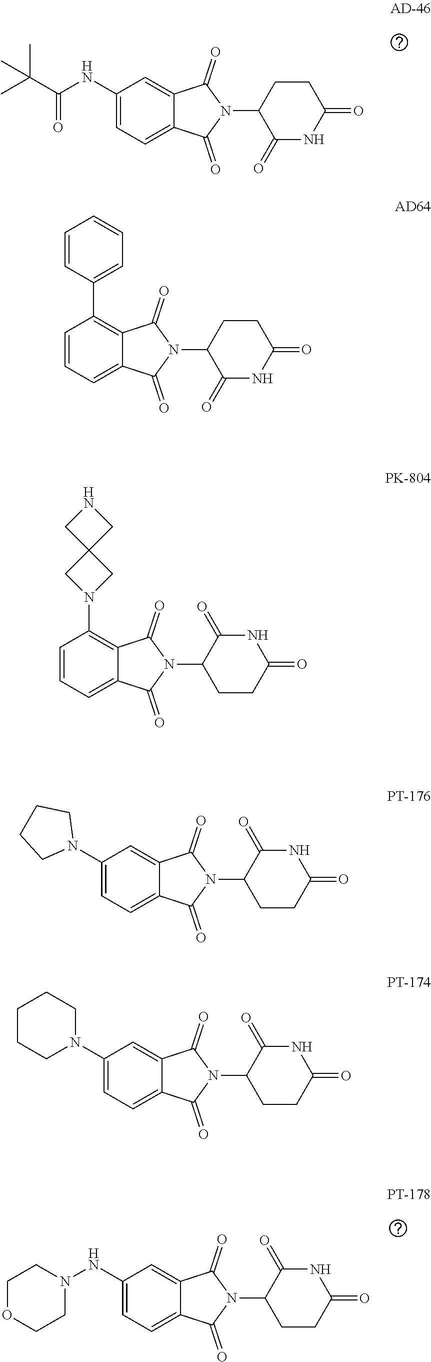 [SMILES: CC(C)(C)C(=O)Nc1ccc2c(c1)C(=O)N(C1CCC(=O)NC1=O)C2=O ; O=C1CCC(N2C(=O)c3cccc(-c4ccccc4)c3C2=O)C(=O)N1 ; O=C1CCC(N2C(=O)c3cccc(N4CC5(CNC5)C4)c3C2=O)C(=O)N1 ; O=C1CCC(N2C(=O)c3ccc(N4CCCC4)cc3C2=O)C(=O)N1 ; O=C1CCC(N2C(=O)c3ccc(N4CCCCC4)cc3C2=O)C(=O)N1 ; O=C1CCC(N2C(=O)c3ccc(NN4CCOCC4)cc3C2=O)C(=O)N1]

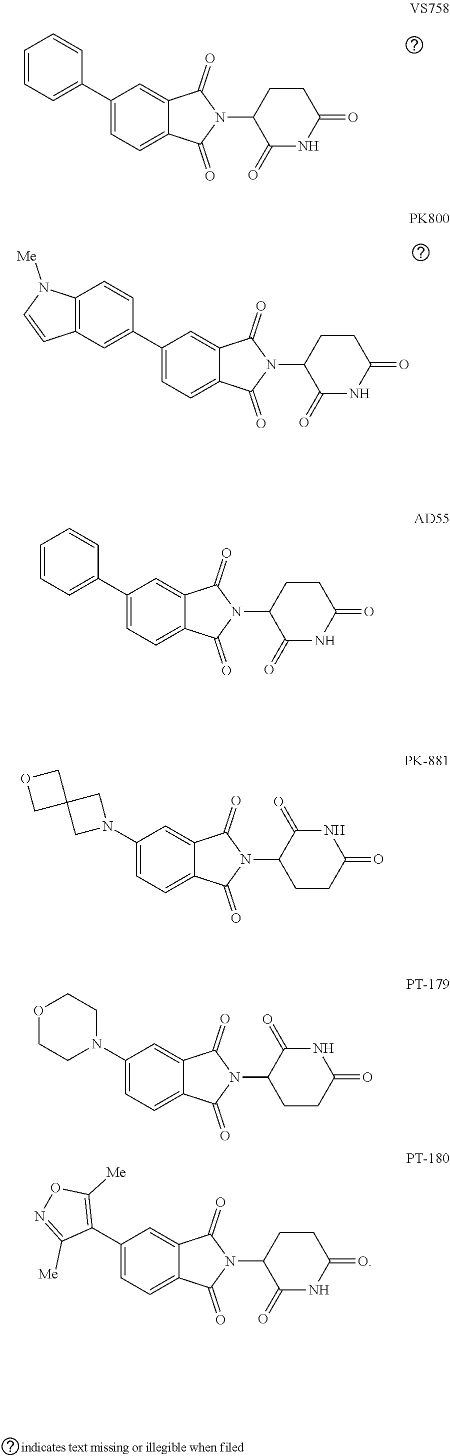 [SMILES: O=C1CCC(N2C(=O)c3ccc(-c4ccccc4)cc3C2=O)C(=O)N1 ; Cn1ccc2cc(-c3ccc4c(c3)C(=O)N(C3CCC(=O)NC3=O)C4=O)ccc21 ; O=C1CCC(N2C(=O)c3ccc(-c4ccccc4)cc3C2=O)C(=O)N1 ; O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1 ; Cc1noc(C)c1-c1ccc2c(c1)C(=O)N(C1CCC(=O)NC1=O)C2=O]

In one example embodiment, the molecule is selected from

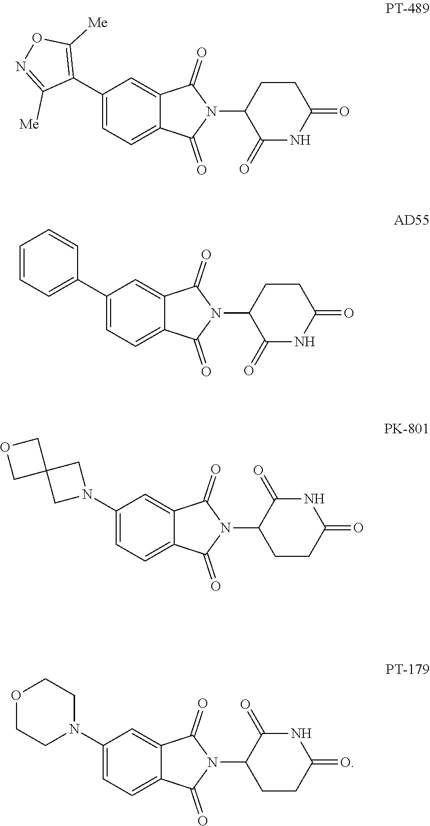 [SMILES: Cc1noc(C)c1-c1ccc2c(c1)C(=O)N(C1CCC(=O)NC1=O)C2=O ; O=C1CCC(N2C(=O)c3ccc(-c4ccccc4)cc3C2=O)C(=O)N1 ; O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1]

In one example embodiment, the molecule is selected from

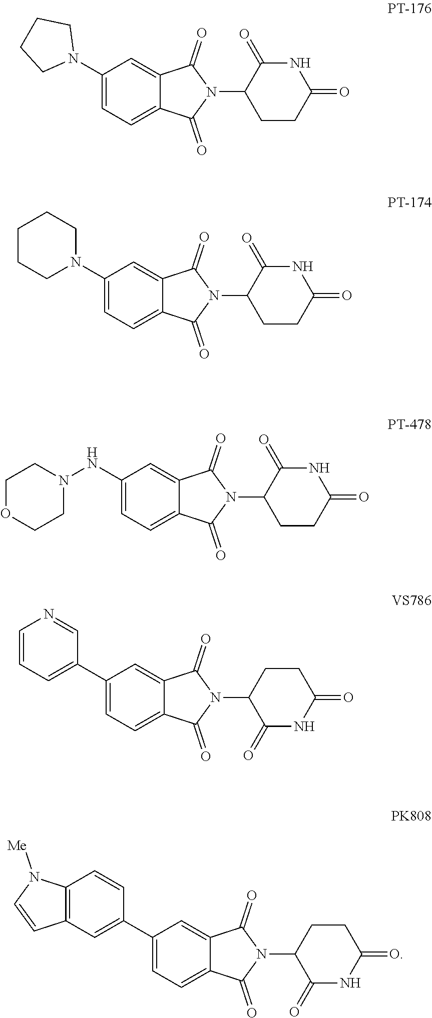 [SMILES: O=C1CCC(N2C(=O)c3ccc(N4CCCC4)cc3C2=O)C(=O)N1 ; O=C1CCC(N2C(=O)c3ccc(N4CCCCC4)cc3C2=O)C(=O)N1 ; O=C1CCC(N2C(=O)c3ccc(NN4CCOCC4)cc3C2=O)C(=O)N1 ; O=C1CCC(N2C(=O)c3ccc(-c4cccnc4)cc3C2=O)C(=O)N1 ; Cn1ccc2cc(-c3ccc4c(c3)C(=O)N(C3CCC(=O)NC3=O)C4=O)ccc21]

In one example embodiment, the molecule is selected from

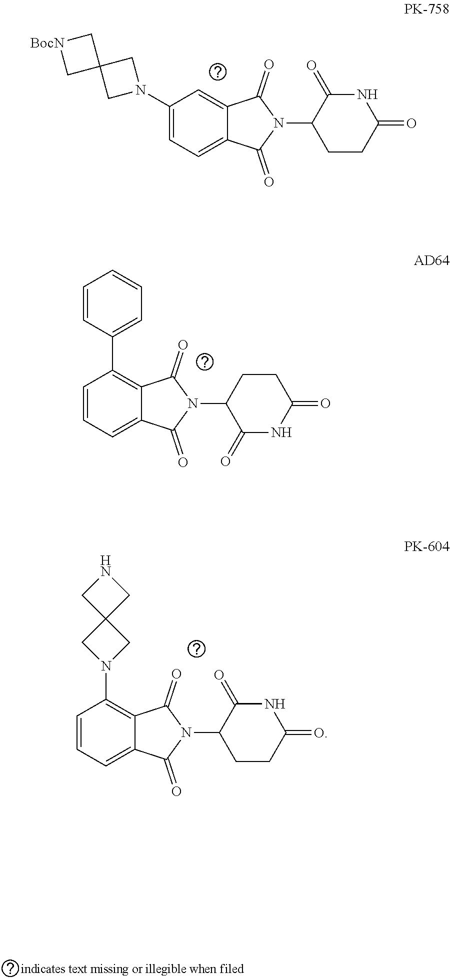 [SMILES: CC(C)(C)OC(=O)N1CC2(C1)CN(c1ccc3c(c1)C(=O)N(C1CCC(=O)NC1=O)C3=O)C2 ; O=C1CCC(N2C(=O)c3cccc(-c4ccccc4)c3C2=O)C(=O)N1 ; O=C1CCC(N2C(=O)c3cccc(N4CC5(CNC5)C4)c3C2=O)C(=O)N1]

In one example embodiment, the molecule is selected from

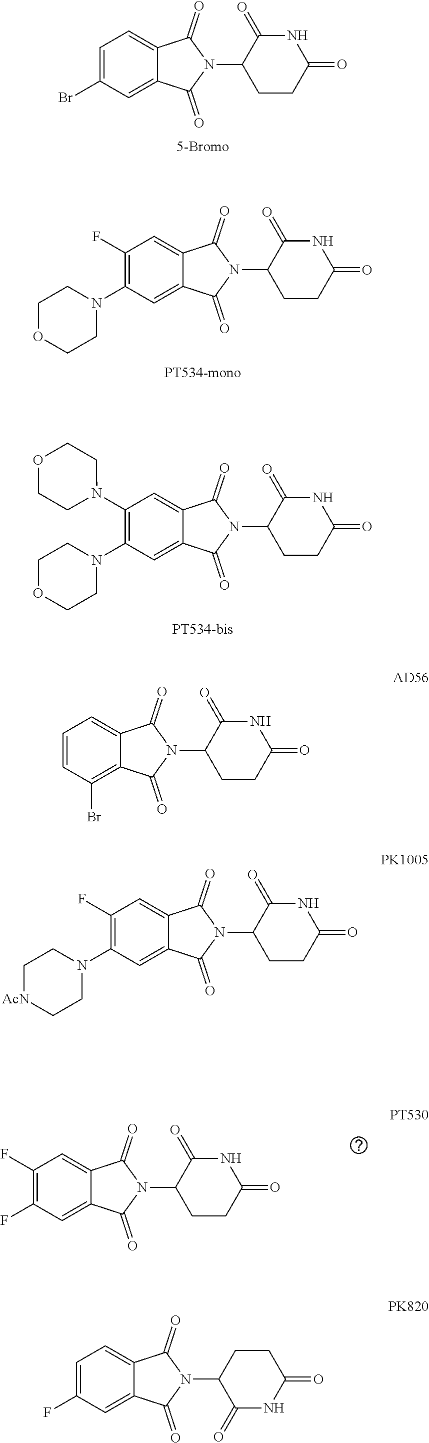

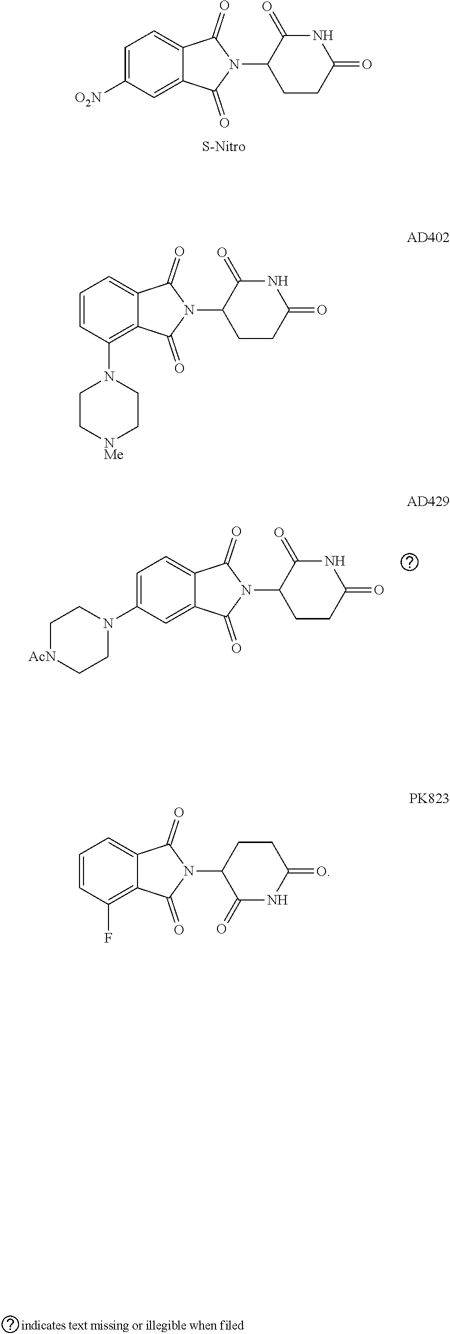

In one example embodiment, the molecule is selected from

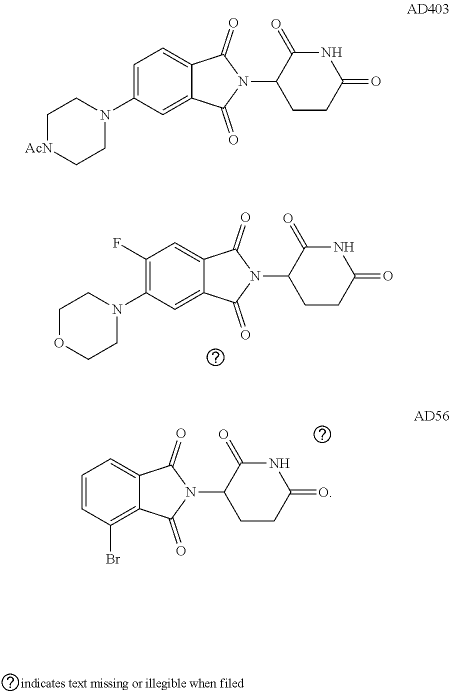

In one example embodiment, the molecule is selected from

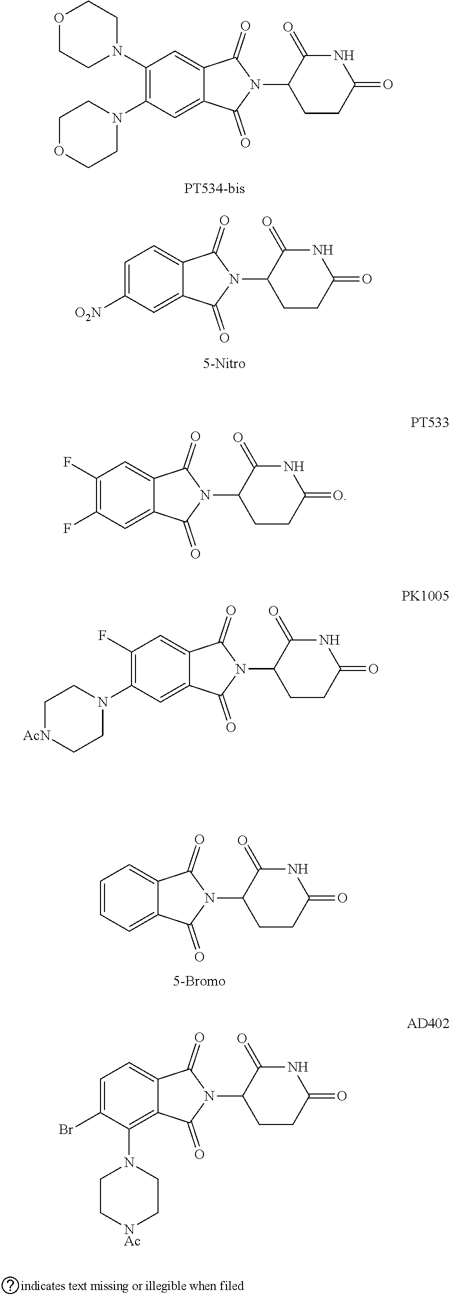

In one example embodiment, the molecule has the following structure

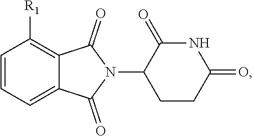

wherein R₁ is selected from

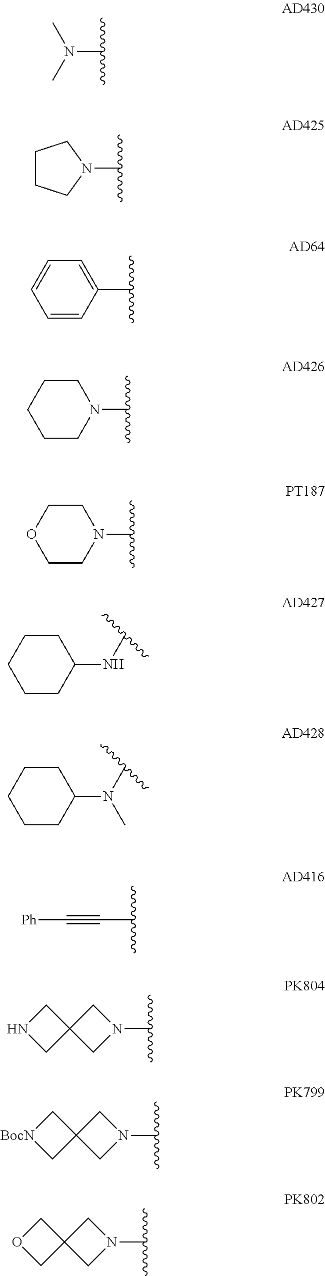

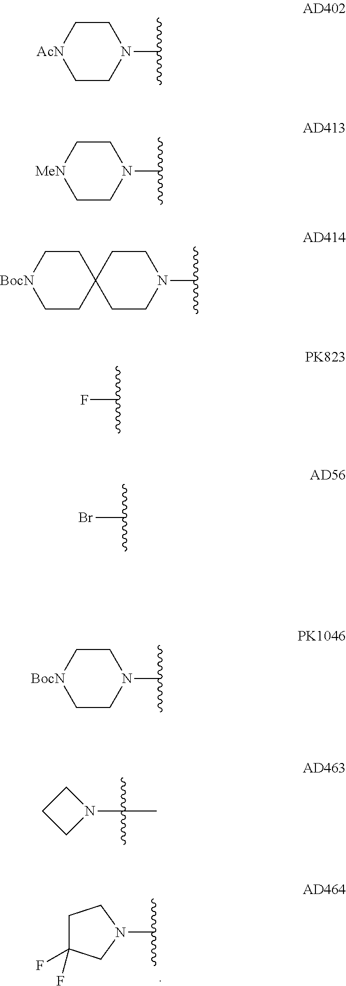

In one example embodiment, R₁ is selected from

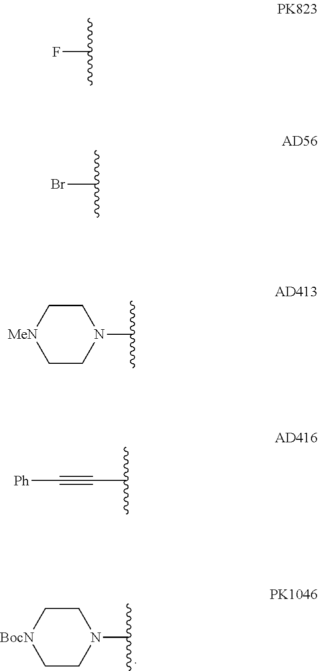

In one example embodiment, the molecule is according to:

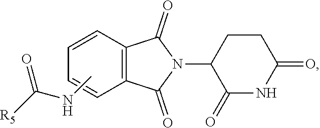

wherein R₅ is selected from

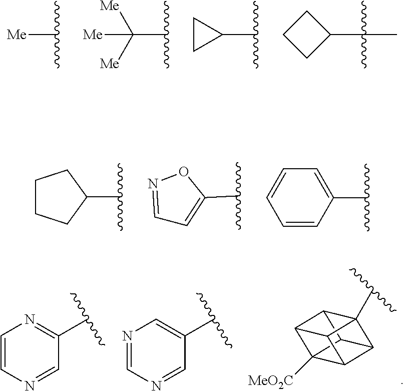

In one example embodiment, the molecule has the following structure

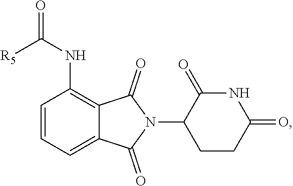

wherein R₅ is selected from

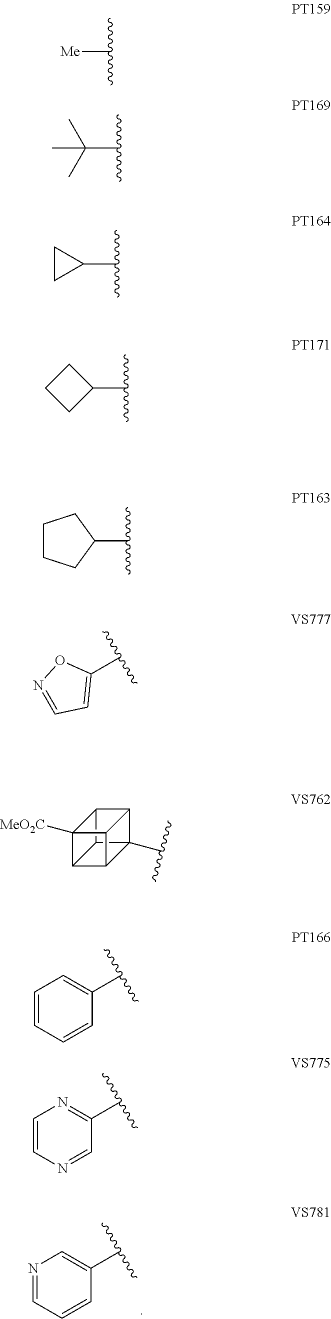

In one example embodiment, the molecule has the following structure

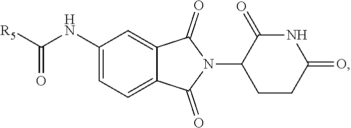

wherein R₅ is selected from

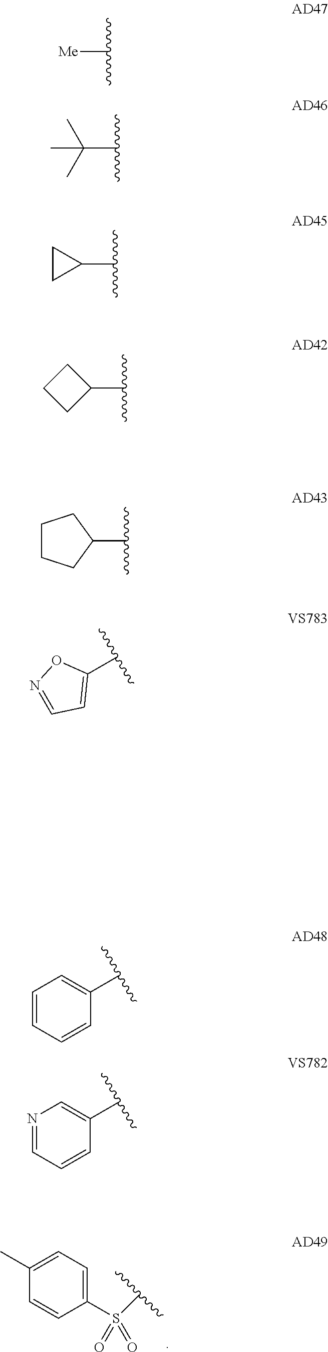

In one example embodiment, the molecule has the following structure

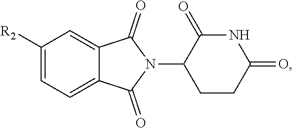

wherein R₂ is selected from

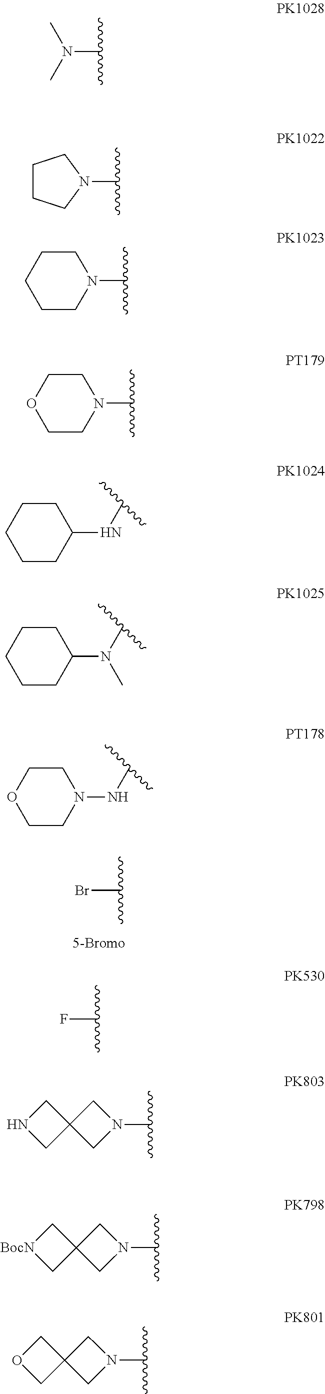

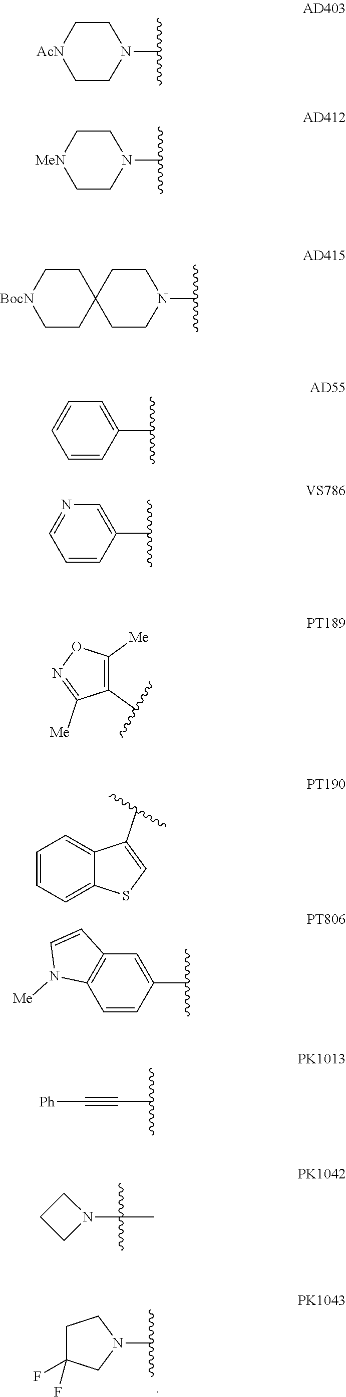

In one example embodiment, R₂ is selected from

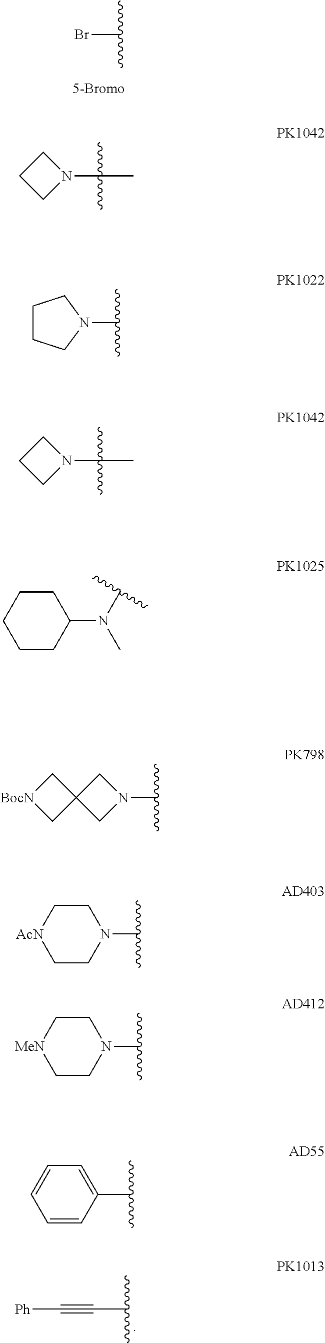

In one embodiment, the molecule is according to the formula

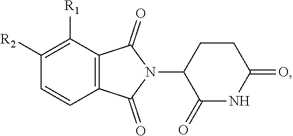

wherein when R₁ is H, R₂ is selected from

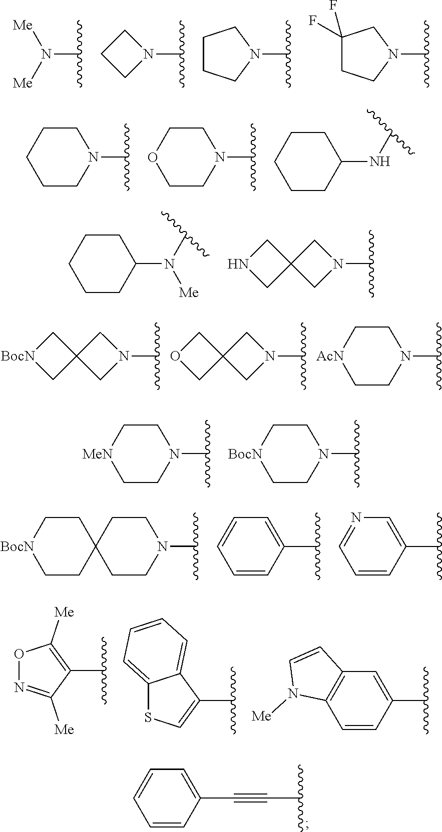

and wherein when R₂ is H, R₁ is selected from

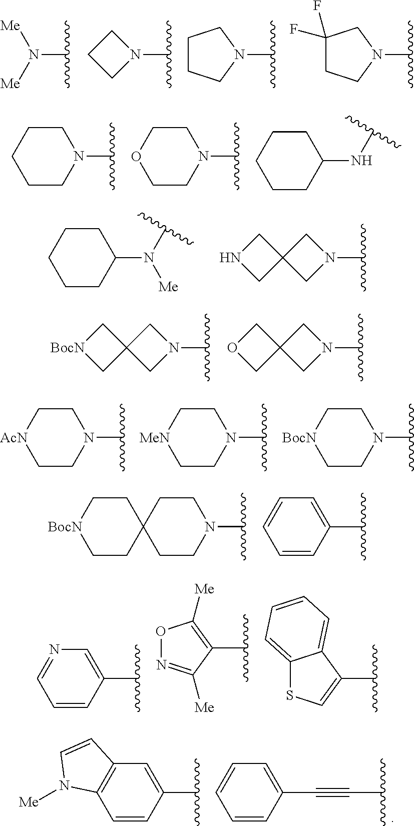

In one example embodiment, the molecule has the following structure

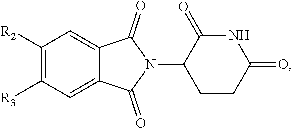

wherein R₃ is a fluorine group and R₂ is selected from

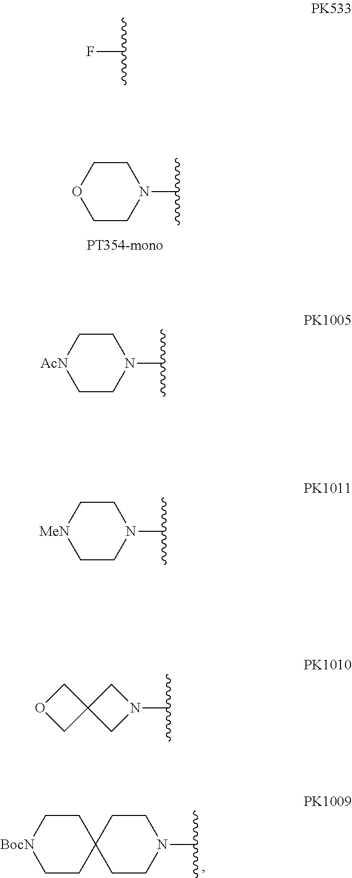

or
wherein R₂ and R₃ are each

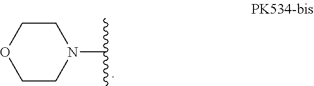 [SMILES: *N1CCOCC1]

In an embodiment, the molecule is according to the formula

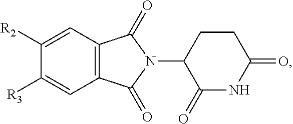

wherein R₂ is F and R₃ is selected from

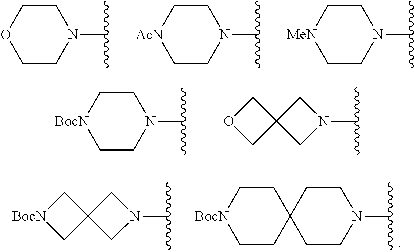

In one aspect, the present invention provides for a molecule according to the formula

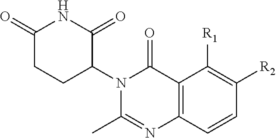

wherein R₁ is selected from —H and nitro groups, and wherein R₂ is selected from —H and halogen groups.

In one example embodiment, the molecule is selected from

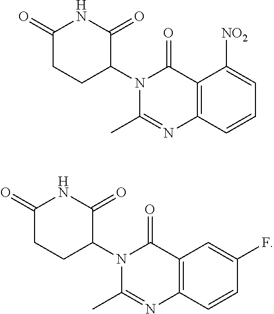 [SMILES: Cc1nc2cccc([N+](=O)[O-])c2c(=O)n1C1CCC(=O)NC1=O ; Cc1nc2ccc(F)cc2c(=O)n1C1CCC(=O)NC1=O]

In one aspect, the present invention provides for a method of inducing degradation of a variant protein in a cell, comprising exposing a cell transfected with a variant protein comprising one or more zinc finger polypeptides at one or more insertion sides on the protein with a molecule according to the present invention, a pharmaceutically acceptable salt thereof, or any pharmaceutical combination of molecules as described herein and/or pharmaceutically acceptable salts thereof. In one example embodiment, the variant protein is a programmable nuclease. In one example embodiment, the protein comprises a zinc finger selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, IKZF1/3 AA145-167/146-168, ZNF692 AA417-439, and combinations thereof. In one example embodiment, the programmable nuclease is selected from a CRISPR-Cas protein, a Zinc finger nuclease, a TALEN or a meganuclease. In one example embodiment, the molecule is selected from

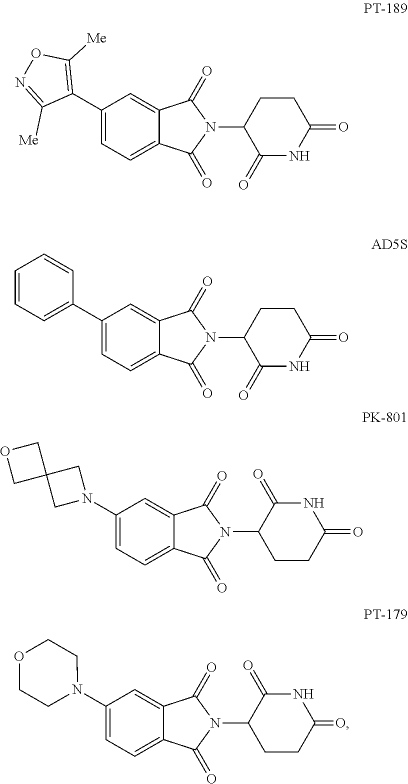 [SMILES: Cc1noc(C)c1-c1ccc2c(c1)C(=O)N(C1CCC(=O)NC1=O)C2=O ; O=C1CCC(N2C(=O)c3ccc(-c4ccccc4)cc3C2=O)C(=O)N1 ; O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1]

and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, and combinations thereof. In one example embodiment, the molecule is selected from

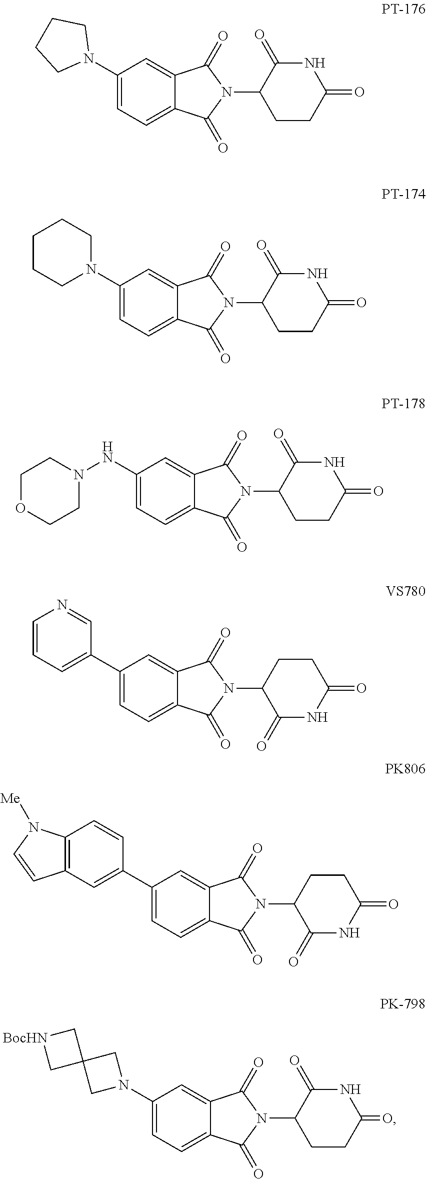

and wherein the cell comprises one or more zinc fingers selected from ZFN653 AA556-578, ZFN517 AA452-474, ZFP91_400_422, E4F1 AA220-242, ZFN654 AA25-47, IKZF3_146_168, ZNF582 AA395-417, IKZF3 AA146-168 Q147E, SALL4 ZF2, and combinations thereof. In one example embodiment, the molecule is selected from

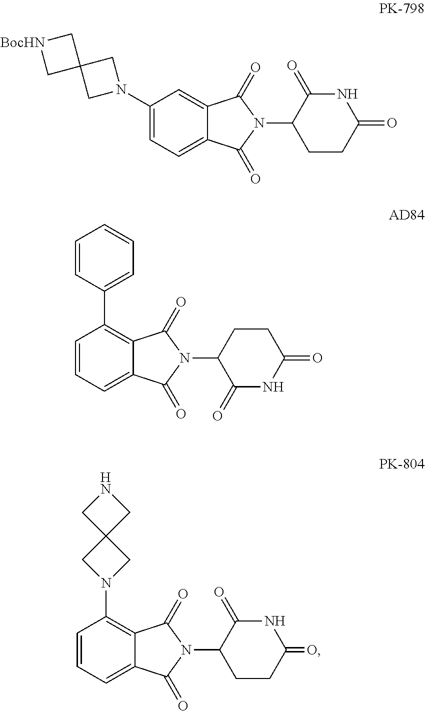

and wherein the cell comprises one or more zinc fingers selected from ZFN276 AA524-576, ZFN653 AA556-578, ZFN787 AA 178-200, ZFN517 AA452-474, ZFP91_400_422, E4F1 AA220-242, ZFN654 AA25-47, IKZF3_146_168, ZNF582 AA395-417, and combinations thereof. In one example embodiment, the molecule is selected from

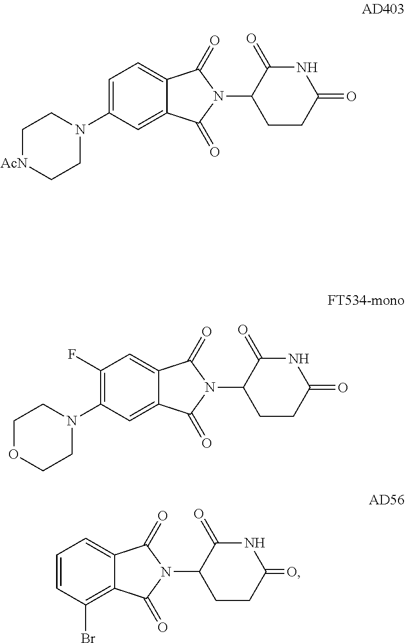

and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, and combinations thereof. In one example embodiment, the molecule is selected from

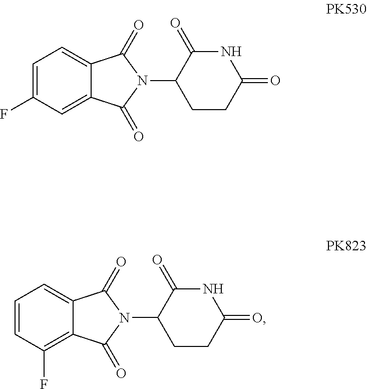 [SMILES: O=C1CCC(N2C(=O)c3ccc(F)cc3C2=O)C(=O)N1 ; O=C1CCC(N2C(=O)c3cccc(F)c3C2=O)C(=O)N1]

and wherein the cell comprises one or more zinc fingers selected from ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, IKZF3_146_168, ZNF582 AA395-417, ZKSC5_430_452, IKZF3 AA146-168 Q147E, SALL4 ZF2, and combinations thereof. In one example embodiment, the molecule is selected from

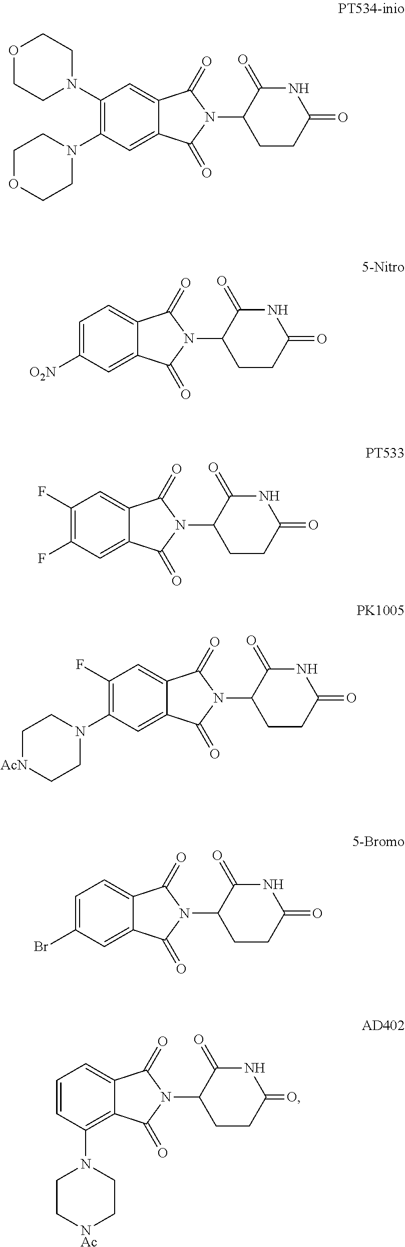

and wherein the cell comprises one or more zinc finger ZFN653 AA556-578, ZFN827 AA374-396, ZFN787 AA 178-200, ZFN517 AA452-474, IKZF3_146_168, ZNF582 AA395-417, IKZF3 AA146-168 Q147E, SALL4 ZF2, and combinations thereof. In one example embodiment, the molecule is according to the formula

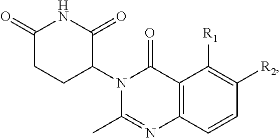

wherein R₁ is selected from —H and nitro groups, and wherein R₂ is selected from —H and halogen groups, and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, and combinations thereof. In one example embodiment, the molecule is selected from

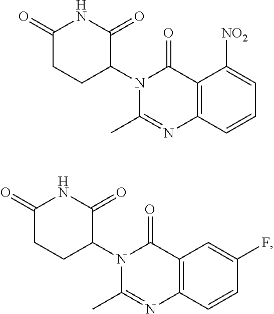 [SMILES: Cc1nc2cccc([N+](=O)[O-])c2c(=O)n1C1CCC(=O)NC1=O ; Cc1nc2ccc(F)cc2c(=O)n1C1CCC(=O)NC1=O]

and wherein the cell comprises one or more zinc fingers selected from ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, and combinations thereof.

In one aspect, the present invention provides for a method inducing degradation of a variant protein in a cell, comprising exposing a cell transfected with variant protein comprising one or more FK506 binding protein (FKBP) domains, with a composition according to the formula

A-(L)ₙ-B, a pharmaceutically acceptable salt thereof, or any pharmaceutical combination of compositions according to the formula A-(L)ₙ-B and/or pharmaceutically acceptable salts thereof, wherein L is a linker, wherein n is between 0 and 12, wherein A is ligand that binds to one of the FKBP domains, wherein B is a molecule according to the present invention, and wherein B is conjugated to A or (L)ₙ via R₁ or R₂.

In one example embodiment, (L)ₙ-B comprises an alkyl, an alkyne, a glycol ether, a polyglycol ether, a heterocyclic, a heteroaryl, or an aryl group. In one example embodiment, (L)ₙ-B comprises a C₄₋₈ alkyl group.

In one example embodiment, (L)ₙ-B comprises a group selected from

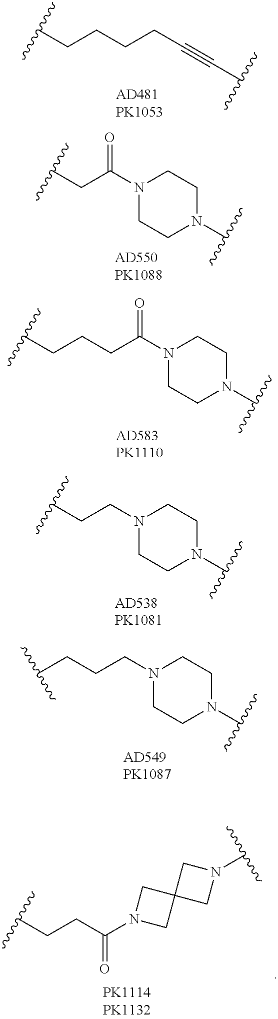 [SMILES: *C#CCCCC* ; *CC(=O)N1CCN(*)CC1 ; *CCCC(=O)N1CCN(*)CC1 ; *CCN1CCN(*)CC1 ; *CCCN1CCN(*)CC1 ; *CCC(=O)N1CC2(CN(*)C2)C1]

In one example embodiment, (L)ₙ-B is selected from

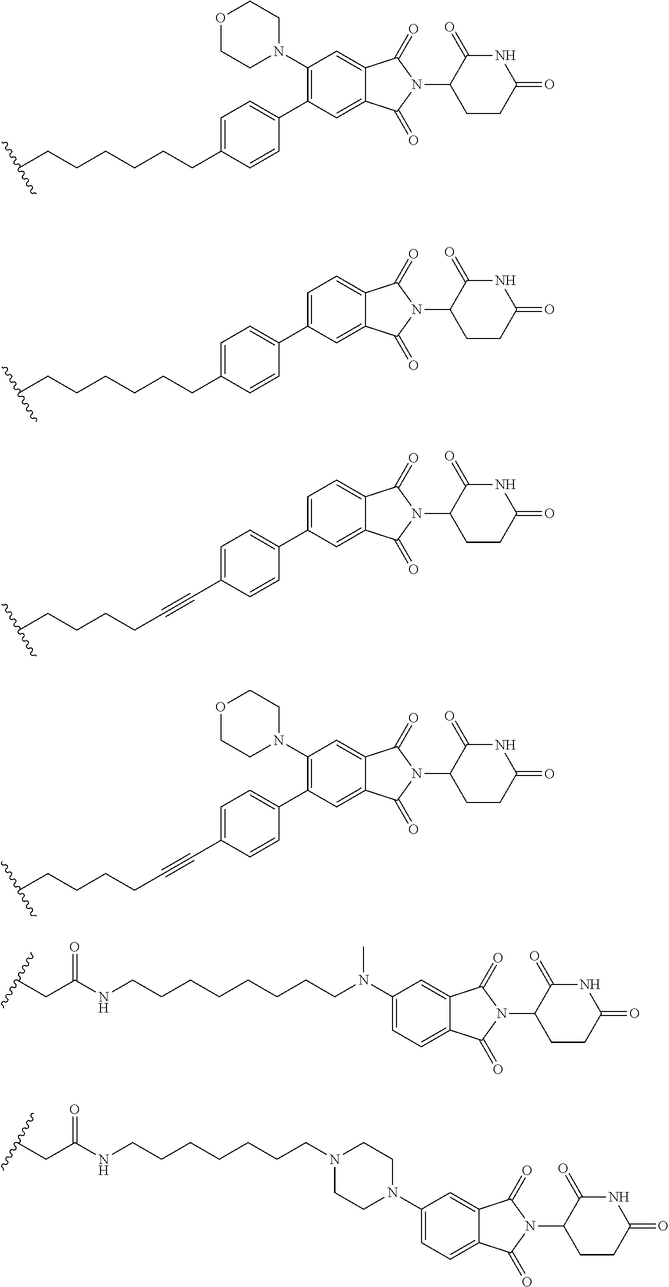 [SMILES: *CCCCCCc1ccc(-c2cc3c(cc2N2CCOCC2)C(=O)N(C2CCC(=O)NC2=O)C3=O)cc1 ; *CCCCCCc1ccc(-c2ccc3c(c2)C(=O)N(C2CCC(=O)NC2=O)C3=O)cc1 ; *CCCCC#Cc1ccc(-c2ccc3c(c2)C(=O)N(C2CCC(=O)NC2=O)C3=O)cc1 ; *CCCCC#Cc1ccc(-c2cc3c(cc2N2CCOCC2)C(=O)N(C2CCC(=O)NC2=O)C3=O)cc1 ; *CC(=O)NCCCCCCCCN(C)c1ccc2c(c1)C(=O)N(C1CCC(=O)NC1=O)C2=O ; *CC(=O)NCCCCCCCN1CCN(c2ccc3c(c2)C(=O)N(C2CCC(=O)NC2=O)C3=O)CC1]

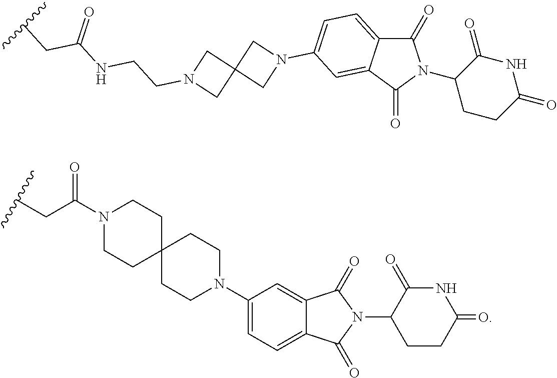 [SMILES: *CC(=O)NCCN1CC2(C1)CN(c1ccc3c(c1)C(=O)N(C1CCC(=O)NC1=O)C3=O)C2 ; *CC(=O)N1CCC2(CC1)CCN(c1ccc3c(c1)C(=O)N(C1CCC(=O)NC1=O)C3=O)CC2]

In one example embodiment, R₁ or R₂ is according to R₄, and wherein R₄ is an ether group according to the formula: —NH—C(O)—CH₂—O— or —O—. In one example embodiment, (L)ₙ-B is

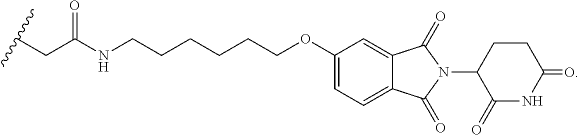 [SMILES: *CC(=O)NCCCCCCOc1ccc2c(c1)C(=O)N(C1CCC(=O)NC1=O)C2=O]

In one aspect, the present invention provides for a method of inducing degradation of a target amine in a cell, comprising: exposing a cell comprising a target amine with a composition according to the formula A-(L)ₙ-B, a pharmaceutically acceptable salt thereof, or any pharmaceutical combination of compositions according to the formula A-(L)ₙ-B and/or pharmaceutically acceptable salts thereof, wherein L is a linker and wherein n is between 0 and 12, wherein A is a ligand selective for the target amine, wherein B is a molecule of the present disclosure, and wherein B is conjugated to A or (L)ₙ via R₁ or R₂. In one example embodiment, (L)ₙ-B comprises an alkyl, an alkyne, a glycol ether, a polyglycol ether, a heterocyclic, a heteroaryl, or an aryl group. In one example embodiment, wherein (L)ₙ-B comprises a C₄₋₈ alkyl group. In one example embodiment, (L)ₙ-B comprises a group selected from

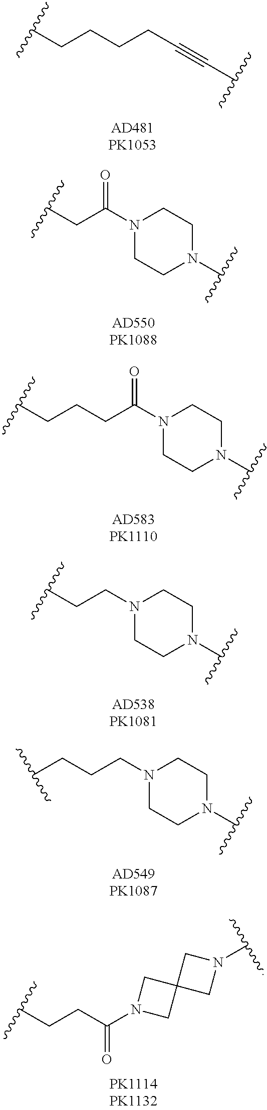

In one example embodiment, (L)ₙ-B is selected from

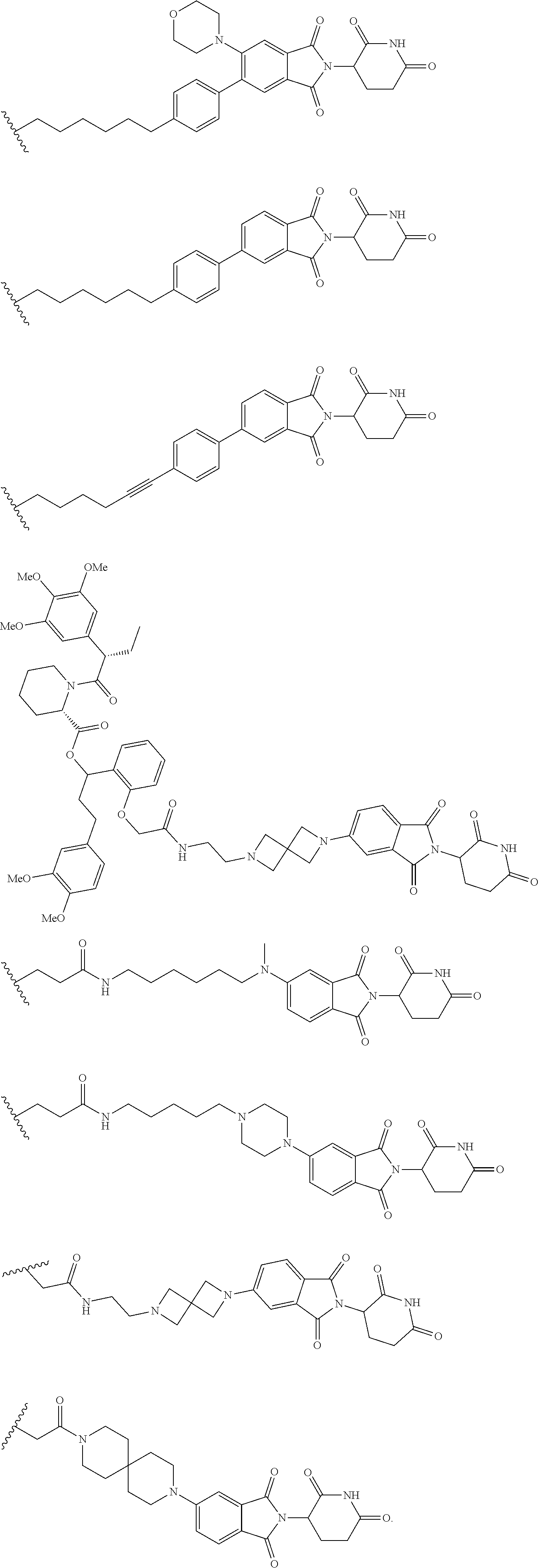

In one example embodiment, R₁ or R₂ is according to R₄, and wherein R₄ is an ether group according to the formula: —NH—C(O)—CH₂—O— or —O—. In one example embodiment, (L)ₙ-B is

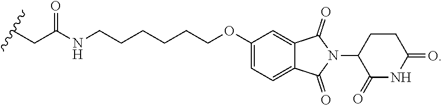 [SMILES: *CC(=O)NCCCCCCOc1ccc2c(c1)C(=O)N(C1CCC(=O)NC1=O)C2=O]

In one example embodiment, the target amine is a programmable nuclease, and wherein the cell is transfected with the programmable nuclease prior to the exposing step. In one example embodiment, the programmable nuclease is selected from a CRISPR-Cas protein, a Zinc finger nuclease, a TALEN or a meganuclease. In one example embodiment, R₁ or R₂ is according to R₄, and wherein R₄ is an ether group according to the formula —NH—C(O)—CH₂—O—, and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, and combinations thereof. In one example embodiment, wherein R₁ or R₂ is according to R₄, and wherein R₄ is an ether group according to the formula —O—, and wherein the cell comprises one or more zinc fingers selected from ZFN787 AA 178-200, IKZF3_146_168, ZKSC5_430_452, and combinations thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIGS. 1A-1F—Development of a high-throughput assay for the evaluation of off-target ZF degradation of pomalidomide-based PROTACs. (FIG. 1A) Schematic of the automated high-content imaging screen for the degradation of validated ZF degrons by pomalidomide analogs and pomalidomide-based PROTACs. Briefly U2OS cells stably expressing 14 ZF degrons fused to eGFP were screened for the image-based degradation assessment upon treatment of IMiDs or PROTACs. (FIG. 1B) In-cell degradation of validated and pomalidomide-sensitive ZF degrons inside cells by commercially available PROTACs in a dosing range of 4.3 nM to 20 μM. (FIGS. 1C-1F) Immunoblots demonstrating off-target degradation of endogenous ZF proteins ZFP91 and IKZF3 by MS4078 (ALK PROTAC) (1C-1D) and dTAG-13 (FKBP12F36V PROTAC) (1E-1F) in a dose-dependent manner in JURKAT cells.

FIGS. 2A-2F—Design, generation, and evaluation of the library of pomalidomide analogs based on alterations of the C4 and C5 positions of the phthalimide ring. (FIG. 2A) Crystal structure showing the deeply buried glutarimide ring in the CRBN exposing the 4-amino group of pomalidomide in making a crucial water mediated hydrogen bonding between CRBN (E377) and IKZF1 (IKZF1). Modification on C5 position would potentially bump-off the ZF degrons (PDB: 6H0F) while C4 substitutions reinforce through water mediated hydrogen bonding. (FIG. 2B) Immunoblots for endogenous ZF proteins ZFP91 and IKZF3 in MM1.S cells treated with pairs of ImiD analogs with C4 (pomalidomide) and C5 amino modifications on the phthalimide ring. (FIG. 2C) Degradation of pomalidomide-sensitive ZF degrons inside cells by the SNAr group of pomalidomide analogs arranged in pairs of C4 and C5 modifications on the phthalimide ring. Data for cells treated with 5 μM of each compound are shown. (FIG. 2D) Box-Whisker plot showing pair-wise comparison of GFP degradation levels induced by pairs of SNAr pomalidomide analogs with C4 and C5 modifications at the same dose, ranging from 4.3 nM to 20 μM. (FIG. 2E) Degradation of pomalidomide-sensitive ZF degrons inside cells by pairs of pomalidomide analogs with and without H-bond donor(s) immediately adjacent to the phthalimide ring. Box plot showing pair-wise comparison of GFP degradation levels induced by the compound pairs at the same dose, ranging from 4.3 nM to 20 μM. (FIG. 2F) Immunoblots for endogenous ZF proteins ZFP91 and IKZF3 in JURKAT cells treated with pomalidomide analogs with and without H-bond donor (NH) immediately attached to the phthalimide ring.

FIGS. 3A-3D—(FIGS. 3A-3B) Scatter dot plots showing ZF degradation scores [i.e., −Log(degradation sum+1)] for individual pomalidomide analogs and pomalidomide-based PROTACs investigated in this study. Structures of compounds with the least degradation (close to 0) are shown. See Example 1 for details on ZF degradation score computation. FIG. 3B shows the cleaner IMiD analogs resulting from the high-throughput imaging assay. See Example 3. (FIG. 3C) Structure of cleaner IMiD analogs of FIG. 3B. (FIG. 3C) Structure of cleaner IMiD analogs resulted from the high throughput imaging assay. (FIG. 3D) Structure of clinical PROTAC candidate ARV-110 highlighted for its cleaner IMiD building block.

FIGS. 4A-4D—Reengineering of the ALK PROTACs based on the new design principles. (FIG. 4A) Structures of rationally redesigned ALK PROTACs to minimize off-target ZF degradation and to enhance the on-target potency. (FIG. 4B) In-cell degradation of validated and pomalidomide-sensitive ZF degrons inside cells by redesigned ALK PROTACs in a dosing range of 4.3 nM to 20 μM. (FIGS. 4C-4D) Study of cell viability (FIG. 4C) and IC50 values (FIG. 4D) of SU-DH-L1 cells using cell titer glo assay upon treatment with redesigned ALK PROTACs.

FIG. 5—Representative readouts of the high-content imaging assay in 384-well plates, demonstrating robust detection of ZF-tagged GFP degradation as induced by pomalidomide. Shown are images of U2OS cells with stable expression of pomalidomide-sensitive ZF, ZFP91-IKZF3 hybrid, degron reporter. Z prime=0.8.

FIG. 6—Structures of commercially available pomalidomide-based PROTACs investigated in this study.

FIG. 7—Immunoblots quantifying off-target degradation of endogenous ZF proteins ZFP91 by MS4078 (ALK PROTAC) in a dose-dependent manner across cell lines SU-DHL-1 and H2228.

FIG. 8—Identification of the same group of exit vectors on pomalidomide with minimal off-target ZF degradation assessed by mass spectrometry-based proteomics. Relative abundance of endogenous ZF proteins in cells treated with pomalidomide-based PROTACs as arranged based on pomalidomide's exit vector groups. Data were extracted from proteomics datasets published in Donovan et al., 2020.

FIG. 9—Structures of 81 pomalidomide analogs synthesized in this study.

FIGS. 10A-10D—(FIGS. 10A-10C) Structural docking of pomalidomide analogs with C4 (FIG. 10A) and C5 (FIG. 10B) modifications on the phthalimide ring. Docking score (FIG. 10C) of each pair of modifications on C4 and C5. Shown is P value using the Wilcoxon test for paired samples. (FIG. 10D) Distribution of the physicochemical properties of the pomalidomide analog library. The topological polar surface area (tPSA) of each molecule is indicated by color and size (see legend). Note that each dot in the scatter plot represents a pair of compounds with the same modification on C4 and C5 positions, except the SNAr fluoro group. Synthetic routes are represented by different shapes (see legend).

FIGS. 11A-11D—Degradation of validated pomalidomide-sensitive ZF degrons induced by the pomalidomide analogs. (FIG. 11A) Normalized GFP intensity in 15 ZF reporter cell lines treated with different doses of 81 pomalidomide analogs ranging from 4.3 nM to 20 μM. Each block of 4.3 nM to 20 μM doses on the x axis represents one ZF reporter cell line. (FIGS. 11B-11D) Box-and-Whisker plots with statistical analysis for pomalidomide analogs arranged in pairs of C4 and C5 modifications such as acylation (FIG. 11B), Suzuki/sonogashira coupling (FIG. 11C) and effect of —F group (FIG. 11D) on the phthalimide ring. Data are shown for cells treated with 5 μM of each compound (for acylated and Suzuki/sonogashira couplings) and 20 μM data of each compound for deciphering the —F group effect.

FIG. 12—Generation of 30 analogs of Pomalidomide (POM) to reduce off-target degradation property of POM without affecting cereblon recruitment.

FIG. 13—High content imaging assay for interrogating degradation of top off-targets of Pomalidomide by POM analogs.

FIG. 14—Meta-modifications and hydrogen on amine groups retain off-target whereas ortho-modifications minimize off-target degradation. >10% GFP degradation cutoff.

FIG. 15—Synthetic scheme for Thalidomide analogs.

FIGS. 16A-16B—(FIG. 16A) Generation of 30 analogs of Pomalidomide (POM) to reduce off-target degradation property of POM for degron evolution. (FIG. 16B) Varying modifications on phthalimide end that bind zinc finger transcription factors but not Cereblon.

FIGS. 17A-17C—High content imaging detects molecular-glue induced degradation of not only Pomalidomide (POM) but also other analogs. (FIG. 17A) Compound plate map (5 μM). 25 images per well. NT=untreated. (FIG. 17B) IKZF3 degron Reporter. (FIG. 17C) Expression normalization reporter.

FIG. 18—POM modifications at position 5 have reduced off-target degradation compared with position 4. IMiDs (5 μM). Brackets: Pairs of 4^{th} and 5^{th} position modifications.

FIG. 19—Modifications with hydrogen on amine group have more off-target degradation). IMiDs (5 μM). Brackets: Modifications with hydrogen on amine group.

FIG. 20—All compounds with bulky groups directly attached to position 5 have minimal off-target degradation as observed across dosages and different off-target zinc finger transcription factors. Brackets: Modifications with bulky groups at position 5.

FIG. 21—Examples of off-target degradation caused by 4^{th} position vs. 5^{th} position modifications.

FIG. 22—Fluoro morpholine and piperazine are as clean as each other, and piperazine alone without fluoro is already very clean.

FIG. 23—PROTACs and dTAGs with hydrogen on amine group at the 4^{th} position retain off-target degradation whereas PROTACs with ether and carbonyl groups at the 4^{th} position have minimal off-target degradation.

FIG. 24—Substituting hydrogens on amine group of avadomide was sufficient to reduce off-target degradation.

FIG. 25—Exemplary schematic for approaches to synthesis of IMiD analogs.

FIG. 26—Exemplary structures of IMiD analogs.

FIG. 27—Exemplary rational design of Anaplastic lymphoma kinase (ALK) PROTACs with reduced zinc finger off-targets.

FIG. 28—An integrated platform to develop PROTACs with reduced off-targets and resistance development.

FIGS. 29A-29C—Validation of screening results by cell painting. (FIG. 29A) Structures of 5 IMiDs, each with high and low degradation scores used in the cell painting study. (FIG. 20B) Heatmap showing the correlation of cell morphological parameters in a cell painting assay. (FIG. 29C) Comparison of intensities of cell painting features for 37 (low score) and 70 (high degradation score). (AGP:Actin, Golgi, Plasma membrane).

FIGS. 30A-30B—Validation of screening results by global proteomics studies showing off target protein degradation by pomalidomide (FIG. 30A), minimal degradation of off-target proteins in MOLT4 cells treated with compound 39 (B).

FIGS. 31A-31C—(FIG. 31A) Synthetic approaches used for the IMiD library generation. (FIG. 31B) Structures of cleaner 9 IMiDs with each with low degradation scores. (FIG. 31C) Structures of glutarimide-based PROTACs.

FIGS. 32A-32F—(FIGS. 32A-32B) Structures of rationally redesigned ALK PROTACs (FIG. 32A) minimized the off-target ZF degradation in the image-based assay (FIG. 32B). (FIGS. 32C-32D) Determination of EC50 values (nM) (FIG. 32C), immunoblots showing the degradation of ALK protein (FIG. 32D) in SU-DH-L1 cells. (FIG. 32E-32F) Global proteomic analysis of dALK-2 in SU-DHL-1 (FIG. 32E) and MOLT4 cells (FIG. 32F).

FIG. 33—Proposed scaffolds, some representative PROTACS.

FIG. 34—CRISPR-scanning workflow: guide RNA (sgRNA) are tiled across CRBN and PROTAC target genes such as BTK, BCR-ABL, CDK4/6, and BRAF.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed disclosure. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

“Diastereoisomers” are stereoisomers that have at least two asymmetric atoms, but which are not mirror-images of each other. The absolute stereochemistry is specified according to the Cahn-Ingold-Prelog R-S system When a compound is an enantiomer, the stereochemistry at each chiral carbon can be specified by either R or S. Resolved compounds whose absolute configuration is unknown can be designated (+) or (−) depending on the direction (dextro- or levo-rotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)— or (S)—. The present chemical entities, pharmaceutical compositions and methods are meant to include all such possible isomers, including racemic mixtures, optically substantially pure forms and intermediate mixtures. In some chemical structures, stereocenters may be identified with “wavy” bonds indicating that the stereocenter may be in the R or S configuration, unless otherwise specified. However, stereocenters without a wavy bond (i.e., a “straight” bond) may also be in the (R) or (S) configuration, unless otherwise specified. Compositions comprising compounds may comprise stereocenters which each may independently be in the (R) configuration, the (S) configuration, or racemic mixtures.

Optically active (R)- and (S)-isomers can be prepared, for example, using chiral synthons or chiral reagents, or resolved using conventional techniques. Enantiomers can be isolated from racemic mixtures by any method known to those skilled in the art, including chiral high pressure liquid chromatography (HPLC), the formation and crystallization of chiral salts, or prepared by asymmetric syntheses.

Optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization or sublimation, and then hydrolyzed to deliver the enantiomerically enriched compound.

Optically active compounds can also be obtained by using active starting materials. In some embodiments, these isomers can be in the form of a free acid, a free base, an ester or a salt. In certain embodiments, a disclosed compound can be a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). Tautomerization includes prototropic or proton-shift tautomerization, which is considered a subset of acid-base chemistry. Prototropic tautomerization or proton-shift tautomerization involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

As used herein, a bond substitution coming out of a ring, e.g,

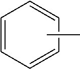

means that the substitution can be at any of the available positions on the ring.

An alkyl generally means a straight or branched chain aliphatic groups. The alkyl groups can be unsubstituted or substituted by halo, hydroxy, alkoxy, amino, alkylamino, dialkylamino, cycloalkyl, aryl, aryloxy, heteroaryl, or heteroaryloxy groups, among other. Alkenyl straight or branched carbon chain having one or more double bonds. Alkynyl comprises a straight or branched carbon chain with at least one triple bond. The alkenyl and alkynyl groups can have one or more double bonds or triple bonds, respectively, or a combination of double and triple bonds. Alkenyl and Alkynyl groups can be unsubstituted or substituted with functional groups as described herein.

As used herein a hydrocarbon substituent means any group exclusively of hydrogen and carbons atoms. This includes alkyls, alkylenes, alkynes as well as saturated and unsaturated rings and fused rings.

As used herein a nitrogen-based substituent means any group comprising one or more nitrogen. Non-limiting examples of nitrogen-based substituent may include aminyl, 4° ammonium cations, amidyl, iminyl, imidyl, azidyl, azo radical, cyano, nitrate, nitrile radical, nitrite radical, nitryl, nitrosyl, oxime, carbamoyl.

As used herein a sulfur-based substituent means any group comprising one or more sulfurs. Non-limiting examples of sulfur-based substituents may include H or R sulfanyl, disulfanyl, sulfinyl, sulfino radical, sulfo radical, alkosulfonyl, thiocyanato radical, isothiocyanato radical, thioyl, sulfanylidene, methanethioyl, mercaptocarbonyl, hydroxy(thiocarbonyl), thioester radical, thionoester radical, dithiocarboxy radical, dithiocarboxylic acid ester radical, dithiocarbamate radical.

As used herein an oxygen-based substituent means any group comprising one or more oxygen. Non-limiting examples of oxygen-based substituents may include hydroxyl, carbonyl, formyl, haloformyl, (alkoxycarbonyl)oxy, carboxyl, carboxylate, carboalkoxyl, hydroperoxyl, peroxyl, alkoxyl, dialkoxyl, trialkoxyl, methylenedioxyl, tetralkoxyl, and carboxylic anhydride radical.

As used herein a boron-based substituent means any group comprising one or more boron. Non-limiting examples of boron-based substituents may include boronyl, borono radical, O-[bis(alkoxy)alkylboronyl], hydroxyborino radical, O-[alkoxydialkylboronyl].

As used herein a halogen-based substituent means any group comprising one or more halogen.

As used herein a heterocycle means any molecule that forms a continuous covalent connection and contains an element that is not hydrogen or carbon. Non-limiting examples of heterocycles may include. oxetane, thietane, azetidine, β-lactam, oxirane, thiirane, aziridine, azirine, diaziridine, diazirine, epoxide, tetrahydrofuran, furan, thiolane, thiophene, pyrrolidine, pyrrole, 3-pyrroline, 2-H-pyrrole, benzofuran, coumarin, isobenzofuran, benzothiophene, dibenzothiophene, indoline, indole, indolinine, oxindole, indoxyl, isatin, isoindole, indolizine, pyrrolizine, carbazole, dioxolane, dithiolane, oxazolidine, oxazolidinone, oxazole, isoxazole, thiazole, isothiazole, imidazolidine, 2-imidazoline, imidazole, pyrazolidine, 2-pyrazoline, pyrazole, benzodioxole, benzoxazole, indoxazine, benzothiazole, benzimidazole, 1H-indazole, purine, azaindole, 1,2,3-oxadiazole, 1,3,4-thiadiazole, 1,2,3-triazole, 1,2,4-triazole, tetrazole, benzotriazole, quinuclidine, diazabicyclooctane, diazabicycloundecane, 4H-pyran, tetrahydropyran, dihydropyran, 2H-pyran, piperidine, pyridine, picoline, lutidine, collidine, pyridone, acridine, chromene, coumarin, isocoumarin, xanthene, tetrahydroquinoline, quinoline, isoquinoline, quinolone, 4H-quinolizine, quinolizinium, 1,4-dioxane, morpholine, paraformaldehyde, 1,4-dithiane, 1,3-dithiane, thiomorpholine, trithiane, piperazine, pyrazine, pyrimidine, pyridazine, 1,3,5-triazine, tetrazine, cinnoline, phthalazine, 1,8-naphthyridine, quinoxaline, quinazoline or any combination thereof including fusing or covalently linking and further optionally substituted with any previously mentioned substituent.

Additional substituents may comprise any combination of the above substituents. Throughout the description, molecules may be represented with an exemplary bonding location indicated by [symbol] , however further optimization of binding location of molecules can be performed, including through methods of screening and computational approaches detailed herein.

Thus, identified binding locations on molecules via depiction with [symbol] are not intended to be limiting, merely exemplary, with further optimizations and locations of binding sites implicitly recognized as being identifiable with the methods and guidance as described herein, including at any position on rings within the structures as well as any other substituents of the molecules.

Carbocycle or Cycloalkyl means a mono or bicyclic carbocyclic ring functional group, and includes both substituted and unsubstituted cycloalkyl groups. Cycloalkyl groups can optionally contain double bonds and is intended to encompass cycloalkenyl groups. Unless otherwise indicated, a reference to a (C3-C8) cycloalkyl refers to a cycloalkyl group containing from 3 to 8 carbons, and is intended to encompass a monocyclic cycloalkyl group containing from 3 to 8 carbons and a bicyclic cycloalkyl group containing from 6 to 8 carbons.

Heterocycloalkyl generally refers to a ring functional group having carbon atoms and one or more heteroatoms independently selected from S, N, or. The heterocycloalkyl is intended to encompass 1 or more double bonds which may be between two carbons or a carbon and a heteroatom. For example, an exemplary 5-membered ring heterocycloalkyl can have one carbon-carbon double bond or one carbon-nitrogen bond in the ring, e.g., dihydropyrazoles, pyrollinyls.

An aryl group as utilized herein refers to an aromatic hydrocarbon radical that encompasses cyclic, and multicyclic, e.g., bicyclic, tricyclic, aromatic ring moiety. Exemplary aryl groups include phenyl and napthyl. A phenyl may be unsubstituted or substituted at one or more positions with a substituent, including but not limited to those substituents described above for alkyl groups.

Heteroaryl group as utilized herein refers to an aromatic moiety that encompasses cyclic and multicyclic, e.g., bicyclic, or tricyclic, moiety having carbon atoms and one or more selected from O, S, or N.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures, or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Proteolysis Targeting Chimeras (PROTACs) have gained considerable traction in recent years as heterobifunctional compounds that induce degradation of traditionally intractable protein targets in disease. However, pomalidomide, a widely used E3 ligase ligand in PROTACs, can independently degrade other targets, such as zinc finger (ZF) proteins, that hold key functions in normal development and disease progression. This off-target degradation of pomalidomide-based PROTACs raises concerns about their therapeutic applicability and long-term side effects. Therefore, there is a crucial need to develop rules for PROTAC design that minimize off-target degradation.

Currently, off-target degradation can be assessed by mass spectrometry-based methods that detect protein levels, but these techniques lack sensitivity for low abundant proteins. In addition to expense, the implementation of mass spectrometry is technically challenging when analyses include profiling the off-target degradation affected by specific PROTACs across multiple tissue types for tissue-specific expression of lineage-specific proteins. These analyses are further complicated by the need to perform these assessments across different levels of PROTAC dosing.

In this disclosure, a high-throughput platform interrogated the off-target degradation of ZF proteins, which is widespread in commercially available PROTACs, to identify new rules for PROTAC design. A rationalized library of pomalidomide analogs with distinct exit vector modifications on the C4 and C5 positions of the phthalimide ring was developed and their propensities for ZF protein degradation were profiled. It was found that exit vector modifications on the C5 position with nucleophilic aromatic substitution (SNAr) (C—N) reduce off-target ZF degradation. These newfound design principles were applied on a previously developed ALK oncoprotein-targeting PROTAC and generated a new group of best-in-class PROTACs with enhanced potency and minimal off-target degradation.

### Molecules

In one aspect, the present invention provides for a molecule comprising an imide group. In one example embodiment, the molecule is an analog of thalidomide, pomalidomide, lenalidomide, avadomide, or iberdomide.

In one aspect, the present invention provides for a molecule according to formula

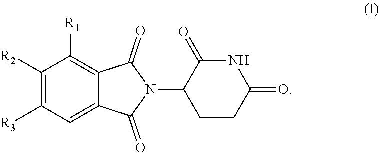

The molecule of formula (I) can comprise various substituent groups. In one example embodiment, R₁ is selected from —H, —R₄, —NHC(O)R₅, —NR₆R₇, —NHR₈, and —NHS(O₂)R₉. In one example embodiment, R₂ is selected from —H, —R₄, —NH₂, —NHC(O)R₅, —NR₆R₇, —NHR₈, and —NHS(O₂)R₉. In one example embodiment, R₃ is selected from —H, —R₄, and —NR₆R₇. In one example embodiment, R₄-R₉ are independently selected from one or more nitrile, nitro, ether, alcohol, thiol, sulfone, sulfonate, halogen, carbonyl, acyl, ketone, carboxylate ester, amide, enone, anhydride, imide, alkyl, alkenyl, alkynyl, saturated cyclic hydrocarbon, unsaturated cyclic hydrocarbon, heteroalkyl, heterocyclic ring, aryl ring, and heteroaryl ring groups, and one or more fused rings thereof. In one example embodiment, R₄-R₉ are more preferably independently selected from alkyl, amide, heteroalkyl, cycloalkyl, heterocyclic, aryl and heteroaryl groups. In one example embodiment, when R₂ and R₃ are —H, R₁ is selected from —R₄, —NHC(O)R₅, —NR₆R₇, —NHR₈, and —NHS(O₂)R₉. In one example embodiment, when R₁ and R₃ are —H, R₂ is selected from —R₄, —NH₂, —NHC(O)R₅, —NR₆R₇, —NHR₈, and —NHS(O₂)R₉. In one example embodiment, when R₂ and R₃ are —H, R₁ is selected from —R₄, —NHC(O)R₅, and —NR₆R₇.

In one example embodiment, R₁ is according to —R₄, and —R₄ is selected from halogen, aryl, heteroaryl, and alkynyl groups. In one example embodiment, the halogen group is a bromine or a fluorine group. In one example embodiment, the aryl group is a phenyl group and the heteroaryl group is a pyridinyl group. In one example embodiment, the heteroaryl group is selected from indolyl, pyridinyl, isoxazolyl, and thiophene groups. In one example embodiment, the indolyl group is a 1-methyl-indolyl

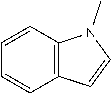

group, the isoxazolyl group is a 3,5-dimethyl-isoxazolyl

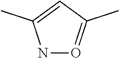

group, or the thiophene group is a benzothiophene group

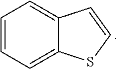

In one example embodiment, the alkynyl group is a 2-phenyl-acetylenyl group.

In one example embodiment, R₁ is —NHC(O)R₅, and R₅ is selected from alkyl, cycloalkyl, heterocyclic, heteroaryl, and aryl groups. In one example embodiment, R₅ is selected from methyl, phenyl, cyclopropyl, cyclobutyl, cyclopentyl, isoxazolyl, pyridinyl, and pyrazinyl groups.

In one example embodiment, R₁ is according to —NR₆R₇, and N, R₆, and R₇ taken together form a heterocyclic amine group. In one example embodiment, the heterocyclic amine group is selected from morpholinyl, azetidinyl, pyrrolidinyl, piperidinyl, piperazinyl, and diazaspiro groups. In one example embodiment, the pyrrolidinyl group is an unsubstituted pyrrolidinyl group or a 3, 3′-difluoro-pyrrolidinyl group. In one example embodiment, the piperazinyl group is a 4-acetyl-1-piperazinyl, a 4-Boc-1-piperazinyl, or a 4-methyl-1-piperazinyl group. In one example embodiment, the diazaspiro group is a 2,6-diazaspiro[3.3]heptan

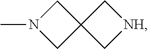

a 2-oxa-6-azaspiro[3.3]heptane

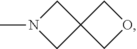

a 2-Boc-2,6-diazaspiro[3.3]heptane

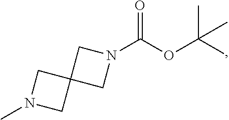

or a 3-Boc-3,9-diazaspiro[5.5]undecane

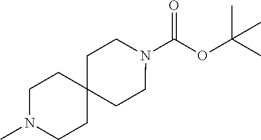 [SMILES: CN1CCC2(CC1)CCN(C(=O)OC(C)(C)C)CC2]

group.

In one example embodiment, R₁ is according to —NR₆R₇ or —NHR₈, R₆ and R₇ are independently selected from alkyl and cycloalkyl groups, and R₈ is a cycloalkyl group. In one example embodiment, —NR₆R₇ is a methylcyclohexyl amine group, and R₈ is a cyclohexyl group or a morpholinyl group.

In one example embodiment, R₁ and R₃ are —H, and R₂ is selected from —R₄, —NH₂, —NHC(O)R₅, —NR₆R₇, —NHR₈, and —NHS(O₂)R₉. In one example embodiment, R₂ is according to —R₄, and —R₄ is selected from halogen, nitro, heteroaryl, aryl, and alkynyl groups. In one example embodiment, the halogen group is a fluorine or bromine group. In one example embodiment, the heteroaryl group is selected from indolyl, pyridinyl, isoxazolyl, and thiophene groups. In one example embodiment, the indolyl group is a 1-methyl-indolyl

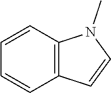

group, the isoxazolyl group is a 3,5-dimethyl-isoxazolyl

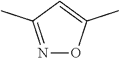

group, and the thiophene group is a benzothiophene group

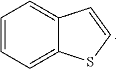

In one example embodiment, the aryl group is selected from phenyl. In one example embodiment, the alkynyl group is a 2-phenyl-acetylenyl group.

In one example embodiment, R₂ is according to —NHC(O)R₅, and R₅ is selected from alkyl, cycloalkyl, heterocyclic, heteroaryl, and aryl groups. In one example embodiment, R₅ is a methyl, a phenyl, a cyclopropyl, a cyclobutyl, a cyclopentyl, an isoxazolyl, a pyridinyl, or a pyrazinyl group.

In one example embodiment, R₂ is according to —NR₆R₇, and N, R₆, and R₇ taken together form a heterocyclic amine group. In one example embodiment, the heterocyclic amine group is selected from morpholinyl, azetidinyl, pyrrolidinyl, piperidinyl, piperazinyl, and diazaspiro groups. In one example embodiment, the pyrrolidinyl group is an unsubstituted pyrrolidinyl group or a 3, 3′-difluoro-pyrrolidinyl group. In one example embodiment, the piperazinyl group is a 4-acetyl-1-piperazinyl, a 4-Boc-1-piperazinyl, or a 4-methyl-1-piperazinyl group. In one example embodiment, the diazaspiro group is a 2,6-diazaspiro[3.3]heptane

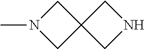

group, a 2-oxa-6-azaspiro[3.3]heptane

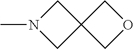

group, a 2-Boc-2,6-diazaspiro[3.3]heptane

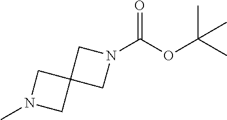

group, or a 3-Boc-3,9-diazaspiro[5.5]undecane

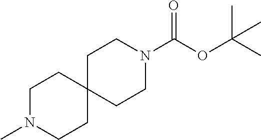 [SMILES: CN1CCC2(CC1)CCN(C(=O)OC(C)(C)C)CC2]

group.

In one example embodiment, R₂ is according to —NR₆R₇ or —NHR₈, R₆ and R₇ are independently selected from alkyl and cycloalkyl groups, and R₈ is selected from cycloalkyl and heterocyclic groups. In one example embodiment, wherein —NR₆R₇ is a methylcyclohexyl amine group, and R₈ is a cyclohexyl group or a morpholinyl group.

In one example embodiment, wherein R₂ is according to —NHS(O₂)R₉, and R₉ is an aryl group.

In one example embodiment, wherein R₁ is —H and R₂ and R₃ are according to the same —R₄ or —NR₆R₇, and N, R₆, and R₇ taken together form a heterocyclic amine group. In one example embodiment, R₂ and R₃ are according to the same —R₄, and —R₄, is a halogen. In one example embodiment, the halogen is a fluorine group. In one example embodiment, R₂ and R₃ are according to the same —NR₆R₇, and —NR₆R₇ is a morpholinyl group.

In one example embodiment, R₁ is —H, R₃ is according to —R₄, R₂ is according to —NR₆R₇, and N, R₆, and R₇ taken together form a heterocyclic amine group. In one example embodiment, —R₄ is a halogen and the heterocyclic amine group is selected from morpholinyl, piperazinyl, and diazaspiro groups. In one example embodiment, the halogen is a fluorine group. In one example embodiment, the piperazinyl group is a 4-acetyl-1-piperazinyl, a 4-Boc-1-piperazinyl, or a 4-methyl-1-piperazinyl group. In one example embodiment, the diazaspiro group is a 2-oxa-6-azaspiro[3.3]heptane

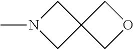

group, a 2-Boc-2,6-diazaspiro[3.3]heptane

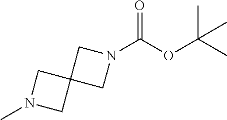

group, or a 3-Boc-3,9-diazaspiro[5.5]undecane

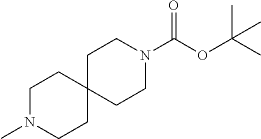 [SMILES: CN1CCC2(CC1)CCN(C(=O)OC(C)(C)C)CC2]

group. In one example embodiment, —R₄ is an aryl group and the heterocyclic amine group is a morpholinyl group. In one example embodiment, the aryl group is a phenyl group.

In one example embodiment, the molecule is selected from

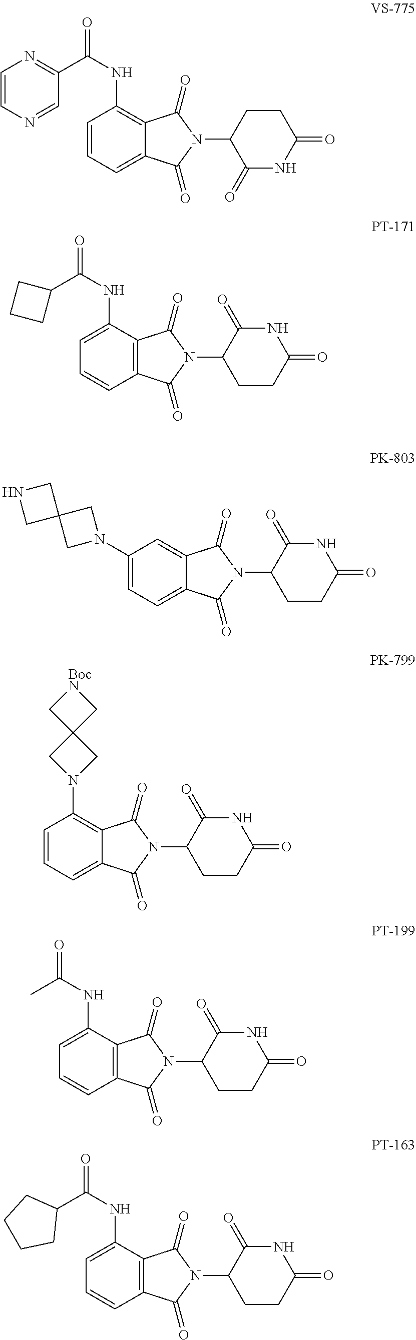 [SMILES: O=C1CCC(N2C(=O)c3cccc(NC(=O)c4cnccn4)c3C2=O)C(=O)N1 ; O=C1CCC(N2C(=O)c3cccc(NC(=O)C4CCC4)c3C2=O)C(=O)N1 ; O=C1CCC(N2C(=O)c3ccc(N4CC5(CNC5)C4)cc3C2=O)C(=O)N1 ; CC(C)(C)OC(=O)N1CC2(C1)CN(c1cccc3c1C(=O)N(C1CCC(=O)NC1=O)C3=O)C2 ; CC(=O)Nc1cccc2c1C(=O)N(C1CCC(=O)NC1=O)C2=O ; O=C1CCC(N2C(=O)c3cccc(NC(=O)C4CCCC4)c3C2=O)C(=O)N1]

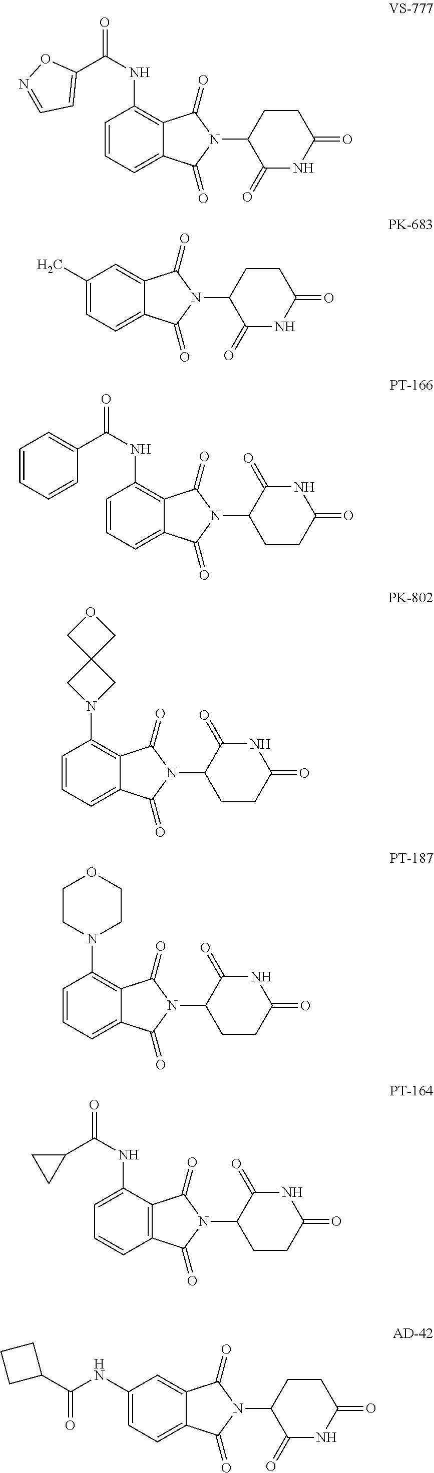

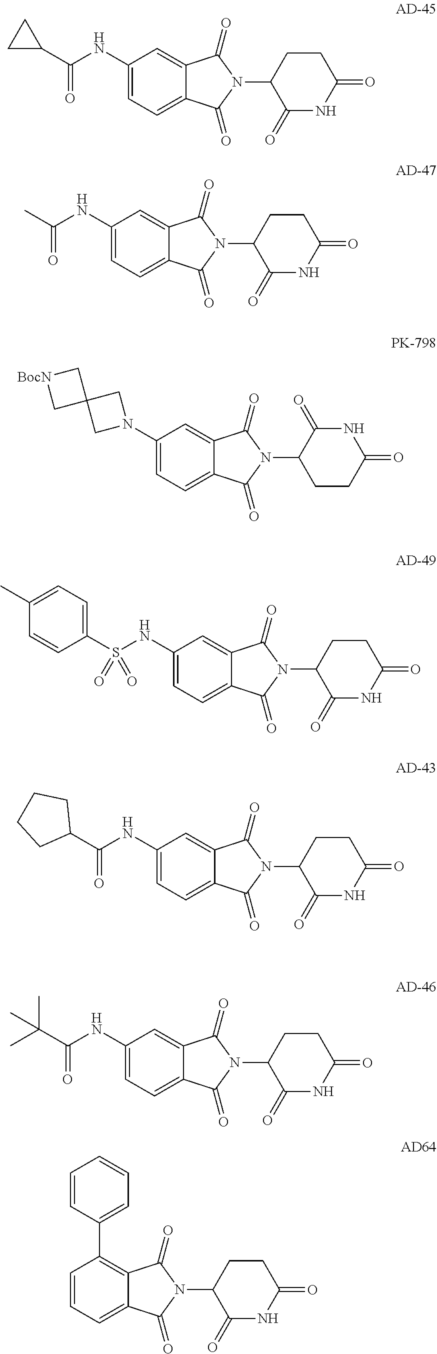

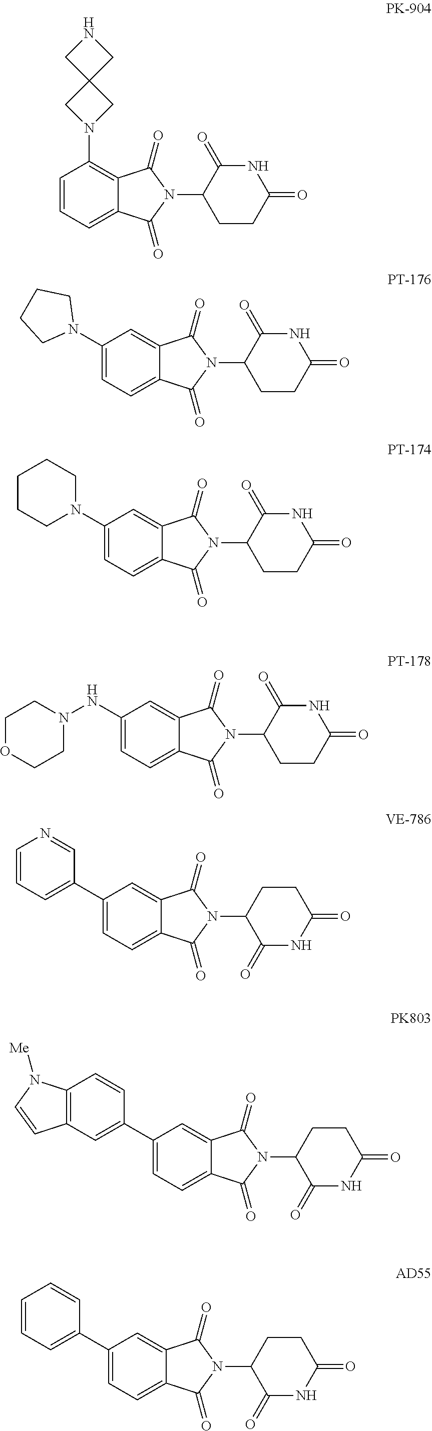

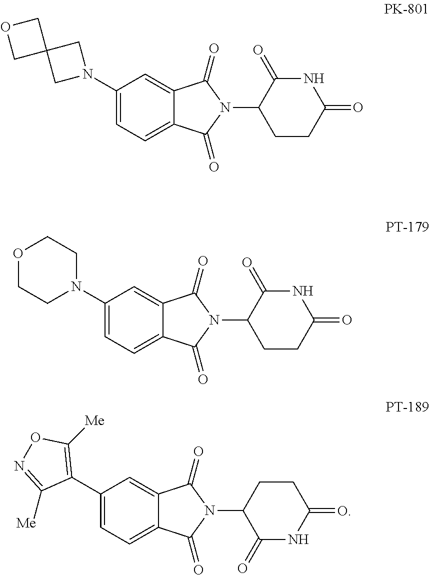 [SMILES: O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1 ; Cc1noc(C)c1-c1ccc2c(c1)C(=O)N(C1CCC(=O)NC1=O)C2=O]

In one example embodiment, the molecule is selected from

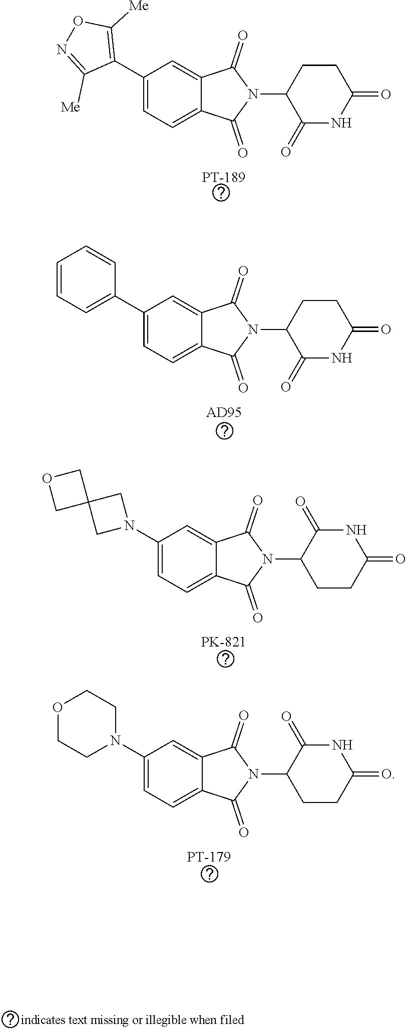

In one example embodiment, the molecule is selected from

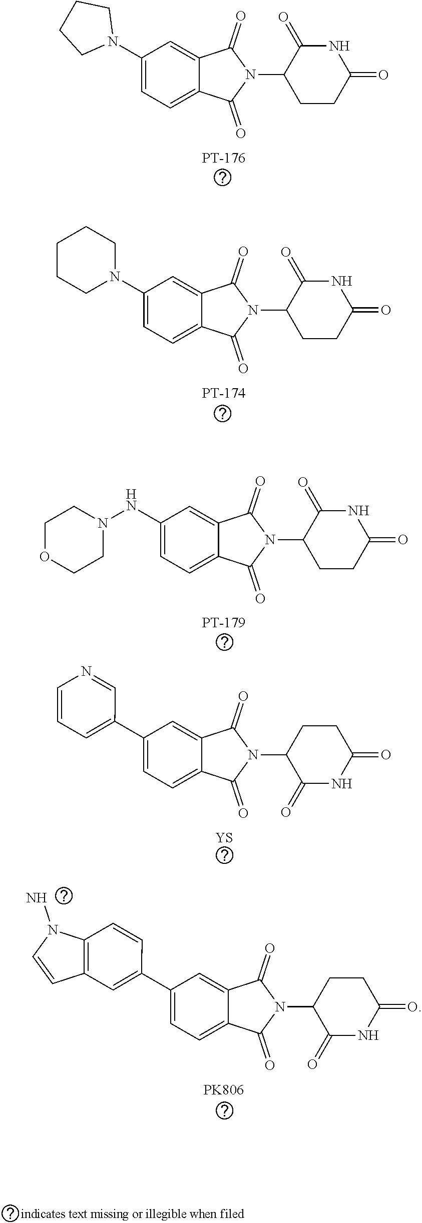

In one example embodiment, the molecule is selected from

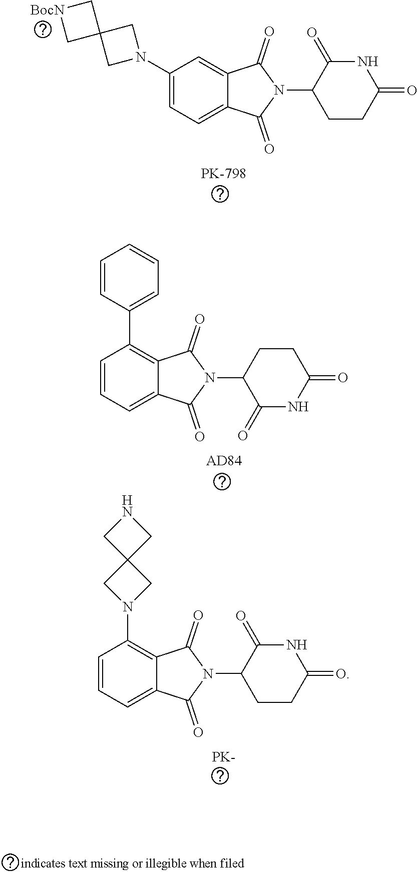

In one example embodiment, the molecule is selected from

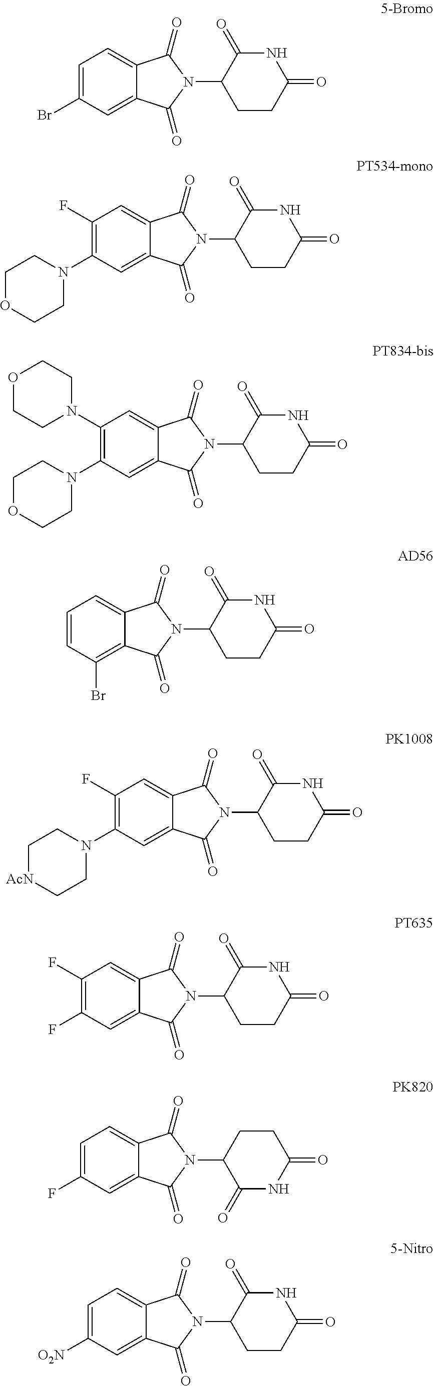

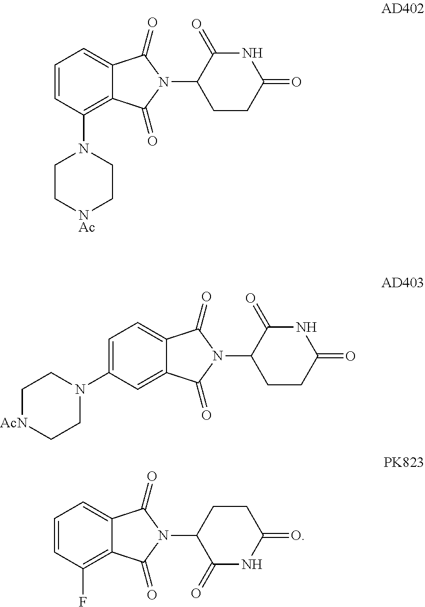

In one example embodiment, the molecule is selected from

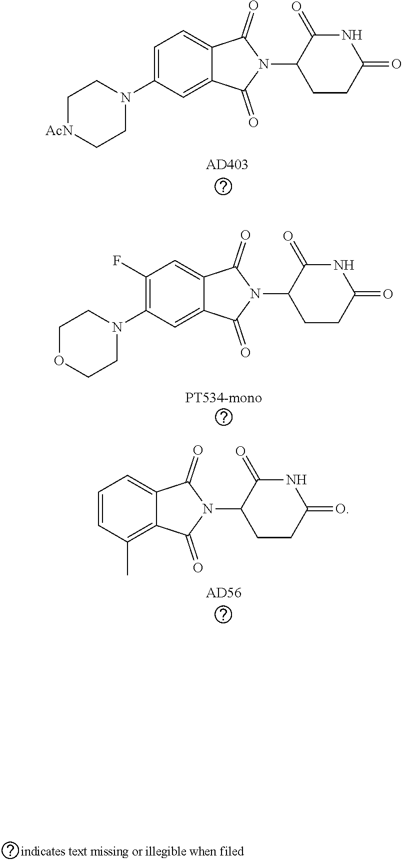

In one example embodiment, the molecule is selected from

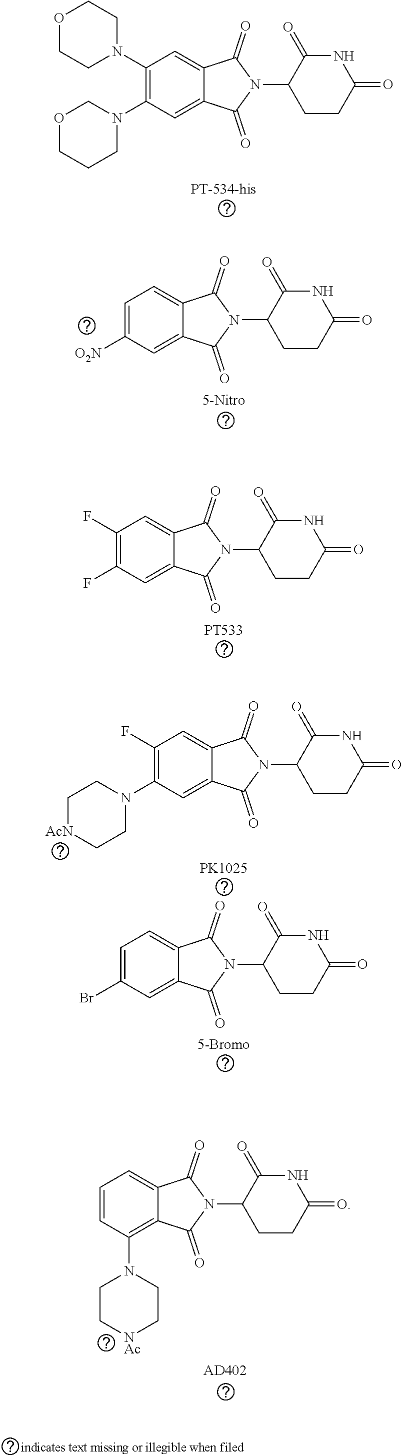

In one example embodiment, the molecule has the following structure

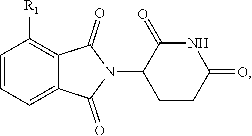

wherein R₁ is selected from

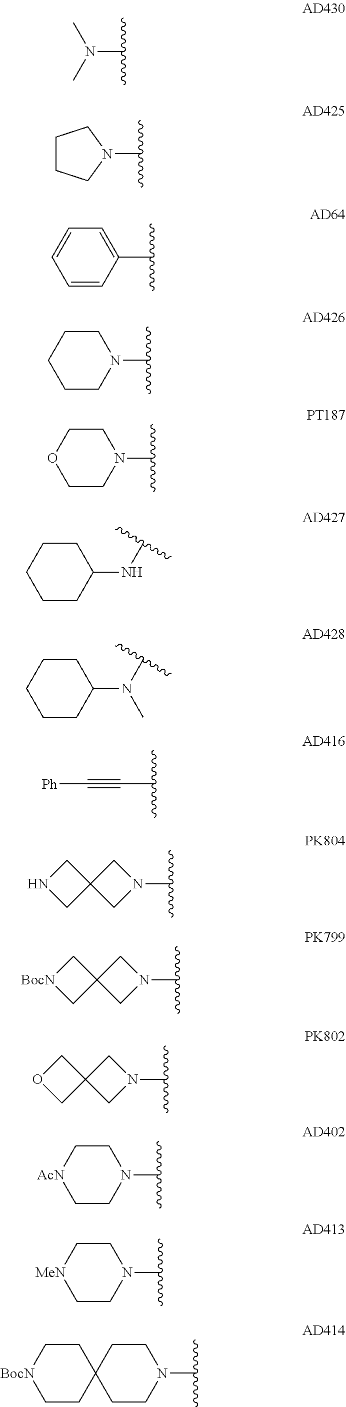

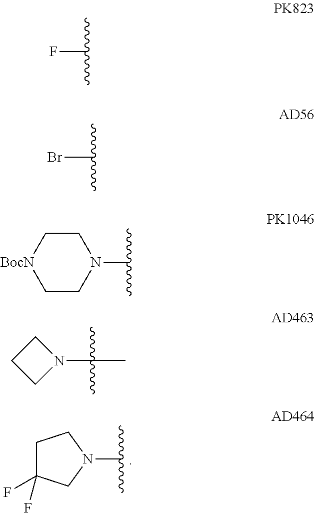

In one example embodiment, R₁ is selected from

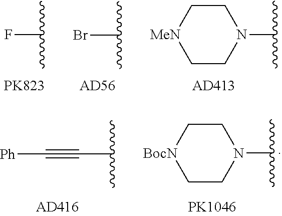

In one embodiment, the molecule is according to the formula

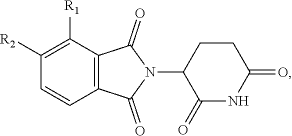

wherein when R₁ is H, R₂ is selected from

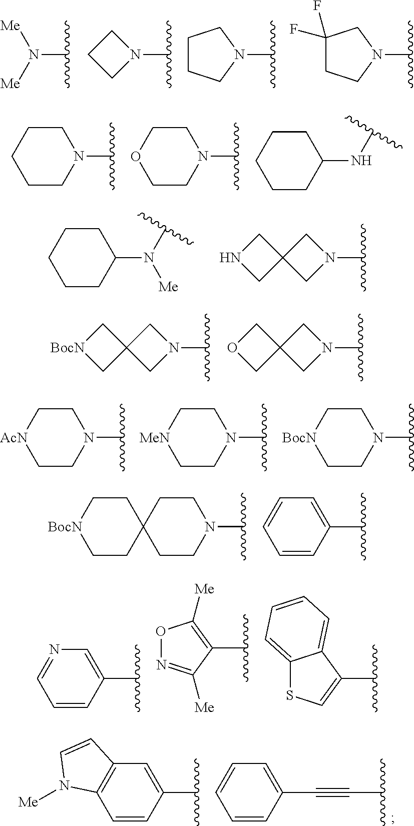

and wherein when R₂ is H, R₁ is selected from

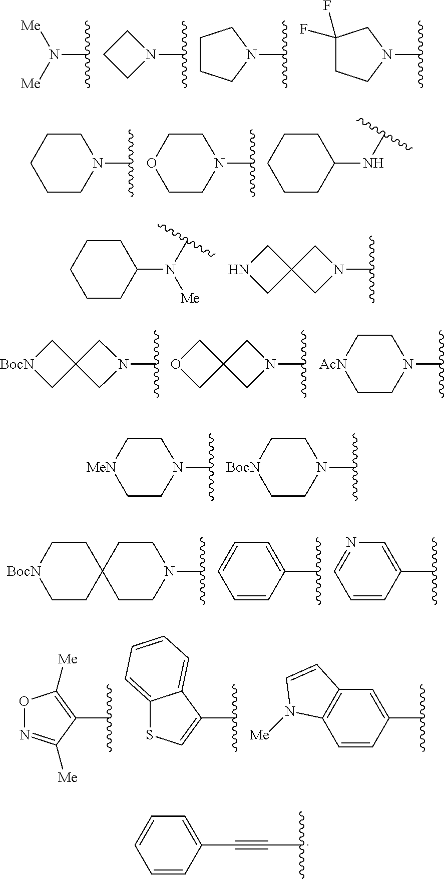

In one example embodiment, the molecule is according to:

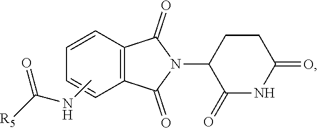

wherein R₅ is selected from

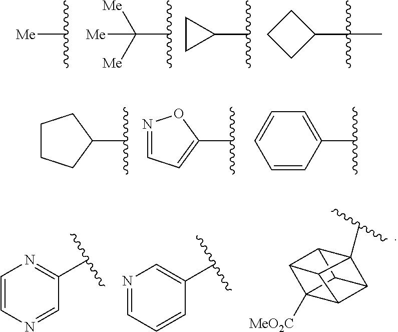

In one example embodiment, the molecule has the following structure

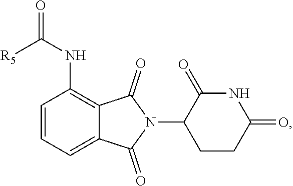

wherein R₅ is selected from

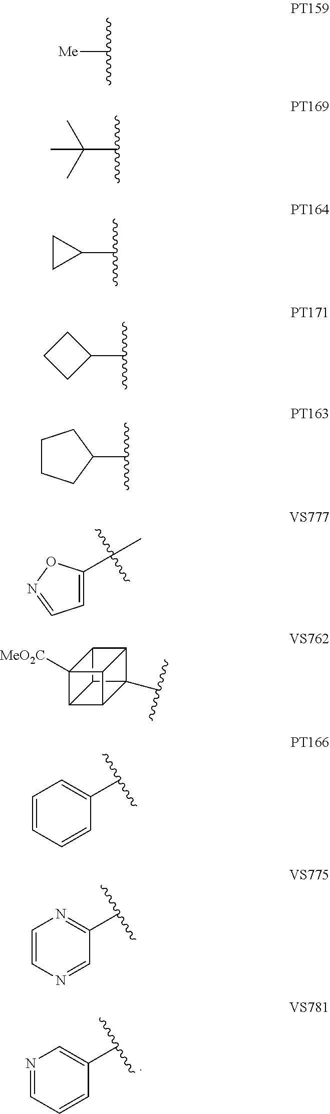

In one example embodiment, the molecule has the following structure

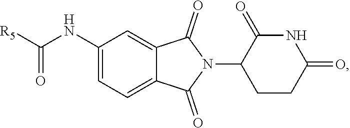

wherein R₅ is selected from

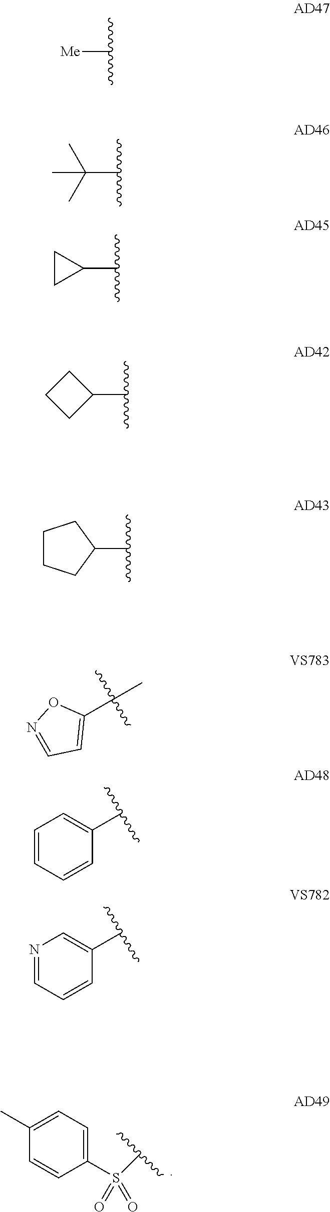

In one example embodiment, the molecule has the following structure

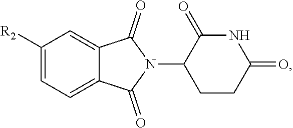

wherein R₂ is selected from

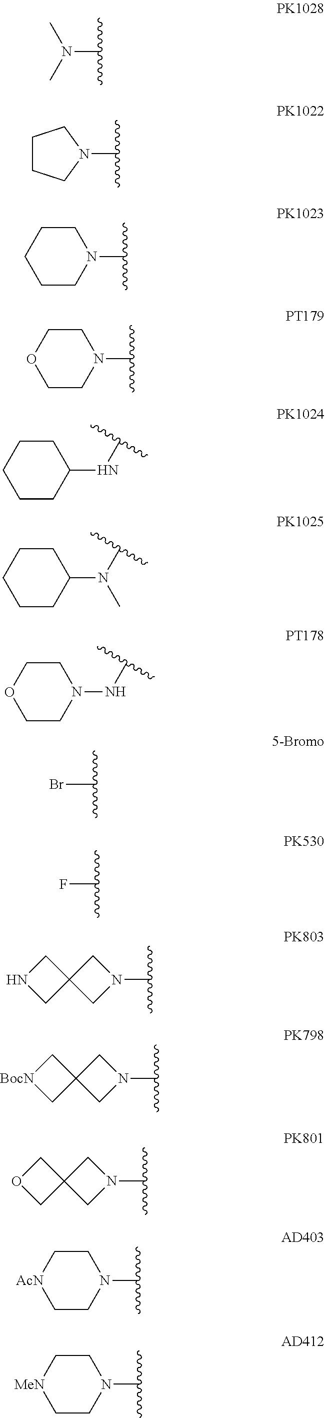

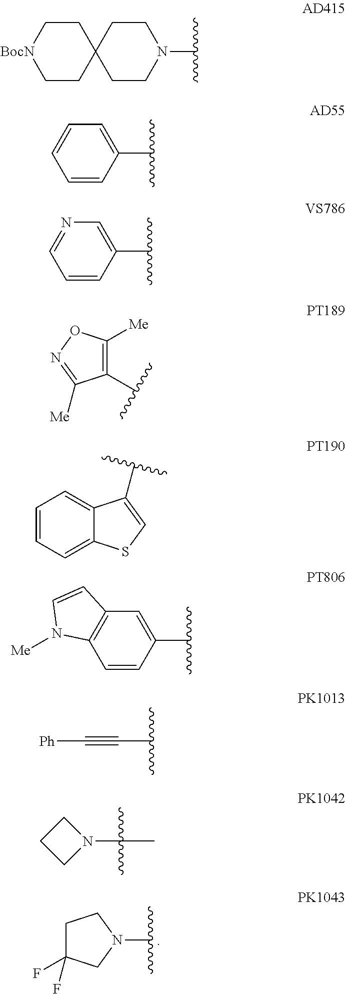

In one example embodiment, R₂ is selected from

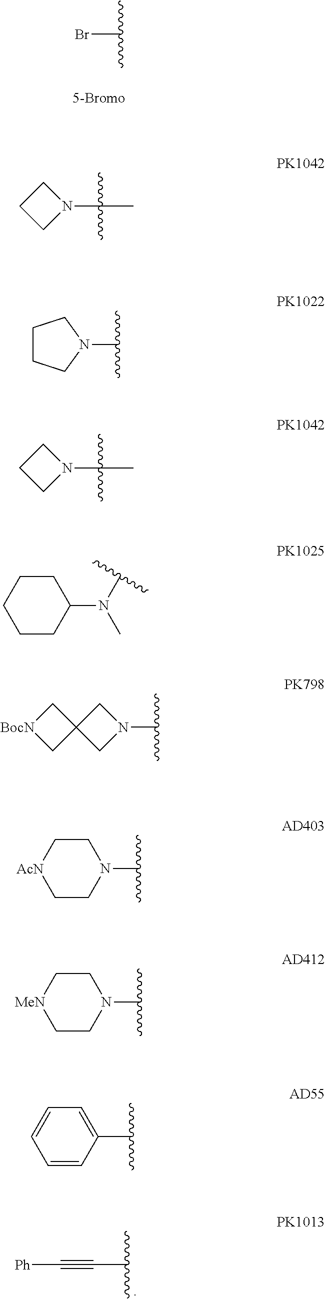

In one example embodiment, the molecule has the following structure

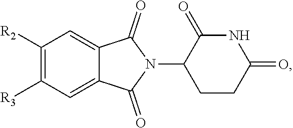

wherein R₃ is a fluorine group and R₂ is selected from

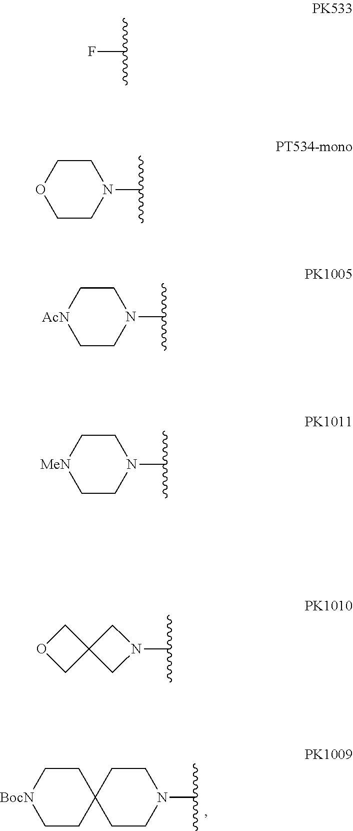

or
wherein R₂ and R₃ are each

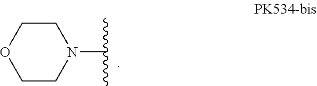 [SMILES: *N1CCOCC1]

In one embodiment, the molecule is according to the formula:

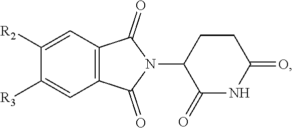

wherein R₂ is F and R₃ is selected from

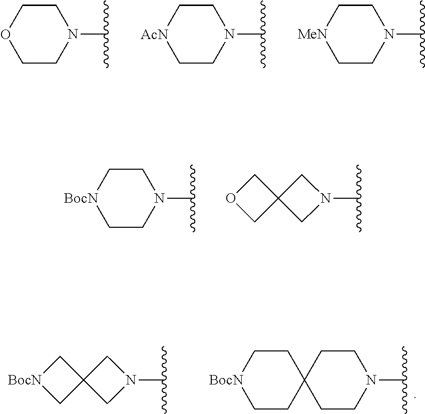

In one aspect, the present invention provides for a molecule according to formula (II)

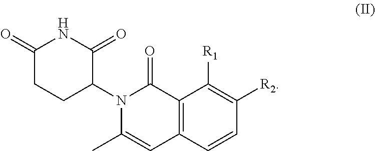

The molecule of formula (II) can comprise various substituent groups. In one example embodiment, R₁ is selected from —H and nitro groups, and wherein R₂ is selected from —H and halogen groups.

In one example embodiment, the molecule is selected from

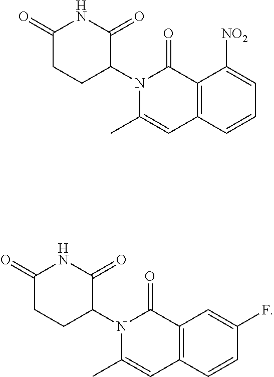 [SMILES: Cc1cc2cccc([N+](=O)[O-])c2c(=O)n1C1CCC(=O)NC1=O ; Cc1cc2ccc(F)cc2c(=O)n1C1CCC(=O)NC1=O]

### Methods of Use

### Degradation of Engineered Systems

The molecules and compositions described herein can be utilized in methods for the control and induced degradation of engineered systems. In an example embodiment, a method of inducing degradation of a target protein comprising a degradation domain is provided. In an aspect, a cell transfected with a variant protein comprising a degradation domain is exposed to a molecule as described herein, a pharmaceutically acceptable salt thereof, or any pharmaceutical combination of molecules as described herein and/or pharmaceutically acceptable salts thereof. In an aspect, a cell transfected with a variant protein comprising a degradation domain is exposed to a composition comprising a molecule as described herein, a pharmaceutically acceptable salt thereof, or any pharmaceutical combination of compositions comprising molecules as described herein and/or pharmaceutically acceptable salts thereof. Methods of inducing degradation may depend on the degradation domain included on a target protein. In example embodiments, the degradation domain is a zinc finger domain or a FK506 binding protein (FKBP) domain. In an aspect, a target protein can be modified to comprise one or more, i.e., two, three, or more, degradation domains.

In an example embodiment, a method of inducing degradation of a programmable nuclease comprising a zinc finger degron comprises administering to a cell or cell population a molecule as described herein.

In an example embodiment, a controllable CAR-T cell can be provided. In an embodiment, a zinc finger degron can be utilized as an ON and OFF-switch on CAR T cells. As was shown by Ebert and colleagues, such engineered systems allow for degradation of the engineered CARS from the cell surface via recruitment to the CRL4^{CRBN} E3 ubiquitin ligase, ubiquitination, and proteasomal degradation. In particular embodiments, the degradation is via the addition of an IMiD analog as described herein. See, Jan et al., Reversible ON and OFF-switch chimeric antigen receptors controlled by lenalidomide, Science Translational Medicine vol. 13, Issue 575, doi: 10.1126/scitranslmed.abb6295, incorporated herein by reference. In an aspect, the molecules disclosed herein may provide improved on-target effects.

In one embodiment, methods of using the compositions and systems herein are provided, allowing for the control, modulation, and/or degradation of systems detailed herein. In an example embodiment, the systems can be utilized for modifying a target nucleic acid by introducing in a cell or organism that comprises the target nucleic acid an engineered programmable nuclease comprising a degradation domain, e.g., Cas protein with zinc finger domain(s) or FKBP domain(s), polynucleotide(s) encoding the engineered Cas protein, the CRISPR-Cas system, or the vector or vector system comprising the polynucleotide(s), such that the engineered programmable nuclease, e.g., Cas protein, modifies the target nucleic acid in the cell or organism. Modulation, control or degradation can be achieved by administration of the composition comprising the molecules described herein to induce degradation. Additional applications of the systems with other proteins, such as activating or repressing translation, base editing, labeling of molecules and their interactions are known in the art and can be utilized with the approaches and degradation domains and molecules detailed herein.

### Zinc Finger Degron

The compositions of the current system may comprise a zinc finger degron. Generally, a degron is a peptide sequence or protein element that confers metabolic instability. A degron may refer to a portion of a protein involved in regulating the degradation rate of a protein. Degrons may include short amino acid sequences, structural motifs, and exposed amino acids (e.g., lysine or arginine). In particular, the currently disclosed system provides variant proteins, for example, programmable nucleases, that comprise one or more degrons. In embodiments, the degron is a zinc finger degron that can be controlled with the thalidomide and pomalidomide analogs described herein. In particular embodiments, the one or more degrons comprise a zinc finger polypeptide. In particular embodiments, the zinc finger comprises a Cys2 His2 (C2H2) domain. The polypeptide, e.g., chimeric antigen receptor, or programmable nuclease, may be engineered to comprise one or more, or two or more zinc finger degron domains. Each zinc finger domain may comprise a hybrid zinc finger, comprising two or more subdomains, each subdomain from a different wild type zinc finger.

The C2H2 zinc finger domain shape has been found to be an important binding determinant, which can be a more important determining factor than the primary amino acid sequence. See, e.g. Sievers et al. 2018, “Defining the human C2H2 zinc-finger degrome targeted by thalidomide analogs through CRBN” Science 2018 Nov. 2: 326(6414): eeat0572; doi: 10.1126/science.aat0572, incorporated herein by reference. Cys2-His2 (C2H2) zinc fingers have emerged as a recurrent degron motif mediating drug-dependent interactions with CRL4^{CRB}. See, e.g. An et al., Nat Commun. 8:15398 (2017), doi: 10.1038/ncomms15398 (showing ZFP91 harbors a zinc finger motif, and is related to the IKZF1/3 ZnF), incorporated herein by reference; Koduri et al., PNAS 116(7) 2539-2544 (2019), doi:10.1073/pnas.1818109116 (finding an IKZF3-derived 25mer constitutes a modular degron that can be used to target heterologous proteins for destruction by IMiDs) incorporated herein by reference, see, e.g., International Patent Publication No. WO 2019/089592, incorporated herein by reference. The C2H2 zinc fingers comprise beta-hairpin and alpha-helix subdomains; a domain typically consisting of about 28 to 30 amino acids comprising an N-terminal beta-hairpin followed by an alpha helix comprising two conserved histidine residues at its C-terminus. See, e.g., Fedotova et al., Acta Nature, 2017 April-June; 9(2): 47-58. The modularity of beta-hairpin and alpha-helix subdomains to build a library of hybrid (also referred to alternately herein as synthetic) zinc fingers has been previously leveraged where a hybrid zinc finger degron is a fusion protein comprising an N-terminal beta hairpin subdomain from one C2H2 zinc finger domain, and a C-terminal alpha helix subdomain from a different zinc finger domain from a library of identified C2H2 zinc finger domains can be provided as described in PCT/US2021/20106, incorporated herein by reference. In an aspect, the molecule has enhanced or increased on-target activity to a zinc finger relative to an IMiD molecule, e.g., thalidomide, pomalidomide, iberdomide, avadomide, or derivatives thereof, including compounds detailed herein.

Variants of the zinc finger degrons can be identified using methods such as, for example, phage assisted continuous evolution (PACE), see, e.g., Esvelt et al. 2011; doi: 10.1038/nature09929. PACE is a system that enables the continuous directed evolution of gene-encoded molecules that can be linked to protein production in Escherichia coli. Other methods of continuous directed evolution can be utilized in the identification of variants. In this manner, variants with increased sensitivity to small molecules other than thalidomide and/or its analogues.

In one embodiment, the enhanced or increased on-target activity of the molecules allows for a reduction in the amount of the molecule administered to induce degradation by 5%, 10%, 15%, 20%, 25%, 30%, 35%, 40%, 45%, 50%, 55%, 60%, 65%, 70%, 75%, 80% or more. In an aspect, the amount of small molecule, e.g., IMiD molecule, administered is reduced by a factor of 2, 3, 4, 5, 6, 7, 8, 9, 10, 20, 50, 100, 110, 120, 130, 140, 150 or more.

In an example embodiment, optimization of the zinc finger can be based on screening methods described herein. The zinc finger may be tailored for use with a molecule described herein.

In preferred embodiments, the molecule may mediate drug-dependent degradation more efficiently, either at a more rapid pace of degradation, more complete degradation, or utilization of a lower dose of the molecule than that of an IMiD.

### Use of a Molecule as Described Herein

In one example embodiment, the methods comprise exposing a cell to a molecule as described herein, a pharmaceutically acceptable salt thereof, or any pharmaceutical combination of molecules as described herein and/or pharmaceutically acceptable salts thereof. In one example embodiment, the variant protein is a programmable nuclease. In one example embodiment, the protein comprises a zinc finger selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, IKZF1/3 AA145-167/146-168, ZNF692 AA417-439, and combinations thereof. In one example embodiment, the programmable nuclease is selected from a CRISPR-Cas protein, a Zinc finger nuclease, a TALEN or a meganuclease.

In one example embodiment, the molecule is selected from

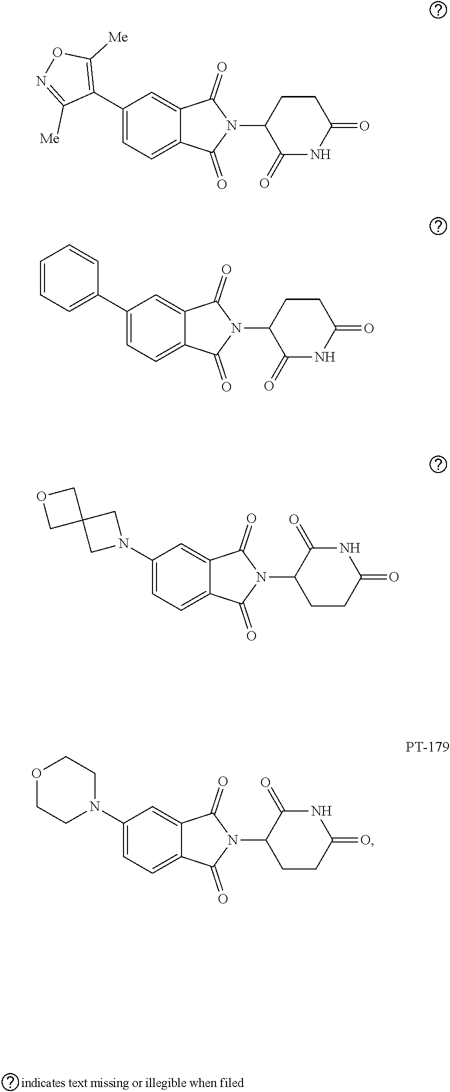 [SMILES: Cc1noc(C)c1-c1ccc2c(c1)C(=O)N(C1CCC(=O)NC1=O)C2=O ; O=C1CCC(N2C(=O)c3ccc(-c4ccccc4)cc3C2=O)C(=O)N1 ; O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1]

and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, and combinations thereof.

In one example embodiment, the molecule is selected from

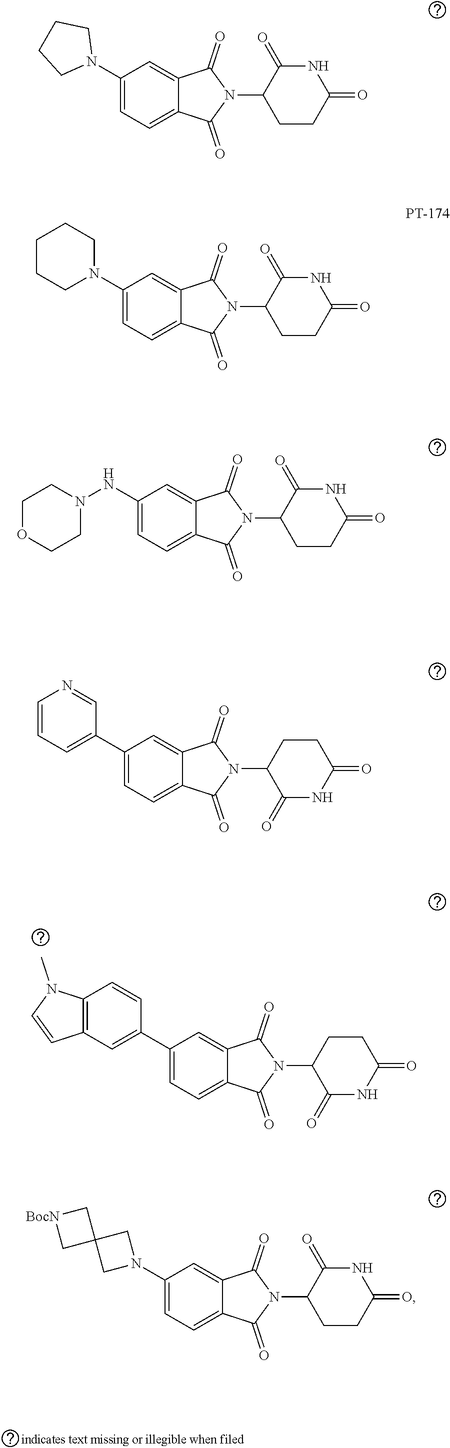 [SMILES: O=C1CCC(N2C(=O)c3ccc(N4CCCC4)cc3C2=O)C(=O)N1 ; O=C1CCC(N2C(=O)c3ccc(N4CCCCC4)cc3C2=O)C(=O)N1 ; O=C1CCC(N2C(=O)c3ccc(NN4CCOCC4)cc3C2=O)C(=O)N1 ; O=C1CCC(N2C(=O)c3ccc(-c4cccnc4)cc3C2=O)C(=O)N1 ; Cn1ccc2cc(-c3ccc4c(c3)C(=O)N(C3CCC(=O)NC3=O)C4=O)ccc21 ; CC(C)(C)OC(=O)N1CC2(C1)CN(c1ccc3c(c1)C(=O)N(C1CCC(=O)NC1=O)C3=O)C2]

and wherein the cell comprises one or more zinc fingers selected from ZFN653 AA556-578, ZFN517 AA452-474, ZFP91_400_422, E4F1 AA220-242, ZFN654 AA25-47, IKZF3_146_168, ZNF582 AA395-417, IKZF3 AA146-168 Q147E, SALL4 ZF2, and combinations thereof.

In one example embodiment, the molecule is selected from

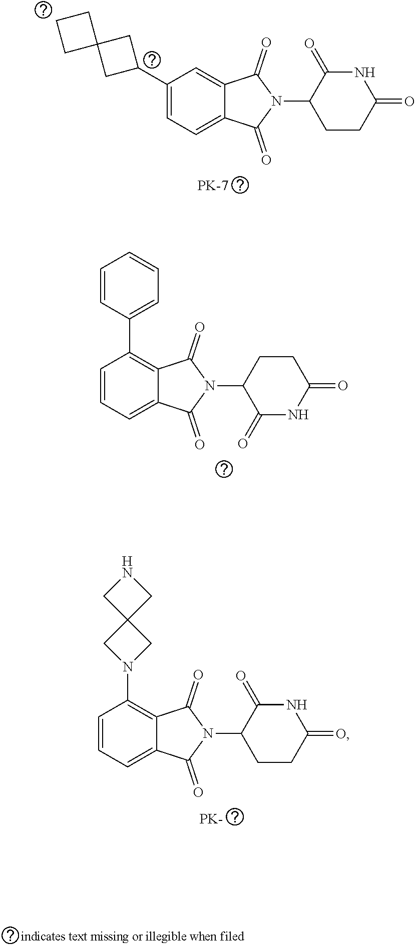

and wherein the cell comprises one or more zinc fingers selected from ZFN276 AA524-576, ZFN653 AA556-578, ZFN787 AA 178-200, ZFN517 AA452-474, ZFP91_400_422, E4F1 AA220-242, ZFN654 AA25-47, IKZF3_146_168, ZNF582 AA395-417, and combinations thereof.

In one example embodiment, the molecule is selected from

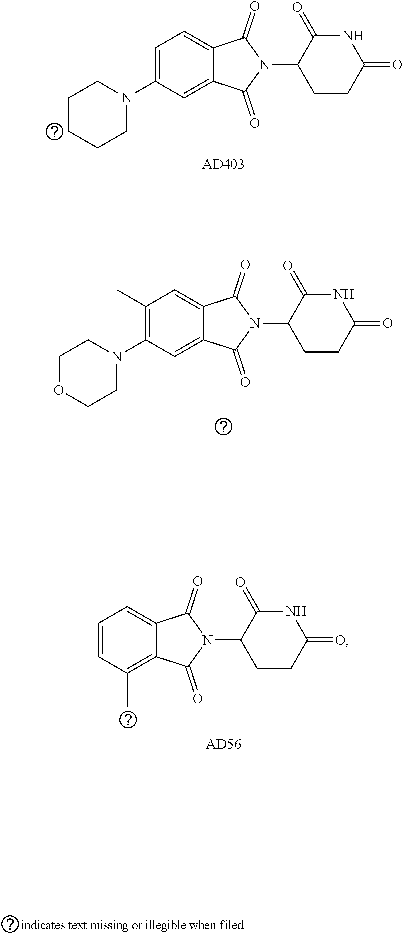

and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, and combinations thereof.

In one example embodiment, the molecule is selected from

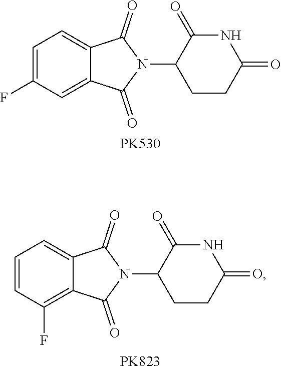

and wherein the cell comprises one or more zinc fingers selected from ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, IKZF3_146_168, ZNF582 AA395-417, ZKSC5_430_452, IKZF3 AA146-168 Q147E, SALL4 ZF2, and combinations thereof.

In one example embodiment, the molecule is selected from

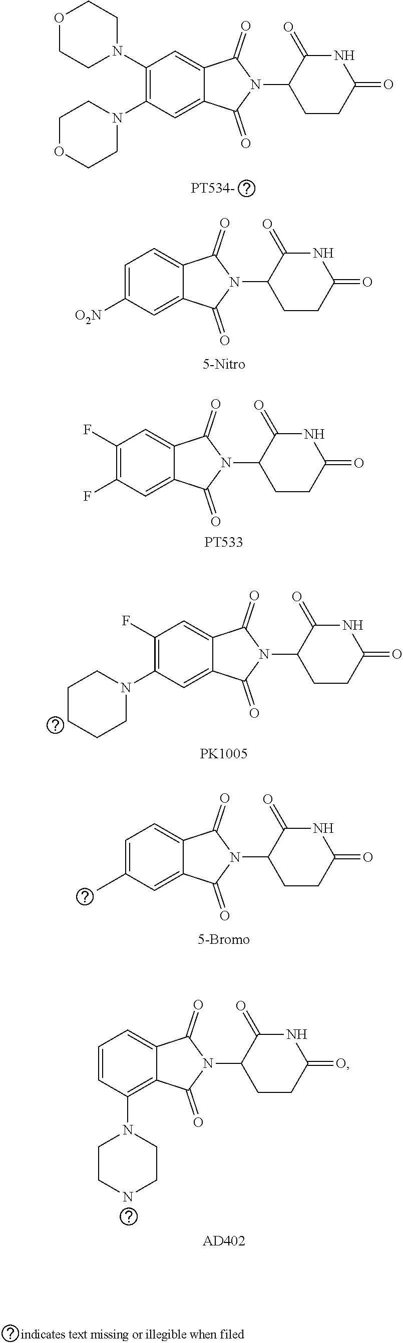

and wherein the cell comprises one or more zine finger ZFN653 AA556-578, ZFN827 AA374-396, ZFN787 AA 178-200, ZFN517 AA452-474, IKZF3_146_168, ZNF582 AA395-417, IKZF3 AA146-168 Q147E, SALL4 ZF2, and combinations thereof.

In one example embodiment, the molecule is according to the formula

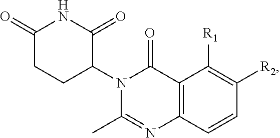

wherein R₁ is selected from —H and nitro groups, and wherein R₂ is selected from —H and halogen groups, and wherein the cell comprises one or more zine fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, and combinations thereof.

In one example embodiment, the molecule is selected from

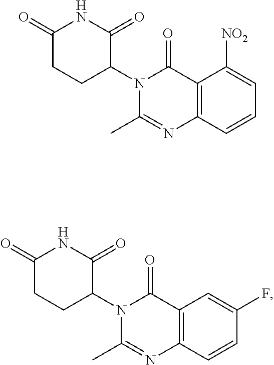 [SMILES: Cc1nc2cccc([N+](=O)[O-])c2c(=O)n1C1CCC(=O)NC1=O ; Cc1nc2ccc(F)cc2c(=O)n1C1CCC(=O)NC1=O]

and wherein the cell comprises one or more zinc fingers selected from ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, and combinations thereof.

### FK506 Binding Protein (FKBP) Domain

The compositions of the current system may be used in a system with a protein comprising one or more FK506 binding protein (FKBP) domains. The system can comprise a degradation tag, (dTAG) for an FKBP protein, that artificially induces the selective degradation of a protein comprising the one or more FKBP protein binding domains by using that dTAG to bring the target protein in proximity to E3 ligase, ubiquitinylating the protein-of interest, that can then be processed through proteasome-mediated degradation. See, e.g., Sreekanth et al., ACS Cen. Sci. 2020 6, 12, 2228-2237; doi: 10.1021/acscentsci.0c00129.

In an exemplary embodiment, a fusion protein comprising one or more FKBP domains, e.g., a Cas protein comprising one or more FKBP12^{F36V} domains. The compositions disclosed herein can find use as a part of a degradation tag, (dTAG) for an FKBP protein, for example, as an FKBP12^{F36V} tag. The dTAG is a heterobifunctional molecule consisting of a binder for the FKBP domain parried to a binder fo the Cereblon E3 ligase (CRBN). While the CRBN binder can comprise thalidomide or its analogs, compositions as described herein may be utilized in a dTAG molecule to optimize its use to control proteasomal degradation of protein fusions with one or more FKBP domains, e.g., (FKBP)12F36V domains.

### Degradation of Target Amines

The molecules and compositions described herein can be utilized in methods for the control and induced degradation of a target amine. In an example embodiment, a method of inducing degradation of a target amine is provided. In an aspect, a cell comprising or transfected with a target amine is exposed to a composition comprising a molecule as described herein, a pharmaceutically acceptable salt thereof, or any pharmaceutical combination of compositions comprising molecules as described herein and/or pharmaceutically acceptable salts thereof. Methods of inducing degradation may depend on the target amine. In example embodiments, the degradation of the target amine has improved on-target degradation of traditionally intractable protein targets in disease with reduced off-target effects. See, e.g., Gadd et al. Nat Chem Biol. 2017, 12, (5), 514-521; International Patent Publication No. WO202114235, incorporate herein by reference in its entirety, see in particular [0370]-[0389].

### Use of a Composition Comprising a Molecule as Described Herein

In one example embodiment, a method of inducing degradation of a target protein or amine in a cell comprises exposing a cell transfected with a target protein or comprising a target amine with a composition comprising a molecule as described herein.

In one example embodiment, a method of inducing degradation of a target protein or amine in a cell comprises exposing a cell transfected with a target protein or comprising a target amine with a composition comprising a molecule as described herein. In one example embodiment, the composition is according to formula (III):

A-(L)ₙ-B. (III).

In one example embodiment, A is a target binding ligand, L is a linker group, B is a molecule according to the present invention, n is between 0 and 12, and B is conjugated to A or (L)ₙ via R₁ or R₂.

### Targets and Target Binding Ligands

Various targets and targeting binding ligands can be used. In one example embodiment, the target protein is a variant protein comprising one or more FK506 binding protein (FKBP) domains and A is a ligand that binds to one of the one or more FK506 binding protein (FKBP) domains. In one example embodiment, the target is a target amine and A is a ligand that binds to the target amine.

### Linker Groups and Conjugates

Various (L)ₙ linker groups and (L)ₙ-B conjugates can be used. In one example embodiment, (L)ₙ-B comprises an alkyl, an alkyne, a glycol ether, a polyglycol ether, a heterocyclic, a heteroaryl, or an aryl group. In one example embodiment, (L)ₙ-B comprises a C₄₋₈ alkyl group.

In one example embodiment, (L)ₙ or (L)ₙ-B comprises a group selected from

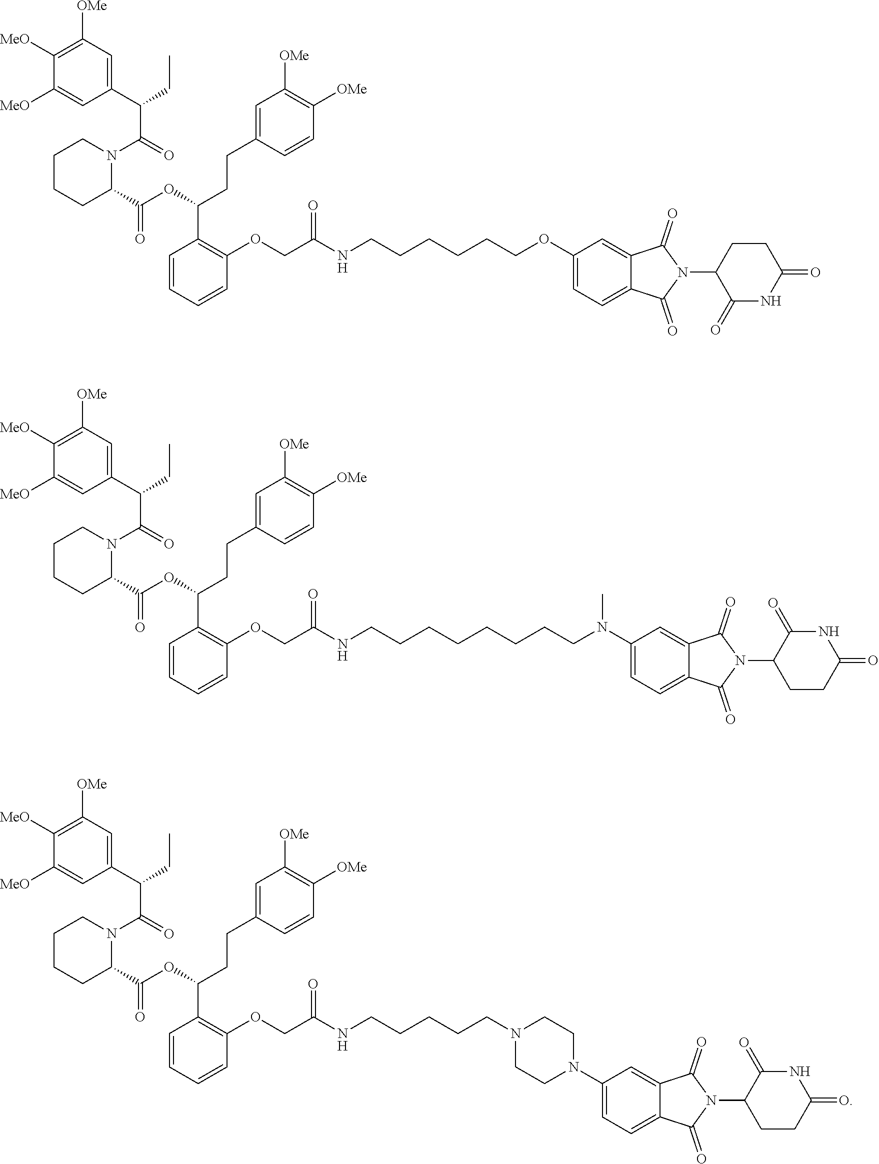 [SMILES: CC[C@H](C(=O)N1CCCC[C@H]1C(=O)O[C@H](CCc1ccc(OC)c(OC)c1)c1ccccc1OCC(=O)NCCCCCCOc1ccc2c(c1)C(=O)N(C1CCC(=O)NC1=O)C2=O)c1cc(OC)c(OC)c(OC)c1 ; CC[C@H](C(=O)N1CCCC[C@H]1C(=O)O[C@H](CCc1ccc(OC)c(OC)c1)c1ccccc1OCC(=O)NCCCCCCCCN(C)c1ccc2c(c1)C(=O)N(C1CCC(=O)NC1=O)C2=O)c1cc(OC)c(OC)c(OC)c1 ; CC[C@H](C(=O)N1CCCC[C@H]1C(=O)O[C@H](CCc1ccc(OC)c(OC)c1)c1ccccc1OCC(=O)NCCCCCN1CCN(c2ccc3c(c2)C(=O)N(C2CCC(=O)NC2=O)C3=O)CC1)c1cc(OC)c(OC)c(OC)c1]

In one example embodiment, (L)ₙ-B is selected from

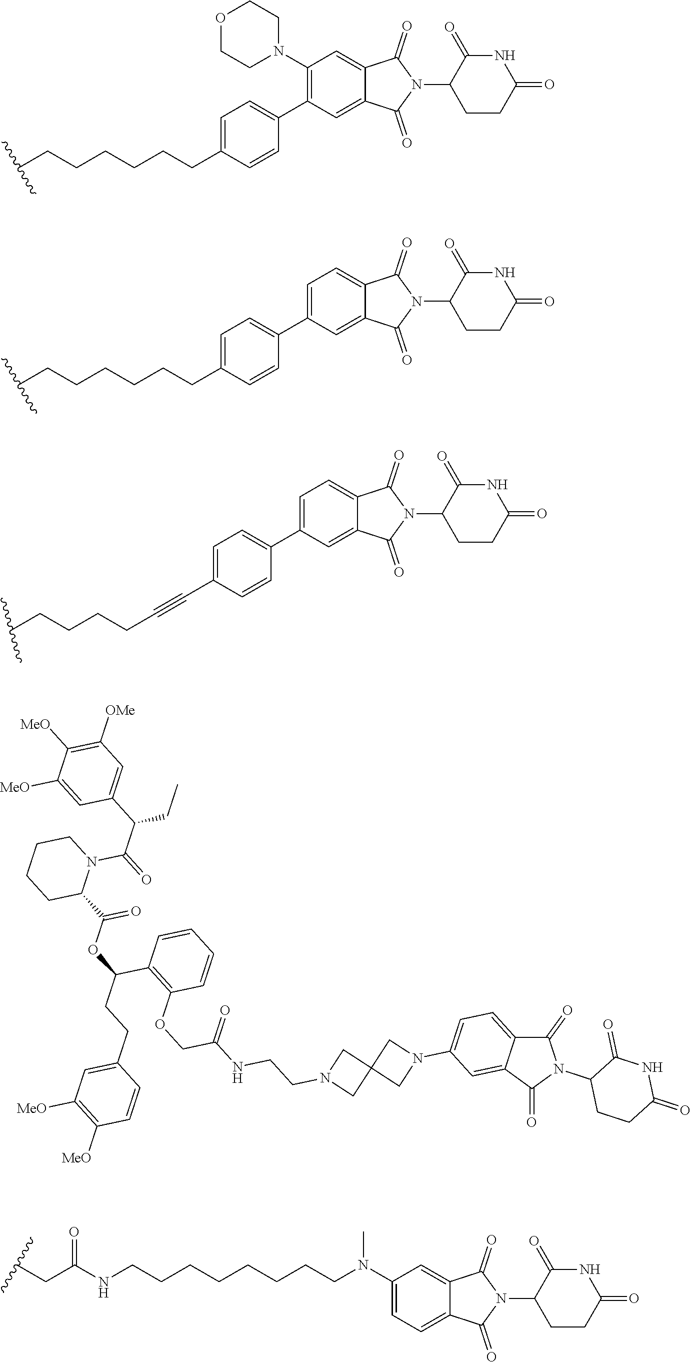 [SMILES: *CCCCCCc1ccc(-c2cc3c(cc2N2CCOCC2)C(=O)N(C2CCC(=O)NC2=O)C3=O)cc1 ; *CCCCCCc1ccc(-c2ccc3c(c2)C(=O)N(C2CCC(=O)NC2=O)C3=O)cc1 ; *CCCCC#Cc1ccc(-c2ccc3c(c2)C(=O)N(C2CCC(=O)NC2=O)C3=O)cc1 ; CC[C@H](C(=O)N1CCCC[C@H]1C(=O)O[C@H](CCc1ccc(OC)c(OC)c1)c1ccccc1OCC(=O)NCCN1CC2(C1)CN(c1ccc3c(c1)C(=O)N(C1CCC(=O)NC1=O)C3=O)C2)c1cc(OC)c(OC)c(OC)c1 ; *CC(=O)NCCCCCCCCN(C)c1ccc2c(c1)C(=O)N(C1CCC(=O)NC1=O)C2=O]

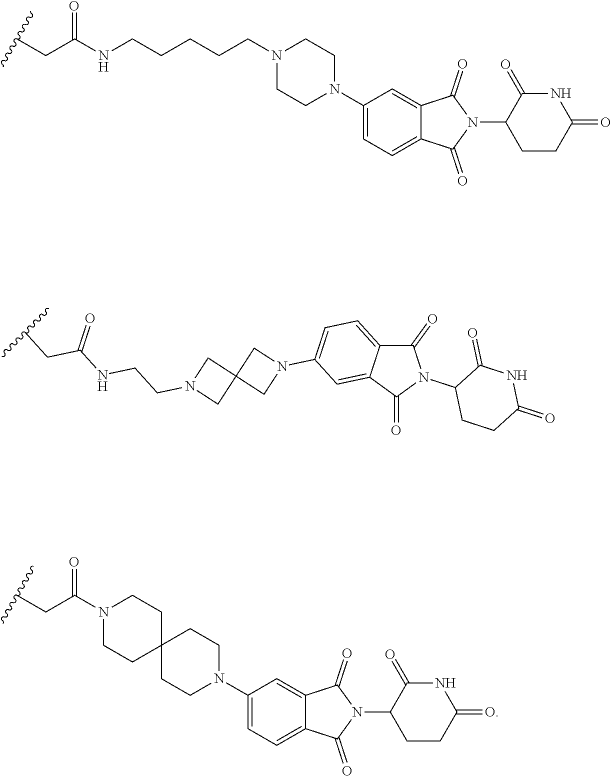 [SMILES: *CC(=O)NCCCCCN1CCN(c2ccc3c(c2)C(=O)N(C2CCC(=O)NC2=O)C3=O)CC1 ; *CC(=O)NCCN1CC2(C1)CN(c1ccc3c(c1)C(=O)N(C1CCC(=O)NC1=O)C3=O)C2 ; *CC(=O)N1CCC2(CC1)CCN(c1ccc3c(c1)C(=O)N(C1CCC(=O)NC1=O)C3=O)CC2]

In one example embodiment, R₁ or R₂ is according to R₄, and wherein R₄ is an ether group according to the formula: —NH—C(O)—CH₂—O— or —O—. In one example embodiment, (L)ₙ-B is

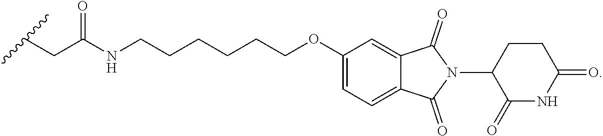 [SMILES: *CC(=O)NCCCCCCOc1ccc2c(c1)C(=O)N(C1CCC(=O)NC1=O)C2=O]

### Methods of Treatment

The present disclosure also contemplates use of the molecules described herein, for treatment in a variety of diseases and disorders. The present disclosure also contemplates use of the molecules and methods described herein, for treatment in a variety of diseases and disorders.

In some embodiments, the disease or disorder is a hematopoietic disease or a symptom thereof. In some embodiments, the disease or disorder is a neurobiological disease or disorder, a psychiatric disease or disorder, a cancer, an autoimmune disease or disorder, a thrombosis disease, a heart disease, a kidney disease, a lung disease, or a blood vessel disease, or a combination thereof.

Methods of modifying a target substrate in a subject in need thereof is provided, the method comprising administering a molecule as disclosed herein to the subject. Delivery can be as described elsewhere herein. In embodiments, the disclosure described herein relates to a method for therapy in which cells are modified ex vivo by the molecules as disclosed herein to modify at least one target substrate, with subsequent administration of the edited cells to a patient in need thereof.

### Pharmaceutical Formulation

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include, such as an active ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a hematopoietic disease or a symptom thereof. In some embodiments, the subject in need thereof has or is suspected of having, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

The therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Lippincott Williams & Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof. Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

In embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

Methods for modifying a programmable nuclease of interest are also provided, the method comprising contacting the programmable nuclease of interest with a molecule or a composition disclosed herein. Methods for the treatment of a disease, disorder, or condition in a subject in need thereof can comprise administering a molecule or a composition disclosed herein to a subject.

### Methods of Screening

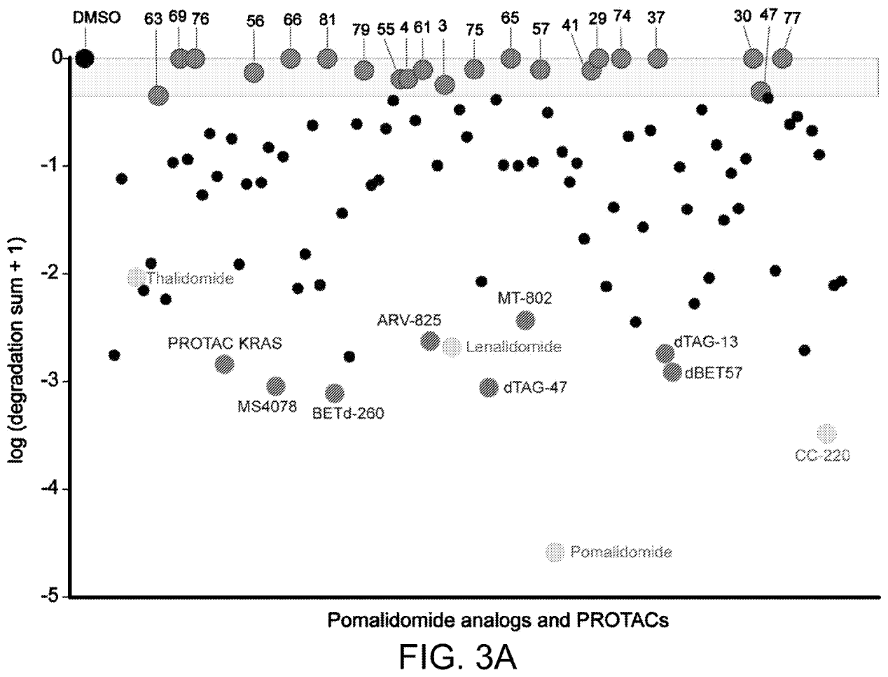

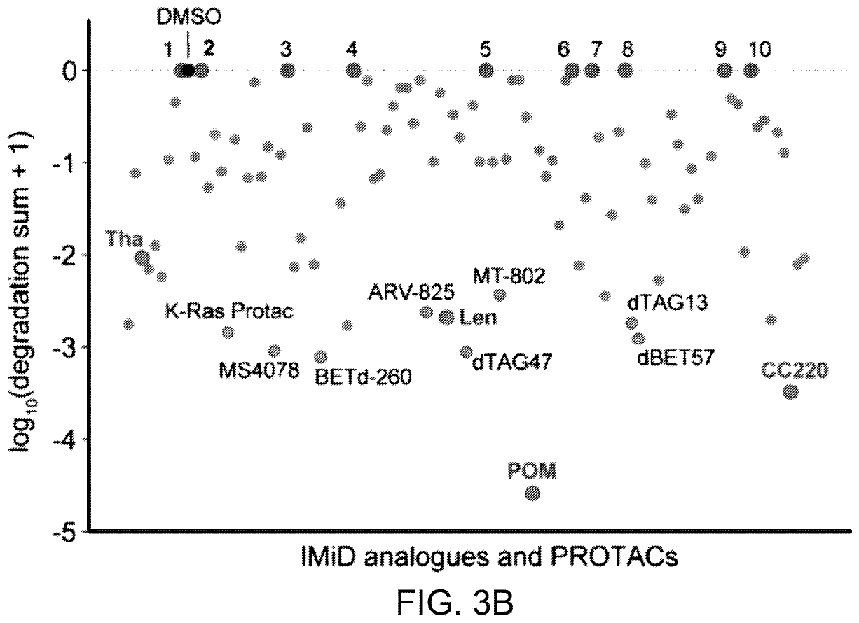

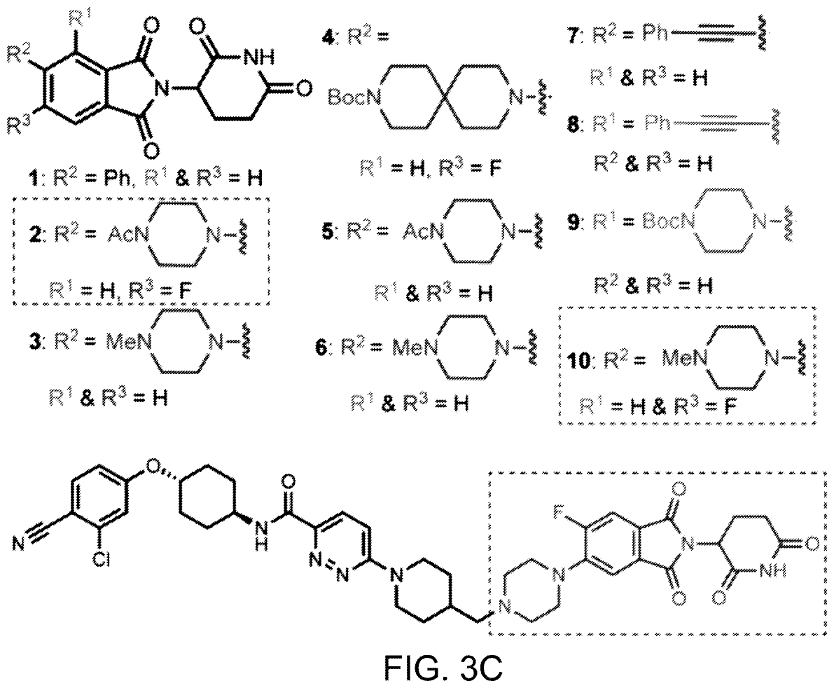

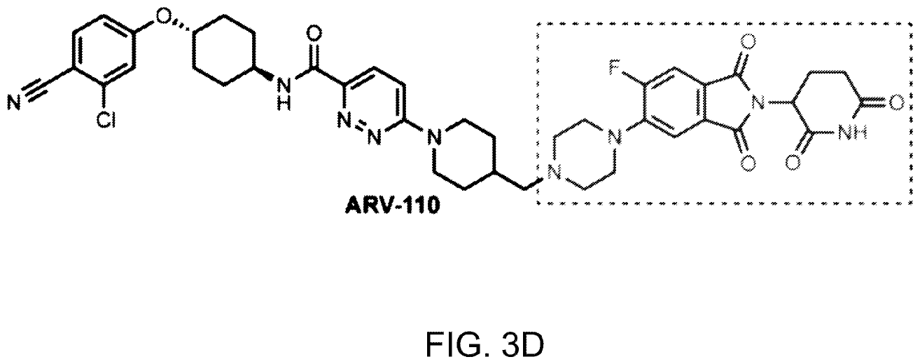

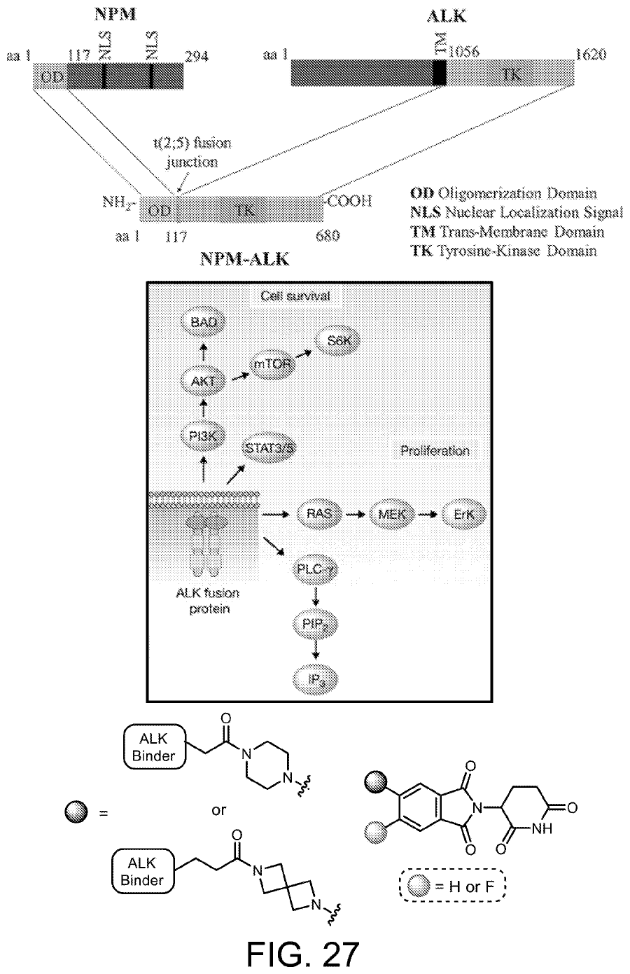

Methods of screening for the combination of moieties to be provided in the molecule are provided herein. In one embodiment, the methods of screening identify molecules with reduced zinc finger off-targets. By way of example, high content confocal microscopy approach for off-target identification of bifunctional molecules is depicted in FIG. 27; however, the screening method described is applicable to identification of off-target activity for other bifunctional moleules as well as the IMiD derivatives detailed herein. In certain methods of screening, molecules are identified based on degradation score as detailed in FIG. 3, and/or other technologies for off-target identification, such as mass spectrometry as described in Donovan et al., ell 2020, 183, 1714-371.e10.

The methods of for determination of off-target activity can be utilized for screening of chemical libraries, and to identify additional exit vectors for use within the context of the embodiments disclosed herein.

In some embodiments, screening of test agents involves testing a combinatorial library containing a large number of potential heterobifunctional molecules, their linkers, ligands, and IMiD derivatives and their exit vectors. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

### Screening for Molecules

A further aspect of the disclosure relates to a method for identifying a molecule capable of on-target proteasomal degradation as disclosed herein, comprising: a) applying a candidate molecule to the cell or cell population; b) detecting degradation by the candidate agent, thereby identifying the agent.

After the molecule is applied, a representative cell sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect via degradation or off-target effects simultaneously in a relatively short amount of time, for example using a high throughput method.

In some embodiments, screening of test agents involves testing a combinatorial library containing a large number of potential molecules. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

Further embodiments are illustrated in the following Examples which are given for illustrative purposes only and are not intended to limit the scope of the disclosure.

### EXAMPLES

### Example 1—Rational Design of PROTACs with Minimal Off-Target Degradation

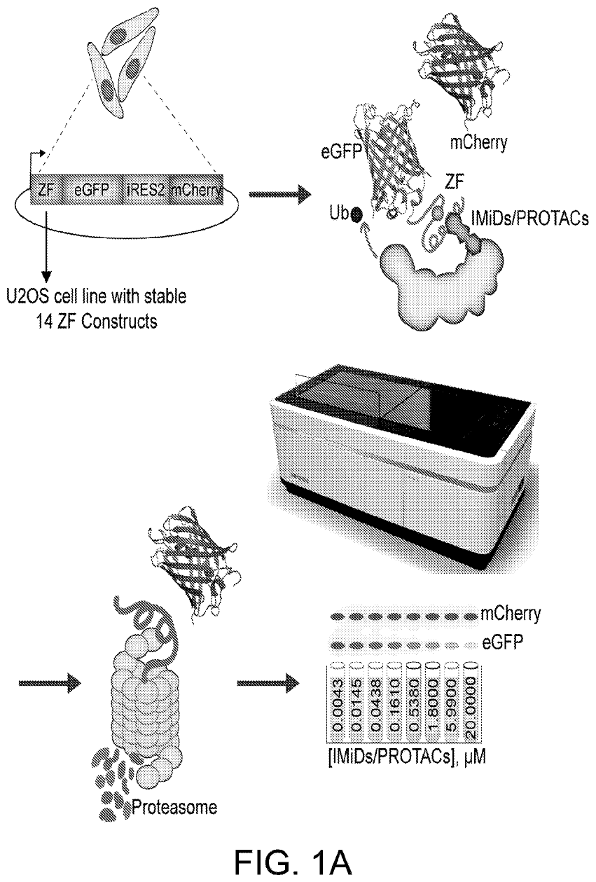

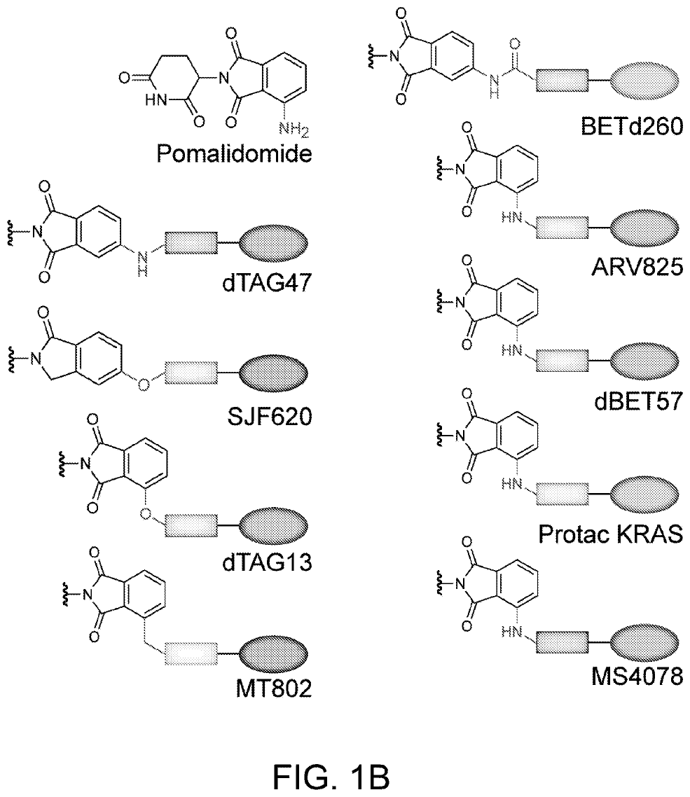

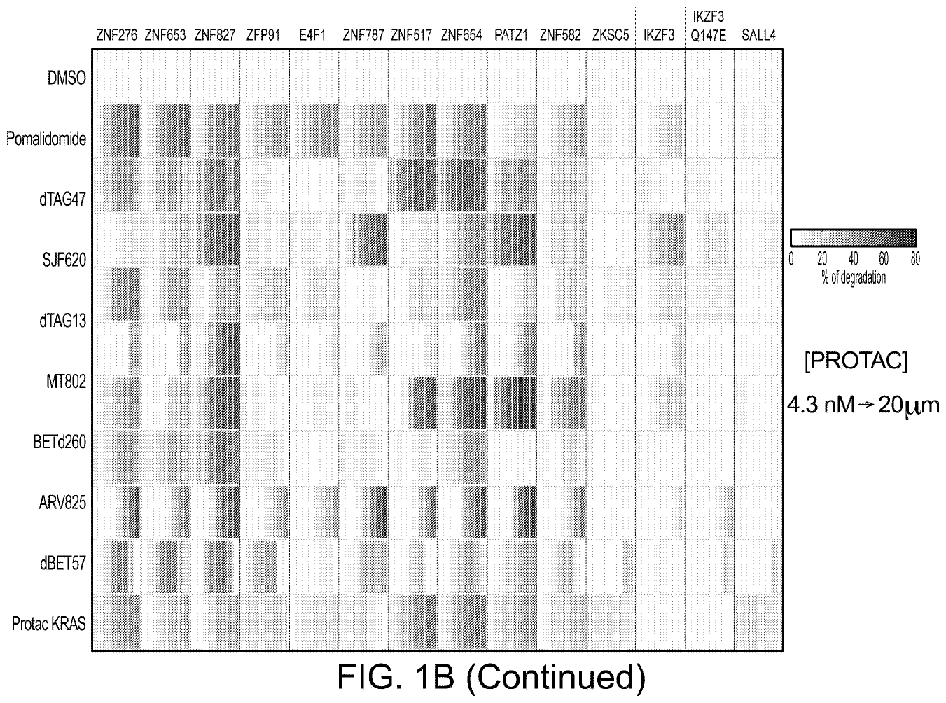

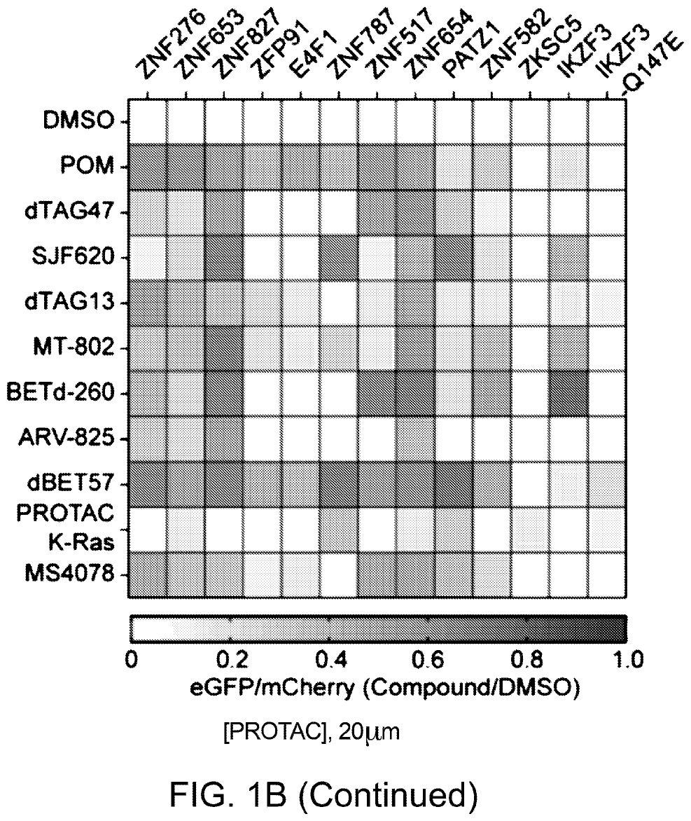

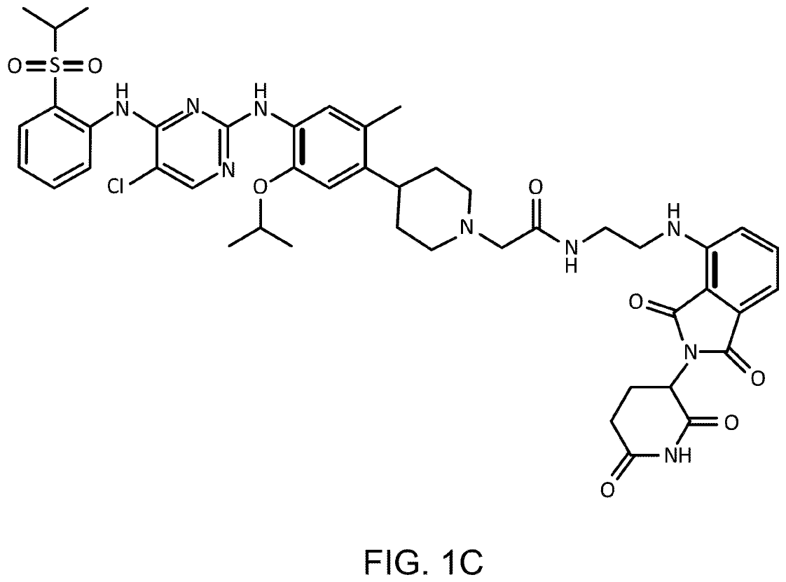

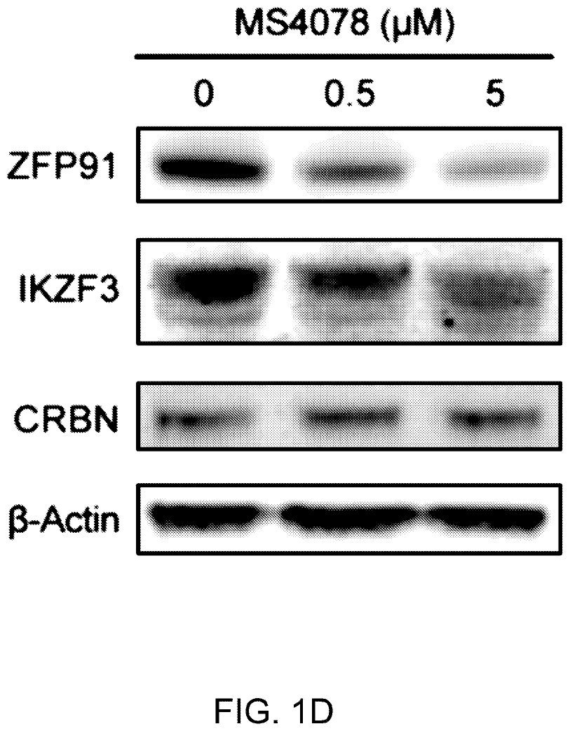

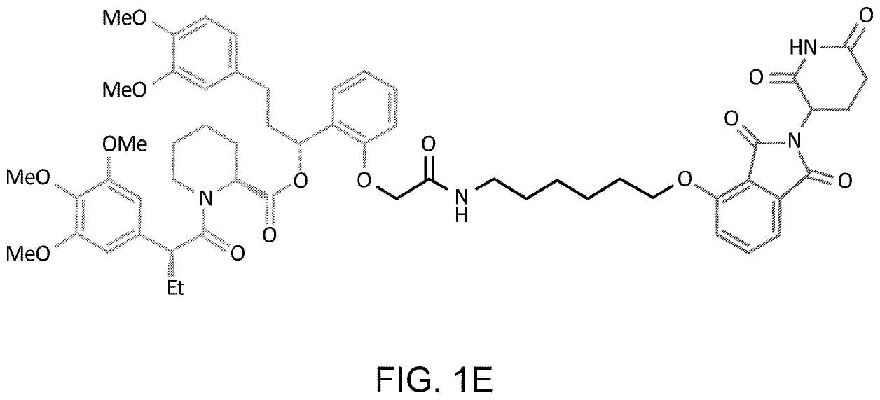

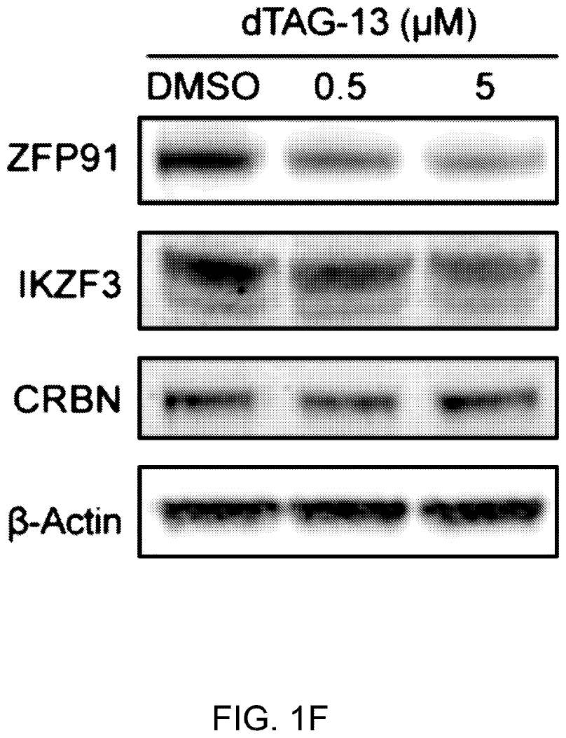

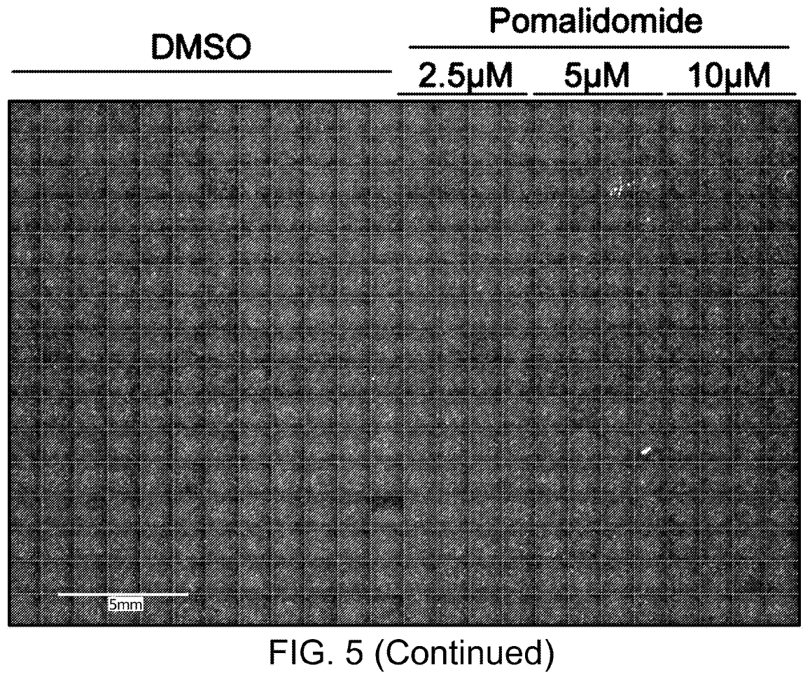

Development and validation of an off-target profiling platform for PROTACs. To profile the ZF degradation propensity of pomalidomide and its analogs, an automated high-content imaging assay was first developed (FIG. 1A). For this platform, 23-amino acid ZF degrons of 12 ZF proteins were selected that are reportedly degraded by pomalidomide and two ZFs that are not.² These ZF degrons were inserted into a lentiviral degradation reporter vector (cilantro 2) to compare the fluorescence of ZF-tagged enhanced green fluorescent protein (eGFP) to untagged mCherry with high-content imaging (FIG. 1A). With this assay, wherein compounds are scanned against the 14 stable cell lines containing our tagged ZF-proteins, dose-dependent degradation of ZF degron-tagged eGFP by pomalidomide was reliably detected with a robust readout (Z-prime value of 0.8) (FIG. 5). As expected, pomalidomide degraded all 12 ZF degrons that are sensitive to pomalidomide in a dose-dependent manner that ranged from 4.3 nM to 20 μM (FIG. 1A). Unlike mass spectrometry, this reporter-based method is not limited by cell-type-specific expression levels of analyte proteins nor by the accessibility to ZFs in the context of full-length proteins that are engaged in protein complexes. Thus, this method has enhanced sensitivity over mass spectrometry-based methods for the detection of pomalidomide-sensitive ZF protein degradation.

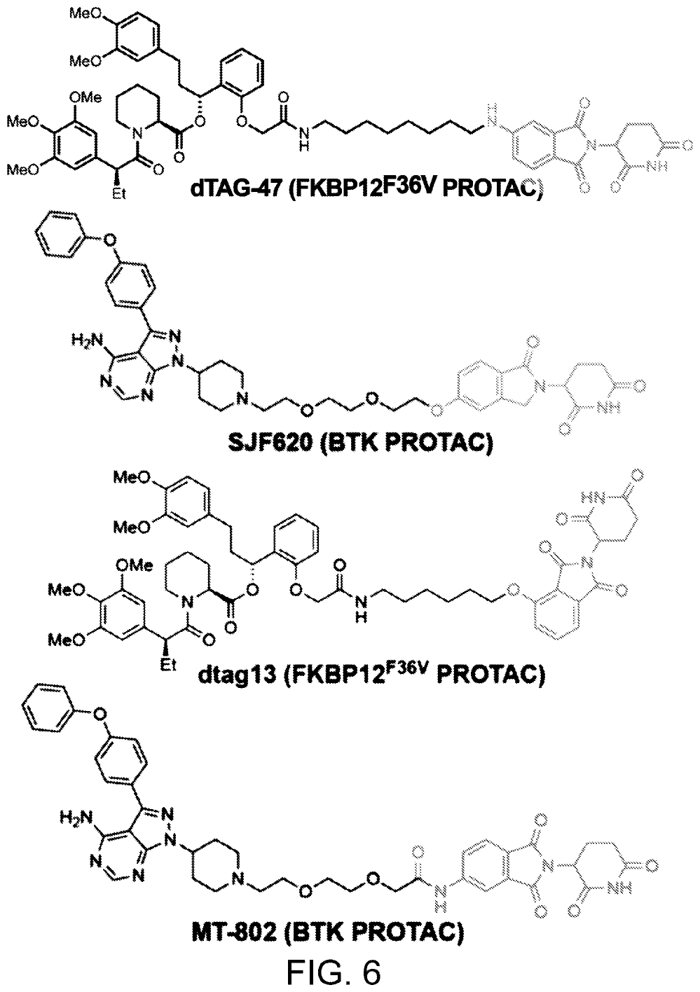

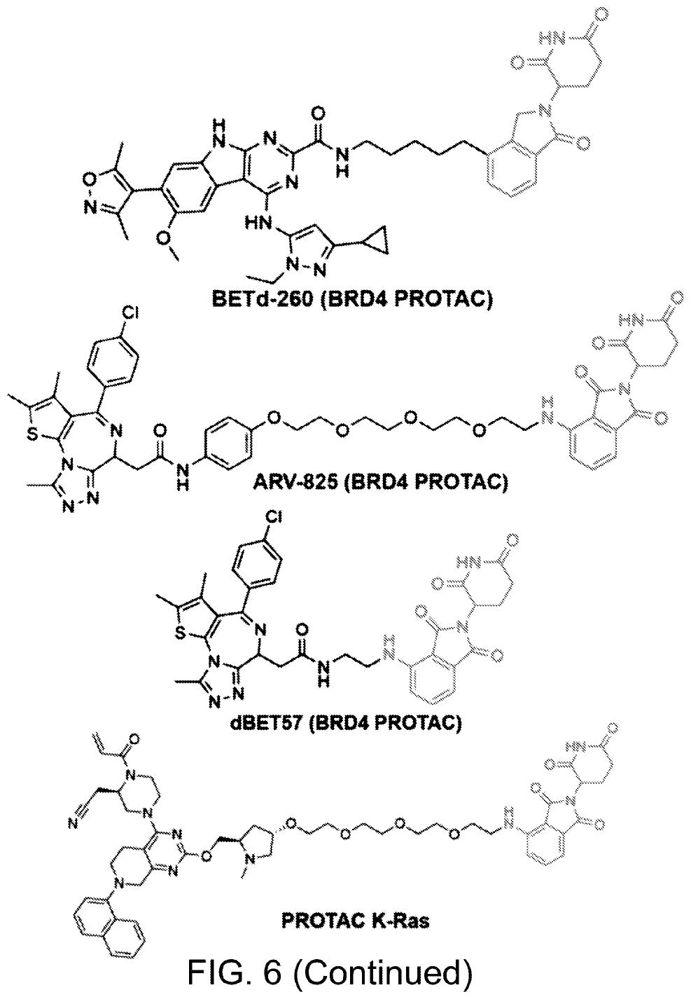

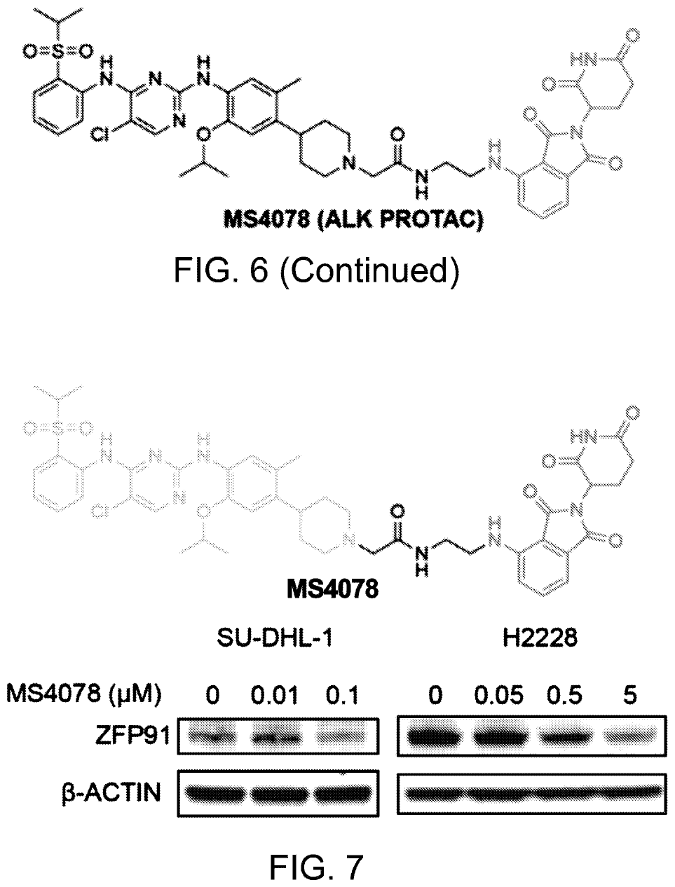

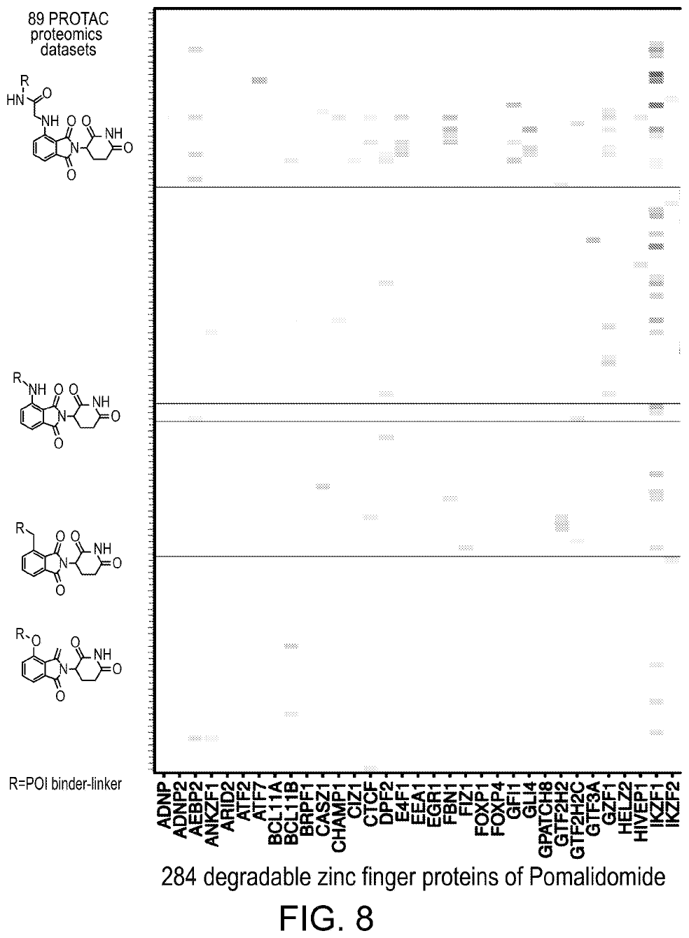

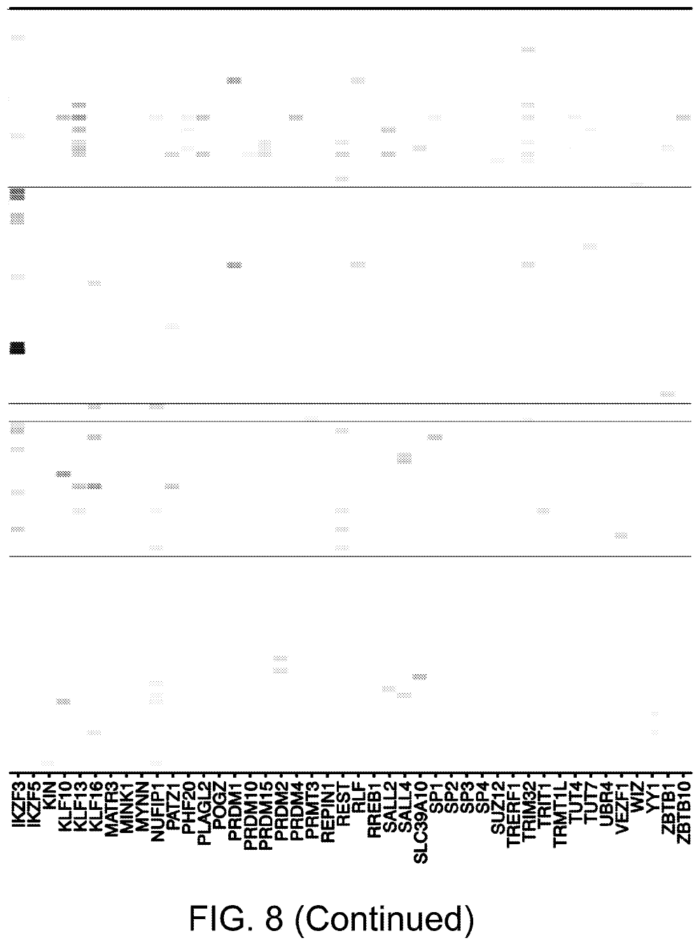

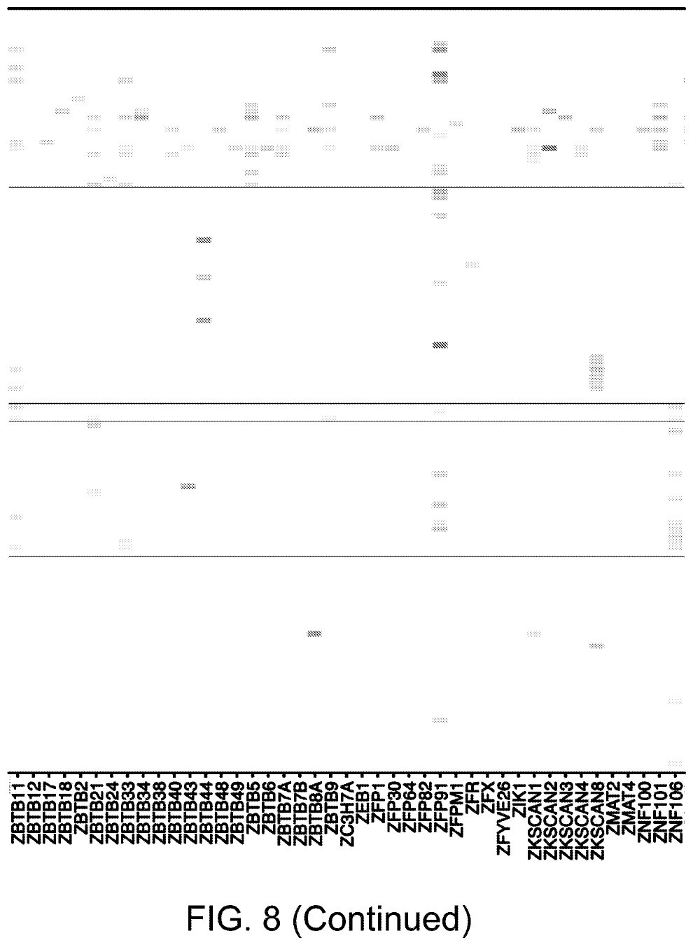

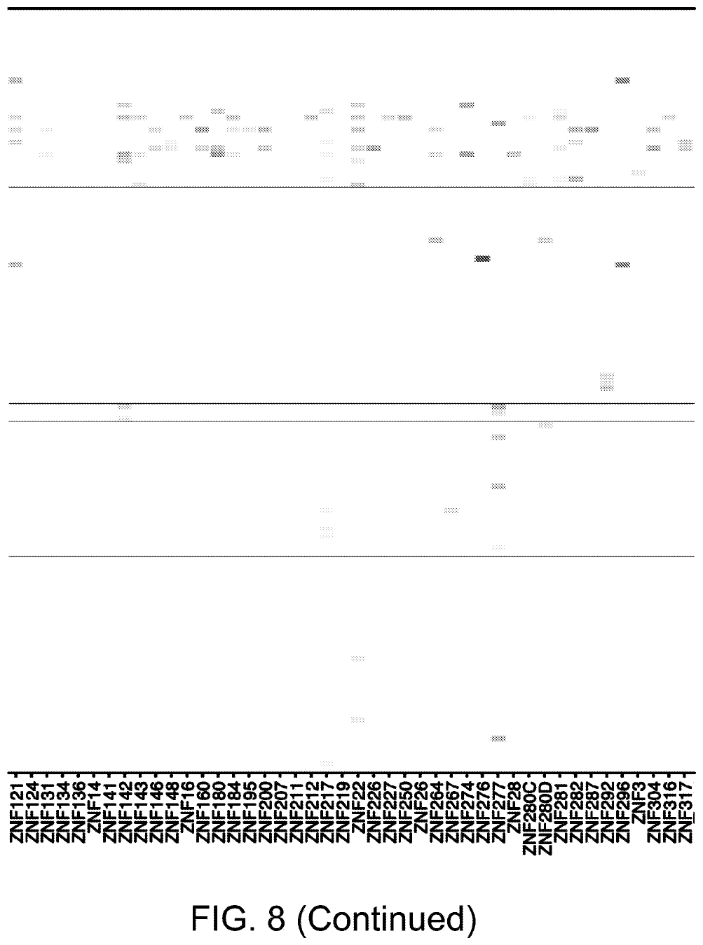

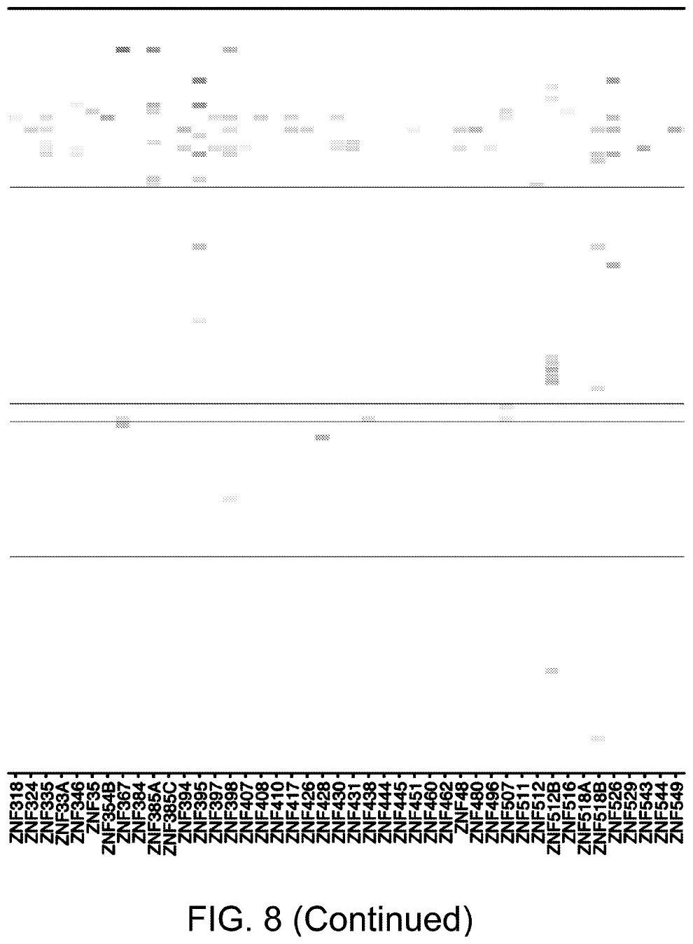

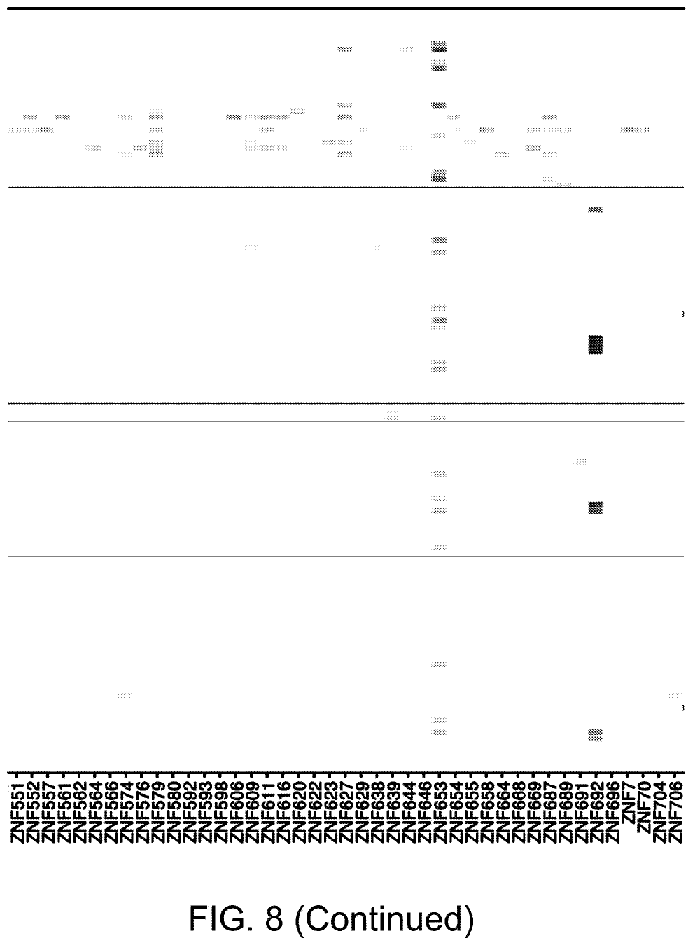

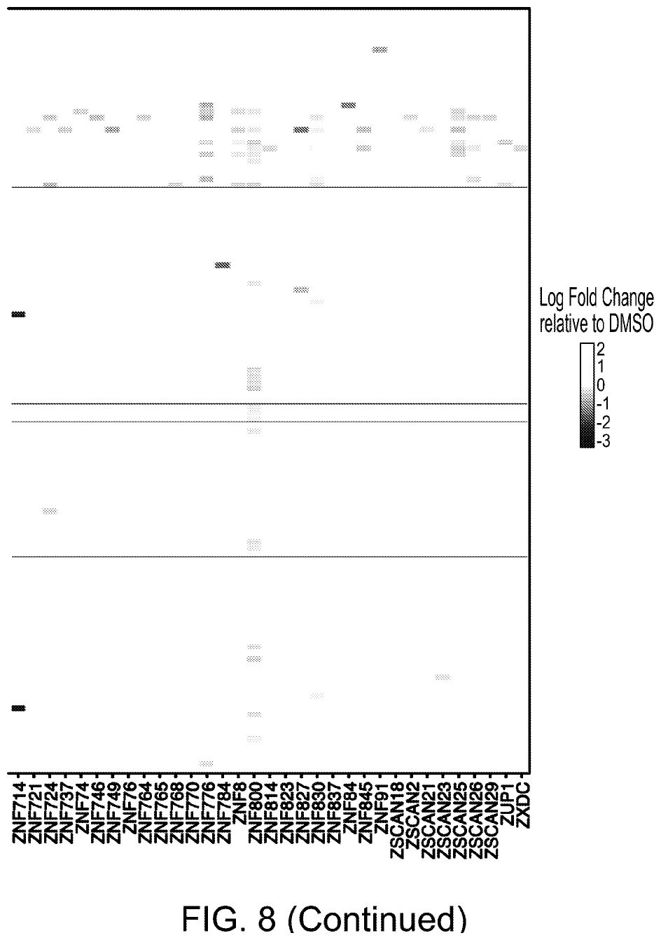

With this assay in hand, off-target activity was profiled of 9 reported PROTACs with varying exit vectors and linker lengths (FIGS. 1B and 6). Significant degradation was observed of many ZF-domains with almost all the 9 PROTACs (FIG. 1B). Notably, PROTACs with common exit vectors such as arylamine, -ether, -carbon, and -amide generally had greater ZF degradation capabilities (FIGS. 1B and 3). This assay also confirmed the off-target degradation of endogenous ZF proteins such as ZFP91 and IKZF3 by commercially available PROTACs MS4078 (ALK PROTAC, FIG. 1C)⁶ and dTAG-13⁷ (FKBP12F36V PROTAC, FIG. 1E), as shown in immuno blots (FIGS. 1D, 1F, and 7). These data suggest that PROTACs with flexible linkers on 4^{th} position of the aryl ring are generally highly degradable. Next, the influence of exit vectors on pomalidomide-based PROTACs and the degradation of ZFs was queried. Towards this goal, changes were analyzed in endogenous ZF proteins from 124 proteomics datasets that were generated for cells treated with pomalidomide-based PROTACs.⁵ The relative abundance of proteins that contained the ZF motif as previously described¹ and were detectable in at least 1 proteomics dataset (i.e., 284 ZF proteins), are shown in Figure S4 (P<0.01). A ZF degradation score was computed for every PROTAC dataset by taking the sum of ZF protein abundance. Analyzing the degradation score distribution confirmed that PROTACs with oxy acetamide exit vectors had significantly reduced ZF protein degradation capacity relative to amino acetamide and arylamine, -ether, and -carbon exit vectors (FIG. 8).

Both the analysis of these proteomic datasets of endogenous proteins and this profiling assay suggests that the flexible exit vectors on position 4 of the aryl ring confers more ZF degradation (FIGS. 1B and 8). The agreement between this automated high-content imaging assay and proteomics suggests that this methodology can be applied in a high-throughput manner to identify new pomalidomide-based PROTACs and pomalidomide analogs that confer minimal ZF degradation. Furthermore, a set of new rules for PROTAC development to minimize off-target degradation of endogenous ZF proteins can determined.

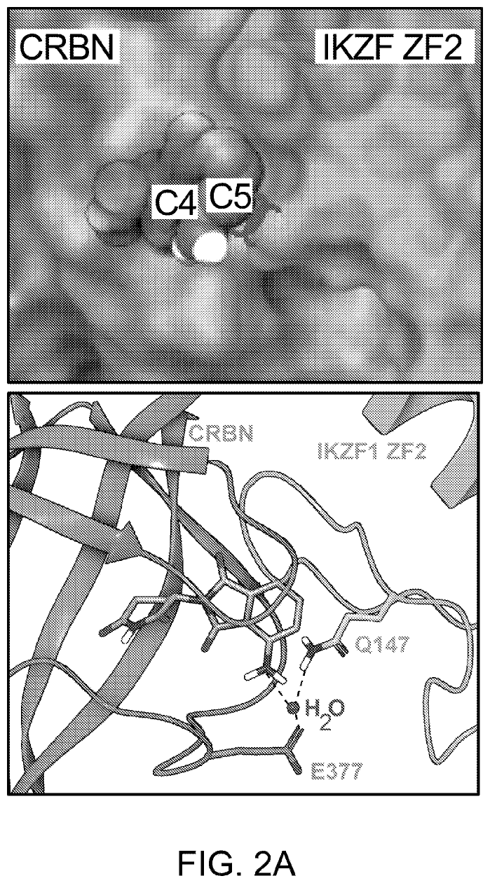

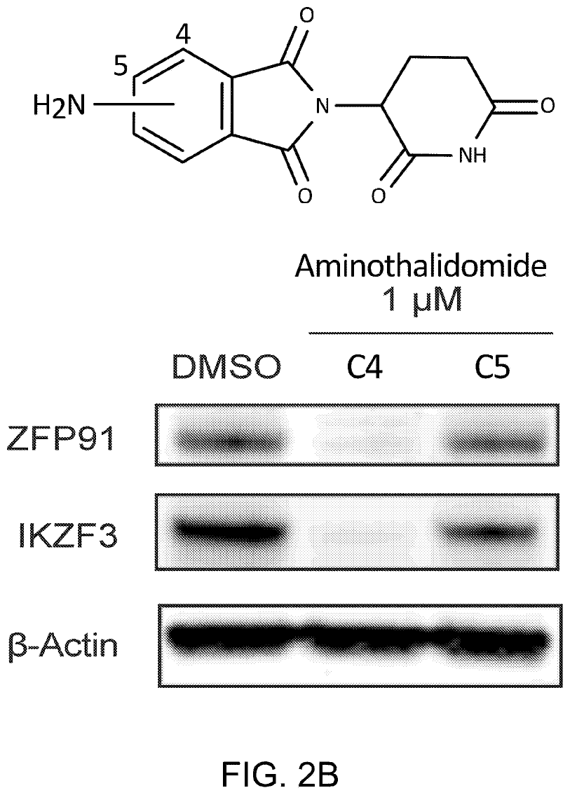

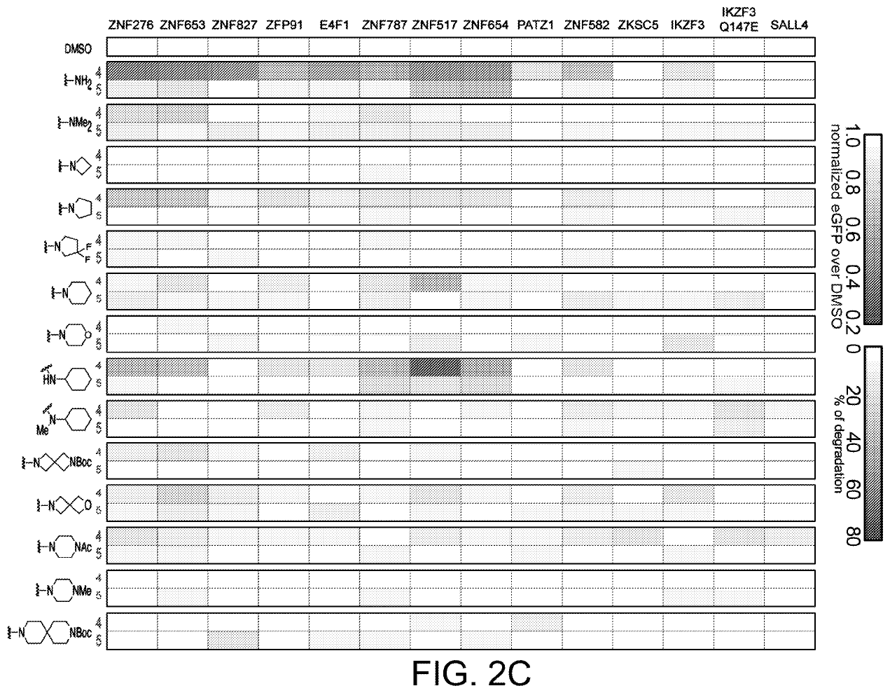

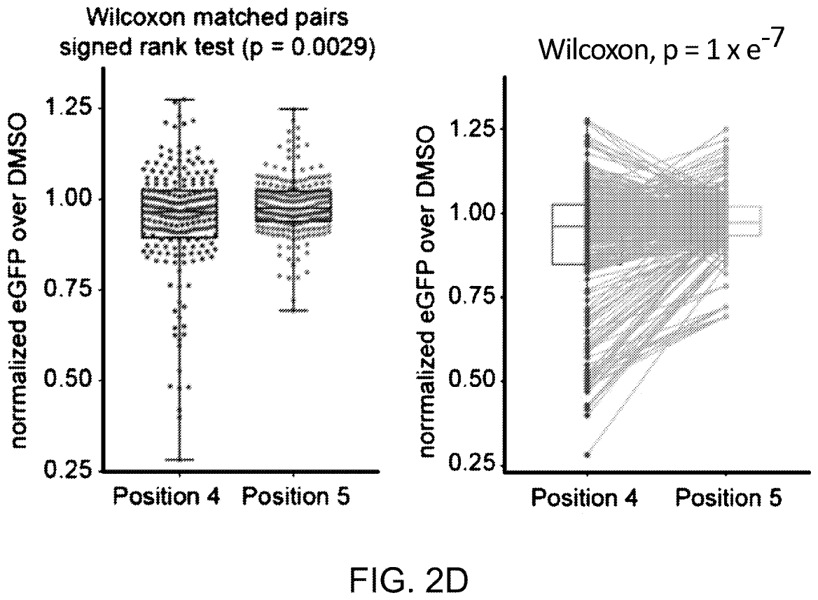

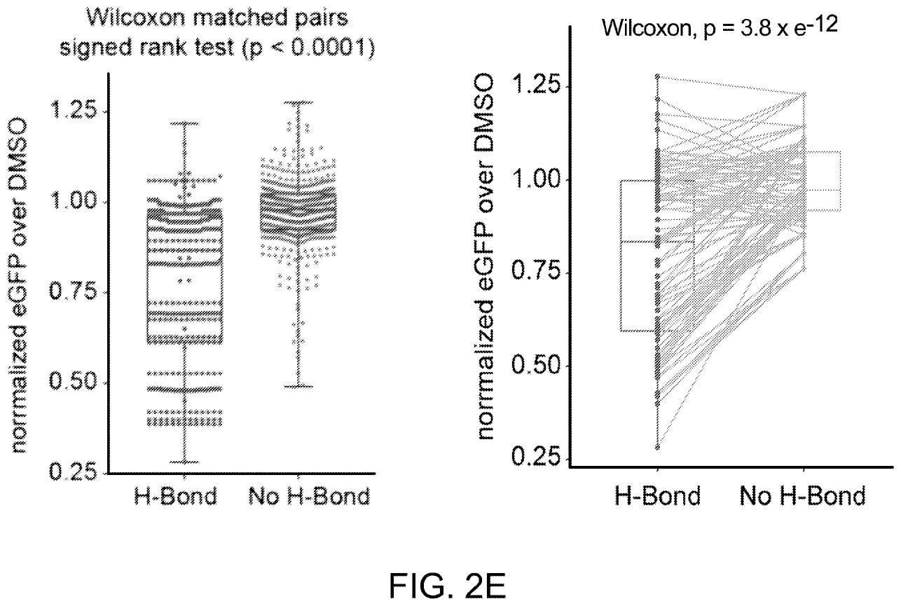

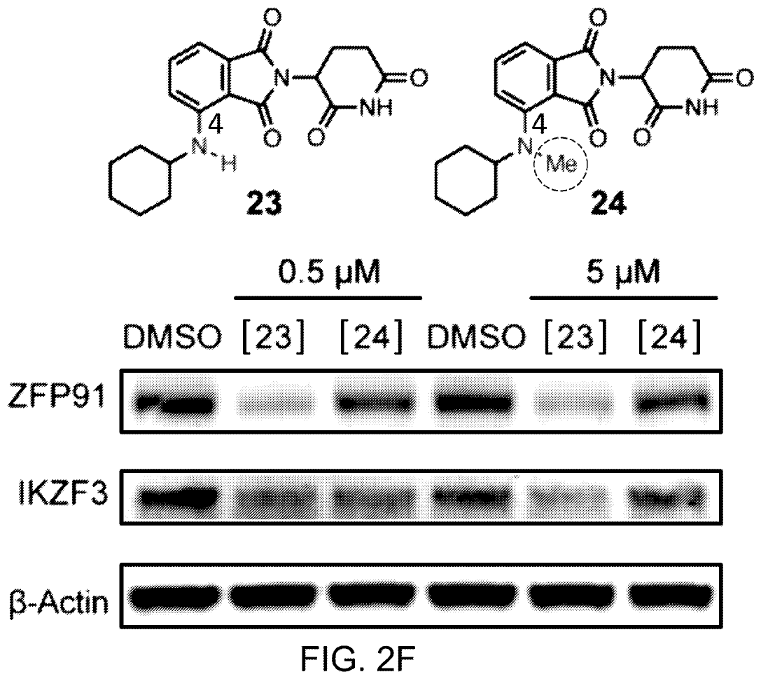

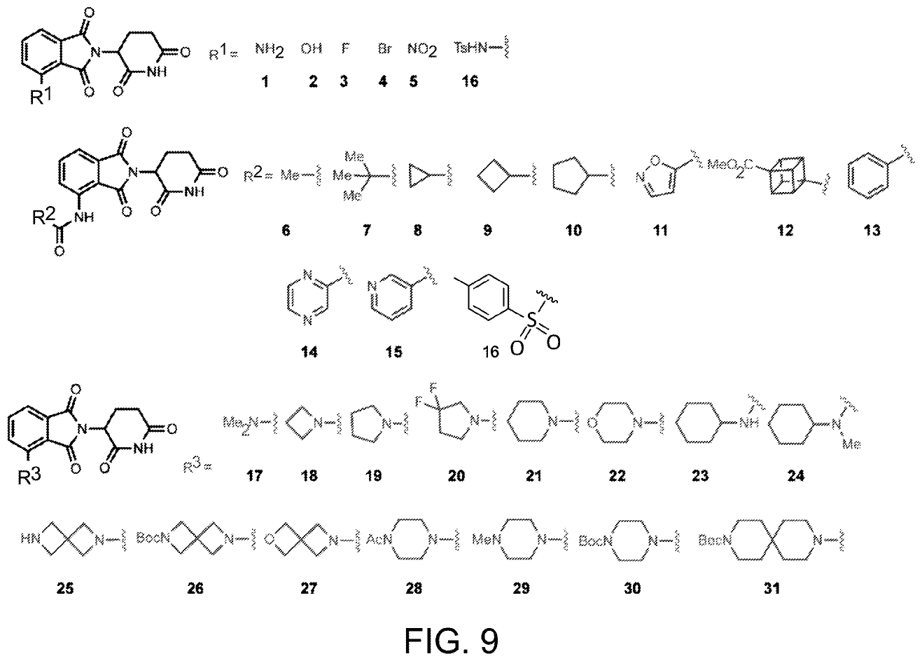

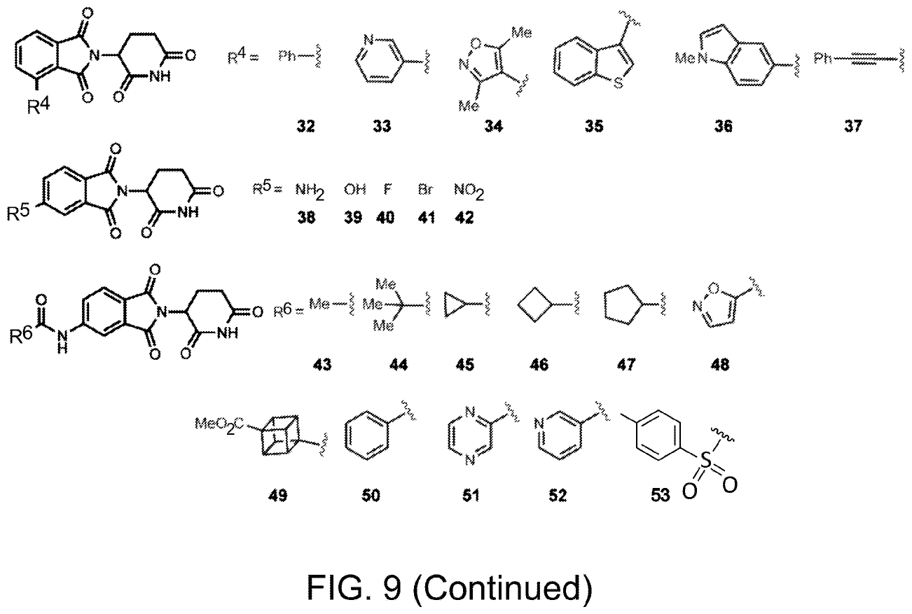

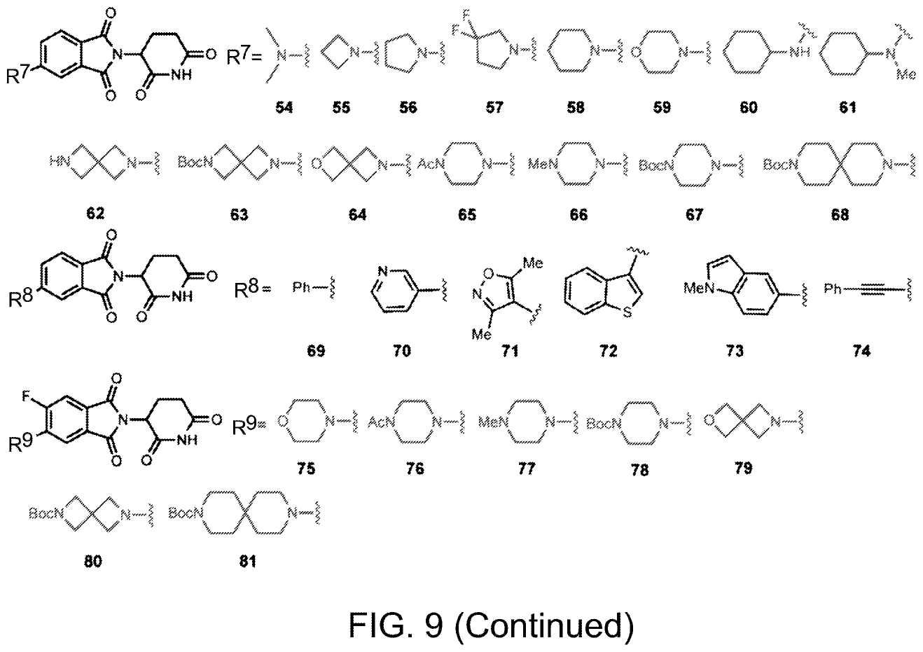

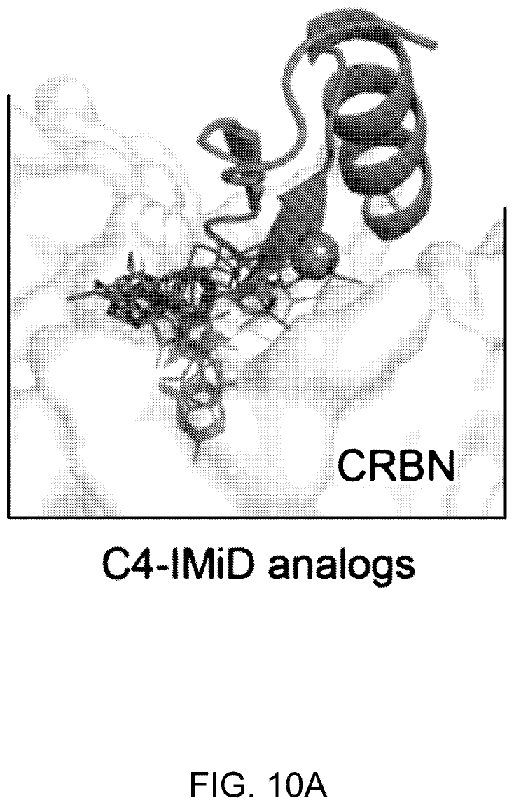

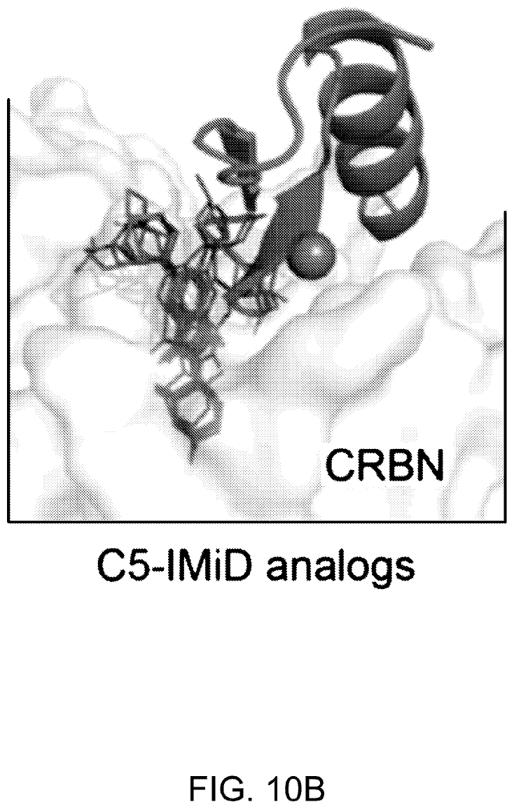

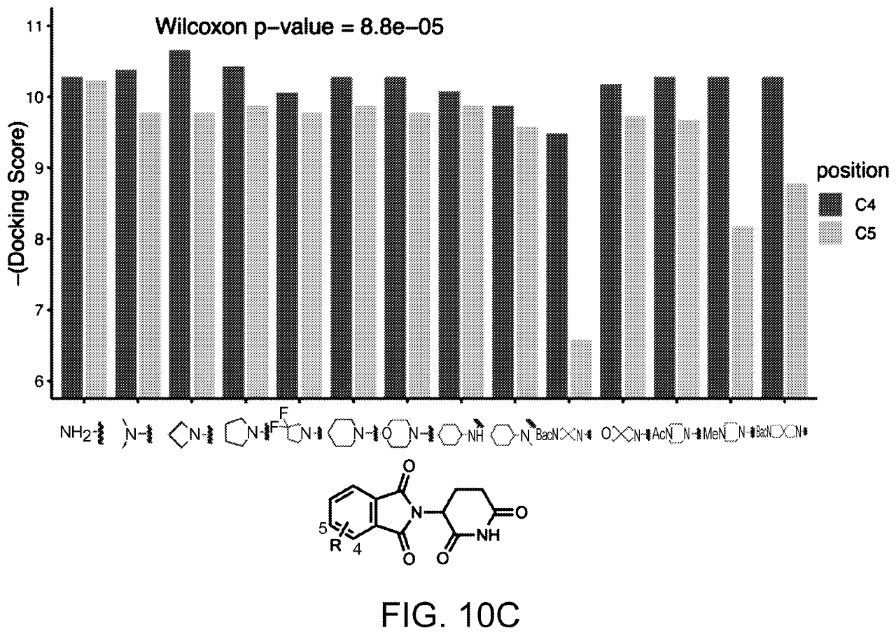

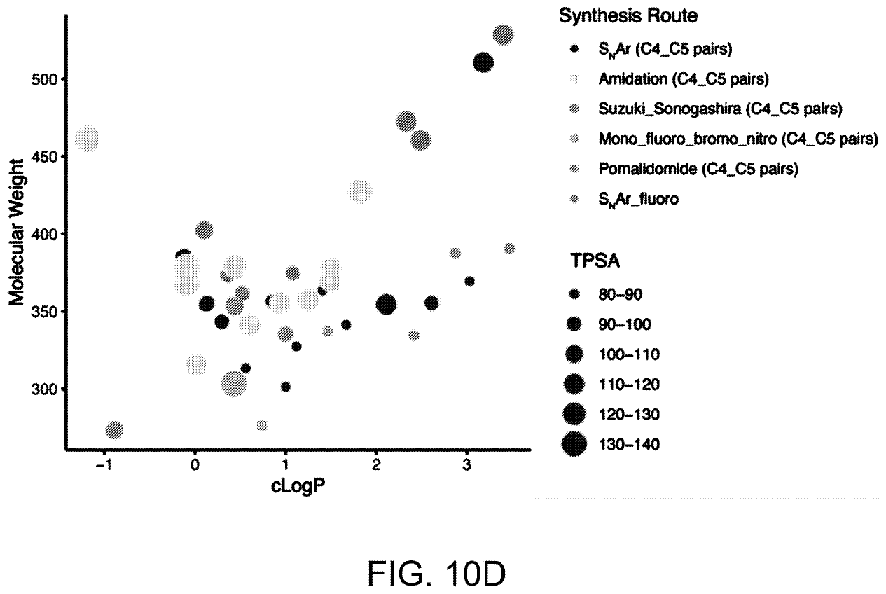

Generation of a library of rationally designed pomalidomide analogs. Next a library of rationally designed pomalidomide analogs was generated that can applied towards the systematic design of pomalidomide-based PROTACs with minimal off-target ZF degradation. Structural insight was gained from the crystal structure of the DDB1-CRBN-pomalidomide complex bound to transcription factor IKZF1 (PDB: 6H0F). In the crystal structure, the glutarimide ring of pomalidomide is deeply buried inside the CRBN while the imide ring is exposed. Q146 of IKZF1 forms a water-mediated hydrogen-bonding interaction with the C4 amino group of the compound, while the C5 position is proximal to the ZF domain (FIG. 2A)². Mutation of Q146, or equivalent residues, has been reported to abrogate IKZF degradation. It was hypothesized that appropriate substitutions at the C4 and/or C5 positions can disrupt the ternary complex of the ZF domains with CRBN. First synthesized was a Thalidomide analog with amino group on C5 position and its investigation in MM1.S cells revealed decrease in the potency of IMiD with C5 amino group for degradation over pomalidomide (FIG. 2B). This suggests that modifications on the C5 positions will bump-off the endogenous ZFs while preserving the CRBN interaction. Therefore, 80 imide analogs (Table 1 and FIG. 9) were generated with varying exit vector modifications on the C4 and C5 positions of the imide ring. The modified analogs should not affect the recruitment of CRBN for induction of protein degradation since the imide ring does not interact with the ubiquitin ligase (FIGS. 2A and 10A-10C).

To rapidly and systematically construct the library of pomalidomide analogs, reactions like amidation, nucleophilic aromatic substitution (SNAr), Suzuki, and Sonogashira cross-couplings were leveraged with commercially available imide synthons for the facile and scalable incorporation of C4 and C5 substitutions on the phthalimide ring. The pomalidomide analogs were synthesized in pairs at C4 and C5 positions a library of 80 compounds was generated that was categorized into three main synthetic groups: N—C (acylation), C—N (SNAr), and C—C (Suzuki/Sonogashira cross-coupling) (Table 1 and FIG. 9). Amidations were carried out with a diverse class of acids varying from aliphatics to heterocyclic cores and varied the sizes of the carboxylic acid to range from acetic acid to the largest cubane carboxylic acid. A number of aliphatic amines were employed with variable sizes high yielding SNAr reactions with 4- and 5-F thalidomides. For the SNAr library, a number of aliphatic amines were incorporated like N-Boc piperazine and N-Boc diazaspiro[3.3]heptane, which can subsequently be used for PROTAC synthesis after validation. Alongside these SNAr libraries, several pomalidomide analogs were synthesized with a fluoro substitution at the 6-position of thalidomide as well as a number of compounds with heterocyclic boronic acids and phenylacetylene coupled with the 4- and 5-bromo thalidomides. The physicochemical properties of the overall library encompass a reasonable distribution of drug-like properties, such as partition coefficient, or c Log P (−2 to 4), molecular weights (250 to 550 g/mol), and topological polar surface area (tPSA, 80 to 140 Å²) (FIG. 10D).

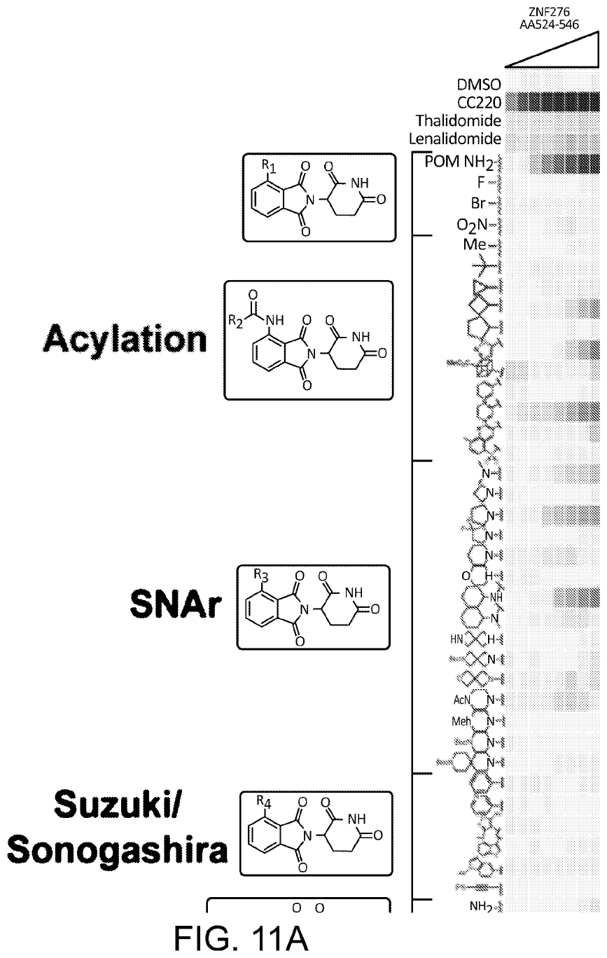

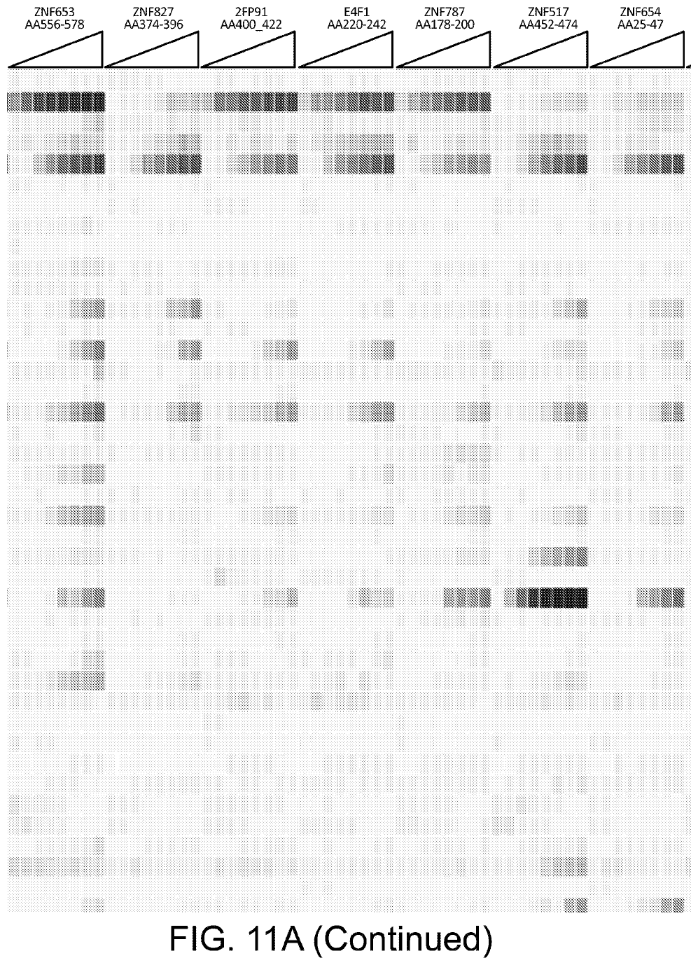

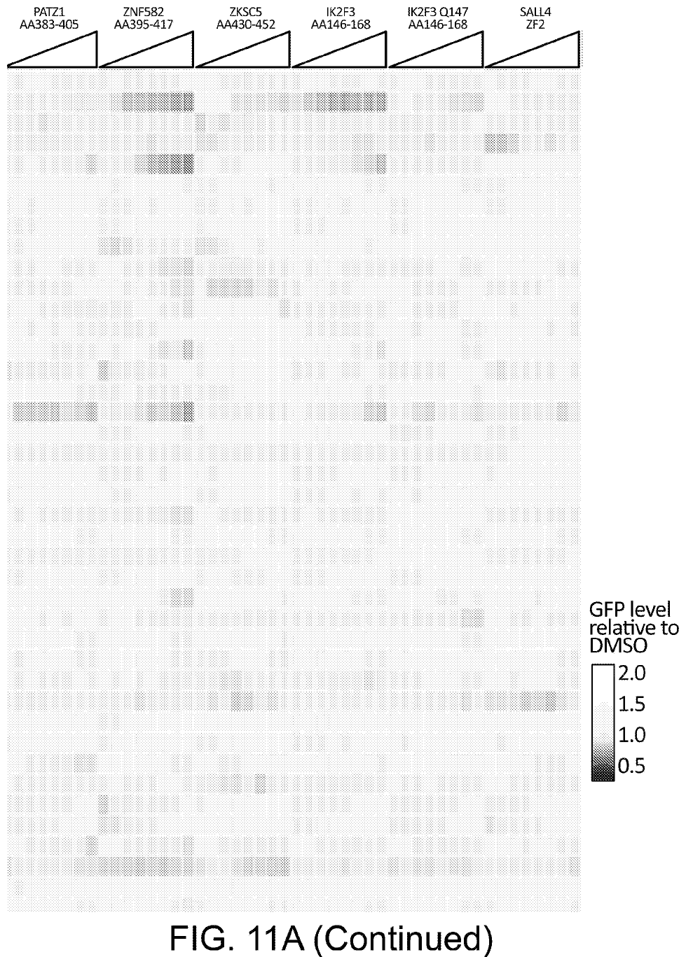

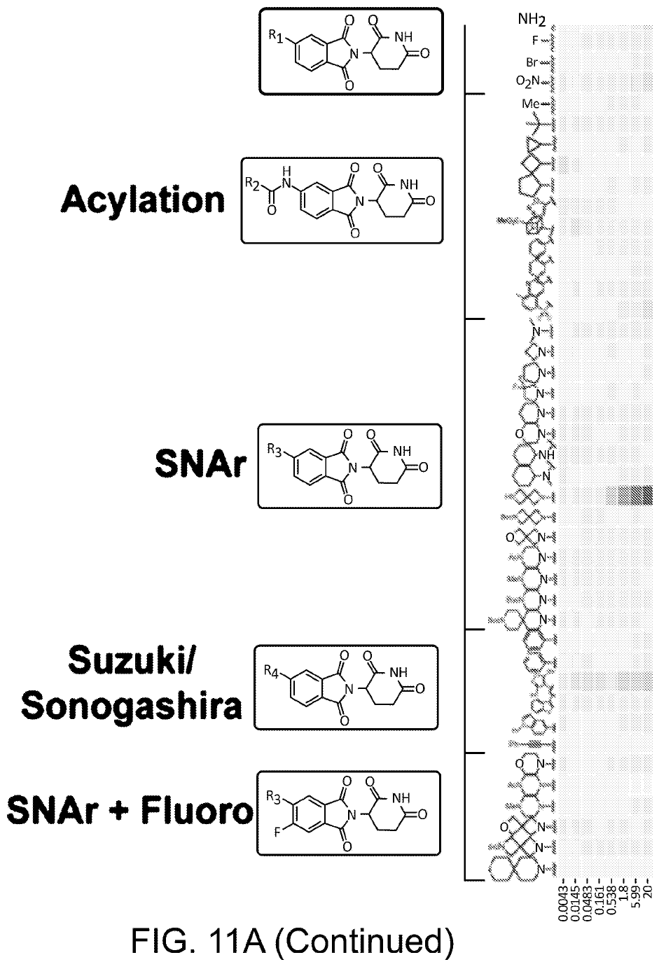

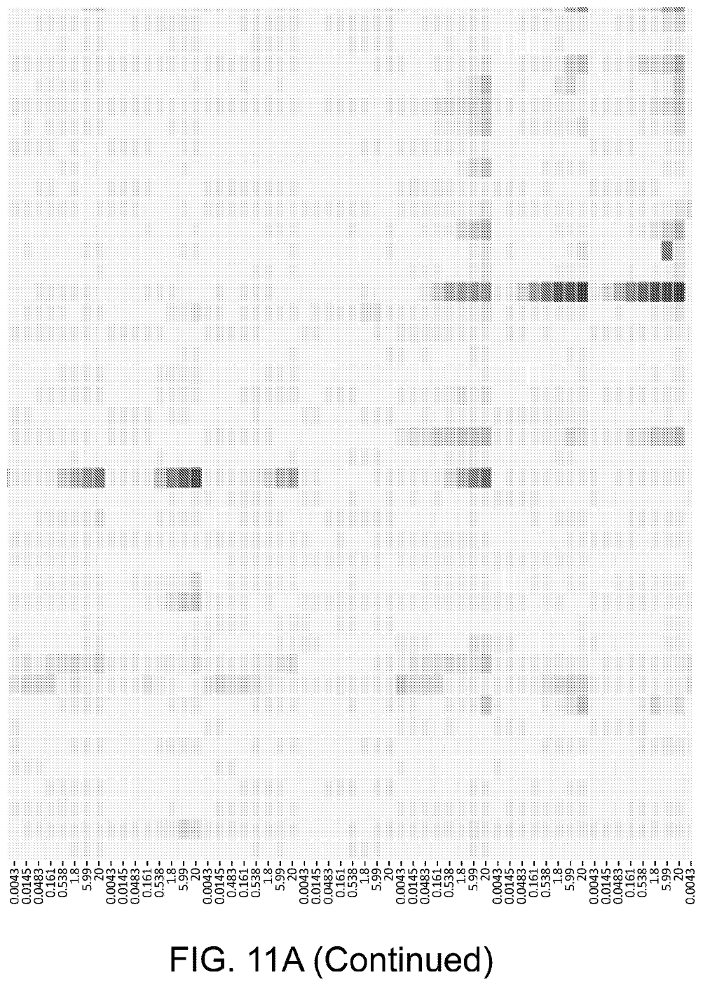

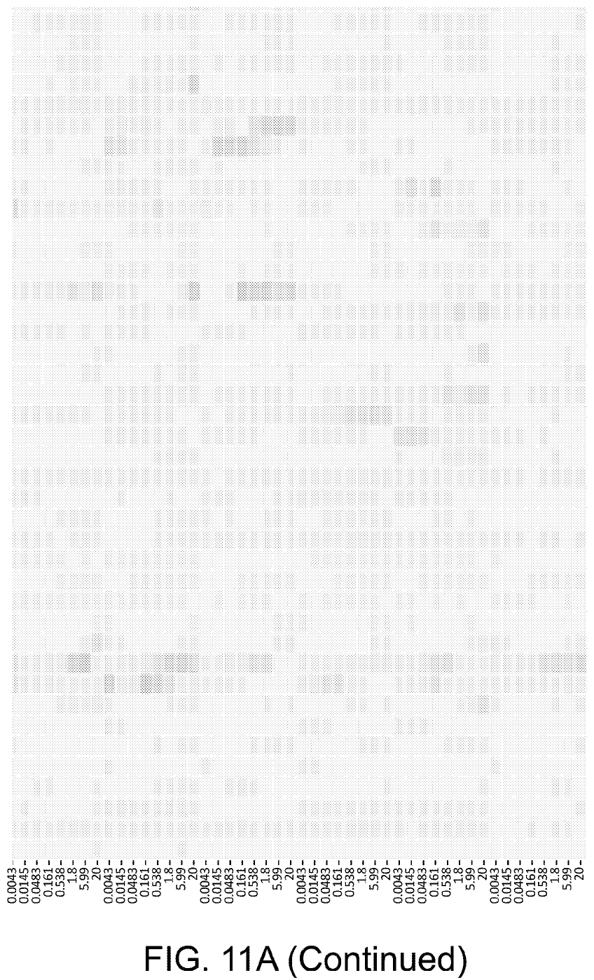

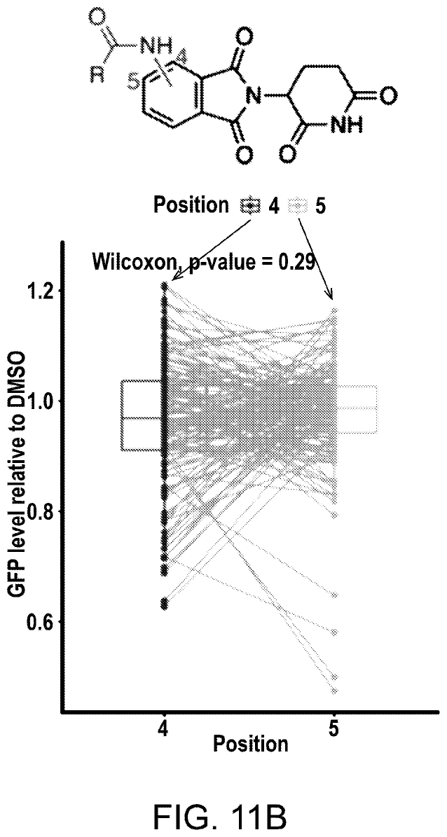

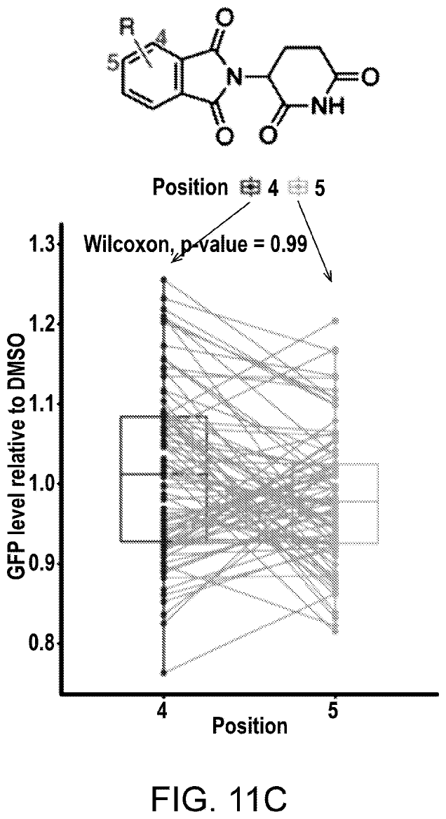

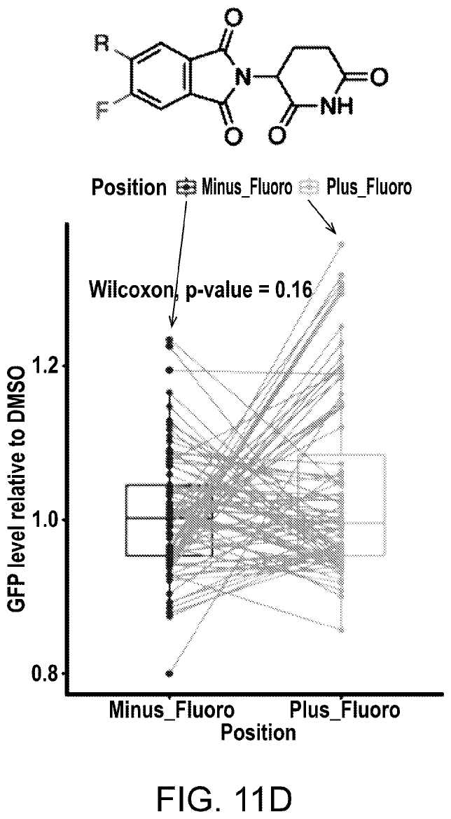

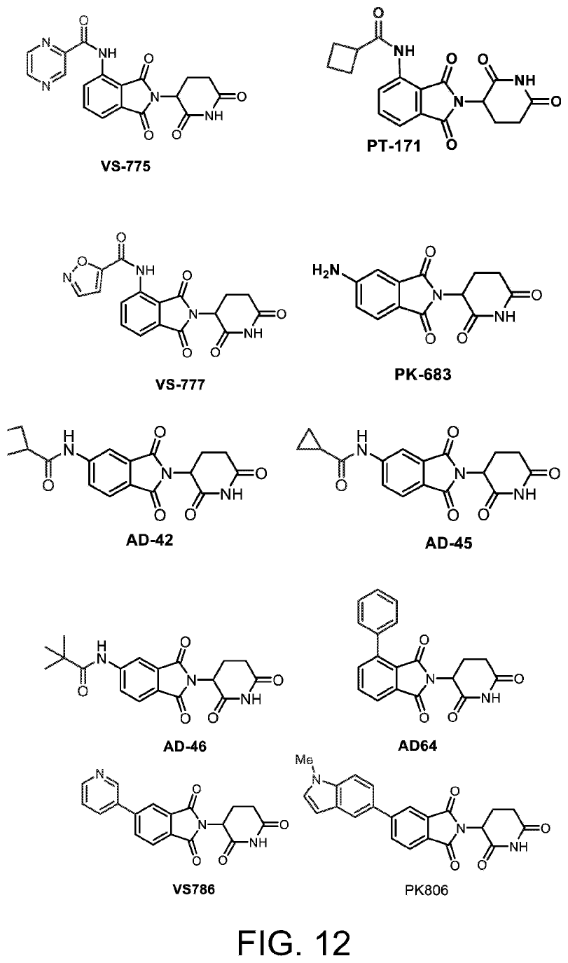

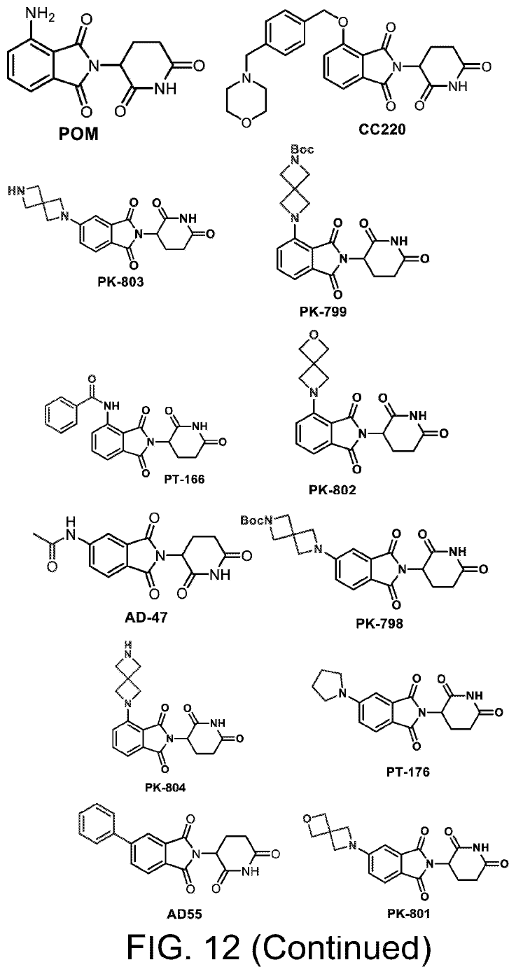

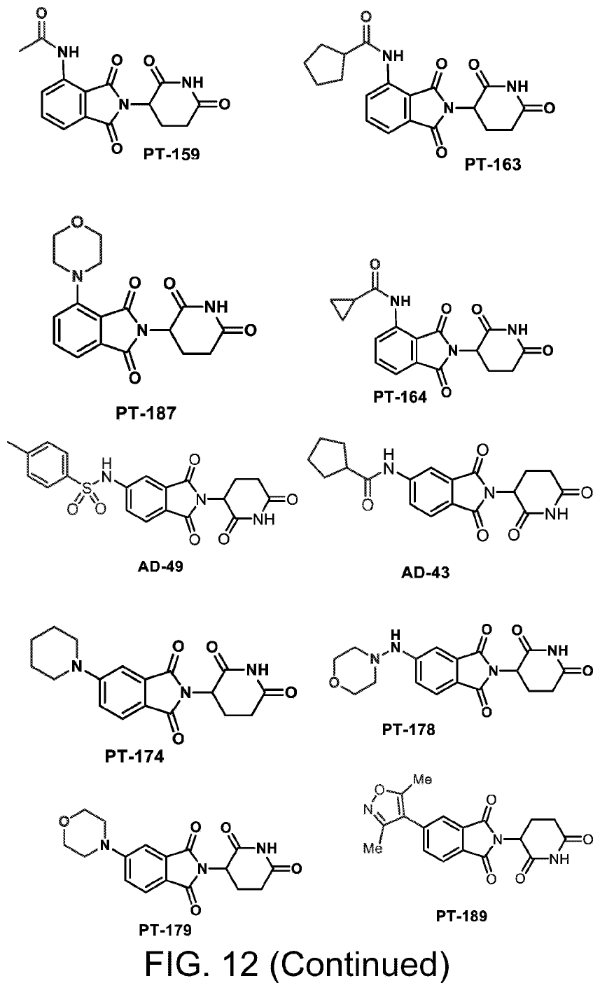

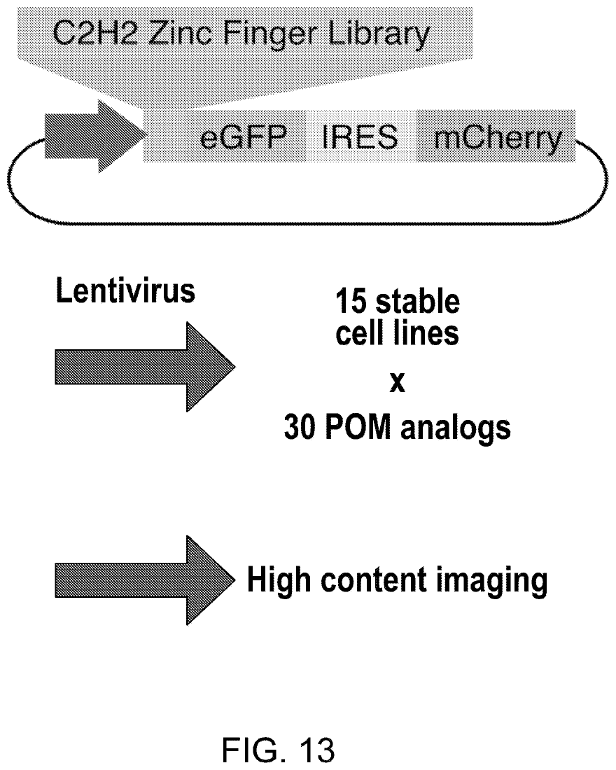

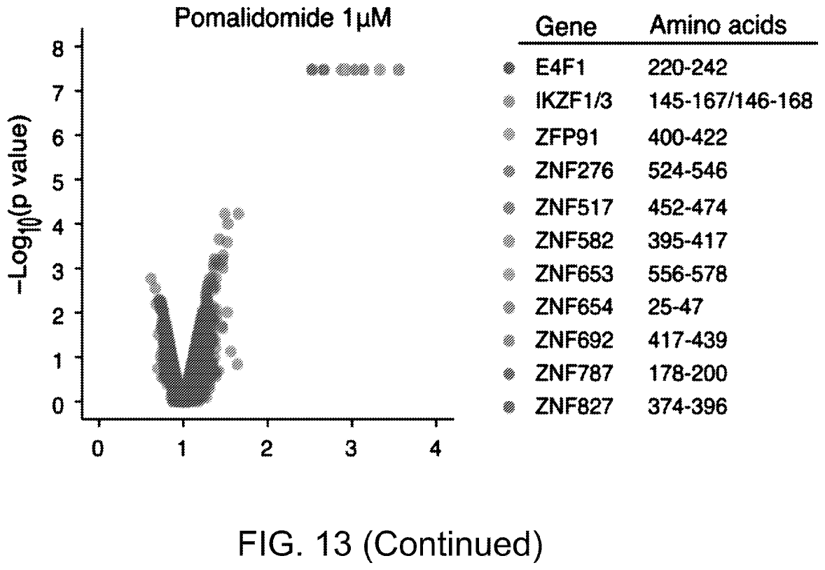

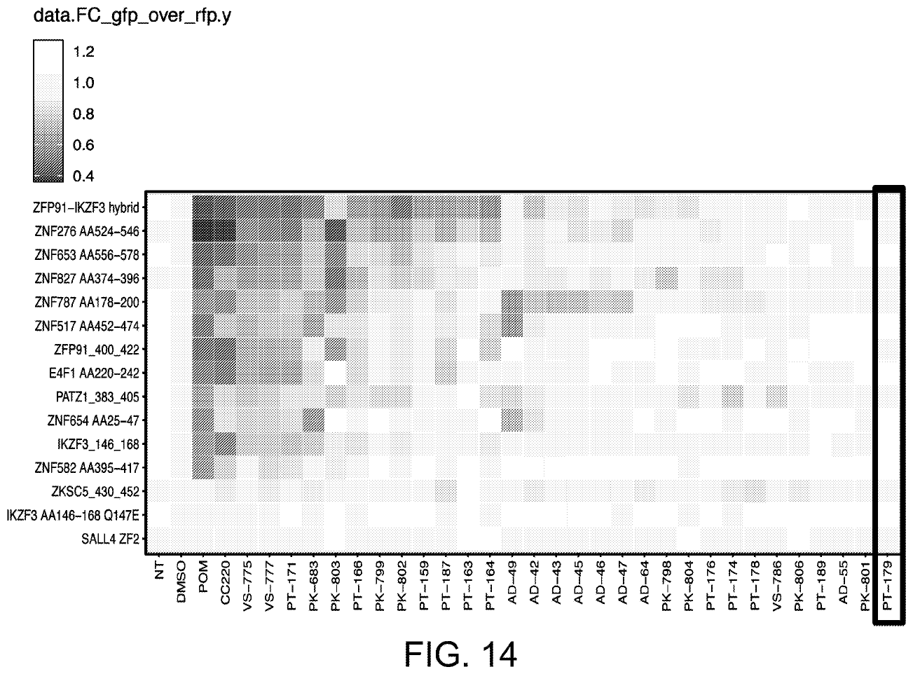

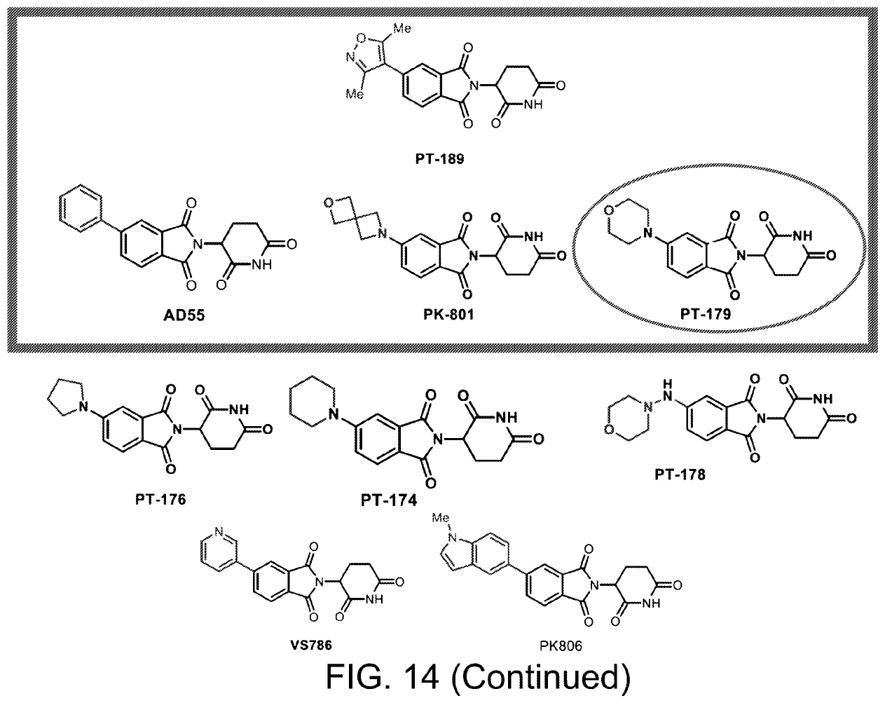

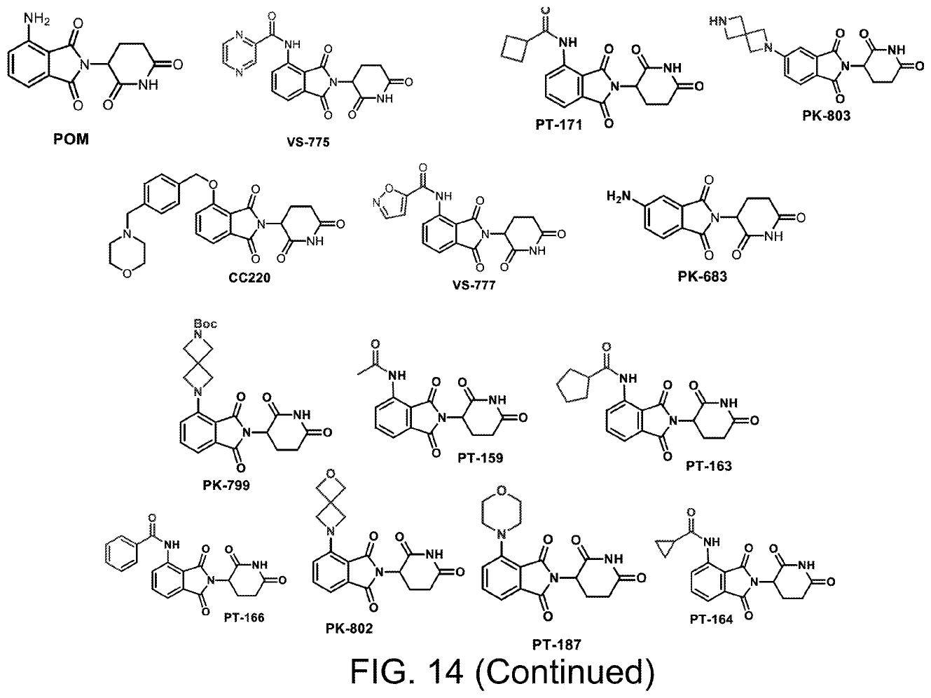

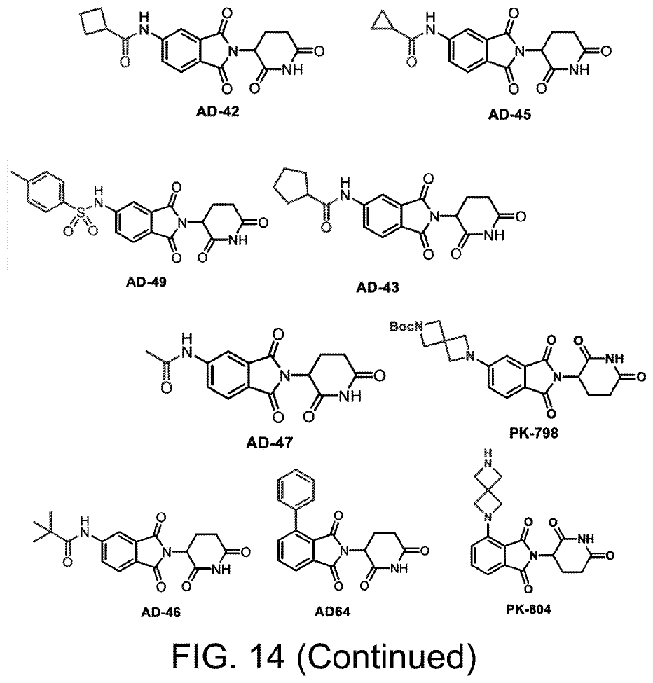

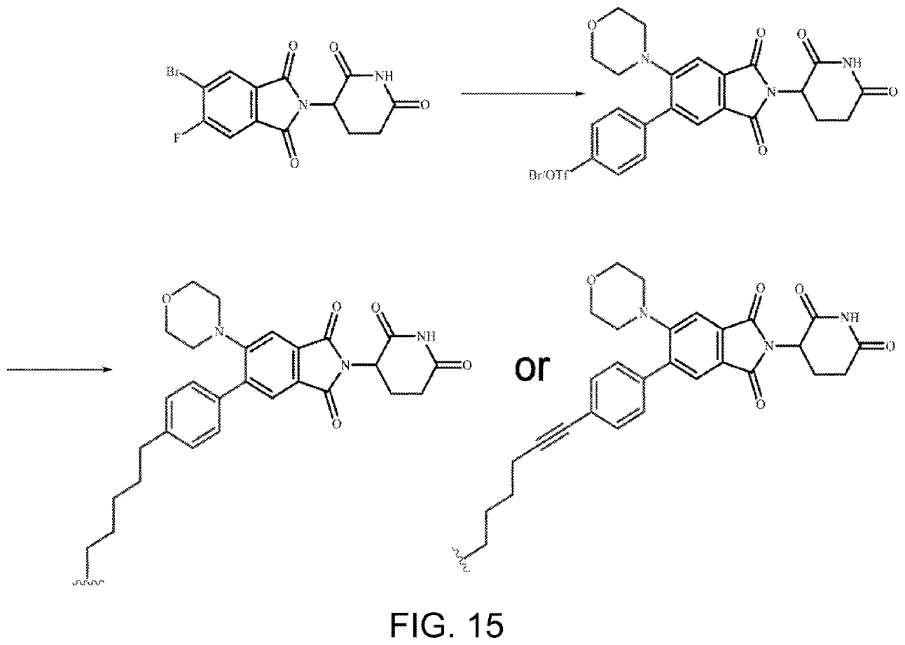

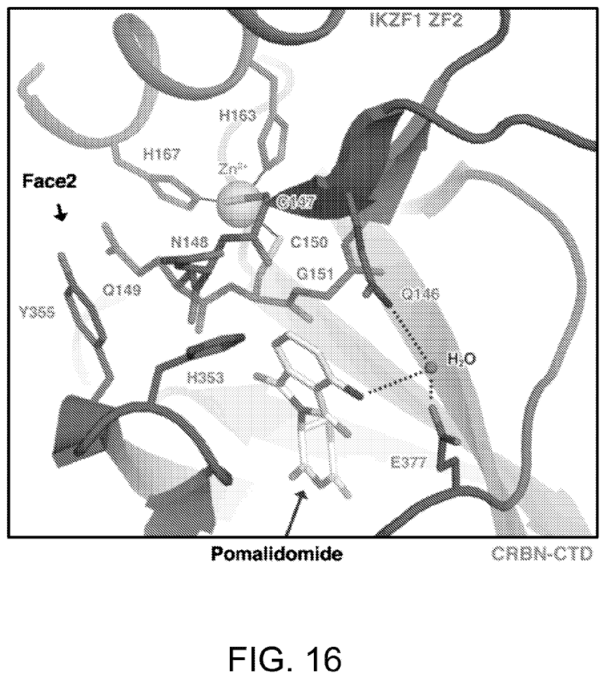

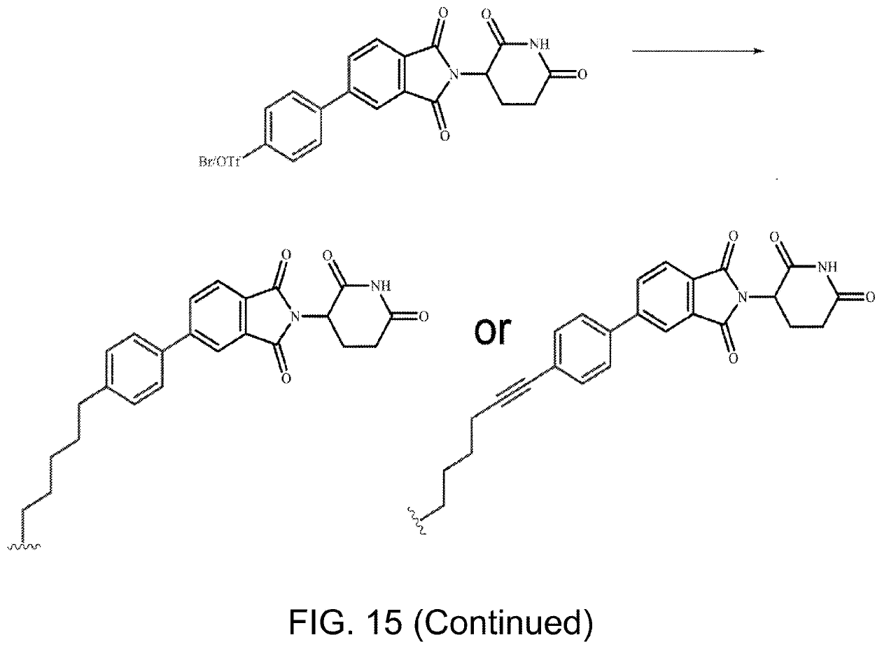

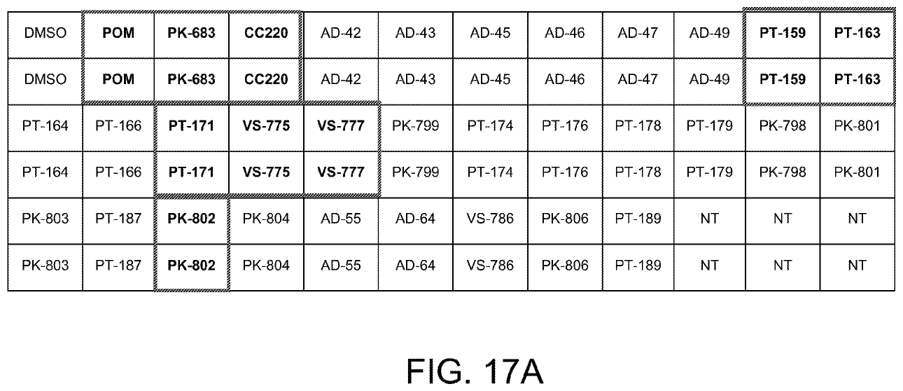

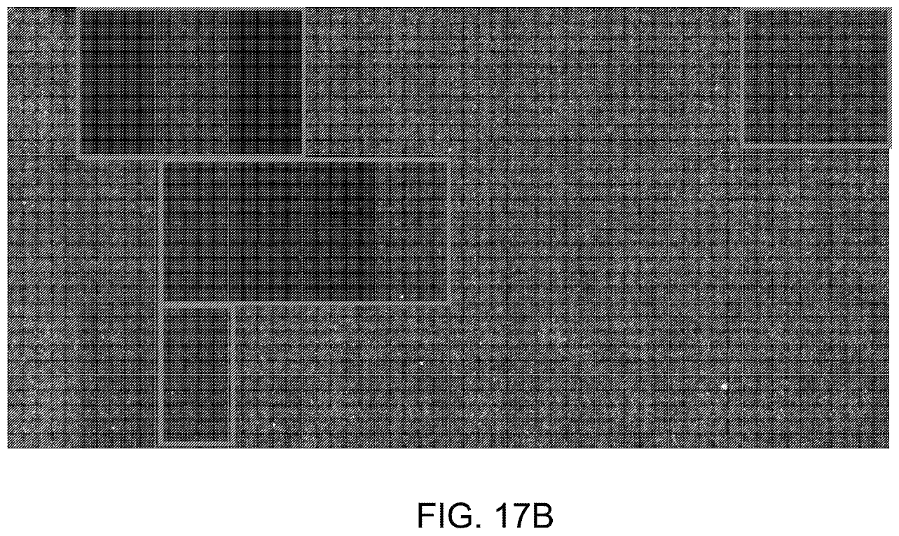

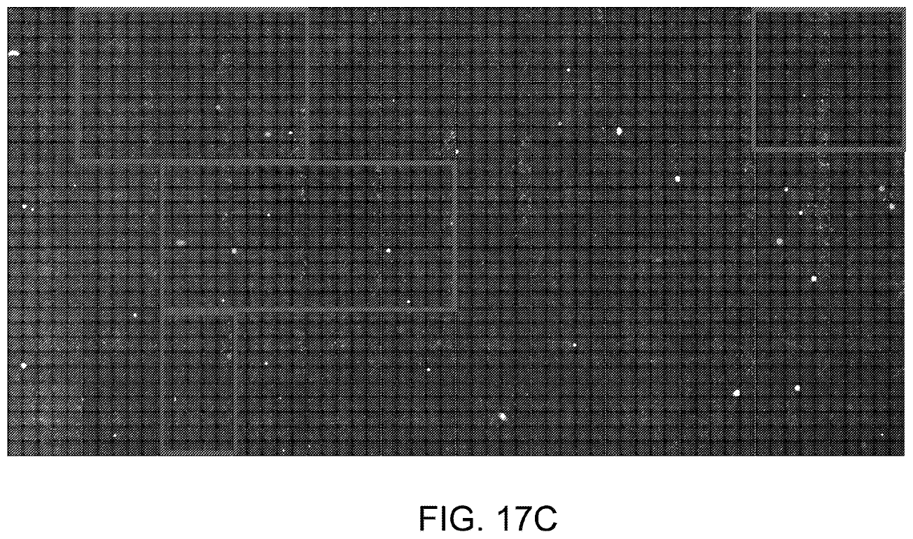

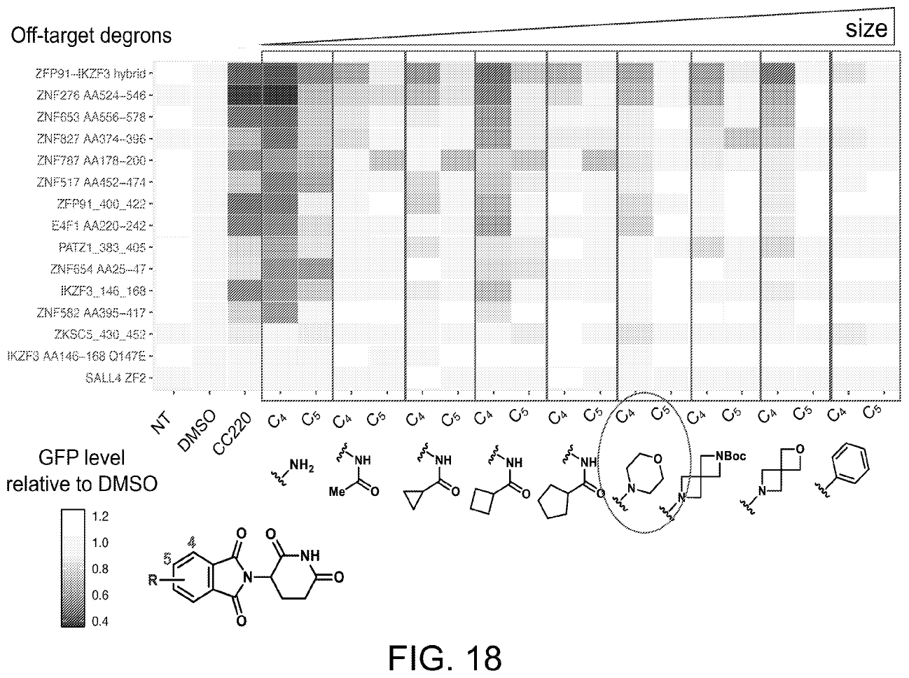

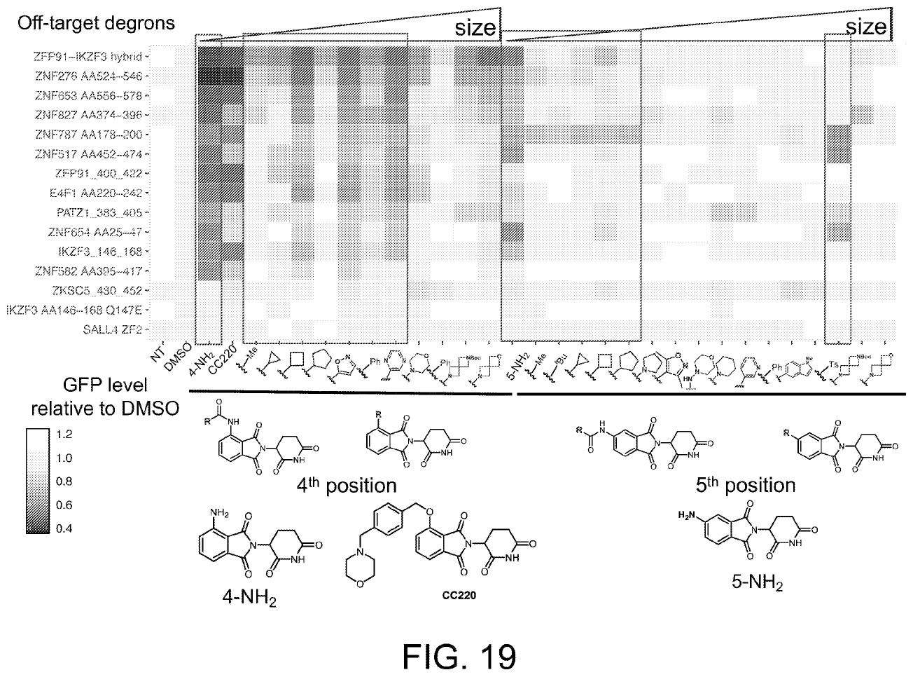

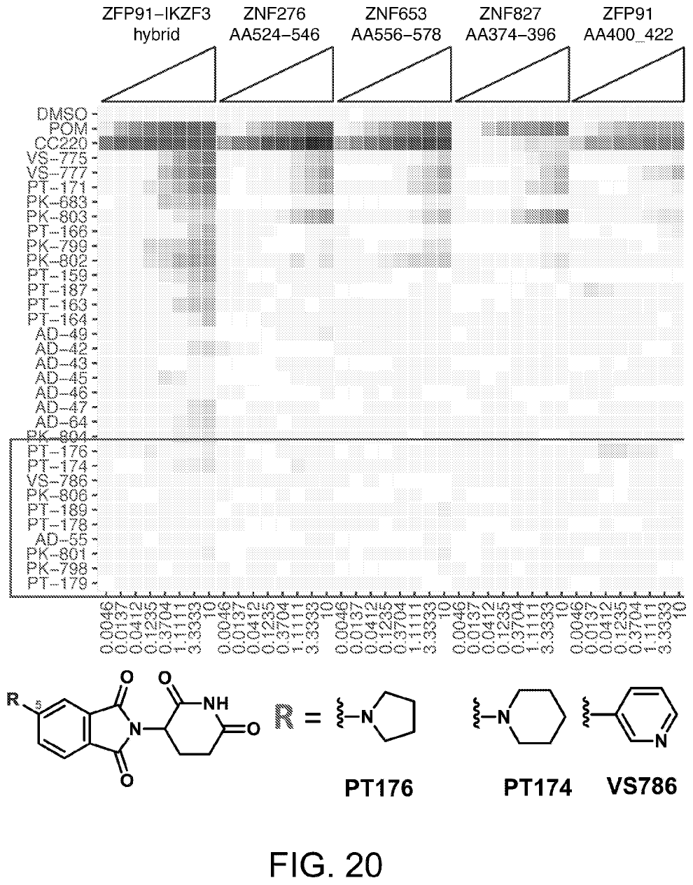

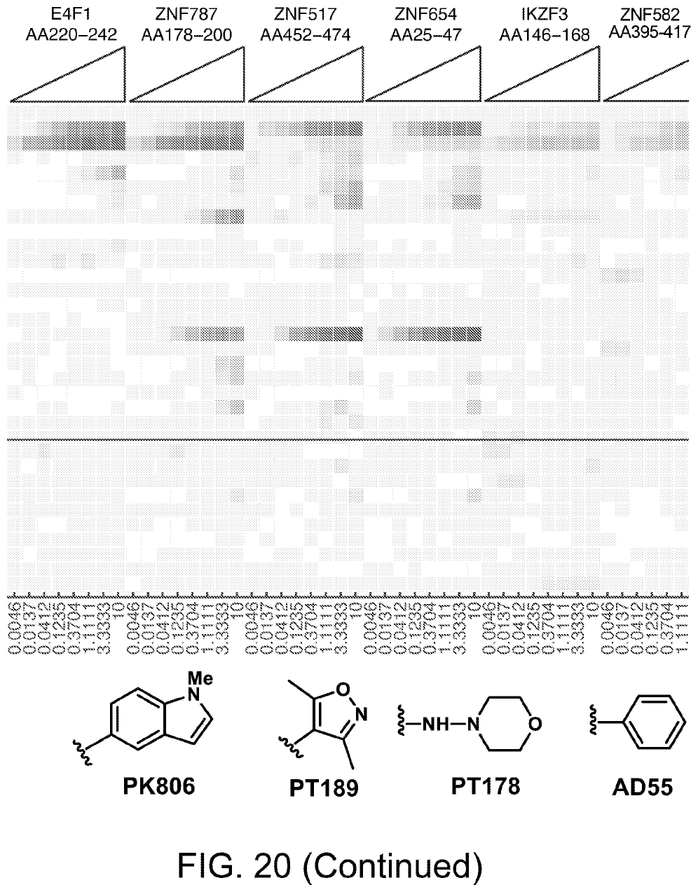

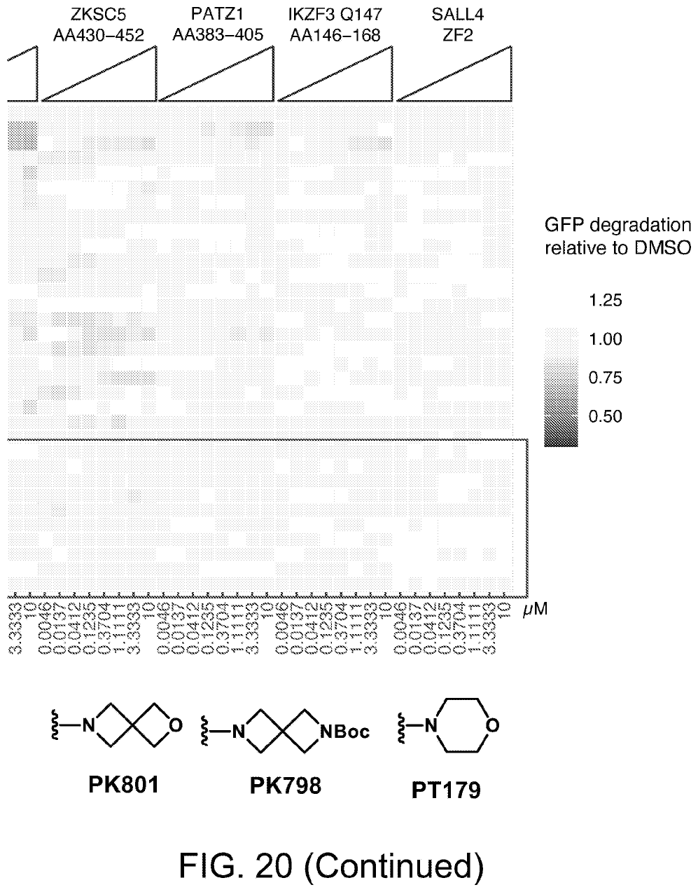

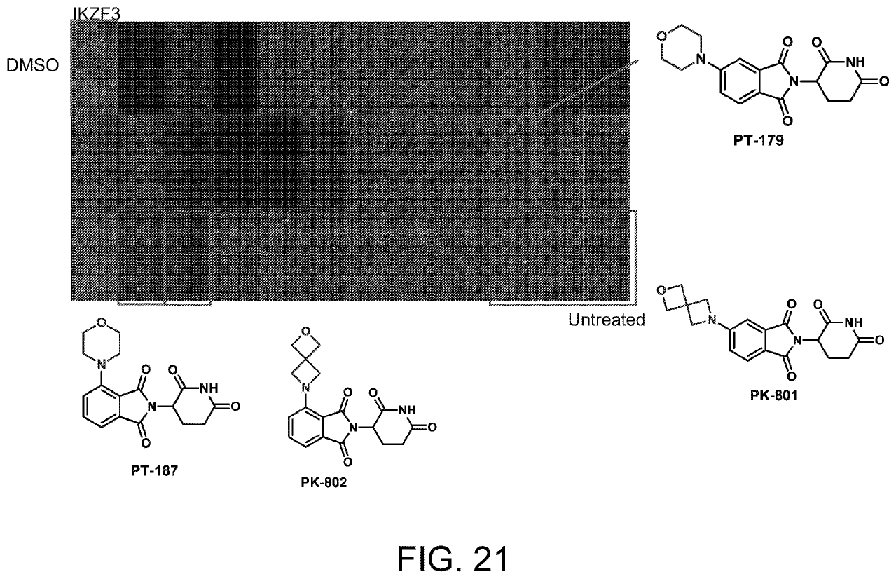

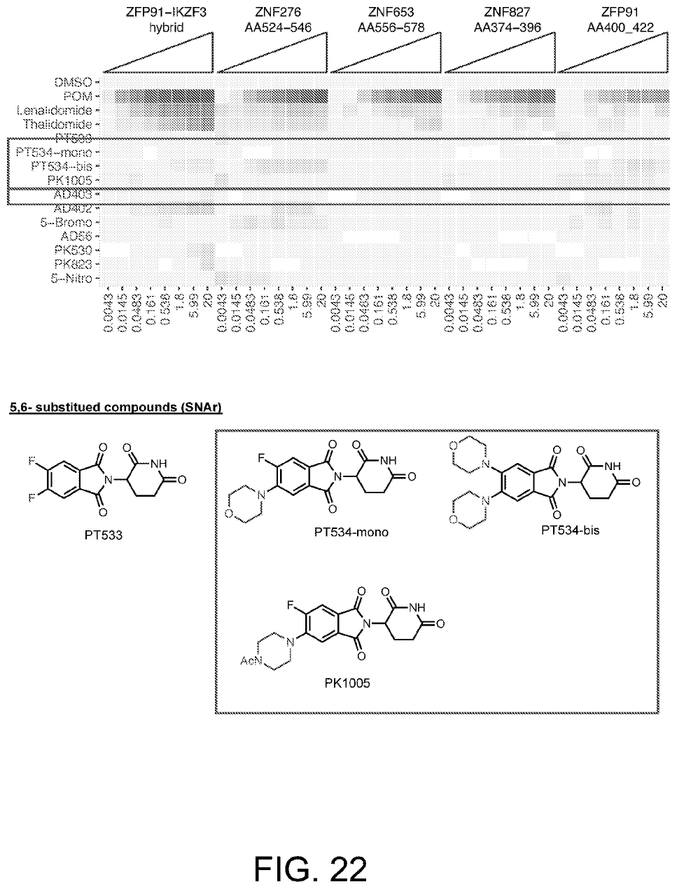

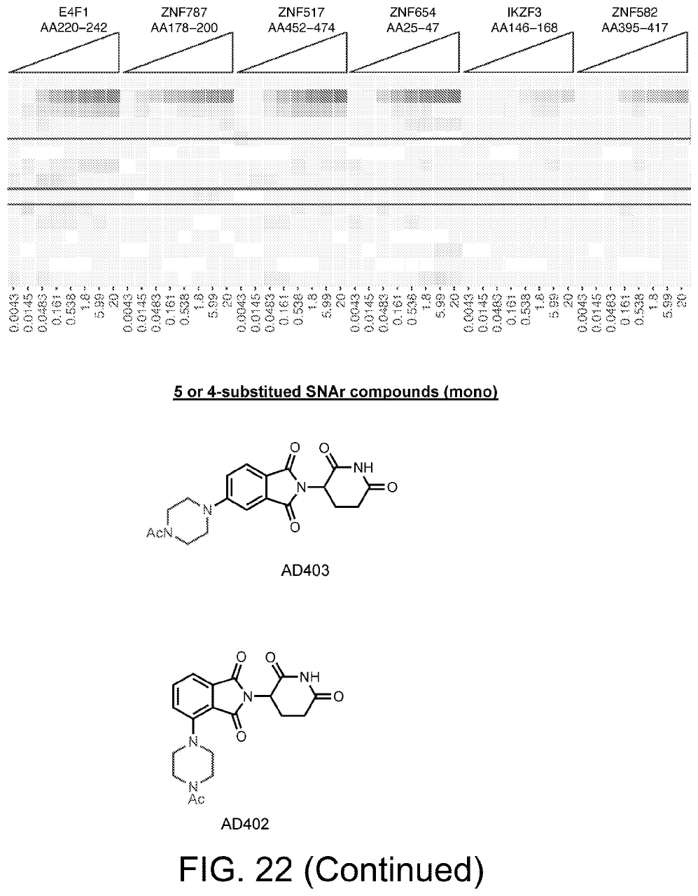

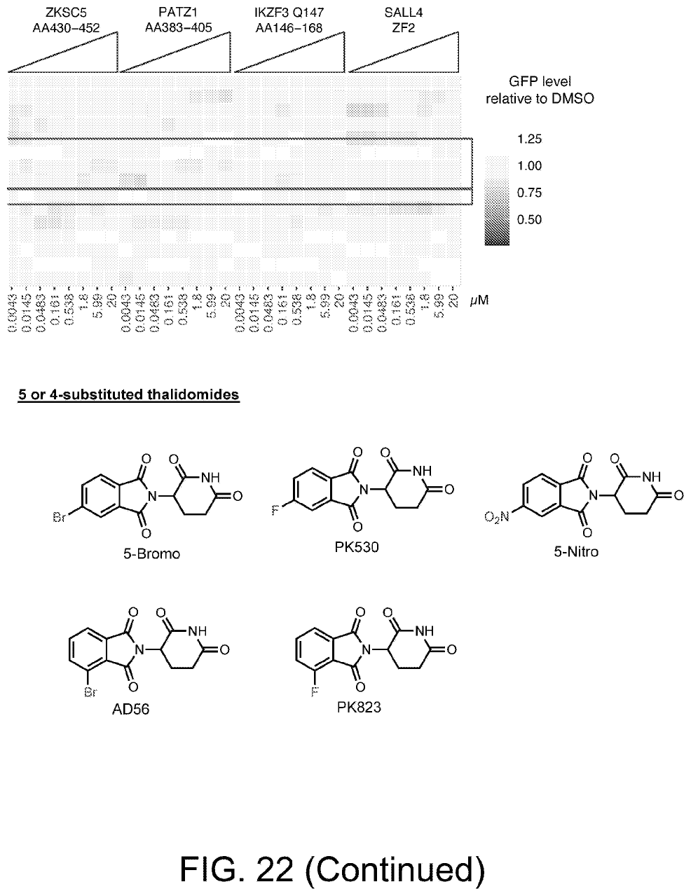

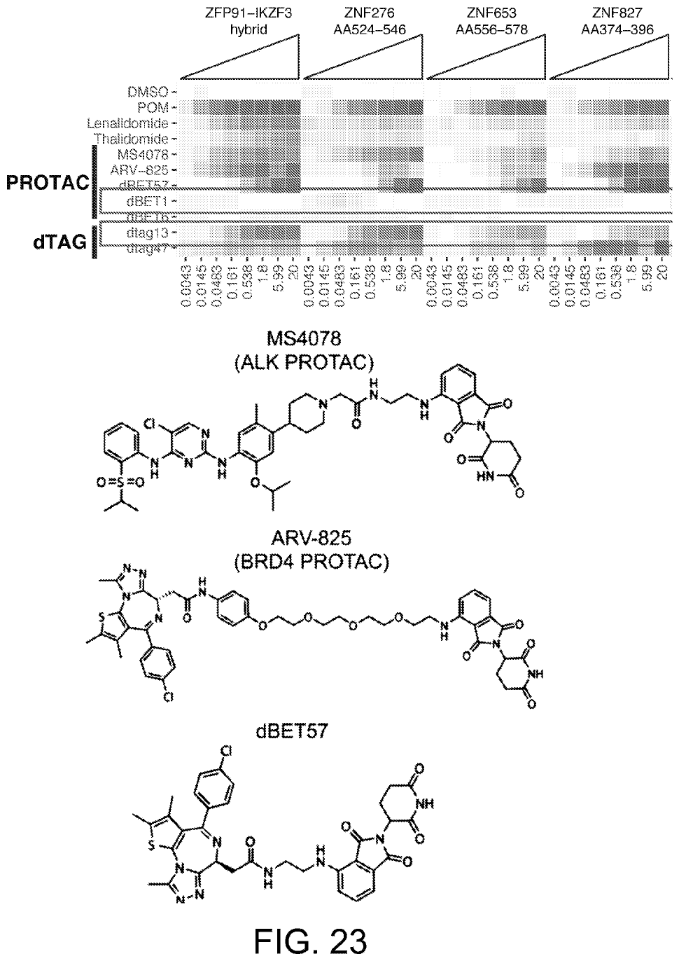

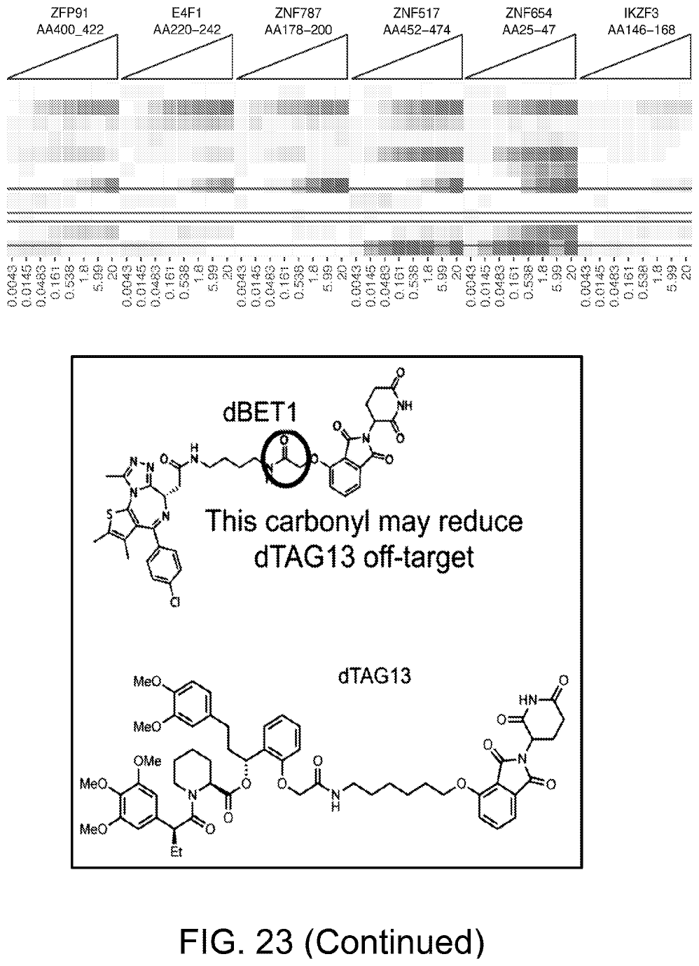

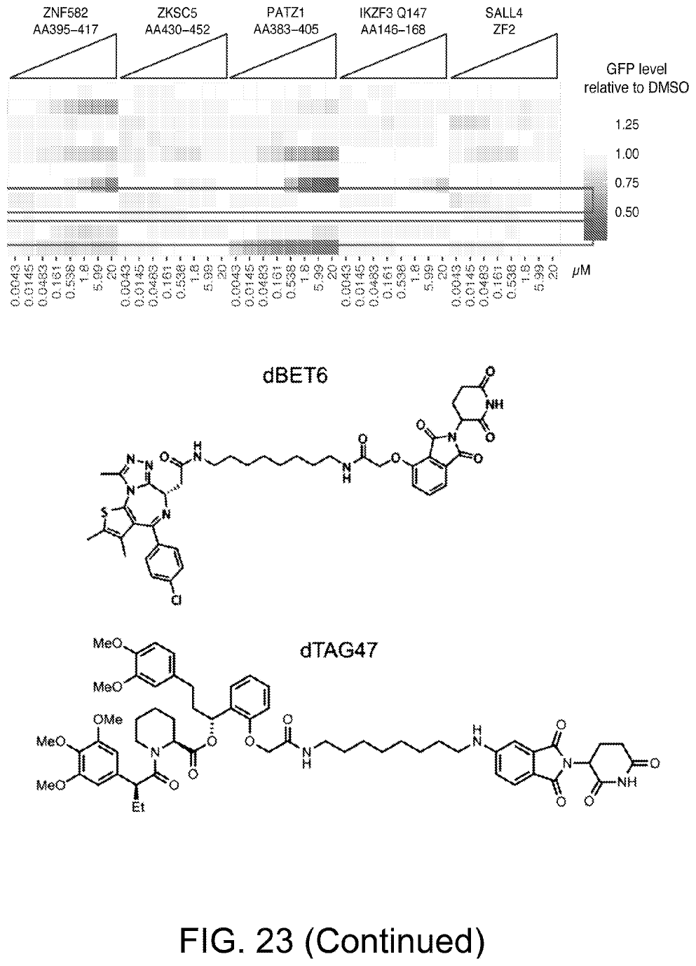

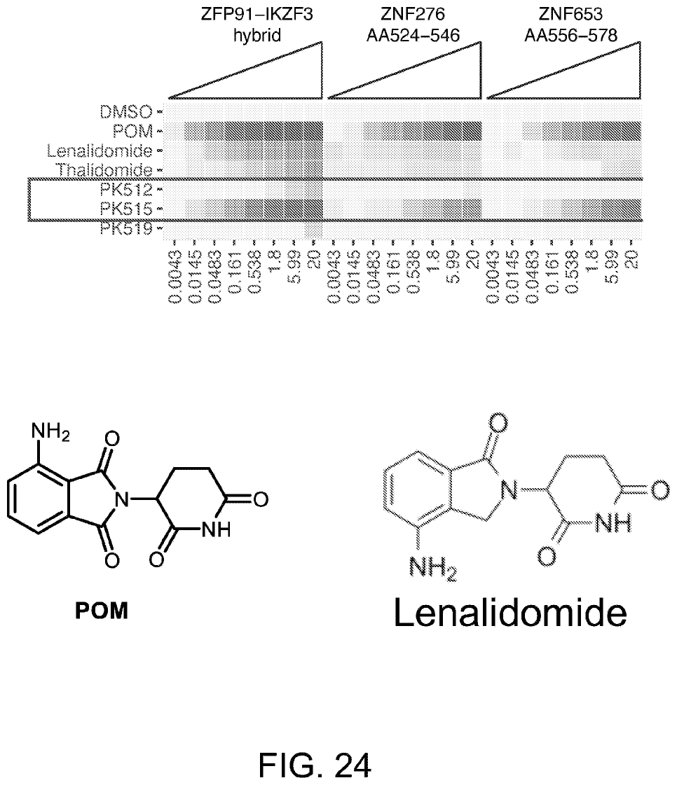

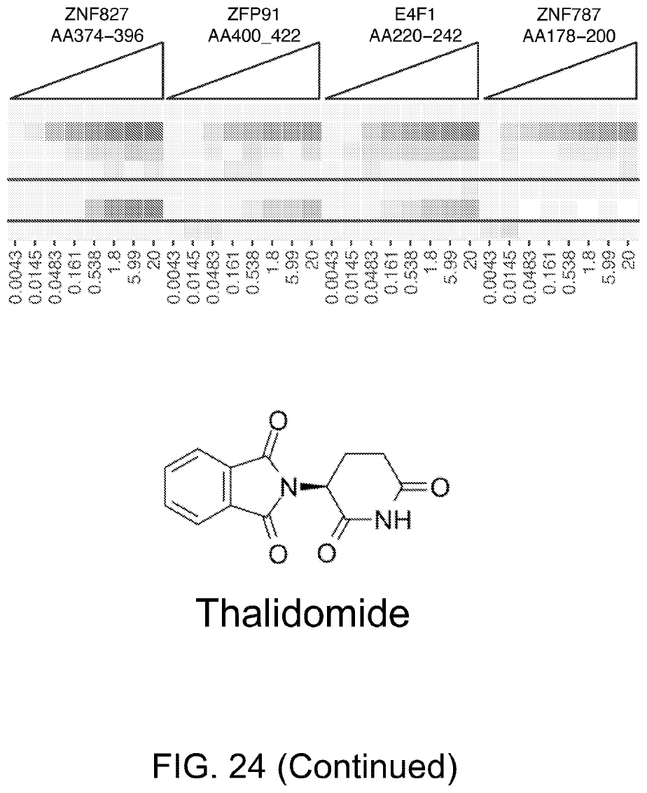

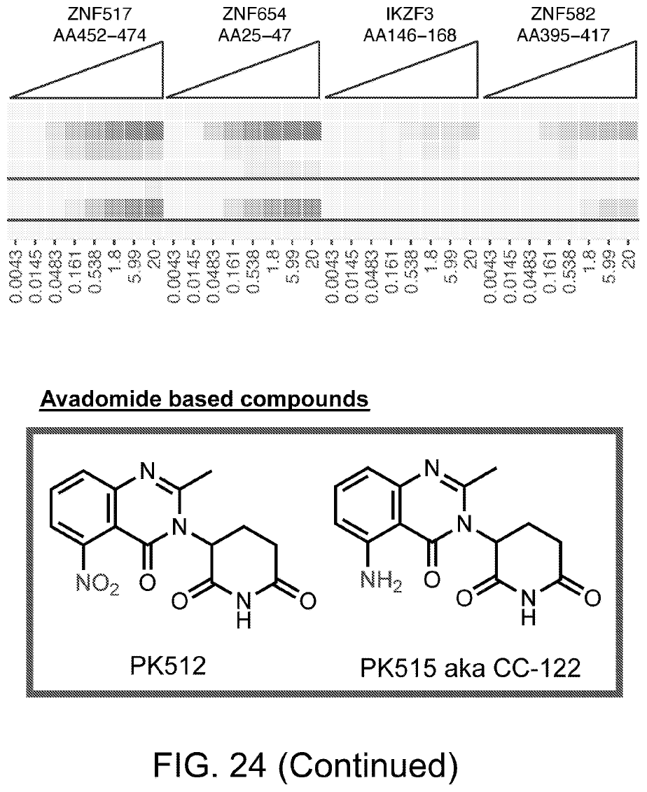

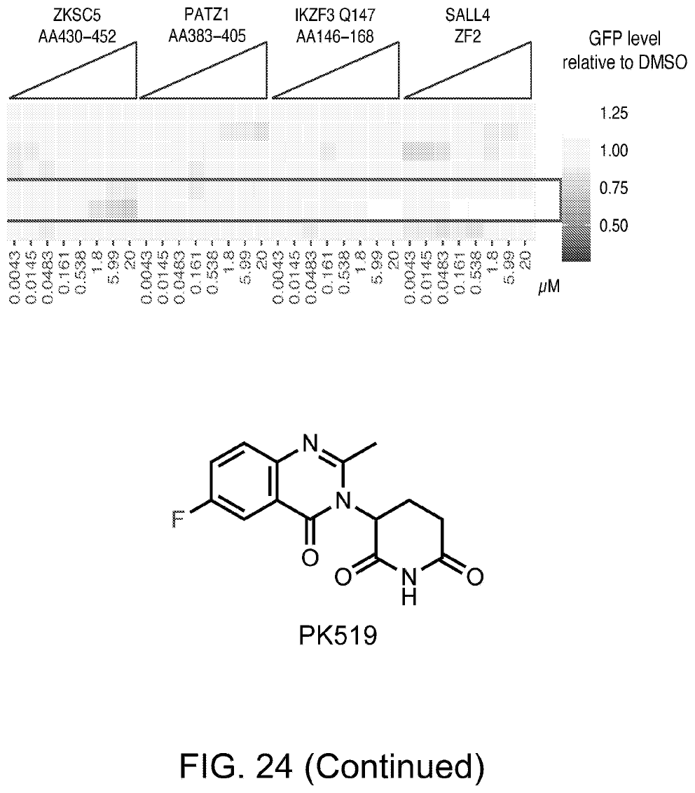

Systematic evaluation of ZF protein degradation propensity of pomalidomide analogs. Using this newly developed off-target profiling platform, the library of pomalidomide analogues were tested to derive rules for the impact of exit vector modifications on pomalidomide and ZF protein degradation. First, analogs with C5 modifications on the phthalimide ring were observed to have reduced ZF degradation relative to identical modifications on the C4 position, particularly for SNAr (C—N) analogs (P=3.4×10⁻¹⁰) (FIGS. 2B, 11A). The trend was less significant for modifications made with acylation (N—C) and Suzuki/Sonogashira couplings (C—C) analogs, partly due to having smaller sets of analogs compared with the SNAr group (FIGS. 11B-11C). Structure modeling and docking data also suggest that C5 modifications are more likely to create a steric clash between the SNAr exit vectors and the ZF domain than C4 position modifications (FIGS. 10A-10C). Second, analogs lacking hydrogen-bond (H-bond) donors immediately attached to the phthalimide ring were observed to have significantly reduced ZF degradation compared to those with H-bond donors, regardless of the position of the modification relative to the phthalimide ring (FIG. 2E) (P<0.0001). These findings are also in agreement with ZF degradation profiling for reported PROTACs (FIG. 1B). Immunoblotting results are also in agreement with the high-throughput imaging data confirming the C5-C4 position-dependent effect on the degradation of endogenous ZF proteins, ZFP91 and IKZF3 (FIG. 2F). Taken together, these data reveal that pomalidomide-based PROTACs with an arylamine exit vector, where NH- is a H-bond donor, induced greater ZF degradation and suggesting PROTACs without H-bond donors would render the clear PROTACs. This finding is consistent with published data, which demonstrates that the ability of pomalidomide to form H-bonds is essential for coordinating the ternary complex between ZF and CRBN for subsequent ZF degradation.¹ With the aim to further minimize off-target ZF degradation. SNAr analogs with C5 position modifications were generated through addition of a fluoro group at the C6 position (FIG. 5, and Table 1). The fluoro group reduced ZF degradation for most SNAr exit vectors, such as acetylpiperazine and morpholine, but not for diazaspiro-heptane (FIGS. 11A and 11D).

With the aim to identify exit vector modifications that confer the least off-target ZF degradation for PROTAC development, a degradation score was derived for each pomalidomide analog, including pomalidomide-based PROTACs, by taking the sum of eGFP degradation values for the ZF degrons at multiple doses for each analog (FIG. 3). Compounds with degradation scores close to zero induced the least ZF degradation, whereas compounds with the most negative scores induced the most ZF degradation (FIG. 3). Analogs with piperazine and alkyne exit vectors predominated the group of compounds with degradation scores of 0 (FIG. 3). Other exit vectors with minimal degradation scores include phenyl, diazaspiro-undecane, azetidine, pyrrolidine, difluoro-pyrrolidine, morpholine, diazaspiro-heptane, methylcyclohexylamine along with fluoro analogs of the morpholine, N-protected piperazine and N-protected diazaspiroheptane (FIG. 3). From this study, two main rules were established for designing pomalidomide-based PROTACs to minimize off-target effects. First, exit vectors should predominantly have SNAr modifications on the C5 position. Second, none of the H-bond donors should be immediately adjacent to the phthalimide ring. Taken together, this systematic analysis of pomalidomide analogs has been applied to identify a collection of exit vectors to guide the development of pomalidomide-based PROTACS with minimal off-target ZF degradation.

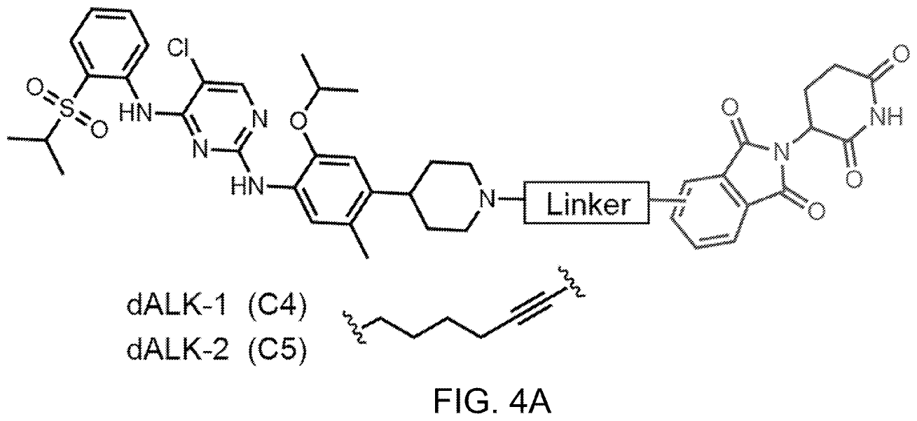

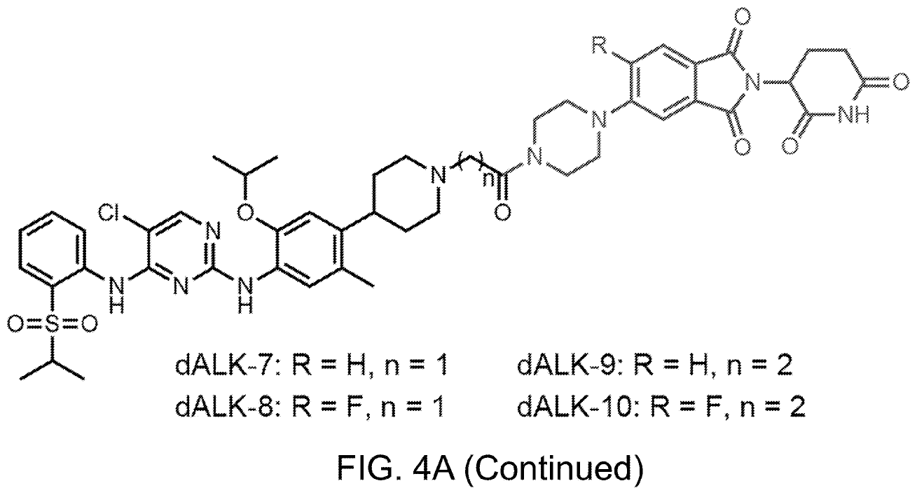

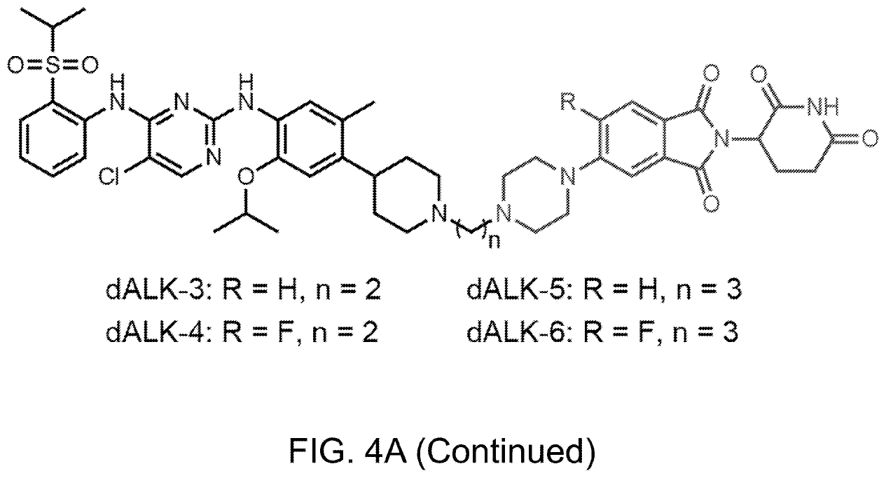

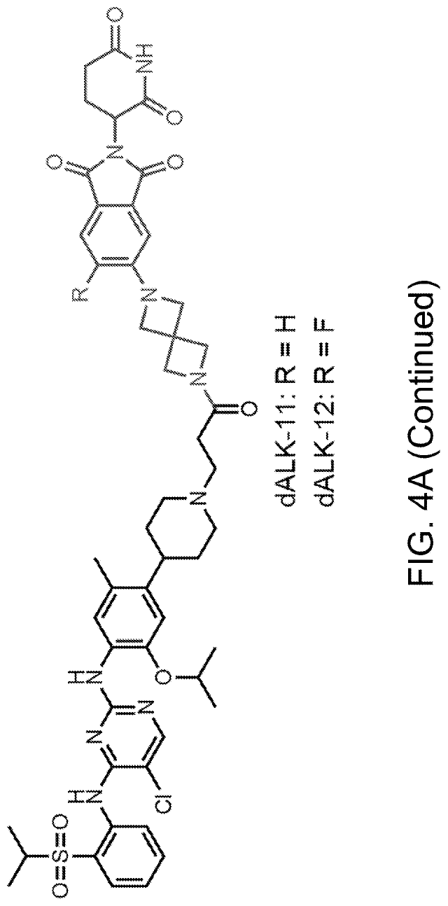

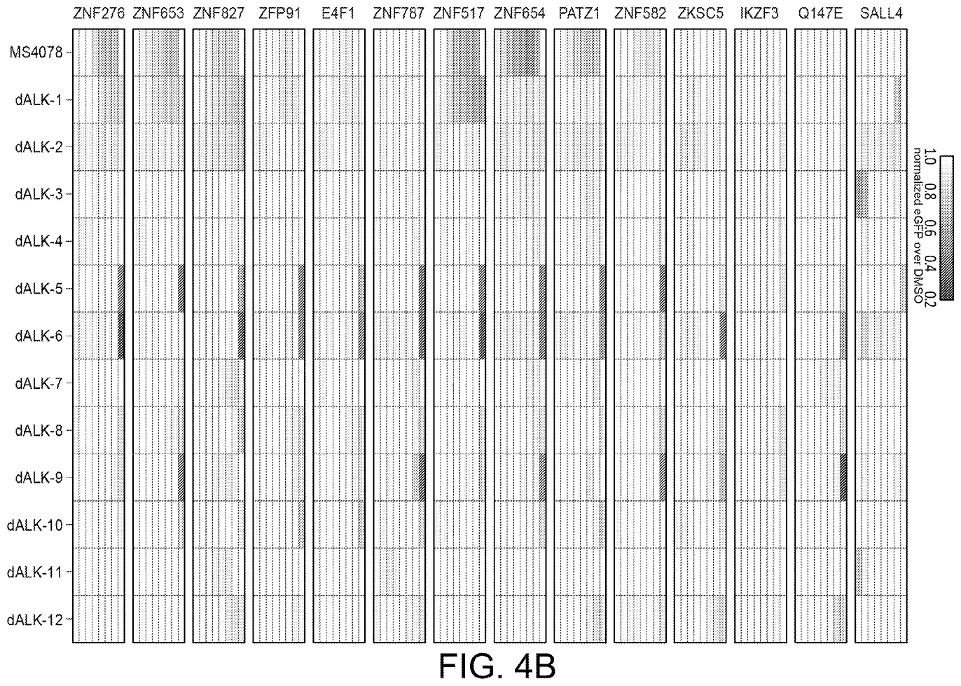

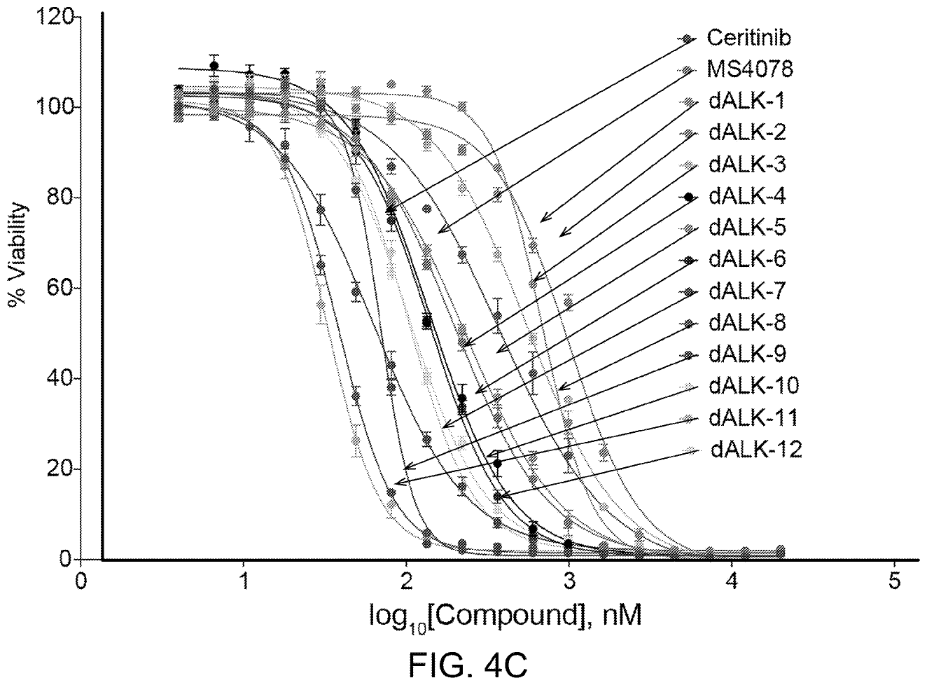

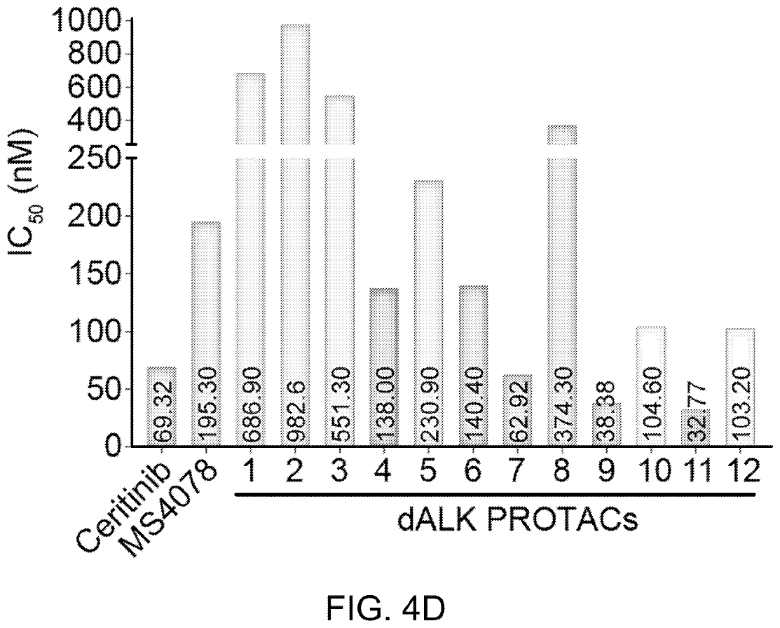

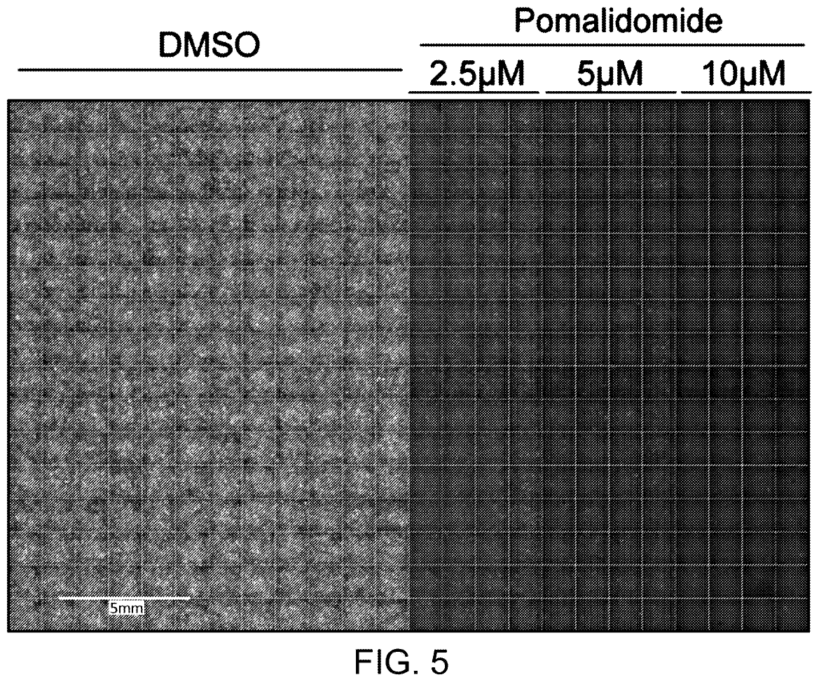

Development of PROTACs with reduced off-target degradation propensities. As a proof-of-concept of these findings, the potent commercially available ALK PROTAC, MS4078, which had a high level of off-target ZF degradation (FIGS. 1B-1C), was reengineered by altering the exit vectors on pomalidomide to reduce off-target ZF degradation while maintain potency. Selected pomalidomide analogs had alkyne and piperazine exit vectors, which had degradation scores closest to zero (FIG. 3), on the C5 positions and included the C5 piperazine with a C6 fluoro modification due to its near-zero ZF degradation score (FIGS. 2C and 3). A group of 12 new ALK PROTACs was synthesized with different exit vectors such as alkyne (C4: dALK-1 and C5: dALK-2), piperazine with varying alkyl (dALK-3 to 6), and acyl linkers (dALK-7 to 10) and diazaspiroheptane (dALK-11 and 12). To investigate the effect of the fluoro group on off-target and on-target propensity, the corresponding fluoro pairs were also synthesized for non-alkyne dALK PROTACs (dALK-4, 6, 8, 10, 12) (FIG. 4A).

The high-throughput off-target analysis of new ALK PROTACs was then performed to investigate the off-target profiles of these compounds. The reengineered ALK PROTAC with C5 alkyne exit vector (dALK2) dramatically reduced the off-target effects MS4078 such as ZNF517, ZNF654, ZNF276, ZNF653, PATZ1 (FIG. 4B). New ALK PROTACs with piperazine exit vectors with and without the —F group exhibited minimized off-target effects (FIG. 4B). However, dALK-5, 6, 9 exhibited off-target effects at higher dose (20 μM) potentially due to the cytotoxic effects. Through this the set of rules was demonstrated for development of new PROTACs with reduced off-targets using cleaner IMiDs as the building blocks for the PROTACs.

Cytotoxicity studies were then performed to investigate any enhancement in the on-target potential. One of the non-IMiD building block of parent ALK PROTAC MS4078 is ceritinib, a potent ALK inhibitor with IC50 of 69.3 nM and its conversion to degrader (MS4078) has lowered its potency by 2.8-fold with high IC₅₀ values (195.3 nM) (FIG. 4C). Though introduction of alkyne exit vectors reduced the off-target effects but increased its IC₅₀ values suggesting the reduction in the on-target potential due to linker length defect to induce the proximity. With the systematic linker length optimization for piperazine and diazaspiroheptane exit vectors, a group of 7 potent dALK PROTACs was identified with IC₅₀ values lower than that of MS4078. Strikingly, dALK PROTACs with C5 piperazine exit vectors with propyl/butyl amide linkers (dALK-7, 9 respectively) and C5 diazaspiroheptane with propylamide linker (dALK-11) found to be the potent and cleaner PROTACs. Among these 7 dALK PROTACs were identified two best-in-class PROTACs with 1.8-2 fold higher potency than MS4078 with IC₅₀ values 32.77 nM (dALK11) and 38.38 nM (dALK12), which renders it more effective in reducing the SUDHL-1 cell viability (FIGS. 4C-4D).

A new and high-throughput off-target profiling platform has been developed for the systematic evaluation of PROTACs that induce off-target degradation of ZF proteins, which play crucial roles in biology and disease progression and validated this platform using reported proteomic data. Leveraging this high-throughput platform, a library of pomalidomide analogs has been designed and tested that was employed to identify new rules for designing pomalidomide-based PROTACs that minimize harmful off-target degradation of ZF proteins. It was discovered that modification of the exit vectors on the C5 position of the phthalimide ring via nucleophilic aromatic substitution (SNAr) (C—N) reduces off-target ZF degradation. Guided by these new designed principles, a proof-of-concept has been disclosed in which a commercially available ALK PROTAC, MS4078) was reengineered for enhanced potency and reduced off-target degradation.

The new rules for pomalidomide-based PROTACs generated in this study can be readily applied to address the crucial need for PROTACs that do not indiscriminately degrade key ZF proteins, which have widespread implications in human health and disease progression. For example, the previously mentioned functional disruption of the pomalidomide-degradable ZF protein ZFP91² can aggravate the severity of colonic inflammation and has been associated with the promotion of inflammation-driven colorectal cancer,⁴ hepatocarcinogenesis,¹⁰ and gastric cancer metastasis acceleration,¹¹ suggesting which all suggest that ZFP91 degradation by pomalidomide-based PROTACs may promote cancer progression. IKZF3, another pomalidomide-degradable ZF protein studied here,² is essential for B cell activation and maturation,¹² and hence plays a critical role in adaptive immune response. As such, degradation of IKZF3 can affect the body's ability to fight cancer.¹³ Furthermore, pomalidomide and immunomodulatory drugs in general are reportedly harmful to fetuses during gestation.

The present disclosure offers opportunities to develop new and safer PROTACs as well as to improve on existent PROTACs with enhanced on-target potency for the treatment of myriad diseases. This collection of synthetic pomalidomide derivatives with varied exit vectors that affect minimal off-target ZF degradation can be widely adopted for the generation of safer and clinically relevant PROTACs. Conclusively, the present disclosure provides further confidence and validation for the potential to apply exit vectors discovered in this work for the benefit of clinical applications and to the PROTAC community at large.

Materials and Methods (Biology Part). Cell lines. U2OS (ATCC, HTB-96) cells stably expressing ZF degrons were cultured in Dulbecco's modified Eagle's medium (DMEM) (ThermoFisher Scientific, 12430062), 10% (v/v) fetal bovine serum (FBS) (ThermoFisher Scientific, 16140071), 1 μg/ml puromycin (ThermoFisher Scientific, A1113803), and 100 U/ml Antibiotic-Antimycotic (ThermoFisher Scientific, 15240062). Similarly, 293T cells (ATCC, CRL-3216) were cultured in the same medium as the U2OS cells without puromycin. MM1.S (ATCC, CRL-2974), SU-DHL-1 (ATCC, CRL-2955), and H2228 cells (ATCC, CRL-5935) were cultured in RPMI 1640 medium (ThermoFisher Scientific, 11875119), 10% (v/v) FBS, and 100 U/ml Antibiotic-Antimycotic.

Plasmids. The 15 lentiviral ZF plasmids were generated using the Cilantro 2 degradation reporter vector (Addgene, 74450) as previously described (Ebert Science paper). Among the 15 plasmids, 12 are validated pomalidomide-sensitive ZF degrons, including E4F1 AA (amino acid) 220-242, ZNF276 AA524-546, ZNF517 AA452-474, ZNF582 AA395-417, ZNF653 AA556-578, ZNF654 AA25-47, ZNF787 AA178-200, ZNF827 AA374-396, PATZ1 AA383-405, ZFP91 AA400-422, IKZF3 AA146-168, and the ZFP91-IKZF3 hybrid. Three of the 15 plasmids are pomalidomide-insensitive ZFs, which served as negative controls, including SALL4 ZF2, ZKSC5 AA430-452, and IKZF3 AA146-168 Q147E.

Lentivirus production and transduction. Viral packaging plasmids psPAX2 (Addgene, 12260) and pMD2.G (Addgene, 12259) together with lentiviral ZF degron plasmids were transfected to 293T cells in a 2:1:3 ratio using Lipofectamine 3000 transfection reagent (ThermoFisher Scientific, L3000015) following the manufacturer's guidelines. Lentiviruses were collected 48 and 72 hrs after transfection and filtered with 0.45-μm filters. For transduction in U2OS cells, the viral supernatant was mixed with U2OS culture media in a 1:1 ratio with 10 μg/ml Polybrene transfection reagent (MilliporeSigma, TR-1003-G), then selected with 2 μg/ml puromycin after at least 24 hrs of transduction.

Automated high-content imaging screening and analysis. U2OS cells stably expressing the ZF degron reporters were seeded in 30 μl of 3000 cells per well in CellCarrier-384 Ultra Microplates (PerkinElmer, 6057302) pre-printed with 10 mM of stock compounds in varying volumes using Tecan D300e Digital Dispenser (Tecan). After 24 hours, the 384-well plates were washed once with PBS then fixed in 4% paraformaldehyde and stained by HCS NuclearMask Blue stain (ThermoFisher Scientific, H10325). Imaging of eGFP and mCherry was performed for each plate using an Opera Phenix high-content imaging system followed by analysis with Harmony Software v4.9 (PerkinElmer).

The fluorescence intensity of eGFP was normalized to that of mCherry for every cell. The mean normalized eGFP intensity of all cells in each well was then normalized by that in DMSO-treated wells to determine the GFP level relative to DMSO for each compound across doses. The GFP level relative to DMSO was used to generate a heatmap using R v4.0.2. Compounds that cause minimal ZF degradation have values close to 1 (i.e., DMSO value), whereas compounds that cause extensive ZF degradation have values close to 0. To compute the degradation score for each compound, all DMSO-normalized GFP values greater than 0.9 were excluded to ensure that only ZF degradation values contribute to the score but not those that were associated with increases in GFP or caused minimal to no change in GFP degradation. The remaining DMSO-normalized GFP values were subtracted from 1 to derive the fraction of GFP degradation caused by the compounds. The degradation score was then calculated by taking the sum of the GFP degradation fractions across all doses for each compound. For the correlation heatmap and principal component analysis (PCA), a ZF degradation profile was generated for each compound using DMSO-normalized GFP values in ZF reporter cells when treated with different doses of pomalidomide analogs. Clustering was then performed using pheatmap v1.0.12 and PCAtools v2.2.0 packages for correlation heatmap and PCA, respectively.

Proteomics analysis of PROTAC degradation of endogenous ZF proteins. The relative abundance of 811 unique pomalidomide-sensitive ZF proteins (Ebert paper) were extracted from 124 publicly available pomalidomide-based PROTAC proteomics datasets published in Donovan et al, 2020 (Eric Fisher paper). Here, 644 out of 811 ZF proteins were detectable in at least one PROTAC proteomics dataset and are shown in Figure S3b. To compute the ZF degradation score for each pomalidomide-based PROTAC dataset, all relative abundance values that are greater than 0 were excluded to ensure that only degradation values contribute to the score but not those that are associated with increases in protein abundance. The degradation score was then calculated by taking the sum of negative abundance values, representing ZF proteins that reduced in abundance when the cells were treated with PROTACs.

Western blot analysis. Lysates of cells treated with different compounds were collected with ice-cold M-PER™ Mammalian Protein Extraction Reagent (ThermoFisher Scientific, 78501) with freshly added phosphatase (Sigma-Aldrich, 04906837001) and protease (Sigma-Aldrich, 04693124001) inhibitors following the manufacturer's instructions. Next, 20-50 μg of proteins were fractionated with NuPAGE™ 4-12% Bis-Tris gels (ThermoFisher Scientific, NP0335BOX) then transferred onto nitrocellulose membranes using iBot™ Transfer Stacks (ThermoFisher Scientific, IB23002) following the manufacturer's instructions. Membranes were then stained with primary antibodies in 1:1000 dilutions and secondary fluorescent antibodies in 1:3000 dilutions using iBind™ Flex Fluorescent Detection Solution Kit (ThermoFisher Scientific, SLF2019) following the manufacturer's instructions.

Primary antibodies used in this study include ZFP91 (Bethyl Laboratories, A303-245A), IKZF3/Aiolos (D1C1E) (Cell Signaling, 15103S), CRBN (D8H3S) (Cell Signaling, 71810S), ALK(D5F3) XP (Cell Signaling, 3633S), phospho-ALK (Tyr1507) (D6F1V) (Cell Signaling, 14678S), β-actin (8H10D10) Mouse mAb (Cell Signaling, 3700S). Fluorescent secondary antibodies used in this study include IRDye 680RD goat anti-mouse IgG (LI-COR Biosciences, 926-68070) and IRDye 800CW goat anti-rabbit IgG (LI-COR Biosciences, 926-32211). Western blot detection was performed using an Odyssey CLx Imaging System (LI-COR Biosciences). Quantification of the relative area and density values of western blot bands were carried out using ImageJ v2.1.0 following the ImageJ User Guide for gel analysis (https://imagej.nih.gov/ij/docs/guide/). Quantified values were normalized by values for loading controls such as β-actin. For phospho NPM-ALK, quantified values were normalized by the values for total ALK.

Cell Viability Assay. SU-DH-L1 cells were seeded in 30 μl of 8000 cells per well in 384-well plates pre-printed with 10 mM of the stock compounds in varying volumes using a Tecan D300e Digital Dispenser (Tecan). After 24 hours, cell viability was determined using a CellTiter-Glo Luminescent Cell Viability Assay (Promega, G7571) following the manufacturer's instructions. Dose-response curve fitting and IC50 quantification were determined with four parameter nonlinear regression analysis using GraphPad Prism v8.4.2.

Statistical analysis. Statistical tests were conducted using suitable underlying assumptions on variance characteristics and data distribution. Unless otherwise noted, two-tailed Student's t-tests were used for comparisons between groups.

Materials and Methods (Chemistry Part). Pomalidomide analogs synthesis and characterization

The chemical synthesis and characterization of pomalidomide analogs and ALK PROTACs is described in the supplemental information.

PROTAC synthesis. Commercially available PROTACs, including BETd-260, BI-3663, BSJ-03-123, MD-224, MT-802, SJF620, and PROTAC K-RAS Degrader-1, were purchased from MedChemExpress, whereas dBET6, dBET1, dBET57, ARV-825, and MS4078 were purchased from Selleck Chemicals. dTAG-13 and dTAG-47 were synthesized in house.

### Example 2—Synthesis and Screening of IMiD Analogs

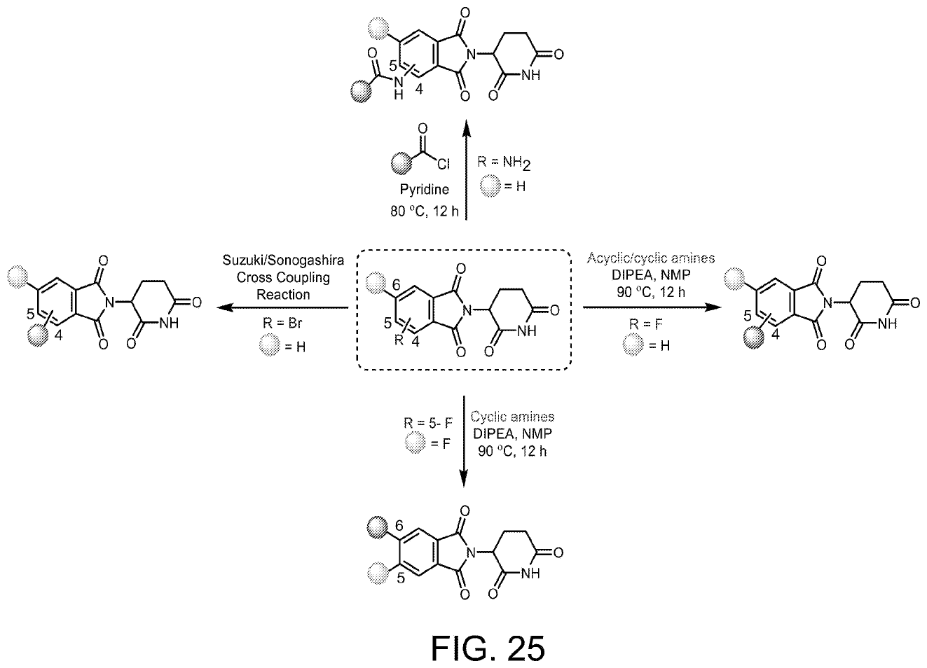

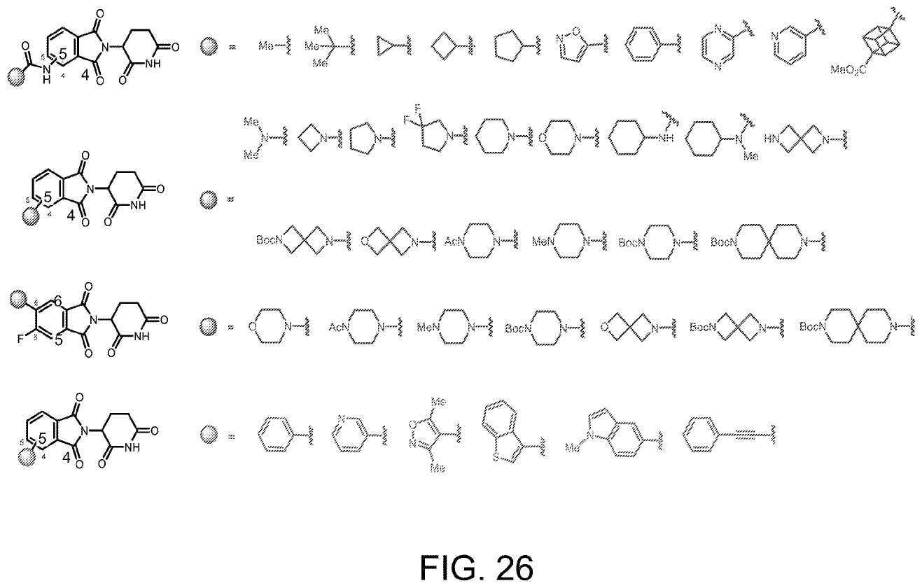

High content confocal microscopy can be utilized to robustly detect ZF-off targets of PROTACs (FIG. 1A). Exemplary synthetic scheme for synthesis of IMiD analogs is depicted in FIG. 25. FIG. 26 includes structures of exemplary IMiD analogs. Metrics to nominate IMiD candidates can include degradation score as described in FIGS. 3A-3B. The approaches allow for the rational design of PROTACs with new exit vectors. Exemplary PROTACs designed with new exit vectors is depicted in FIG. 27 for Anaplastic lymphoma kinase (ALK) PROTACs.

### Example 3—Synthesis and Screening of IMiD Analogs

Specific Aims. Proteolysis Targeting Chimeras (PROTACs), a class of heterobifunctional molecules that recruit target proteins to E3 ligases, are emerging as a novel therapeutic modality for targeted protein degradation.¹⁻³ Pomalidomide is an Immunomodulatory drug (IMiD) that induces proximity between cereblon (CRBN), a component of E3 ubiquitin ligase, and proteins with Zinc-finger (ZF) motifs to trigger ubiquitination, followed by degradation.⁴⁻⁶ However, Pomalidomide is a widely used E3 ligase recruiting building block in PROTACs, and can independently degrade other targets, such as zinc-finger (ZF) proteins, that hold key functions in normal development and disease progression.⁷⁻¹⁰ For example, tissue-specific deletion of pomalidomide-degradable ZF protein, ZFP91, in regulatory T cells (Tregs) leads to Treg dysfunction. Also, it increases the severity of inflammation-driven colorectal cancer.¹¹ Additionally, numerous other proteins with essential roles in cellular function, such as transcription factors, also harbor ZF domains.^{12,13} The off-target degradation of these critical ZF-containing proteins may have long-term implications for developing new cancers, dysregulation of lymphocyte development, and teratogenic effects.¹⁴⁻¹⁷ Finally, several PROTACs are being used to develop molecular switches for the synthetic genetic circuit, including those for controlling Chimeric Antigen Receptor T (CAR-T) cell technologies.^{18,19} Therefore, there is crucial to establish the rules for PROTAC design that minimize off-target degradation apart from the degradation of an intended target protein. The design of cleaner PROTACs/IMiD analogs is contingent on accurate, modular, robust detection of the degradation of proteins.²⁰

Currently, mass-spectrometry-based methods can assess off-target degradation to detect protein levels.²¹⁻²⁴ However, these techniques lack sensitivity for low abundant proteins.²⁵ In addition to the expense, mass spectrometry is technically challenging when analyses include profiling the off-target degradation affected by specific PROTACs across multiple tissue types for tissue-specific expression of lineage-specific proteins.²⁶ These analyses are further complicated by the need to perform these assessments across different levels of PROTAC dosing. Thus, there is an unmet need for robust, sensitive, multi-platform, and high-throughput methods to determine off-target degradation in such PROTACs. Applicant developed a high throughput image-guided ZF-off target detection platform, screened a small library of IMiD analogs, and nominated ˜20 cleaner IMiDs for PROTAC design.²⁷ Interestingly, the cleanest CRBN recruiter identified by this platform is isostructural to those in PROTACs under clinical trials.^{28,29} This high-throughput imaging assay measures mains upon the compound treatment.³⁰ Building on these studies, Applicant will develop an integrated platform for the off-target analysis of PROTACs.

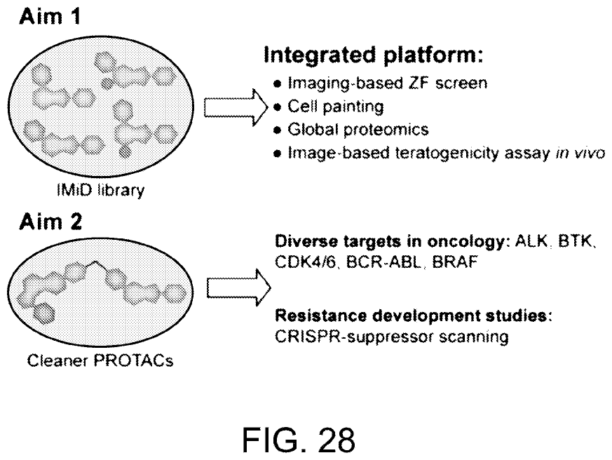

Aim 1 (FIG. 28). An integrated platform for the off-target analysis of IMiDs. Applicant has rationally designed a diverse library of pomalidomide analogs with various linkers (exit vectors) and structurally/stereochemically diverse modifications. Applicant will expand the capabilities of the high-throughput imaging platform to enhance sensitivity and include additional ZF targets and use this assay to test pomalidomide analogs. Next, Applicant will utilize the unbiased, high-content image-based platform, Cell painting, to identify phenotypically and correlate the features of the cells with the ZF screens and integrate the different image-guided platforms for off-target identification. Applicant will cross-validate data from these two image-based platforms using TMT-based global proteomics experiments to develop a degradation score for each analog. Finally, since many IMiDs suffer from teratogenic effects, Applicant will examine the developmental toxicities of these new cleaner molecules using a high-content imaging-based Zebrafish embryo teratogenic assay.

Aim 2 (FIG. 28). Generalization and resistance development to the new PROTACs. Applicant will utilize cleaner pomalidomide analogs to build cleaner PROTACs of high-value therapeutic targets in cancer, including anaplastic lymphoma kinase (ALK), Bruton's tyrosine kinase (BTK), Cyclin-dependent Kinases (CDK4/2/6), breakpoint cluster region fusion ABL (BCR-ABL), and BRAF. Finally, Applicant will investigate the resistant development to existing/new PROTACs for the above-mentioned targets by employing the CRISPR-based mutagenesis approach to deprioritize analogs for which resistance is easy to develop.

Applicant's integrated approach will fundamentally advance understanding of PROTAC off-targets by leveraging tools and principles from high-content imaging, bioengineering, chemical biology, cancer pharmacology, and systems biology.

Significance. The degradation of cellular proteins is necessary for routine maintenance of cellular function, including proliferation, differentiation, and cell death. Immunomodulatory imide drugs (IMiD)-based molecular glues (e.g., Pomalidomide) induce proximity between cereblon (CRBN), the substrate receptor for an E3 ubiquitin ligase, and proteins with Zn-finger (ZF) motifs to trigger ubiquitination and degradation of the latter.³¹⁻³³ Pomalidomide is often appended to target protein binders to generate CRBN-based Proteolysis Targeting Chimeras (PROTACs) that induce proximity-mediated target protein degradation.³⁴⁻³⁶ However, these pomalidomide-based PROTACs can also recruit other proteins with or without ZF motifs that serve critical biological functions in normal development and disease progression.^{37,38,13,39} For example, tissue-specific deletion of ZFP91 in regulatory T cells (Tregs) leads to Treg dysfunction and increases the severity of inflammation-driven colorectal cancer.¹¹ Several transcription factors such as SALL4 and IKZFs that contain C2H2 ZF domains have essential roles in cellular function.⁴⁰⁻⁴² Thus, the application of this pomalidomide-based PROTAC-induced degradation of these vital ZF-containing proteins may have long-term implications such as the development of new cancers, dysregulation of lymphocyte development, and teratogenic effects.^{40,15,11,43,39} The ability of Pomalidomide to degrade other proteins in a PROTAC-independent manner raises concerns about the precariousness of off-target ubiquitination and degradation of these compounds, several of which are already in clinical trials

To profile the ZF degradation propensity of pomalidomide and PROTACs, Applicant first developed an automated imaging assay (FIG. 1A). Applicant selected 23-amino-acid ZF degrons of 11 ZF proteins that are reportedly degraded by pomalidomide and 3 ZFs that are not (see Table S1 in Ref. 27).³⁰ Applicant inserted these ZF degrons into a lentiviral degradation reporter vector (cilantro 2)³⁰ to compare the fluorescence of ZF-tagged enhanced green fluorescent protein (eGFP) to untagged mCherry (FIG. 1A). With this assay, Applicant tested pomalidomide against the 14 stable U2OS cell lines containing tagged ZF-proteins, Applicant reliably detected dose-dependent degradation of ZF degron-tagged eGFP by pomalidomide with a robust readout (Z′=0.8) (see Fig. S1 in ref. 27). As expected, pomalidomide degraded all 12 ZF degrons that are sensitive to it in a dose-dependent manner (FIG. 2A and FIG. 1 in ref. 27). Unlike mass spectrometry, this reporter-based method is not limited by cell-type-specific expression levels of analyte proteins nor by the accessibility to ZFs in the context of full-length proteins that are engaged in protein complexes. Thus, this method may have enhanced sensitivity over mass spectrometry-based methods for detecting pomalidomide-sensitive ZF protein degradation.

With this assay in hand, Applicant profiled the off-target activity of 9 reported PROTACs with varying exit vectors from pomalidomide end and linker lengths (FIG. 1B; full dose data in FIG. 1 of ref. 27). We observed significant degradation of many ZF-domains with almost all the 9 PROTACs (FIG. 1B). Notably, PROTACs with common exit vectors, such as arylamine, -ether, -carbon, and -amide, generally had greater ZF degradation capabilities in a similar fashion to pomalidomide. Applicant's assay also confirmed the off-target degradation of endogenous ZF proteins such as ZFP91 and IKZF3 by reported PROTACs MS4078 (see FIG. 1C in ref 27)⁴⁷ and dTAG-13 (see FIG. 1E in ref. 27)^{48,49}, as validated by immunoblotting (see FIGS. 1D, 1F, and S3 in ref. 27).

Applicant analyzed changes in endogenous ZF proteins from 124 proteomics datasets that were generated for cells treated with pomalidomide-based PROTACs.²⁶ In Figure S4 of ref. 27, the relative abundance was shown of proteins that contained the ZF motif as previously described³⁰ and were detectable in at least one proteomics dataset (i.e., 284 ZF proteins). A ZF degradation score was computed for every PROTAC dataset by taking the sum of ZF protein abundance. Analyzing the degradation score distribution confirmed that PROTACs had significant ZF protein degradation activity for amino acetamide and arylamine, -ether, and -carbon exit vectors (See figure S4 in ref. 27). Both the analysis of these proteomic datasets and the image-based profiling point to significant off-targets of PROTACs.

Innovation. This proposal brings together cutting-edge technologies from multiple disciplines, including medicinal chemistry, cellular pharmacology, zebrafish developmental toxicology, high-throughput imaging platform, and CRISPR-mutagenesis to resolve the off-target effects of PROTACs. Applicant proposes to develop a sensitive, robust, and high-throughput imaging platform to profile off-target activity and integrate those results with those from imaging platforms (i.e., cell painting), global proteomics profiling, and phenotypic teratogenicity studies. As resistance development to PROTACs is a potential liability source in aggressive cancers, Applicant has added the CRISPR-based mutagenesis platform to the PROTAC off-target analysis.

Approach. Aim 1. An integrated platform for the off-target analysis of IMiDs and PROTACs. Preliminary data. Generation and off-target profiling of ˜80 pomalidomide analogs. Applicant next endeavored to create a library of rationally designed pomalidomide analogs that could be applied to the systematic design of pomalidomide-based PROTACs with minimal off-target ZF degradation. Applicant gained structural insight from the crystal structure of the DDB1-CRBN-pomalidomide complex bound to transcription factor IKZF1 (PDB: 6H0F; FIG. 2A).³⁰ In the crystal structure, the glutarimide ring of Pomalidomide is deeply buried inside CRBN. In contrast, the phthalimide ring is accessible for modification. Q146 of IKZF1 forms a water-mediated hydrogen-bonding interaction with the C4 amino group of the compound. At the same time, the C5 position is proximal to the ZF domain (FIG. 2A). Mutation of Q146, has been reported to abolish IKZF degradation. Applicant hypothesized that appropriate substitutions at the C4 and/or C5 positions could disrupt the ternary complex of the ZF domains with CRBN while maintaining its interaction with CRBN through the glutarimide ring. To investigate this, Applicant first synthesized a thalidomide analog, 5-aminothalidomide (FIG. 2B). The treatment of MM1.S cells with this C5-amino analog revealed a decrease in overall degradation potency compared to Pomalidomide (FIG. 2B), suggesting that modifications on the C5 position will “bump off” or eliminate the endogenous ZFs while preserving the CRBN interaction. Therefore, Applicant generated ˜80 imide analogs with various modifications on the C4 and C5 positions of the phthalimide ring (See FIG. 3A; FIG. 5 in ref 27). These analogs should not affect recruitment of CRBN for inducing protein degradation since the imide ring does not interact with the ubiquition ligase.

Rules for design of new pomalidomide analogs with reduced off-targets. Applicant used this assay to identify a collection of pomalidomide analogs with minimal ZF degradation and derived new rules to design cleaner PROTACs. Applicant derived a degradation score for each pomalidomide analog, including pomalidomide-based PROTACs, by taking the sum of eGFP degradation values for the ZF degrons at multiple doses for each analog (FIG. 3B). Compounds with degradation scores close to zero induced the least ZF degradation, whereas compounds with the most negative scores induced the most ZF degradation (FIGS. 3B-3C). Analogs with piperazine (3, 5, 6, and 9) and alkyne (7 and 8) exit vectors prevailed in the group of compounds with degradation scores of 0. Other exit vectors with minimal degradation scores include phenyl (1), as well as 6-fluoro substituted analogs of N-protected piperazines (2 and 10), and diazaspiro[5.5]undecane (4).

From this study, Applicant established two main rules for designing pomalidomide-based PROTACs to minimize off-target effects. First, exit vectors should predominantly have modifications on the C5 position. Second, none of the H-bond donors should be immediately adjacent to the phthalimide ring (see FIG. 2D-F in ref. 27). Taken together, Applicant has applied this systematic analysis of pomalidomide analogs and identified a collection of exit vectors to guide the development of pomalidomide-based PROTACS with minimal off-target ZF degradation. Preliminary studies suggest that IMiD molecules with close to zero degradation scores also exhibited moderate-to-high permeability and are not substrates of drug efflux protein (p-gp) in Caco-2 cell-based bi-directional cell permeability assay (data not shown).

Cell painting. Cell painting is a high-content image-based morphological profiling assay.⁵⁰ In a typical cell painting procedure, cells are plated in multiwall plates, perturbed with the treatments of chemical compounds, stained using multiplexed fluorescent dyes for various organelles and structures, and imaged in multiple channels on a high-throughput microscope. An automated image analysis software then identifies individual cells and measures about 1,500 morphological features of cell components, such as nuclei, nucleoli, actin, Golgi, and mitochondria, in terms of size, shape, texture, intensity to yield a rich profile for the detection of subtle

phenotypes. Cell painting captures subtle patterns in the combination of morphological labels, detecting cellular effects of chemical compounds even if their targets are not directly stained. Unlike typical image-based profiling that is only applied to particular types of interesting phenotypes, cell painting represents an unbiased manner to cover a much more comprehensive range of phenotypes for rapid screening of changes in cell shape and function in response to drug toxins and other factors.

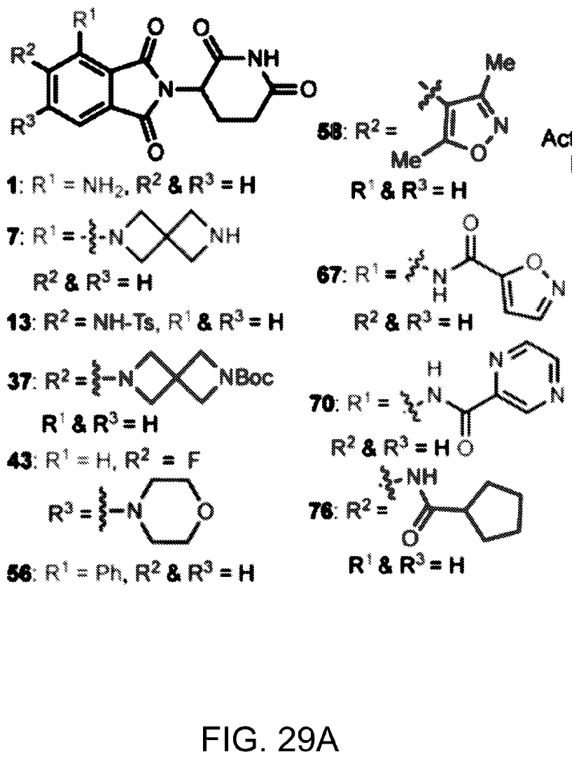

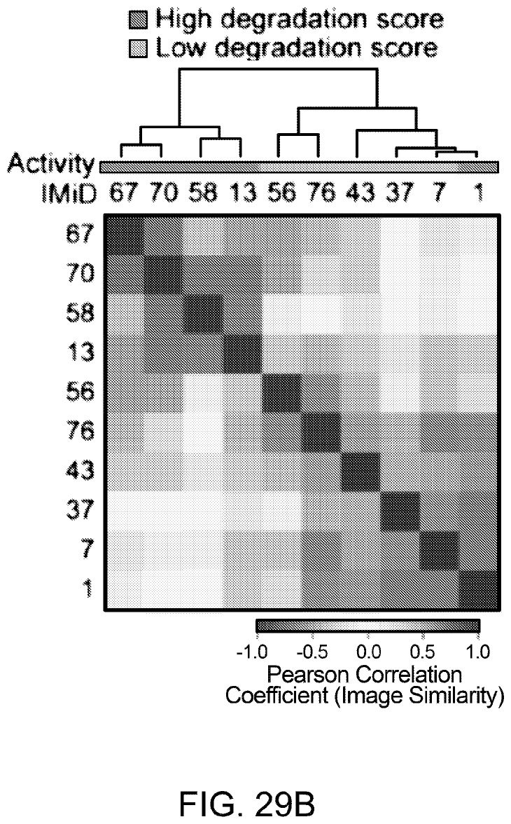

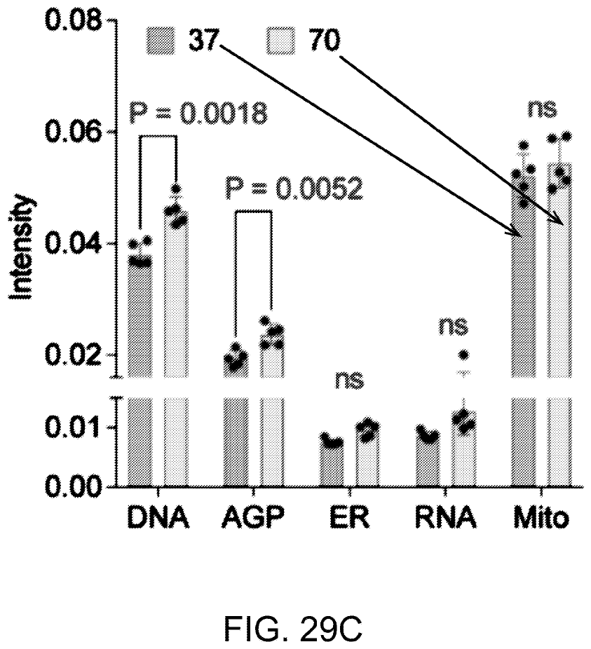

Applicant performed cell painting on some members of our IMiD library. Cells were treated with compounds, fixed, and stained with six fluorescent dyes used to label different components of the cell, including the nucleus, endoplasmic reticulum, mitochondria, cytoskeleton, Golgi apparatus, and RNA. Cell painting image similarity data was visualized, and hierarchal clustering was performed on Morpheus. Images were colored and overlayed using the ImageJ merge channels function.⁵¹ Feature extraction and image-level comparisons were performed using CellProfiler.⁵² For each image, features were computed for individual cells, individual nuclei, the cytoplasm of individual cells, and at the image level. The median value of a feature across all cells, nuclei, or cytoplasm was used for downstream analysis. P-values comparing image features between two compounds were computed by heteroscedastic two-tailed Student's t-test, with five replicate images for each compound. Applicant took the top 5 Pomalidomide analogs with the highest and lowest degradation score (FIG. 29A) and clustered the remaining library members looking for correlation. Applicant observed separate clustering for compounds with high off-targets compared to compounds with low off-targets (FIGS. 29A-29C). Applicant observed statistically significant differences between compounds 37 (low degradation score) and 70 for the intensities at DNA and AGP while no differences at ER, mitochondria, RNA (FIG. 29C). Applicant hypothesized that compound 70 causes changes in the nucleus, Golgi, and cytoskeleton features that could possibly affect cell proliferation, transcription, protein/lipid trafficking.

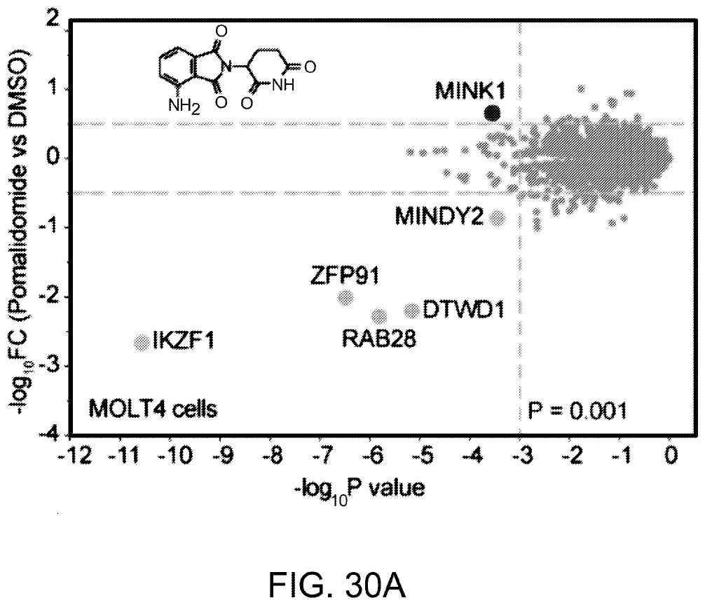

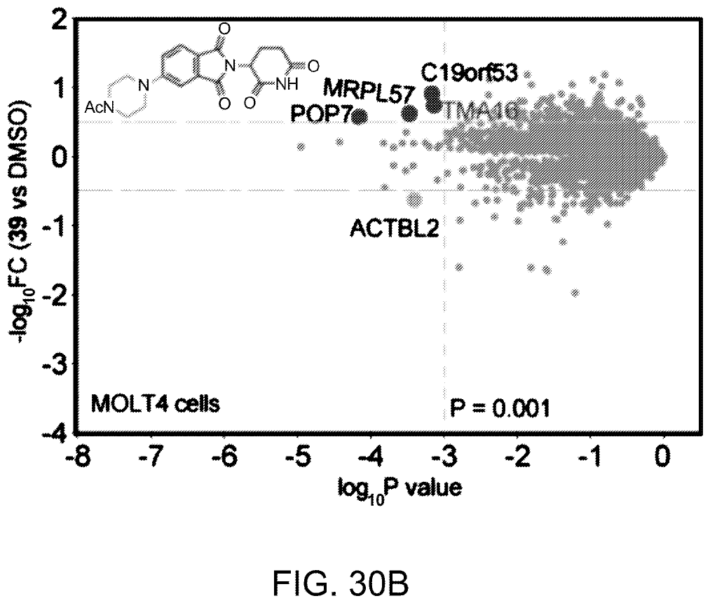

Global proteomic studies. To validate the results from the imagining-based experiments, Applicant performed proteomics studies in two ZF protein-relevant cell lines (MOLT4 and KELLY).²⁶ Briefly, Applicant treated the ZF protein-relevant cells (MOLT4 and KELLY) with IMiDs or PROTACs at 1 μM concentrations, and the protein lysates were prepared from the cells were digested, and labeled using a TMT-based quantification kit. Labeled samples were subjected to LC/MS on an Orbitrap Eclipse Tribrid mass spectrometer. Applicant used the Proteome Discoverer software pipeline and identified the final hits by searching against the uniport database. These studies suggest that the piperazine containing cleaner compound 39 exhibited no/minimal off-target profile in two cell lines (FIG. 30B) while Pomalidomide exhibited the commonly reported off-targets (FIG. 30A).

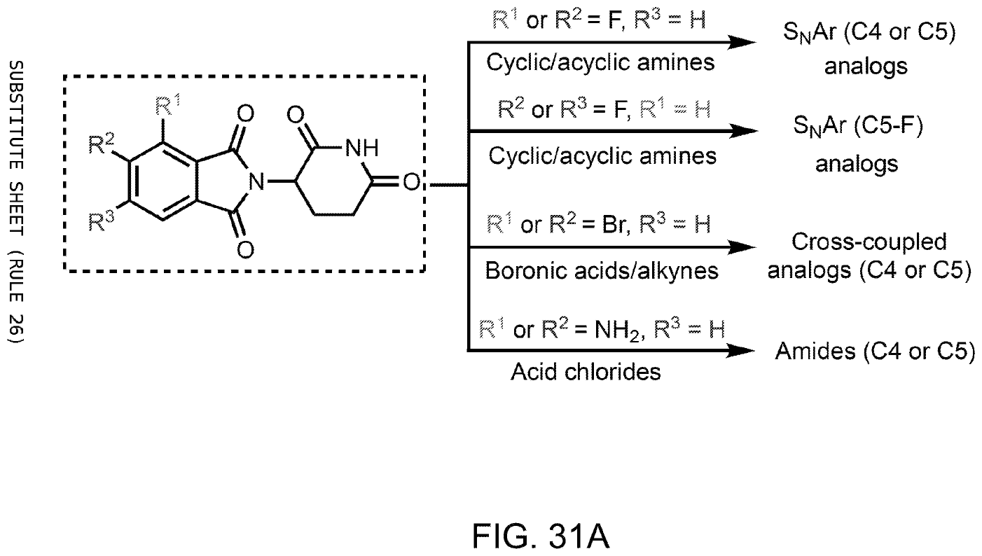

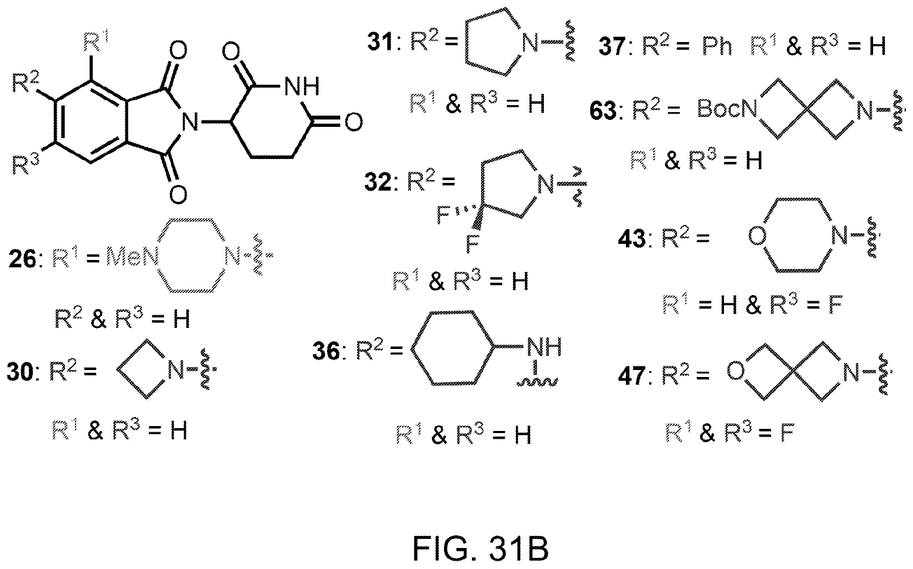

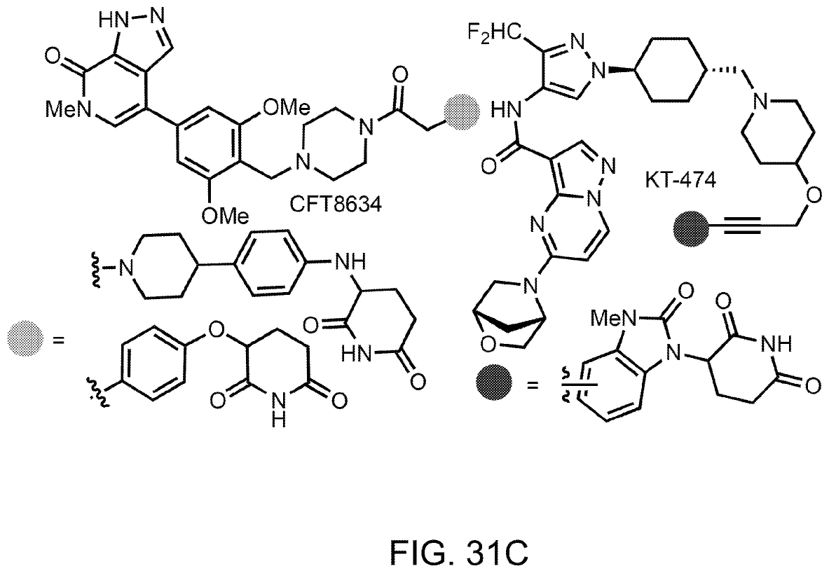

Experimental approach. Off-target profiling of pomalidomide analogs available in scientific literature and patents. Applicant will scale up production, and exhaustive characterization of ˜80 pomalidomide library described in C.1.1.1 and deeply characterize the performance of these compounds using the imaging assay (FIG. 1A) to confirm the reproducibility of our findings. Briefly, this library of pomalidomide analogs will be constructed using reactions like nucleophilic aromatic substitution (S_{N}Ar), Suzuki, Sonogashira cross-couplings, and amidation with commercially available imide compounds for the facile and scalable incorporation of C4 and C5 substitutions on the phthalimide ring (FIGS. 29A and 31A-31B). The physiochemical properties of the library (e.g., c Log P, molecular weight distribution, topological polar surface) will be evaluated as well as Caco-2 cell permeability and drug efflux properties for the top-performing compounds. Finally, Applicant has also searched literature/patents to identify glutarimide scaffolds with N-aryl/aryloxy glutarimides and benzimidazolone derivatives and their PROTACs, such as CFT8634 and KT-474 (FIG. 31C).⁴⁵ Off-target

identification for these newer scaffolds is not known. First, Applicant will synthesize N-aryl/aryloxy glutarimides⁵³ and benzimidazolone⁵⁴ derivatives and screen their off-target propensities in Applicant's high throughput imaging assay.

Cell painting assay. Applicant will use the cell painting assay to compare the morphological features of the cells treated with the library described above which have various degradation scores. The data acquisition and analysis will be similar to that described in the previously described cell painting assay. Compound performance in this assay will be compared with those in the ZF-based imaging assay.

Proteomic studies.²⁶ These studies will be performed as previously described global proteomic studies for Applicant's analogs and Applicant will devise a metric score similar to that in FIG. 3B. Applicant will compare compound performance with those from previous sections of the experimental approach (off-target profiling of pomalidomide analogs available in scientific literature and patents and the cell painting assay) to devise a weighted score for compound performance.

Zebrafish teratogenicity studies.^{55-57,31} Zebrafish teratogenicity experiments will be performed. Briefly, zebrafish embryos (2 hpf) will be dechorionated prior to IMiD treatment using Protease type XIV and then washed with E3 medium. After dechorionation, embryos will be immediately incubated with pomalidomide analogs for 24-72 h, replacing the media with freshly prepared pomalidomide analogs every 12 h. Later, embryos will be stained by Alcian blue staining and imaged to compare IMiD-induced developmental abnormalities in pectoral fins and auditory vesicles—the equivalent of arms and ears in humans.³¹

Expected outcomes and potential pitfalls. The weighted score from comparing compound performance in ZF-screen, cell painting, and proteomics studies will help identify clean IMiDs. Applicant will investigate off-target profiles of libraries and compare and cross-validate the results of the best ˜20 molecules to identify the cleaner chemical matter which can be used in the generation of PROATCs. Applicant is aware that some compounds may not correlate between different assay formats. Based on Applicant's preliminary data on a few IMiDs, it suggests that there is a strong correlation across these assay platforms. Sensitivity for SALL4 detection is low in the current ZF-degradation assay, and Applicant plans to improve it by installing multiple ZF motifs of SALL4.

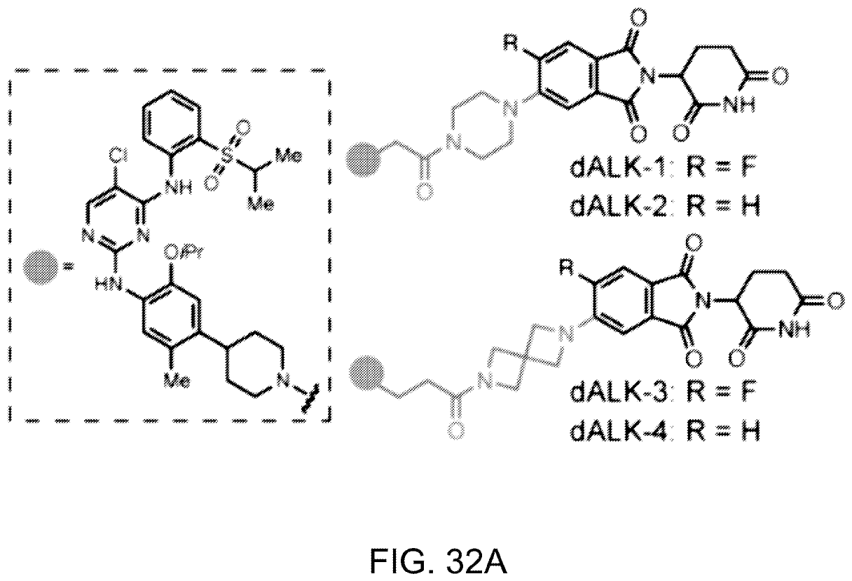

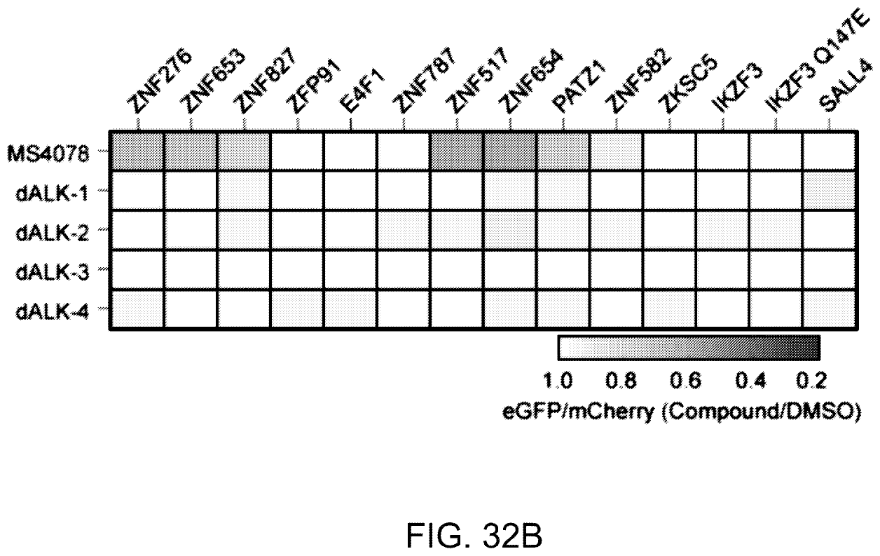

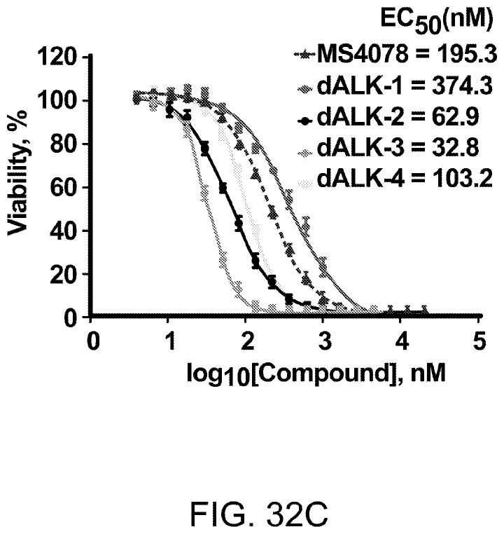

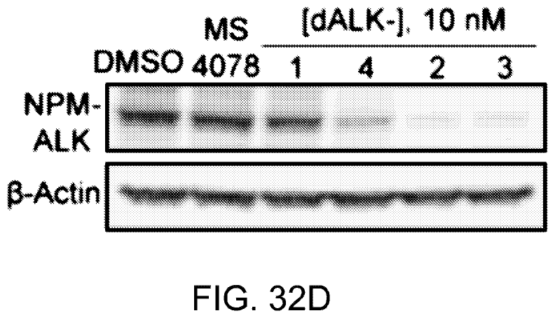

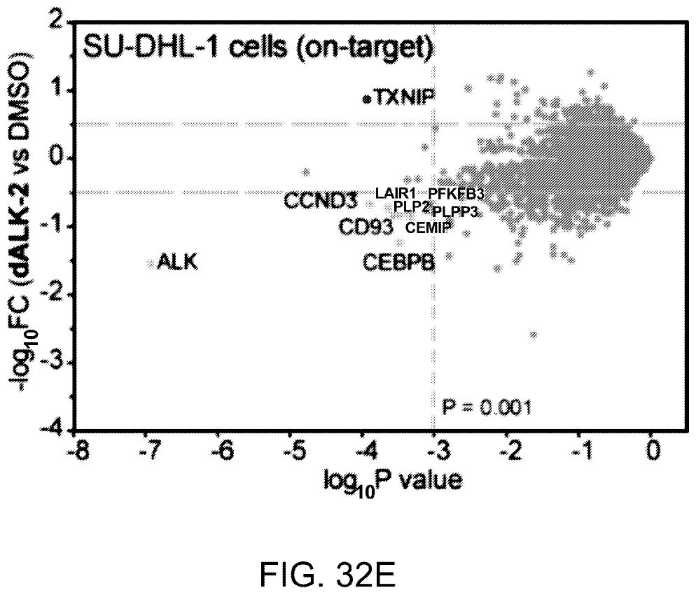

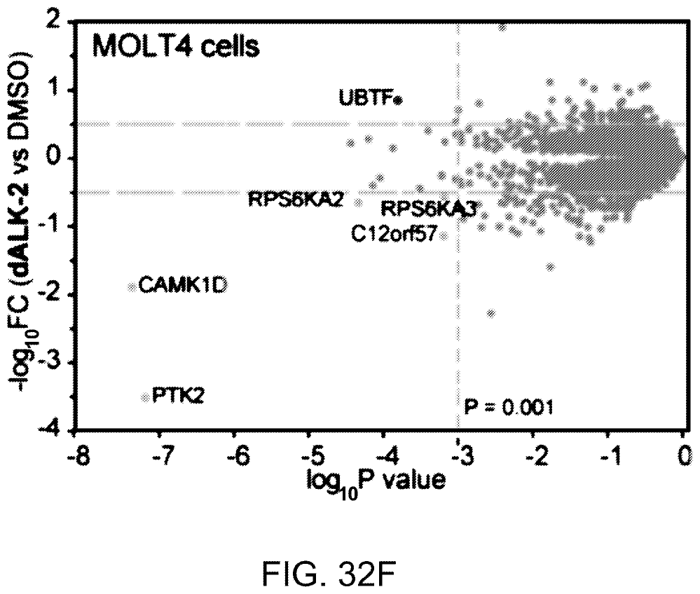

Generalization and resistance development to the new PROTACs. Design of ALK PROTACs with reduced off-targets. Anaplastic lymphoma kinase (ALK) is a tyrosine kinase receptor that forms a 2;5 chromosomal translocations in anaplastic large-cell non-Hodgkin's lymphoma (ALCL). Due to this translocation, nucleophosmin (NPM)-ALK fusion protein is produced and results in constitutive activation of the ALK kinase followed by uncontrolled cell proliferation.^{58,59} Inspired by Applicant's preliminary findings on ImiD analogs, Applicant reengineered the potent reported ALK PROTAC (MS4078)47, which had a high level of off-target ZF degradation by altering the exit vectors on Pomalidomide to reduce off-target ZF degradation while maintaining potency. Applicant selected pomalidomide analogs piperazine and 2,6-diazaspiro[3.3]heptane exit vectors on the C5 positions, which had degradation scores close to zero (FIG. 32A) and included C6 fluoro modification due to its near-zero ZF degradation score (FIG. 3B). Applicant rationally designed and synthesized four new ALK PROTACs with different exit vectors, such as piperazine with acyl linkers (dALK-1 and dALK-2) and diazaspiro[3.3]heptane (dALK-3 and dALK-4) with Propanoyl linkers by employing amidation chemistry (exit vectors shown by dotted ovals in FIG. 32A). Applicant then performed the high-throughput imaging analysis of new ALK PROTACs to investigate their off-targets. The original PROTAC MS4078 has an affinity for proteins such as ZNF517, ZNF654, ZNF276, ZNF653, and PATZ1 (FIG. 32B). New ALK PROTACs with piperazine and 2,6-diazaspiro[3.3]heptane exit vectors with and without the fluoro group exhibited minimized off-target effects (FIG. 32B). Notably, recently reported PROTACs in advanced clinical trials at Arvinas, Inc. have a similar exit vector with piperazine, which agreed with our findings of minimal ZF degradation.

Cellular potency and target degradation. Applicant then performed cytotoxicity studies to investigate any enhancement in the on-target activity. MS4078 has EC₅₀=195.3 nM, and introducing piperazine and 2,6-diazaspiro[3.3]heptane exit vectors reduced the off-target effects, thereby its EC₅₀ values. Strikingly, Applicant's ALK PROTACs with C5 piperazine exit vectors containing propyl amide linkers (dALK-1, 2, respectively) and a C5 diazaspiro[3.3]heptane linker containing propylamide (dALK-3, 4) were found to be the most potent and the best PROTACs. Among these four ALK PROTACs, Applicant identified two best-in-class PROTACs with 1.8-2-fold higher potency than MS4078 and EC₅₀ values of 32.8 nM (dALK-3) and 62.9 nM (dALK-2), which renders them more effective in reducing the SU-DHL-1 cell viability (FIG. 32C) by selectively degrading ALK protein (FIG. 32D). Furthermore, the immunoblot analysis of lysates from SU-DHL-1 cells revealed dALK-11 and dALK-12 as the potent degrader of ALK protein even at 10 nM concentration (FIG. 32D), corroborating the cytotoxicity studies.

Global proteomic studies. Applicant performed global proteomic studies in SU-DHL-1 and MOLT4 cells and observed the degradation of ceritinib binding kinase proteins but not the IMiD targets such as ZF proteins (FIGS. 32E-32F) with reduced kinases off-targets.

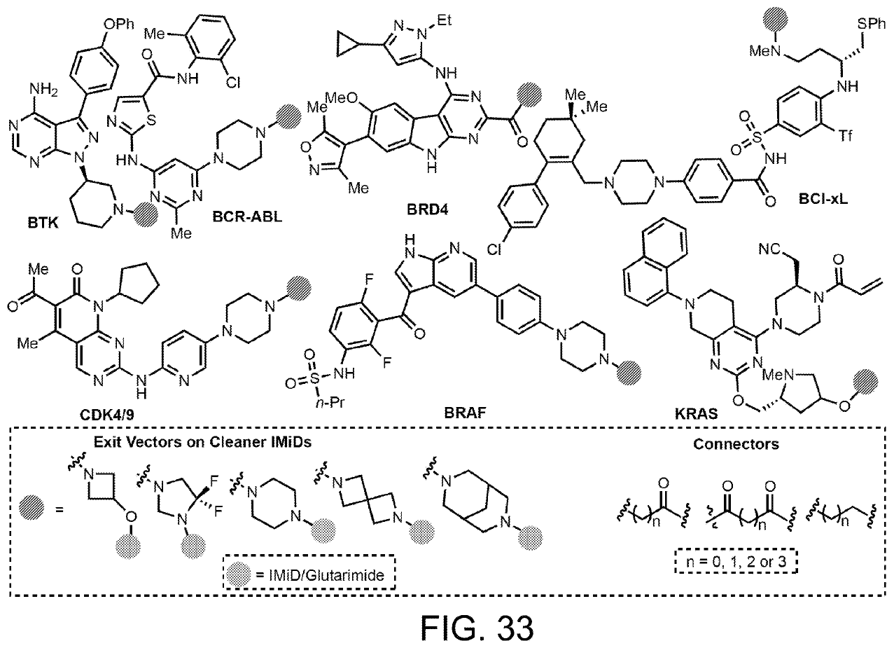

Design and evaluation of new PROTAC molecules for on-target selectivity and off-target reduction. As part of Applicant's preliminary study, Applicant has already screened and observed off-target profiles of the PROTACs (FIG. 1B), BETd-260⁶⁰ (BRD4), SJF620⁶¹ (BTK), and PROTAC K-Ras Degrader-1⁶² (KRAS). In addition, Applicant choose important cancer targets and their PROTACs (FIG. 33) and will screen for off-targets of Dasatinib-Pomalidomide-based BCR-ABL targeting PROTACs, SIAIS629050, and SIAIS629051⁶³ PROTACs that degrade CDK4/6 (MS140),⁶⁴ Bcl-xL (XZ739)⁶⁵, and BRAF⁶⁶ (Pomalidomide-based SJF-0628). Next, Applicant will prepare cleaner PROTACs for the targets BTK, BCR-ABL, CDK4, and BRAF based on IMiDs and Glutarimides with varying exit vectors and different connector types such as amides or alkyls at different lengths, as shown in FIG. 33. As investigating several new PROTACs is a resource and time-limiting, Applicant lists Bcl-xL and KRAS as potential targets. In all cases, Applicant will compare imaging results with global proteomics and screen in zebrafish-based phenotypic teratogenicity assay. Finally, Applicant will assess the PROTACs on-target activity with cell viability and immunoblot analysis. In addition, Applicant will execute a NanoBRET assay for all cleaner PROTACs to measure target occupancy & PROTACs affinity in live cells quantitatively.

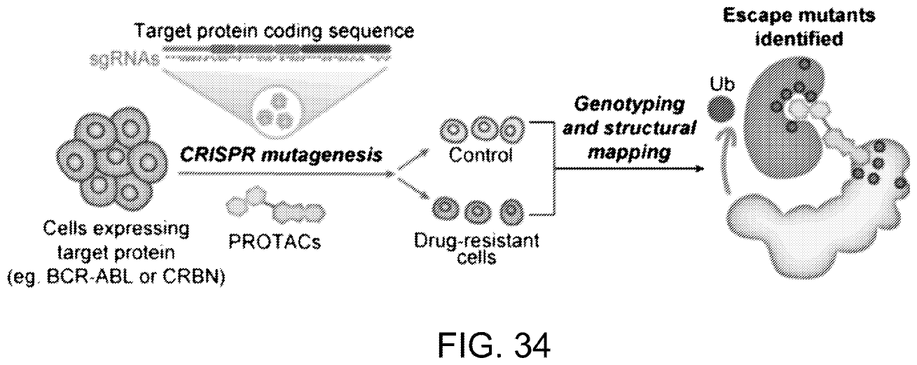

Determination of resistance evolution to PROTACs using CRISPR-scanning mutagenesis. Resistance development in cancer cell lines is often a slow process and studying such process with PROTACs gives invaluable information about the PROTACs to deprioritize or to develop PROTACs with slower rate of resistance. Resistance studies for CDK12 PROTAC revealed G-loop mutations in the CDK12 conferred the loss of binding.⁶⁷ Similarly, loss of activity was observed for CRBN-based PROTACs due to reduced expression of CRBN and UBE2G1.⁶⁸ To accelerate this process, CRISPR-Cas9 is used as a mutagen, and gRNAs are tiled across the gene to introduce different mutations in protein targets, and the process is also known as CRISPR-scanning mutagenesis.⁶⁹ These mutagenesis-induced cells will be selected by PROTAC treatment, and the cells that survived will be analyzed by high-throughput sequencing for their resistant mutations. The resistant mutations in GSPT1 and RBM39 neosubstrates targeted by cereblon and DCAF15 ligands, respectively, have been profiled.⁷⁰ Applicant will perform CRISPR-scanning mutagenesis screening.^{69,70} Applicant will tile the gRNAs across genes for the target proteins, viz. BTK, BCR-ABL, CDK4, BRAF. The selection pressure introduced by their respective PROTACs allows the emergence of escape mutants identified by the barcode appended to the guide RNA (sgRNA, FIG. 34). These studies will provide mutational structure insights of target proteins and E3 ligases and will be informative in prioritizing the IMiD/PROTAC scaffolds that do not contribute to the resistance.

Expected outcomes and potential pitfalls. There is a possibility that the synthesized PROTACs may not demonstrate efficacy compared to existing PROTACs. In that case, Applicant will investigate the cause of inferior efficacy, including linker type/length optimization, the ability of the PROTACs to form an effective ternary complex, and ubiquitination. Applicant will reengineer PROTACS using the newly designed cleaner IMiD/glutarimide building blocks to form an effective ternary complex and degradation. Applicant will also perform toxicity profiling, and further characterization of the metabolism of the PROTACs will be conducted to develop safer and more potent PROTACs in vivo.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### REFERENCES

### Example 1

1. Dale, B.; Cheng, M.; Park, K.-S.; Kaniskan, H. Ü.; Xiong, Y.; Jin, J., Advancing targeted protein degradation for cancer therapy. Nature Reviews Cancer 2021.
2. Sievers, Q. L.; Petzold, G.; Bunker, R. D.; Renneville, A.; Slabicki, M.; Liddicoat, B. J.; Abdulrahman, W.; Mikkelsen, T.; Ebert, B. L.; Thomä, N. H., Defining the human C2H2 zinc finger degrome targeted by thalidomide analogs through CRBN. Science 2018, 362 (6414), eaat0572.
3. Matyskiela, M. E.; Lu, G.; Ito, T.; Pagarigan, B.; Lu, C.-C.; Miller, K.; Fang, W.; Wang, N.-Y.; Nguyen, D.; Houston, J.; Carmel, G.; Tran, T.; Riley, M.; Nosaka, L. A.; Lander, G. C.; Gaidarova, S.; Xu, S.; Ruchelman, A. L.; Handa, H.; Carmichael, J.; Daniel, T. O.; Cathers, B. E.; Lopez-Girona, A.; Chamberlain, P. P., A novel cereblon modulator recruits GSPT1 to the CRL4CRBN ubiquitin ligase. Nature 2016, 535(7611), 252-257.
4. Wang, A.; Ding, L.; Wu, Z.; Ding, R.; Teng, X.-L.; Wang, F.; Hu, Z.; Chen, L.; Yu, X.; Zou, Q., ZFP91 is required for the maintenance of regulatory T cell homeostasis and function. Journal of Experimental Medicine 2020, 218(2).
5. Donovan, K. A.; Ferguson, F. M.; Bushman, J. W.; Eleuteri, N. A.; Bhunia, D.; Ryu, S.; Tan, L.; Shi, K.; Yue, H.; Liu, X.; Dobrovolsky, D.; Jiang, B.; Wang, J.; Hao, M.; You, I.; Teng, M.; Liang, Y.; Hatcher, J.; Li, Z.; Manz, T. D.; Groendyke, B.; Hu, W.; Nam, Y.; Sengupta, S.; Cho, H.; Shin, I.; Agius, M. P.; Ghobrial, I. M.; Ma, M. W.; Che, J.; Buhrlage, S. J.; Sim, T.; Gray, N. S.; Fischer, E. S., Mapping the Degradable Kinome Provides a Resource for Expedited Degrader Development. Cell 2020,183(6), 1714-1731.e10.
6. Zhang, C.; Han, X.-R.; Yang, X.; Jiang, B.; Liu, J.; Xiong, Y.; Jin, J., Proteolysis Targeting Chimeras (PROTACs) of Anaplastic Lymphoma Kinase (ALK). European Journal of Medicinal Chemistry 2018, 151, 304-314.
7. Nabet, B.; Roberts, J. M.; Buckley, D. L.; Paulk, J.; Dastjerdi, S.; Yang, A.; Leggett, A. L.; Erb, M. A.; Lawlor, M. A.; Souza, A.; Scott, T. G.; Vittori, S.; Perry, J. A.; Qi, J.; Winter, G. E.; Wong, K.-K.; Gray, N. S.; Bradner, J. E., The dTAG system for immediate and target-specific protein degradation. Nature Chemical Biology 2018, 14(5), 431-441.
8. Kwak, E. L.; Bang, Y.-J.; Camidge, D. R.; Shaw, A. T.; Solomon, B.; Maki, R. G.; Ou, S.-H. I.; Dezube, B. J.; Jänne, P. A.; Costa, D. B.; Varella-Garcia, M.; Kim, W.-H.; Lynch, T. J.; Fidias, P.; Stubbs, H.; Engelman, J. A.; Sequist, L. V.; Tan, W.; Gandhi, L.; Mino-Kenudson, M.; Wei, G. C.; Shreeve, S. M.; Ratain, M. J.; Settleman, J.; Christensen, J. G.; Haber, D. A.; Wilner, K.; Salgia, R.; Shapiro, G. I.; Clark, J. W.; Iafrate, A. J., Anaplastic Lymphoma Kinase Inhibition in Non-Small-Cell Lung Cancer. New England Journal of Medicine 2010, 363(18), 1693-1703.
9. Gambacorti Passerini, C.; Farina, F.; Stasia, A.; Redaelli, S.; Ceccon, M.; Mologni, L.; Messa, C.; Guerra, L.; Giudici, G.; Sala, E.; Mussolin, L.; Deeren, D.; King, M. H.; Steurer, M.; Ordemann, R.; Cohen, A. M.; Grube, M.; Bernard, L.; Chiriano, G.; Antolini, L.; Piazza, R., Crizotinib in Advanced, Chemoresistant Anaplastic Lymphoma Kinase-Positive Lymphoma Patients. JNCI: Journal of the National Cancer Institute 2014, 106(2).
10. Chen, D.; Wang, Y.; Lu, R.; Jiang, X.; Chen, X.; Meng, N.; Chen, M.; Xie, S.; Yan, G.-R., E3 ligase ZFP91 inhibits Hepatocellular Carcinoma Metabolism Reprogramming by regulating PKM splicing. Theranostics 2020, 10(19), 8558-8572.
11. Xu, S.-H.; Zhu, S.; Wang, Y.; Huang, J.-Z.; Chen, M.; Wu, Q.-X.; He, Y.-T.; Chen, D.; Yan, G.-R., ECD promotes gastric cancer metastasis by blocking E3 ligase ZFP91-mediated hnRNP F ubiquitination and degradation. Cell Death & Disease 2018, 9(5), 479.
12. Wang, J.-H.; Avitahl, N.; Cariappa, A.; Friedrich, C.; Ikeda, T.; Renold, A.; Andrikopoulos, K.; Liang, L.; Pillai, S.; Morgan, B. A.; Georgopoulos, K., <em>Aiolos</em> Regulates B Cell Activation and Maturation to Effector State. Immunity 1998, 9(4), 543-553.
13. Gajewski, T. F.; Schreiber, H.; Fu, Y.-X., Innate and adaptive immune cells in the tumor microenvironment Nature Immunology 2013, 14(10), 1014-1022.

### Example 3

1. Recognition and processing of ubiquitin-protein conjugates by the proteasome. Finley, D. Annu Rev Biochem 2009, 78, 477-513.PMC3431160.
2. Cereblon modulators: Low molecular weight inducers of protein degradation. Chamberlain, P. P.; Cathers, B. E. Drug Discov Today Technol 2019, 31, 29-34.
3. Haven't got a glue: Protein surface variation for the design of molecular glue degraders. Kozicka, Z.; Thomä, N. H. Cell Chem Biol 2021, 28, 1032-1047.
4. Protacs: chimeric molecules that target proteins to the Skp1-Cullin-F box complex for ubiquitination and degradation. Sakamoto, K. M.; Kim, K. B.; Kumagai, A.; Mercurio, F.; Crews, C. M.; Deshaies, R. J. Proc Natl Acad Sci USA 2001, 98, 8554-9.PMC3747.
5. DRUG DEVELOPMENT. Phthalimide conjugation as a strategy for in vivo target protein degradation. Winter, G. E.; Buckley, D. L.; Paulk, J.; Roberts, J. M.; Souza, A.; Dhe-Paganon, S.; Bradner, J. E. Science 2015, 348, 1376-81.PMC4937790.
6. Development of targeted protein degradation therapeutics. Chamberlain, P. P.; Hamann, L. G. Nat Chem Biol 2019, 15, 937-944.
7. Lenalidomide induces ubiquitination and degradation of CK1α in del(5q) MDS. Krönke, J.; Fink, E. C.; Hollenbach, P. W.; MacBeth, K. J.; Hurst, S. N.; Udeshi, N. D.; Chamberlain, P. P.; Mani, D. R.; Man, H. W.; Gandhi, A. K.; Svinkina, T.; Schneider, R. K.; McConkey, M.; Järås, M.; Griffiths, E.; Wetzler, M.; Bullinger, L.; Cathers, B. E.; Carr, S. A.; Chopra, R.; Ebert, B. L. Nature 2015, 523, 183-188.PMC4853910.
8. Structural basis of lenalidomide-induced CK1α degradation by the CRL4(CRBN) ubiquitin ligase. Petzold, G.; Fischer, E. S.; Thomä, N. H. Nature 2016, 532, 127-30.
9. A novel cereblon modulator recruits GSPT1 to the CRL4(CRBN) ubiquitin ligase. Matyskiela, M. E.; Lu, G.; Ito, T.; Pagarigan, B.; Lu, C. C.; Miller, K.; Fang, W.; Wang, N. Y.; Nguyen, D.; Houston, J.; Carmel, G.; Tran, T.; Riley, M.; Nosaka, L.; Lander, G. C.; Gaidarova, S.; Xu, S.; Ruchelman, A. L.; Handa, H.; Carmichael, J.; Daniel, T. O.; Cathers, B. E.; Lopez-Girona, A.; Chamberlain, P. P. Nature 2016, 535, 252-7.
10. Development of Dual and Selective Degraders of Cyclin-Dependent Kinases 4 and 6. Jiang, B.; Wang, E. S.; Donovan, K. A.; Liang, Y.; Fischer, E. S.; Zhang, T.; Gray, N. S. Angew Chem Int Ed Engl 2019, 58, 6321-6326.PMC7678623.
11. ZFP91 is required for the maintenance of regulatory T cell homeostasis and function. Wang, A.; Ding, L.; Wu, Z.; Ding, R.; Teng, X. L.; Wang, F.; Hu, Z.; Chen, L.; Yu, X.; Zou, Q. J Exp Med 2021, 218.PMC7769166.
12. RNA-binding proteins in immune regulation: a focus on CCCH zinc finger proteins. Fu, M.; Blackshear, P. J. Nat Rev Immunol 2017, 17, 130-143. PMC5556700.
13. Zinc-finger proteins in health and disease. Cassandri, M.; Smirnov, A.; Novelli, F.; Pitolli, C.; Agostini, M.; Malewicz, M.; Melino, G.; Raschellà, G. Cell Death Discov 2017, 3, 17071.PMC5683310.
14. Thalidomide induces limb defects by preventing angiogenic outgrowth during early limb formation. Therapontos, C.; Erskine, L.; Gardner, E. R.; Figg, W. D.; Vargesson, N. Proc Natl Acad Sci USA 2009, 106, 8573-8.PMC2688998.
15. Teratogenic effects of thalidomide: molecular mechanisms. Ito, T.; Ando, H.; Handa, H. Cell Mol Life Sci 2011, 68, 1569-79.
16. Thalidomide promotes degradation of SALL4, a transcription factor implicated in Duane Radial Ray syndrome. Donovan, K. A.; An, J.; Nowak, R. P.; Yuan, J. C.; Fink, E. C.; Berry, B. C.; Ebert, B. L.; Fischer, E. S. Elife 2018, 7.PMC6156078.
17. Acute pharmacological degradation of Helios destabilizes regulatory T cells. Wang, E. S.; Verano, A. L.; Nowak, R. P.; Yuan, J. C.; Donovan, K. A.; Eleuteri, N. A.; Yue, H.; Ngo, K. H.; Lizotte, P. H.; Gokhale, P. C.; Gray, N. S.; Fischer, E. S. Nat Chem Biol 2021, 17, 711-717.PMC8162940.
18. Reversible ON- and OFF-switch chimeric antigen receptors controlled by lenalidomide. Jan, M.; Scarfò, I.; Larson, R. C.; Walker, A.; Schmidts, A.; Guirguis, A. A.; Gasser, J. A.; Słabicki, M.; Bouffard, A. A.; Castano, A. P.; Kann, M. C.; Cabral, M. L.; Tepper, A.; Grinshpun, D. E.; Sperling, A. S.; Kyung, T.; Sievers, Q. L.; Birnbaum, M. E.; Maus, M. V.; Ebert, B. L. Sci Transl Med 2021, 13.PMC8045771.
19. An IMiD-inducible degron provides reversible regulation for chimeric antigen receptor expression and activity. Carbonneau, S.; Sharma, S.; Peng, L.; Rajan, V.; Hainzl, D.; Henault, M.; Yang, C.; Hale, J.; Shulok, J.; Tallarico, J.; Porter, J.; Brogdon, J. L.; Dranoff, G.; Bradner, J. E.; Hild, M.; Guimaraes, C. P. Cell Chem Biol 2021, 28, 802-812.e6.
20. Rationalizing Secondary Pharmacology Screening Using Human Genetic and Pharmacological Evidence. Deaton, A. M.; Fan, F.; Zhang, W.; Nguyen, P. A.; Ward, L. D.; Nioi, P. Toxicol Sci 2019, 167, 593-603.PMC6358245.
21. Advanced proteomics approaches to unravel protein homeostasis. Grandi, P.; Bantscheff, M. Drug Discov Today Technol 2019, 31, 99-108.
22. Native Mass Spectrometry Can Effectively Predict PROTAC Efficacy. Beveridge, R.; Kessler, D.; Rumpel, K.; Ettmayer, P.; Meinhart, A.; Clausen, T. ACS Cent Sci 2020, 6, 1223-1230.PMC7379389 P.E. were employees of Boehringer Ingelheim at the time of this work.
23. The Vital Role of Proteomics in Characterizing Novel Protein Degraders. Zhang, A. X.; Cassidy, K.; Dahl, G.; Moreau, K.; Pachl, F.; Zuhl, A. M. SLAS Discov 2021, 26, 518-523.
24. A proteomic platform to identify off-target proteins associated with therapeutic modalities that induce protein degradation or gene silencing. Liu, X.; Zhang, Y.; Ward, L. D.; Yan, Q.; Bohnuud, T.; Hernandez, R.; Lao, S.; Yuan, J.; Fan, F. Sci Rep 2021, 11, 15856.PMC8338952
25. Challenges in mass spectrometry-based proteomics. Reinders, J.; Lewandrowski, U.; Moebius, J.; Wagner, Y.; Sickmann, A. Proteomics 2004, 4, 3686-703.
26. Mapping the Degradable Kinome Provides a Resource for Expedited Degrader Development. Donovan, K. A.; Ferguson, F. M.; Bushman, J. W.; Eleuteri, N. A.; Bhunia, D.; Ryu, S.; Tan, L.; Shi, K.; Yue, H.; Liu, X.; Dobrovolsky, D.; Jiang, B.; Wang, J.; Hao, M.; You, I.; Teng, M.; Liang, Y.; Hatcher, J.; Li, Z.; Manz, T. D.; Groendyke, B., Hu, W.; Nam, Y.; Sengupta, S.; Cho, H.; Shin, I.; Agius, M. P.; Ghobrial, I. M.; Ma, M. W.; Che, J.; Buhrlage, S. J.; Sim, T.; Gray, N. S.; Fischer, E. S. Cell 2020, 183, 1714-1731.e10.
27. Proteolysis Targeting Chimeras With Reduced Off-targets. Nguyen, T. M., Deb, A.; Kokkonda, P.; Sreekanth, V.; Tiwari, P. K.; Shoba, V.; Lai, S.; Chaudhary, S. K.; Mercer, J. A. M.; Jan, M.; Liu, D. R.; Ebert, B. L.; Choudhary, A. bioRxiv 2021, 2021.11.18.468552.
28. Arvinas unveils PROTAC structures. C&EN Global Enterprise 2021, 99, 5-5
29. Phase 1/2 study of ARV-110, an androgen receptor (AR) PROTAC degrader, in metastatic castration-resistant prostate cancer (mCRPC). Gao, X.; III, H. A. B.; Vuky, J.; Dreicer, R.; Sartor, A. O.; Sternberg, C. N.; Percent, I. J.; Hussain, M. H. A.; Kalebasty, A. R.; Shen, J.; Heath, E. I.; Abesada-Terk, G.; Gandhi, S. G.; McKean, M.; Lu, H.; Berghorn, E.; Gedrich, R.; Chirnomas, S. D.; Vogelzang, N. J.; Petrylak, D. P. Journal of Clinical Oncology 2022, 40, 17-17.
30. Defining the human C2H2 zinc finger degrome targeted by thalidomide analogs through CRBN. Sievers, Q. L.; Petzold, G.; Bunker, R. D.; Renneville, A.; Słabicki, M.; Liddicoat, B. J.; Abdulrahman, W.; Mikkelsen, T.; Ebert, B. L.; Thomä, N. H. Science 2018, 362, eaat0572.
31. Identification of a primary target of thalidomide teratogenicity. Ito, T.; Ando, H.; Suzuki, T.; Ogura, T.; Hotta, K.; Imamura, Y.; Yamaguchi, Y.; Handa, H. Science 2010, 327, 1345-50.
32. Lenalidomide causes selective degradation of IKZF1 and IKZF3 in multiple myeloma cells. Krönke, J.; Udeshi, N. D.; Narla, A.; Grauman, P.; Hurst, S. N.; McConkey, M.; Svinkina, T.; Heckl, D.; Comer, E.; Li, X.; Ciarlo, C.; Hartman, E.; Munshi, N.; Schenone, M.; Schreiber, S. L.; Carr, S. A.; Ebert, B. L. Science 2014, 343, 301-5.PMC4077049.
33. The myeloma drug lenalidomide promotes the cereblon-dependent destruction of Ikaros proteins. Lu, G.; Middleton, R. E.; Sun, H.; Naniong, M.; Ott, C. J.; Mitsiades, C. S.; Wong, K. K.; Bradner, J. E.; Kaelin, W. G., Jr. Science 2014, 343, 305-9.PMC4070318.
34. Targeted Protein Degradation by Small Molecules. Bondeson, D. P.; Crews, C. M. Annu Rev Pharmacol Toxicol 2017, 57, 107-123.PMC5586045.
35. PROTAC targeted protein degraders: the past is prologue. Békés, M.; Langley, D. R.; Crews, C. M. Nat Rev Drug Discov 2022, 21, 181-200.PMC8765495 which support research in his lab. M.B. and D.R.L. are employees and shareholders of Arvinas, Inc, which is developing drug candidates in the targeted protein degradation space.
36. E3 ligase ligand chemistries: from building blocks to protein degraders. Sosič, I.; Bricelj, A.; Steinebach, C. Chem Soc Rev 2022, 51, 3487-3534.
37. Multifunctional zinc finger proteins in development and disease. Ladomery, M.; Dellaire, G. Ann Hum Genet 2002, 66, 331-42.
38. Zinc finger transcription factors in skeletal development. Ganss, B.; Jheon, A. Crit Rev Oral Biol Med 2004, 15, 282-97.
39. Zinc finger protein Zfp335 controls early T-cell development and survival through β-selection-dependent and -independent mechanisms. Wang, X.; Jiao, A.; Sun, L.; Li, W.; Yang, B.; Su, Y.; Ding, R.; Zhang, C.; Liu, H.; Yang, X.; Sun, C.; Zhang, B. Elife 2022, 11.PMC8871394.
40. Shedding light on an old mystery: thalidomide suppresses survival pathways to induce limb defects. Knobloch, J.; Rüther, U. Cell Cycle 2008, 7, 1121-7.
41. Ikaros Zinc Finger Transcription Factors: Regulators of Cytokine Signaling Pathways and CD4(+) T Helper Cell Differentiation. Powell, M. D.; Read, K. A.; Sreekumar, B. K.; Oestreich, K. J. Front Immunol 2019, 10, 1299.PMC6563078.
42. Recent advances in the molecular mechanism of thalidomide teratogenicity. Gao, S.; Wang, S.; Fan, R.; Hu, J. Biomed Pharmacother 2020, 127, 110114.
43. Target and tissue selectivity of PROTAC degraders. Guenette, R. G.; Yang, S. W.; Min, J.; Pei, B.; Potts, P. R. Chem Soc Rev 2022.
44. Phenotypes associated with genes encoding drug targets are predictive of clinical trial side effects. Nguyen, P. A.; Born, D. A.; Deaton, A. M.; Nioi, P.; Ward, L. D. Nat Commun 2019, 10, 1579.PMC6450952 former employees of Amgen, Inc.
45. Targeted protein degraders crowd into the clinic. Mullard, A. Nat Rev Drug Discov 2021, 20, 247-250.
46. Advancing targeted protein degradation for cancer therapy. Dale, B.; Cheng, M.; Park, K. S.; Kaniskan, H.; Xiong, Y.; Jin, J. Nat Rev Cancer 2021, 21, 638-654.PMC8463487.
47. Proteolysis Targeting Chimeras (PROTACs) of Anaplastic Lymphoma Kinase (ALK). Zhang, C.; Han, X. R.; Yang, X.; Jiang, B.; Liu, J.; Xiong, Y.; Jin, J. Eur J Med Chem 2018, 151, 304-314.PMC5924614.
48. The dTAG system for immediate and target-specific protein degradation. Nabet, B.; Roberts, J. M.; Buckley, D. L.; Paulk, J.; Dastjerdi, S.; Yang, A.; Leggett, A. L.; Erb, M. A.; Lawlor, M. A.; Souza, A.; Scott, T. G.; Vittori, S.; Perry, J. A.; Qi, J.; Winter, G. E.; Wong, K.-K.; Gray, N. S.; Bradner, J. E. Nature Chemical Biology 2018, 14, 431-441.
49. Chemogenetic System Demonstrates That Cas9 Longevity Impacts Genome Editing Outcomes. Sreekanth, V.; Zhou, Q.; Kokkonda, P.; Bermudez-Cabrera, H. C.; Lim, D.; Law, B. K.; Holmes, B. R.; Chaudhary, S. K.; Pergu, R.; Leger, B. S.; Walker, J. A.; Gifford, D. K.; Sherwood, R. I.; Choudhary, A. ACS Cent Sci 2020, 6, 2228-2237.PMC7760466 has filed patents claiming inventions to genome editing methods in this manuscript.
50. Cell Painting, a high-content image-based assay for morphological profiling using multiplexed fluorescent dyes. Bray, M. A.; Singh, S.; Han, H.; Davis, C. T.; Borgeson, B.; Hartland, C.; Kost-Alimova, M.; Gustafsdottir, S. M.; Gibson, C. C.; Carpenter, A. E. Nat Protoc 2016, 11, 1757-74.PMC5223290.
51. NIH Image to ImageJ: 25 years of image analysis. Schneider, C. A.; Rasband, W. S.; Eliceiri, K. W. Nat Methods 2012, 9, 671-5.PMC5554542.
52. CellProfiler 4: improvements in speed, utility and usability. Stirling, D. R.; Swain-Bowden, M. J.; Lucas, A. M.; Carpenter, A. E.; Cimini, B. A.; Goodman, A. BMC Bioinformatics 2021, 22, 433.PMC8431850
53. Novel Compounds for Targeted Degradation of BRD9 and Their Use for Treating Cancer. Sabnis, R. W. ACS Medicinal Chemistry Letters 2022, 13, 17-18.
54. Preparation of novel bifunctional compounds as IRAK degraders and uses thereof. Weiss, M. M.; Zheng, X.; Kymera Therapeutics, Inc., USA. 2021, p 320pp.
55. Chemical suppression of a genetic mutation in a zebrafish model of aortic coarctation. Peterson, R. T.; Shaw, S. Y.; Peterson, T. A.; Milan, D. J.; Zhong, T. P.; Schreiber, S. L.; MacRae, C. A.; Fishman, M. C. Nat Biotechnol 2004, 22, 595-9.
56. Dorsomorphin inhibits BMP signals required for embryogenesis and iron metabolism. Yu, P. B.; Hong, C. C.; Sachidanandan, C.; Babitt, J. L.; Deng, D. Y.; Hoyng, S. A.; Lin, H. Y.; Bloch, K. D.; Peterson, R. T. Nat Chem Biol 2008, 4, 33-41.PMC2727650.
57. BMP type I receptor inhibition reduces heterotopic [corrected] ossification. Yu, P. B.; Deng, D. Y.; Lai, C. S.; Hong, C. C.; Cuny, G. D.; Bouxsein, M. L.; Hong, D. W.; McManus, P. M.; Katagiri, T.; Sachidanandan, C.; Kamiya, N.; Fukuda, T.; Mishina, Y.; Peterson, R. T.; Bloch, K. D. Nat Med 2008, 14, 1363-9.PMC2846458.
58. The anaplastic lymphoma kinase in the pathogenesis of cancer. Chiarle, R.; Voena, C.; Ambrogio, C.; Piva, R.; Inghirami, G. Nat Rev Cancer 2008, 8, 11-23.
59. Mechanistic insight into ALK receptor tyrosine kinase in human cancer biology. Hallberg, B.; Palmer, R. H. Nat Rev Cancer 2013, 13, 685-700.
60. Discovery of a Small-Molecule Degrader of Bromodomain and Extra-Terminal (BET) Proteins with Picomolar Cellular Potencies and Capable of Achieving Tumor Regression. Zhou, B.; Hu, J.; Xu, F.; Chen, Z.; Bai, L.; Fernandez-Salas, E.; Lin, M.; Liu, L.; Yang, C.-Y.; Zhao, Y.; McEachern, D.; Przybranowski, S.; Wen, B.; Sun, D.; Wang, S. Journal of Medicinal Chemistry 2018, 61, 462-481.
61. Design, synthesis and biological evaluation of Proteolysis Targeting Chimeras (PROTACs) as a BTK degraders with improved pharmacokinetic properties. Jaime-Figueroa, S.; Buhimschi, A. D.; Toure, M.; Hines, J.; Crews, C. M. Bioorg Med Chem Lett 2020, 30, 126877.PMC7318425.
62. Preparation of bifunctional compounds as modulators of proteolysis useful for treating cancer. Crew, A. P.; Hornberger, K. R.; Wang, J.; Dong, H.; Berlin, M.; Crews, C. M.; Arvinas Operations, Inc., USA; Yale University. 2019, p 1213pp.
63. Construction of an IMiD-based azide library as a kit for PROTAC research. Liu, H.; Sun, R.; Ren, C.; Qiu, X.; Yang, X.; Jiang, B. Org Biomol Chem 2021, 19, 166-170.
64. Distinct CDK6 complexes determine tumor cell response to CDK4/6 inhibitors and degraders. Wu, X.; Yang, X.; Xiong, Y.; Li, R.; Ito, T.; Ahmed, T. A.; Karoulia, Z.; Adamopoulos, C.; Wang, H.; Wang, L.; Xie, L.; Liu, J.; Ueberheide, B.; Aaronson, S. A.; Chen, X.; Buchanan, S. G.; Sellers, W. R.; Jin, J.; Poulikakos, P. I. Nat Cancer 2021, 2, 429-443.PMC8462800.
65. Discovery of PROTAC BCL-X(L) degraders as potent anticancer agents with low on-target platelet toxicity. Zhang, X.; Thummuri, D.; Liu, X.; Hu, W.; Zhang, P.; Khan, S.; Yuan, Y.; Zhou, D.; Zheng, G. Eur J Med Chem 2020, 192, 112186.PMC7433031.
66. Mutant-selective degradation by BRAF-targeting PROTACs. Alabi, S.; Jaime-Figueroa, S.; Yao, Z.; Gao, Y.; Hines, J.; Samarasinghe, K. T. G.; Vogt, L.; Rosen, N.; Crews, C. M. Nat Commun 2021, 12, 920.PMC7876048.
67. Discovery and resistance mechanism of a selective CDK12 degrader. Jiang, B.; Gao, Y.; Che, J.; Lu, W.; Kaltheuner, I. H.; Dries, R.; Kalocsay, M.; Berberich, M. J.; Jiang, J.; You, I.; Kwiatkowski, N.; Riching, K. M.; Daniels, D. L.; Sorger, P. K.; Geyer, M.; Zhang, T.; Gray, N. S. Nat Chem Biol 2021, 17, 675-683.PMC8590456.
68. Cellular Resistance Mechanisms to Targeted Protein Degradation Converge Toward Impairment of the Engaged Ubiquitin Transfer Pathway. Ottis, P.; Palladino, C.; Thienger, P.; Britschgi, A.; Heichinger, C.; Berrera, M.; Julien-Laferriere, A.; Roudnicky, F.; Kam-Thong, T.; Bischoff, J. R.; Martoglio, B.; Pettazzoni, P. ACS Chem Biol 2019, 14, 2215-2223.
69. CRISPR-suppressor scanning reveals a nonenzymatic role of LSD1 in AML. Vinyard, M. E.; Su, C.; Siegenfeld, A. P.; Waterbury, A. L.; Freedy, A. M.; Gosavi, P. M.; Park, Y.; Kwan, E. E.; Senzer, B. D.; Doench, J. G.; Bauer, D. E.; Pinello, L.; Liau, B. B. Nat Chem Biol 2019, 15, 529-539.PMC7679026.
70. Profiling the Landscape of Drug Resistance Mutations in Neosubstrates to Molecular Glue Degraders. Gosavi, P. M.; Ngan, K. C.; Yeo, M. J. R.; Su, C.; Li, J.; Lue, N. Z.; Hoenig, S. M.; Liau, B. B. ACS Cent Sci 2022, 8, 417-429.PMC9052798.

What is claimed is:

1. A molecule according to the formula 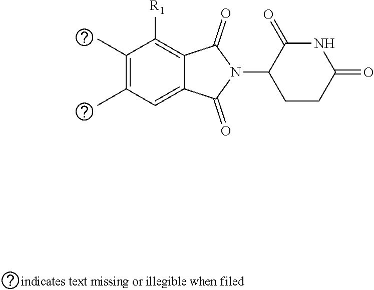
wherein R₁ is selected from —H, —R₄, —NHC(O)R₅, —NR₆R₇, —NHR₈, and —NHS(O₂)R₉;
wherein R₂ is selected from —H, —R₄, —NH₂, —NHC(O)R₅, —NR₆R₇, —NHR₈, and —NHS(O₂)R₉;
wherein R₃ is selected from —H, —R₄, and —NR₆R₇;
wherein R₄-R₉ are independently selected from one or more nitrile, nitro, ether, alcohol, thiol, sulfone, sulfonate, halogen, carbonyl, acyl, ketone, carboxylate ester, amide, enone, anhydride, imide, alkyl, alkenyl, alkynyl, saturated cyclic hydrocarbon, unsaturated cyclic hydrocarbon, heteroalkyl, heterocyclic ring, aryl ring, and heteroaryl ring groups, and one or more fused rings thereof, more preferably selected from alkyl, amide, heteroalkyl, cycloalkyl, heterocyclic, aryl and heteroaryl groups; and
wherein:
when R₂ and R₃ are —H, R₁ is selected from —R₄, —NHC(O)R₅, —NR₆R₇, —NHR₈, and —NHS(O₂)R₉; and
when R₁ and R₃ are —H, R₂ is selected from —R₄, —NH₂, —NHC(O)R₅, —NR₆R₇, —NHR₈, and —NHS(O₂)R₉.

2. The molecule of claim 1, wherein R₂ and R₃ are —H, and R₁ is selected from —R₄, —NHC(O)R₅, and —NR₆R₇.

3. The molecule of claim 1 or 2, wherein R₁ is according to —R₄, and —R₄ is selected from halogen, aryl, heteroaryl, and alkynyl groups.

4. The molecule of claim 3, wherein the halogen group is a bromine or a fluorine group.

5. The molecule of claim 3, wherein the aryl group is a phenyl group and the heteroaryl group is a pyridinyl group.

6. The molecule of claim 3, wherein the heteroaryl group is selected from indolyl, pyridinyl, isoxazolyl, and thiophene groups.

7. The molecule of claim 6, wherein the indolyl group is a 1-methyl-indolyl 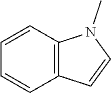
group, the isoxazolyl group is a 3,5-dimethyl-isoxazolyl 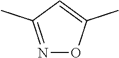
group, or the thiophene group is a benzothiophene group

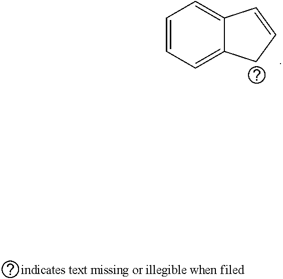

8. The molecule of claim 3, wherein the alkynyl group is a 2-phenyl-acetylenyl group.

9. The molecule of claim 1 or 2, wherein R₁ is —NHC(O)R₅, and R₅ is selected from alkyl, cycloalkyl, heterocyclic, heteroaryl, and aryl groups.

10. The molecule of claim 9, wherein R₅ is selected from methyl, phenyl, cyclopropyl, cyclobutyl, cyclopentyl, isoxazolyl, pyridinyl, and pyrazinyl groups.

11. The molecule of claim 1 or 2, wherein R₁ is according to —NR₆R₇, and N, R₆, and R₇ taken together form a heterocyclic amine group.

12. The molecule of claim 11, wherein the heterocyclic amine group is selected from morpholinyl, azetidinyl, pyrrolidinyl, piperidinyl, piperazinyl, and diazaspiro groups.

13. The molecule of claim 12, wherein the pyrrolidinyl group is an unsubstituted pyrrolidinyl group or a 3, 3′-difluoro-pyrrolidinyl group.

14. The molecule of claim 12, wherein the piperazinyl group is a 4-acetyl-1-piperazinyl, a 4-Boc-1-piperazinyl, or a 4-methyl-1-piperazinyl group.

15. The molecule of claim 12, wherein the diazaspiro group is a 2,6-diazaspiro[3.3]heptane 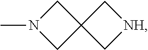
a 2-oxa-6-azaspiro[3.3]heptane 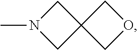
a 2-Boc-2,6-diazaspiro[3.3]heptane 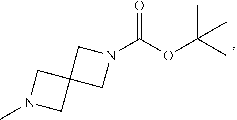
or a 3-Boc-3,9-diazaspiro[5.5]undecane 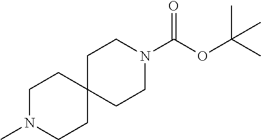 [SMILES: CN1CCC2(CC1)CCN(C(=O)OC(C)(C)C)CC2]
group.

16. The molecule of claim 1 or 2, wherein R₁ is according to —NR₆R₇ or —NHR₈, R₆ and R₇ are independently selected from alkyl and cycloalkyl groups, and R₈ is a cycloalkyl group.

17. The molecule of claim 16, wherein —NR₆R₇ is a methylcyclohexyl amine group, and R₈ is a cyclohexyl group or a morpholinyl group.

18. The molecule of claim 1, wherein R₁ and R₃ are —H, and R₂ is selected from —R₄, —NH₂, —NHC(O)R₅, —NR₆R₇, —NHR₈, and —NHS(O₂)R₉.

19. The molecule of claim 1 or 18, wherein R₂ is according to —R₄, and —R₄ is selected from halogen, nitro, heteroaryl, aryl, and alkynyl groups.

20. The molecule of claim 19, wherein the halogen group is a fluorine or bromine group.

21. The molecule of claim 19, wherein the heteroaryl group is selected from indolyl, pyridinyl, isoxazolyl, and thiophene groups.

22. The molecule of claim 21, wherein the indolyl group is a 1-methyl-indolyl 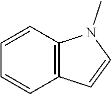
group, the isoxazolyl group is a 3,5-dimethyl-isoxazolyl 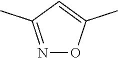
group, and the thiophene group is a benzothiophene group

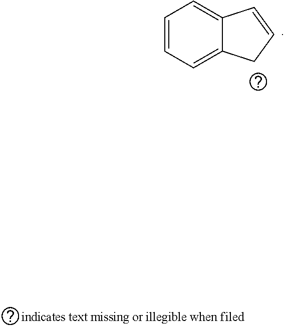

23. The molecule of claim 19, wherein the aryl group is selected from phenyl.

24. The molecule of claim 19, wherein the alkynyl group is a 2-phenyl-acetylenyl group.

25. The molecule of claim 1 or 18, wherein R₂ is according to —NHC(O)R₅, and R₅ is selected from alkyl, cycloalkyl, heterocyclic, heteroaryl, and aryl groups.

26. The molecule of claim 25, wherein R₅ is a methyl, a phenyl, a cyclopropyl, a cyclobutyl, a cyclopentyl, an isoxazolyl, a pyridinyl, or a pyrazinyl group.

27. The molecule of claim 1 or 18, wherein R₂ is according to —NR₆R₇, and N, R₆, and R₇ taken together form a heterocyclic amine group.

28. The molecule of claim 27, wherein the heterocyclic amine group is selected from morpholinyl, azetidinyl, pyrrolidinyl, piperidinyl, piperazinyl, and diazaspiro groups.

29. The molecule of claim 28, wherein the pyrrolidinyl group is an unsubstituted pyrrolidinyl group or a 3, 3′-difluoro-pyrrolidinyl group.

30. The molecule of claim 28, wherein the piperazinyl group is a 4-acetyl-1-piperazinyl, a 4-Boc-1-piperazinyl, or a 4-methyl-1-piperazinyl group.

31. The molecule of claim 28, wherein the diazaspiro group is a 2,6-diazaspiro[3.3]heptane 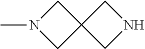
group, a 2-oxa-6-azaspiro[3.3]heptane 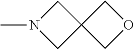
group, a 2-Boc-2,6-diazaspiro[3.3]heptane 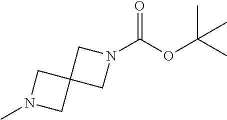
group, or a 3-Boc-3,9-diazaspiro[5.5]undecane 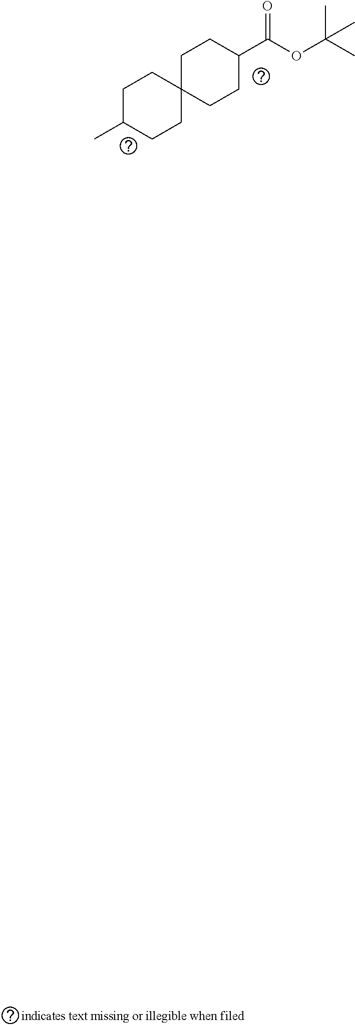 [SMILES: CC1CCC2(CC1)CCC(C(=O)OC(C)(C)C)CC2]
group.

32. The molecule of claim 1 or 18, wherein R₂ is according to —NR₆R₇ or —NHR₈, R₆ and R₇ are independently selected from alkyl and cycloalkyl groups, and R₈ is selected from cycloalkyl and heterocyclic groups.

33. The molecule of claim 32, wherein —NR₆R₇ is a methylcyclohexyl amine group, and R₈ is a cyclohexyl group or a morpholinyl group.

34. The molecule of claim 1 or 18, wherein R₂ is according to —NHS(O₂)R₉, and R₉ is an aryl group.

35. The molecule of claim 1, wherein R₁ is —H and R₂ and R₃ are according to the same —R₄ or —NR₆R₇, and N, R₆, and R₇ taken together form a heterocyclic amine group.

36. The molecule of claim 1 or 35, wherein R₂ and R₃ are according to the same —R₄, and —R₄, is a halogen.

37. The molecule of claim 36, wherein the halogen is a fluorine group.

38. The molecule of claim 1 or 35, wherein R₂ and R₃ are according to the same —NR₆R₇, and —NR₆R₇ is a morpholinyl group.

39. The molecule of claim 1, wherein R₁ is —H, R₃ is according to —R₄, R₂ is according to —NR₆R₇, and N, R₆, and R₇ taken together form a heterocyclic amine group.

40. The molecule of claim 39, wherein —R₄ is a halogen and the heterocyclic amine group is selected from morpholinyl, piperazinyl, and diazaspiro groups.

41. The molecule of claim 40, wherein the halogen is a fluorine group.

42. The molecule of claim 40, wherein the piperazinyl group is a 4-acetyl-1-piperazinyl, a 4-Boc-1-piperazinyl, or a 4-methyl-1-piperazinyl group.

43. The molecule of claim 40, wherein the diazaspiro group is a 2-oxa-6-azaspiro[3.3]heptane 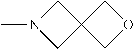
group, a 2-Boc-2,6-diazaspiro[3.3]heptane 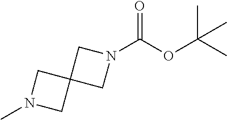
group, or a 3-Boc-3,9-diazaspiro[5.5]undecane 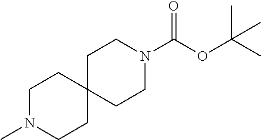 [SMILES: CN1CCC2(CC1)CCN(C(=O)OC(C)(C)C)CC2]
group.

44. The molecule of claim 39, wherein —R₄ is an aryl group and the heterocyclic amine group is a morpholinyl group.

45. The molecule of claim 44, wherein the aryl group is a phenyl group.

46. The molecule of claim 1, wherein the molecule is according to: 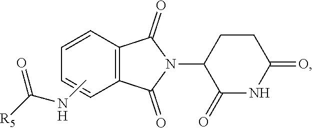
wherein R₅ is selected from

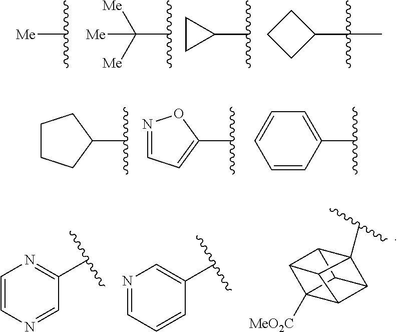

47. The molecule of claim 1, wherein the molecule is according to the formula 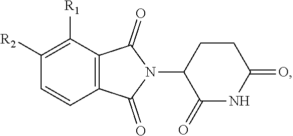
wherein when R₁ is H, R₂ is selected from 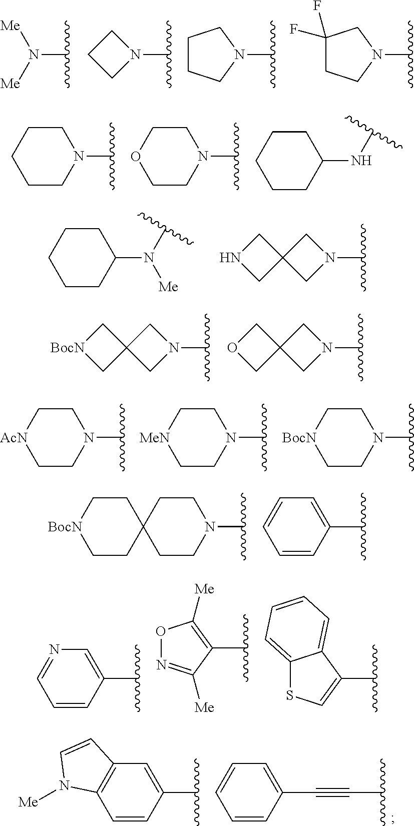
and wherein when R₂ is H, R₁ is selected from

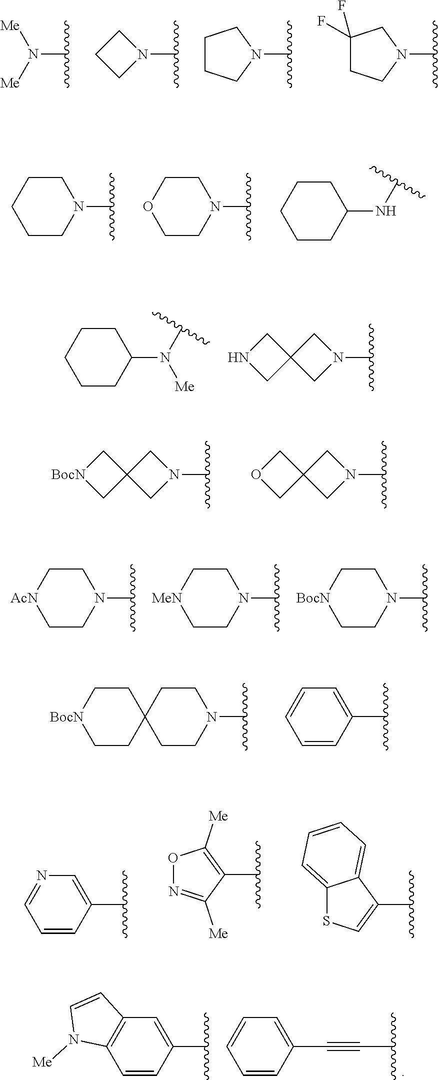

48. The molecule of any one of claims 1-45 selected from

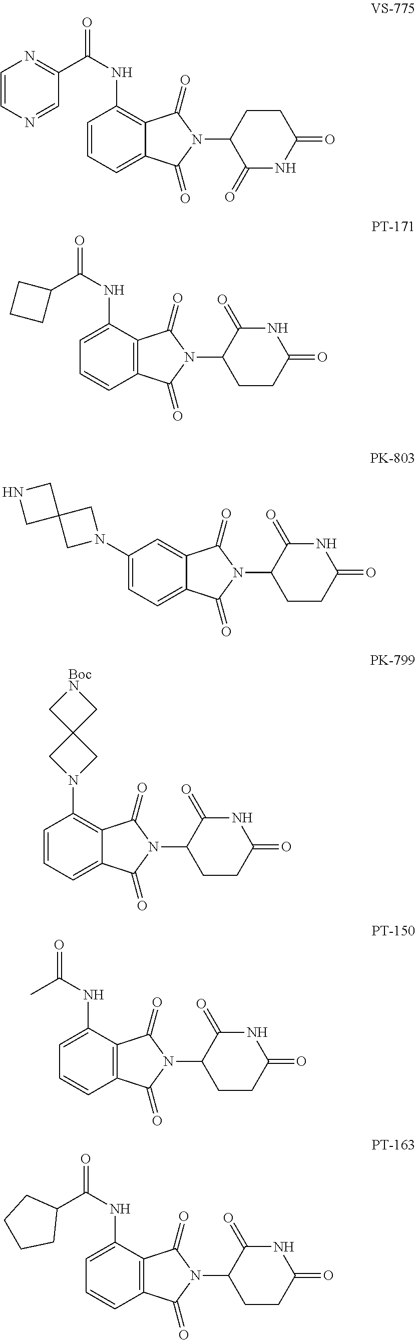 [SMILES: O=C1CCC(N2C(=O)c3cccc(NC(=O)c4cnccn4)c3C2=O)C(=O)N1 ; O=C1CCC(N2C(=O)c3cccc(NC(=O)C4CCC4)c3C2=O)C(=O)N1 ; O=C1CCC(N2C(=O)c3ccc(N4CC5(CNC5)C4)cc3C2=O)C(=O)N1 ; CC(C)(C)OC(=O)N1CC2(C1)CN(c1cccc3c1C(=O)N(C1CCC(=O)NC1=O)C3=O)C2 ; CC(=O)Nc1cccc2c1C(=O)N(C1CCC(=O)NC1=O)C2=O ; O=C1CCC(N2C(=O)c3cccc(NC(=O)C4CCCC4)c3C2=O)C(=O)N1]

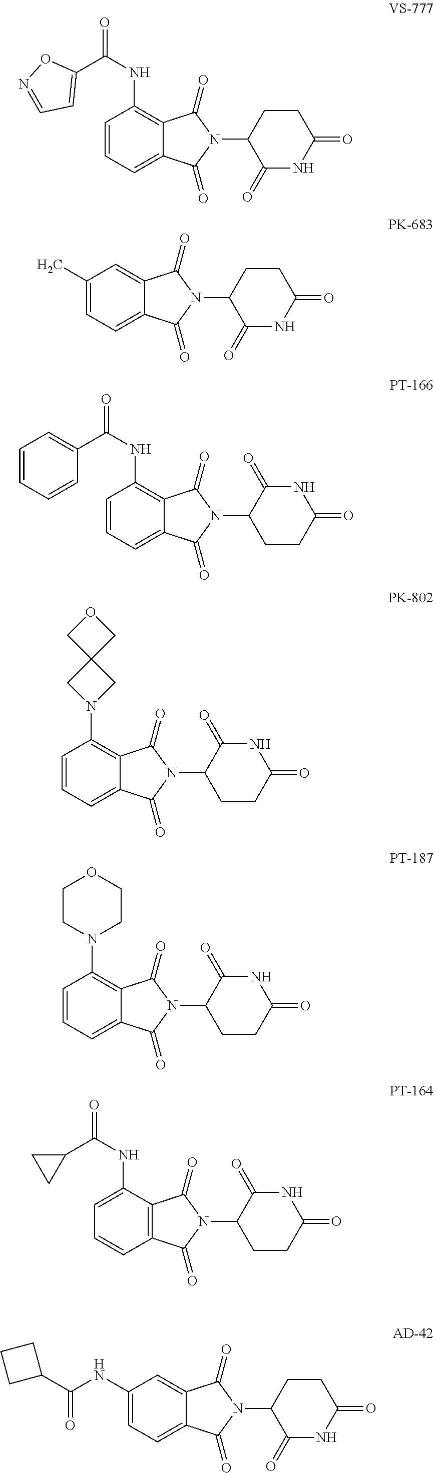

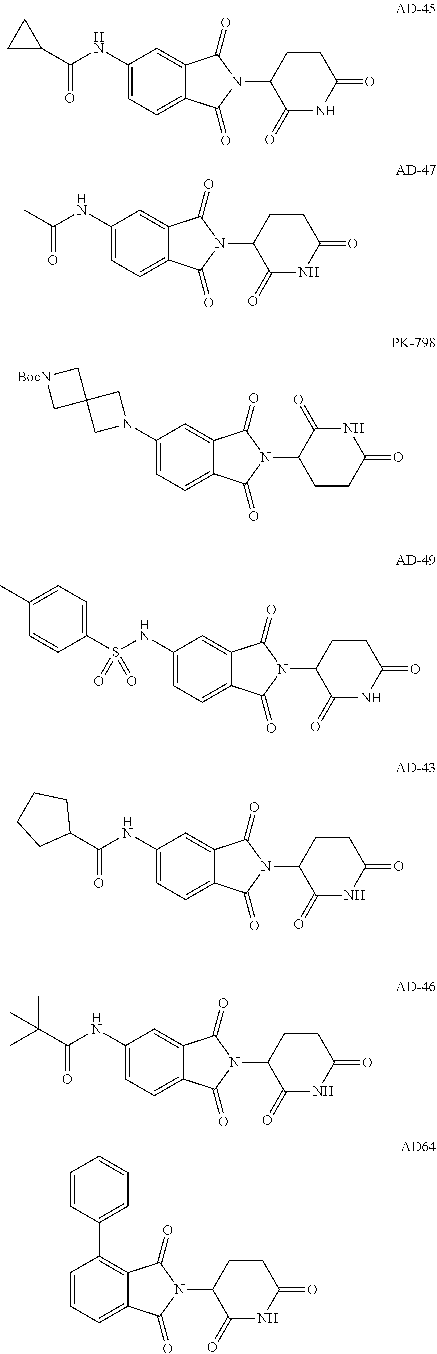

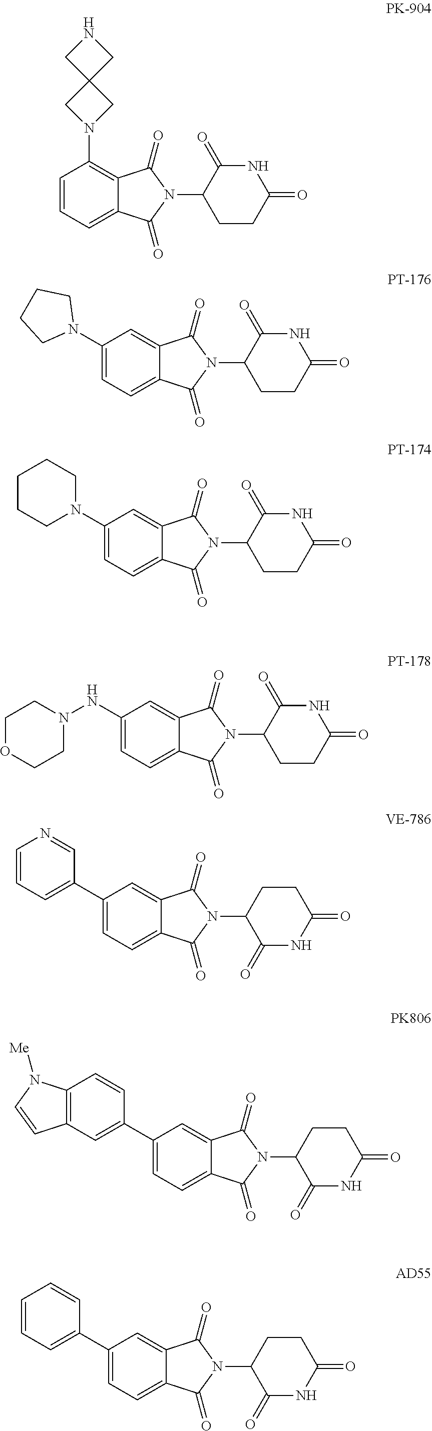

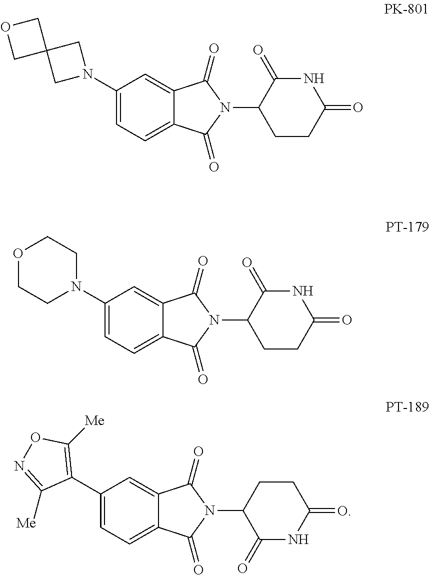 [SMILES: O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1 ; Cc1noc(C)c1-c1ccc2c(c1)C(=O)N(C1CCC(=O)NC1=O)C2=O]

49. The molecule of claim 46, selected from

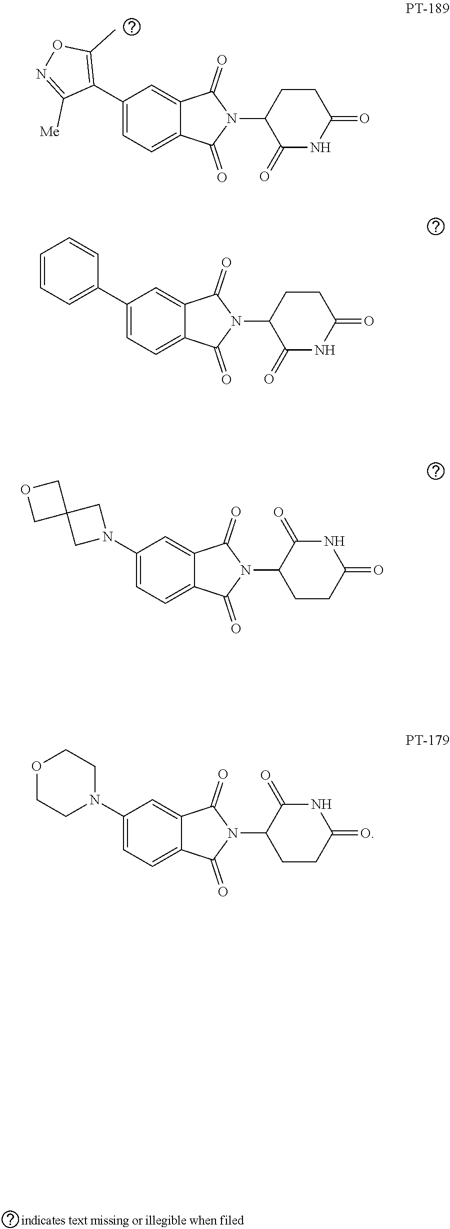 [SMILES: Cc1noc(C)c1-c1ccc2c(c1)C(=O)N(C1CCC(=O)NC1=O)C2=O ; O=C1CCC(N2C(=O)c3ccc(-c4ccccc4)cc3C2=O)C(=O)N1 ; O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1]

50. The molecule of claim 46, selected from

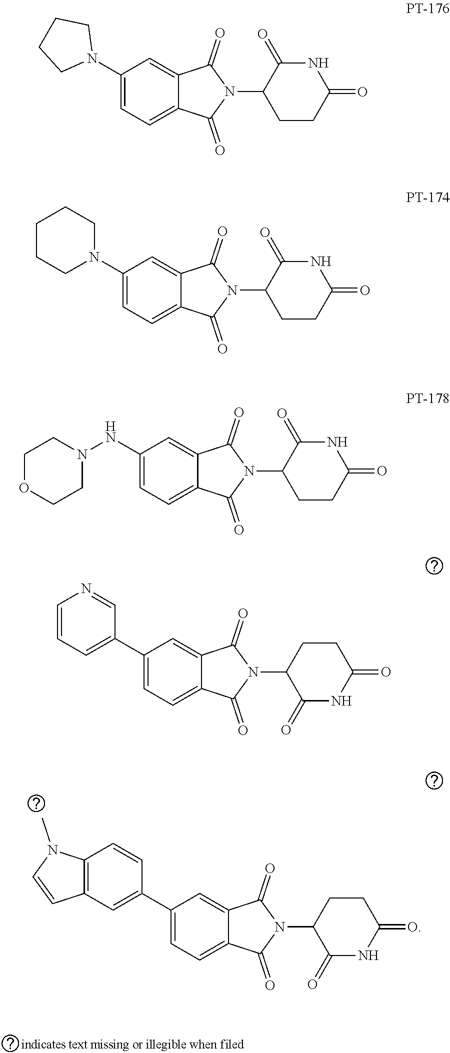 [SMILES: O=C1CCC(N2C(=O)c3ccc(N4CCCC4)cc3C2=O)C(=O)N1 ; O=C1CCC(N2C(=O)c3ccc(N4CCCCC4)cc3C2=O)C(=O)N1 ; O=C1CCC(N2C(=O)c3ccc(NN4CCOCC4)cc3C2=O)C(=O)N1 ; O=C1CCC(N2C(=O)c3ccc(-c4cccnc4)cc3C2=O)C(=O)N1 ; Cn1ccc2cc(-c3ccc4c(c3)C(=O)N(C3CCC(=O)NC3=O)C4=O)ccc21]

51. The molecule of claim 46, selected from

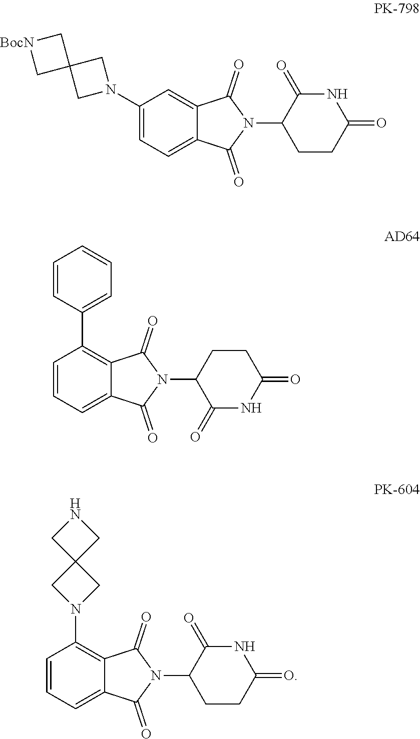 [SMILES: CC(C)(C)OC(=O)N1CC2(C1)CN(c1ccc3c(c1)C(=O)N(C1CCC(=O)NC1=O)C3=O)C2 ; O=C1CCC(N2C(=O)c3cccc(-c4ccccc4)c3C2=O)C(=O)N1 ; O=C1CCC(N2C(=O)c3cccc(N4CC5(CNC5)C4)c3C2=O)C(=O)N1]

52. The molecule of any one of claims 1-45, selected from

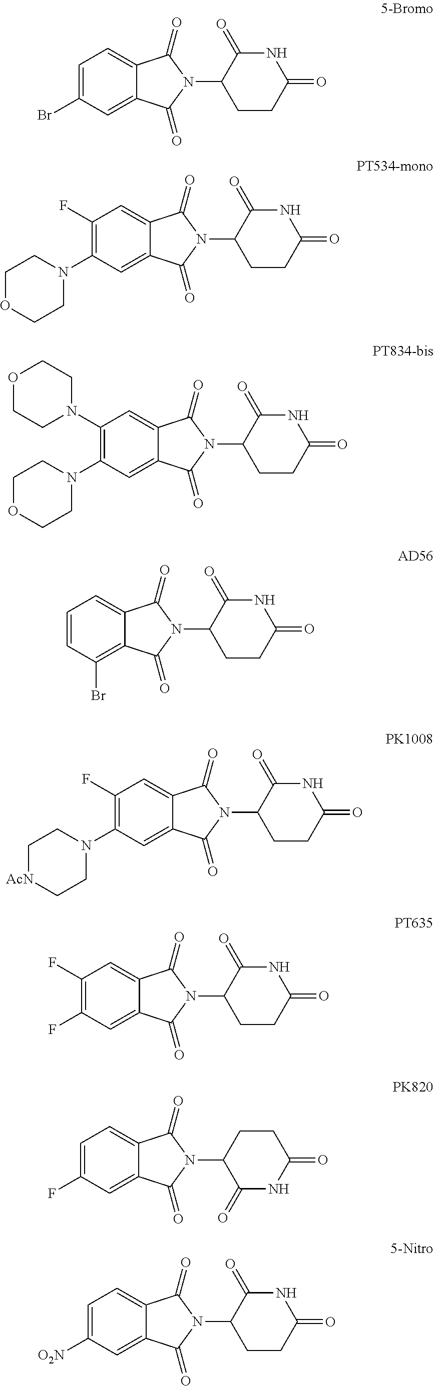

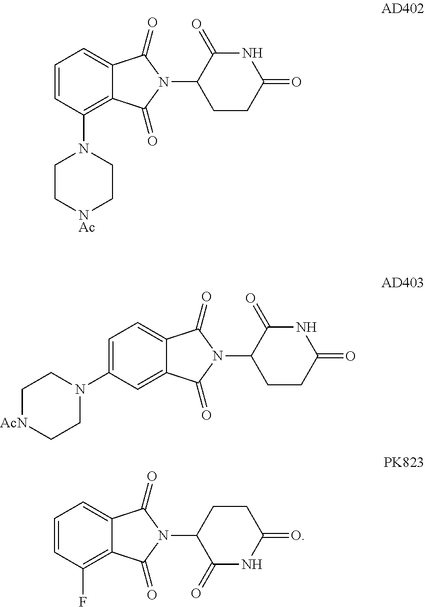

53. The molecule of claim 52 selected from

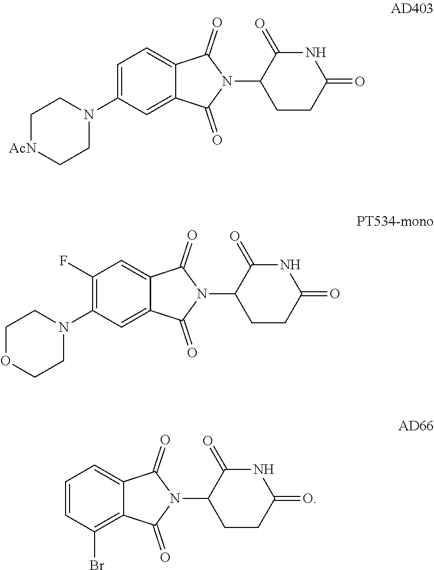

54. The molecule of claim 52, selected from

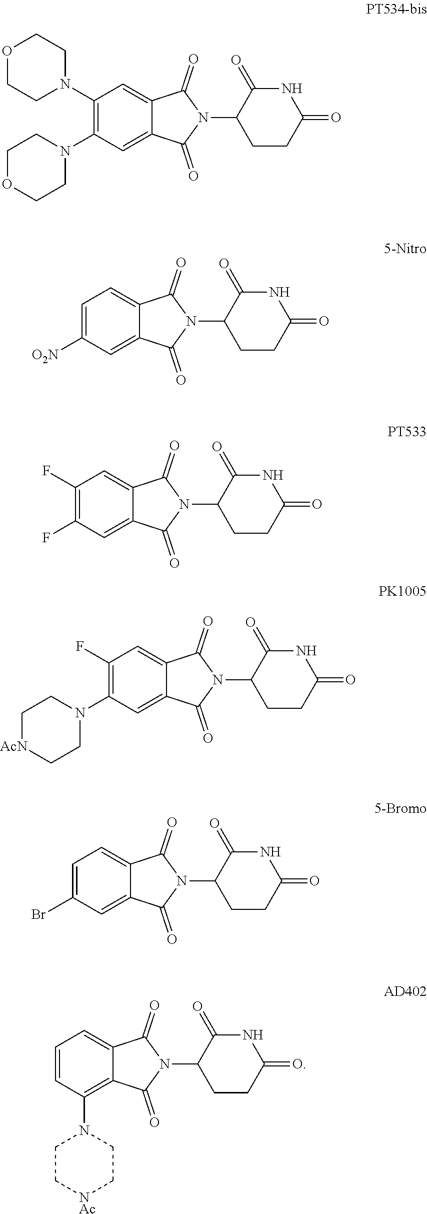

55. The molecule of any of claims 1-45, having the following structure 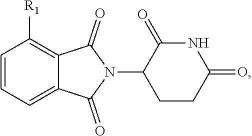
wherein R₁ is selected from

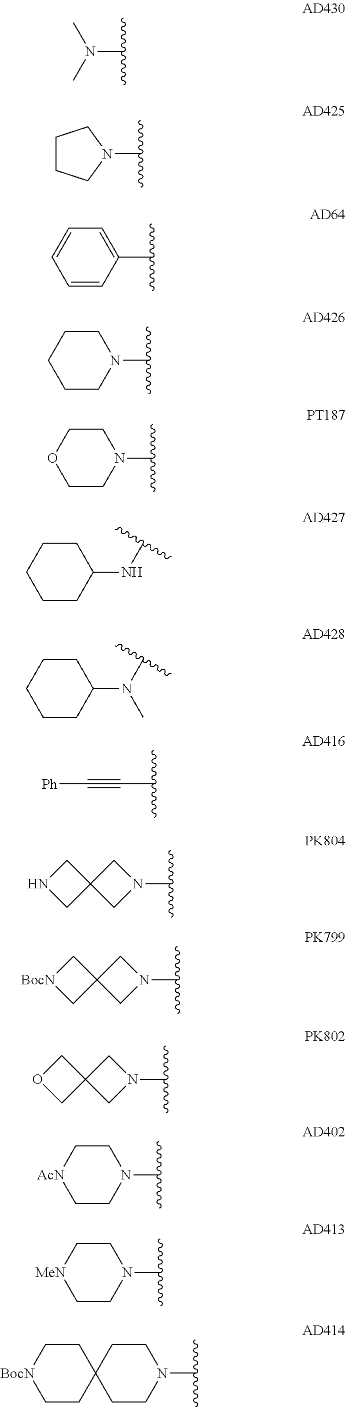

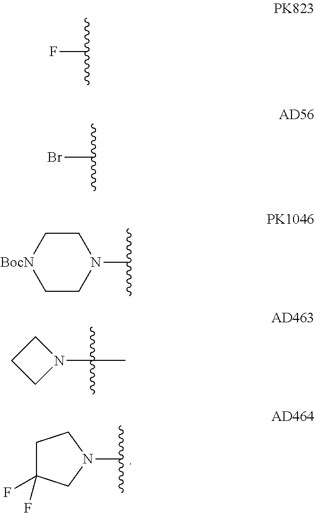

56. The molecule of claim 55, wherein R₁ is selected from

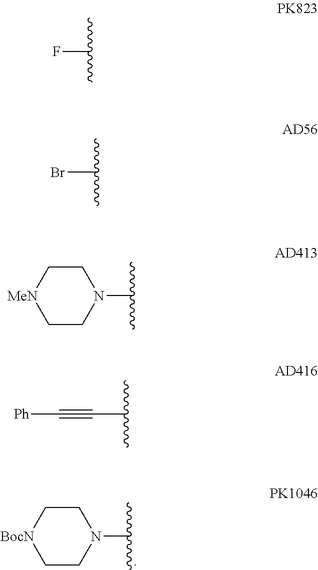

57. The molecule of any one of claims 1-45, having the following structure 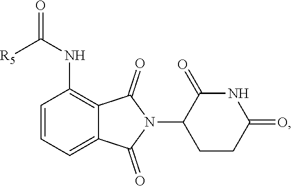
wherein R₅ is selected from

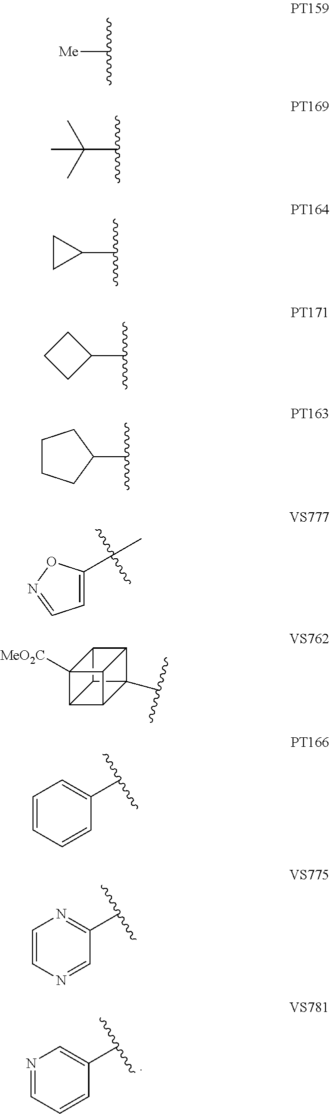

58. The molecule of any one of claims 1-45, having the following structure 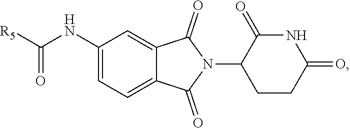
wherein R₅ is selected from

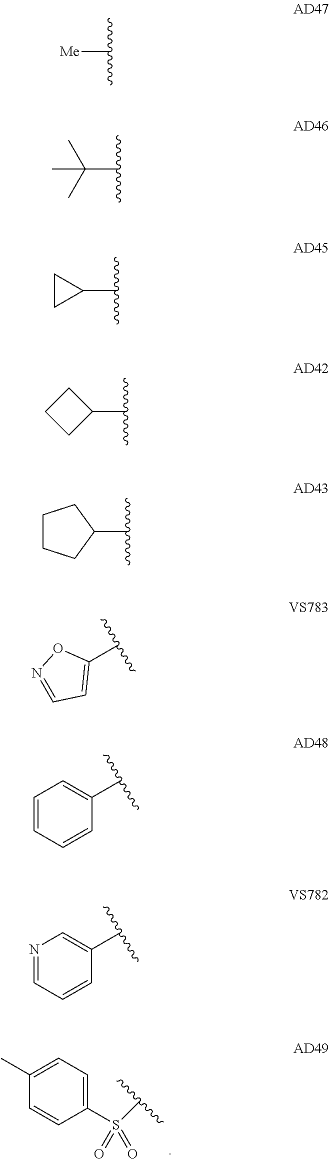

59. The molecule of any one of claims 1-45, having the following structure 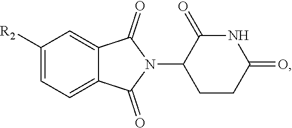
wherein R₂ is selected from

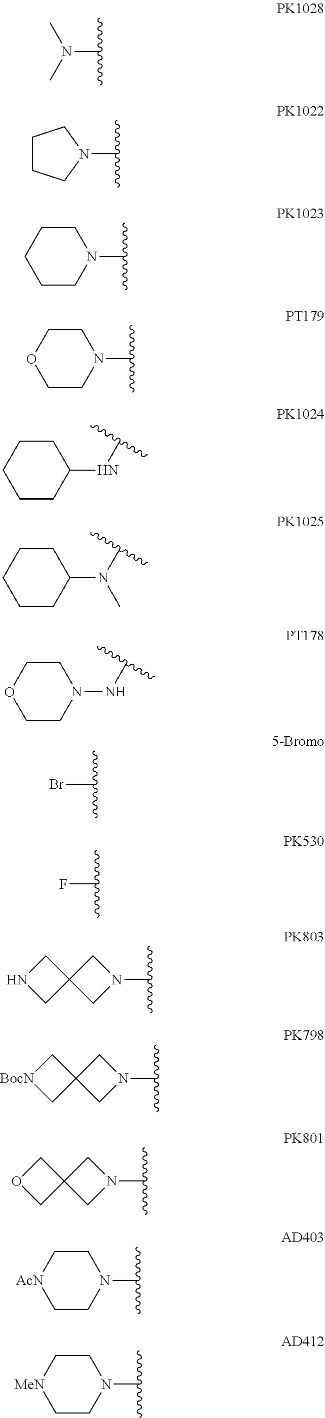

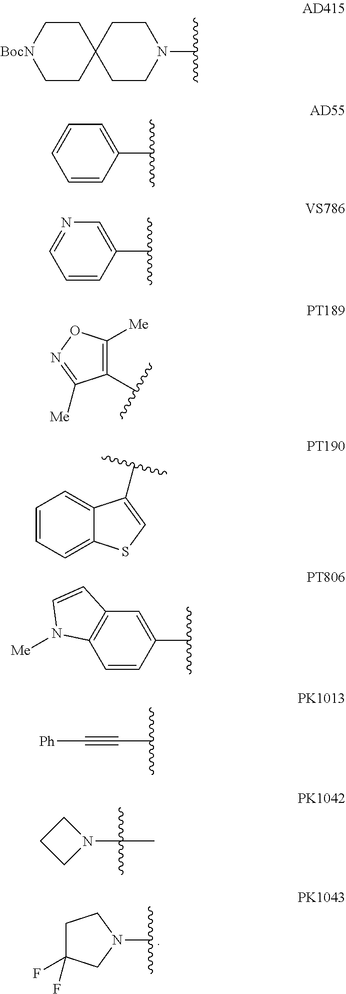

60. The molecule of claim 59, wherein R₂ is selected from

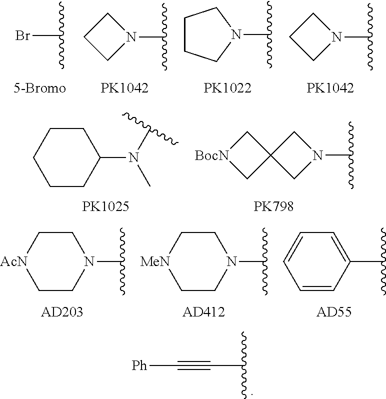

61. The molecule of any one of claims 1-45, having the following structure 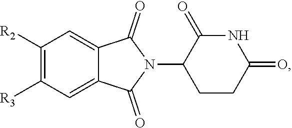
wherein R₃ is a fluorine group and R₂ is selected from 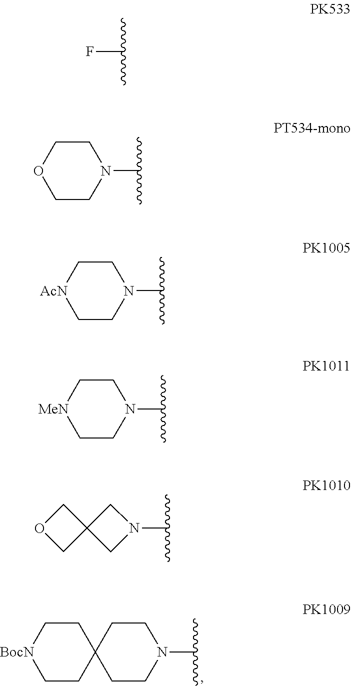
or
wherein R₂ and R₃ are each

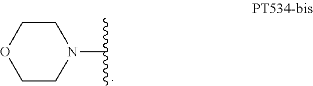 [SMILES: *N1CCOCC1]

62. The molecule of claim 1, according to the formula 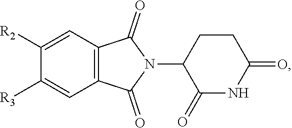
wherein R₂ is F and R₃ is selected from

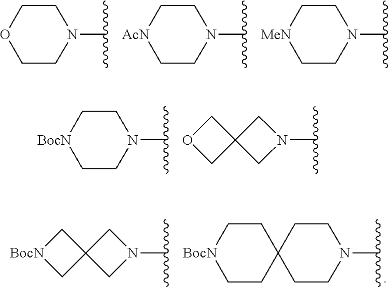

63. A molecule according to the formula 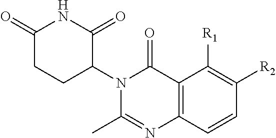
wherein R₁ is selected from —H and nitro groups, and
wherein R₂ is selected from —H and halogen groups.

64. The molecule according to claim 62, selected from

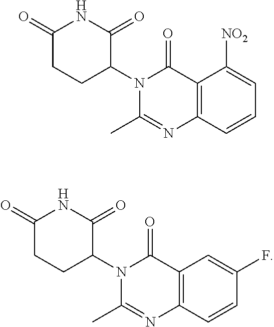 [SMILES: Cc1nc2cccc([N+](=O)[O-])c2c(=O)n1C1CCC(=O)NC1=O ; Cc1nc2ccc(F)cc2c(=O)n1C1CCC(=O)NC1=O]

65. A method of inducing degradation of a variant protein in a cell, comprising exposing a cell transfected with a variant protein comprising one or more zinc finger polypeptides at one or more insertion sides on the protein with a molecule according to any one of claims 1-64, a pharmaceutically acceptable salt thereof, or any pharmaceutical combination of molecules as described herein and/or pharmaceutically acceptable salts thereof.

66. The method of claim 65, wherein the variant protein is a programmable nuclease.

67. The method of claim 65 or 66, wherein the protein comprises a zinc finger selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, IKZF1/3 AA145-167/146-168, ZNF692 AA417-439, and combinations thereof.

68. The method of any one of claims 65-67, wherein the programmable nuclease is selected from a CRISPR-Cas protein, a Zinc finger nuclease, a TALEN or a meganuclease.

69. The method of claim 65, wherein the molecule is selected from 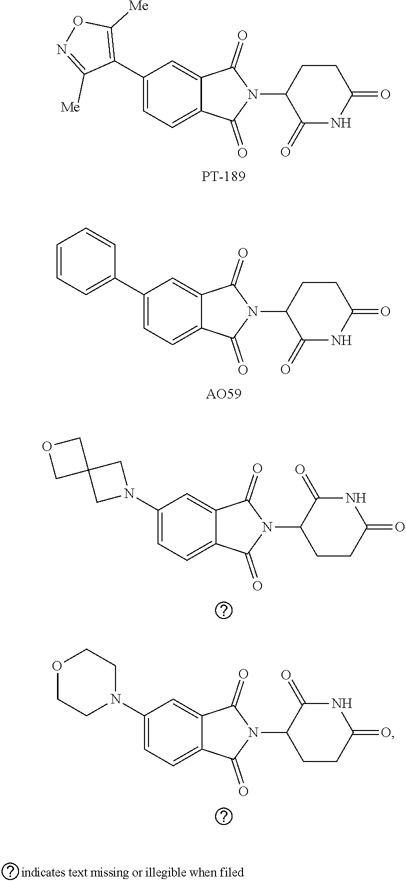 [SMILES: Cc1noc(C)c1-c1ccc2c(c1)C(=O)N(C1CCC(=O)NC1=O)C2=O ; O=C1CCC(N2C(=O)c3ccc(-c4ccccc4)cc3C2=O)C(=O)N1 ; O=C1CCC(N2C(=O)c3ccc(N4CC5(COC5)C4)cc3C2=O)C(=O)N1 ; O=C1CCC(N2C(=O)c3ccc(N4CCOCC4)cc3C2=O)C(=O)N1]
and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, and combinations thereof.

70. The method of claim 65, wherein the molecule is selected from 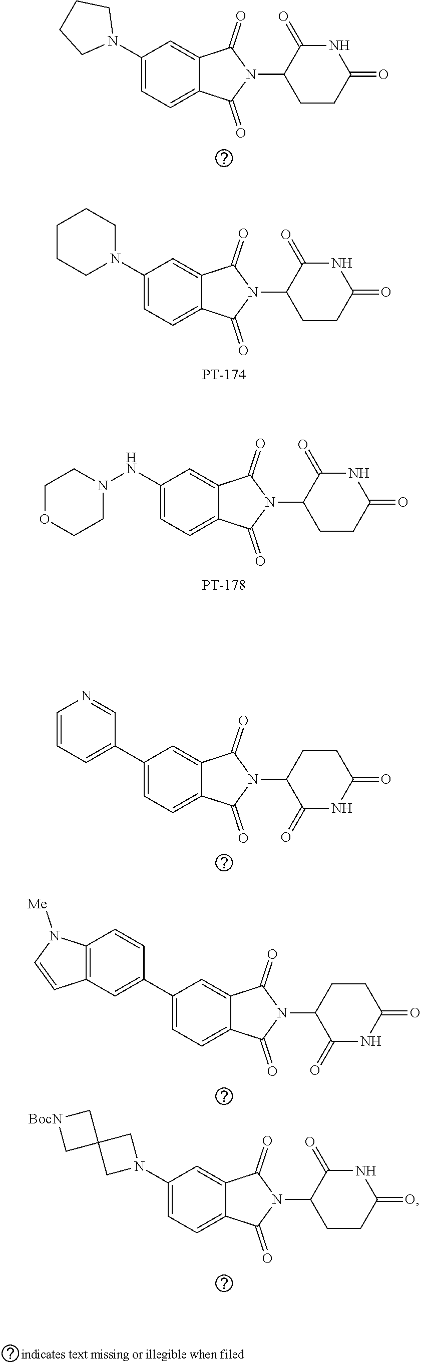 [SMILES: O=C1CCC(N2C(=O)c3ccc(N4CCCC4)cc3C2=O)C(=O)N1 ; O=C1CCC(N2C(=O)c3ccc(N4CCCCC4)cc3C2=O)C(=O)N1 ; O=C1CCC(N2C(=O)c3ccc(NN4CCOCC4)cc3C2=O)C(=O)N1 ; O=C1CCC(N2C(=O)c3ccc(-c4cccnc4)cc3C2=O)C(=O)N1 ; Cn1ccc2cc(-c3ccc4c(c3)C(=O)N(C3CCC(=O)NC3=O)C4=O)ccc21 ; CC(C)(C)OC(=O)N1CC2(C1)CN(c1ccc3c(c1)C(=O)N(C1CCC(=O)NC1=O)C3=O)C2]
and wherein the cell comprises one or more zinc fingers selected from ZFN653 AA556-578, ZFN517 AA452-474, ZFP91_400_422, E4F1 AA220-242, ZFN654 AA25-47, IKZF3_146_168, ZNF582 AA395-417, IKZF3 AA146-168 Q147E, SALL4 ZF2, and combinations thereof.

71. The method of claim 65, wherein the molecule is selected from 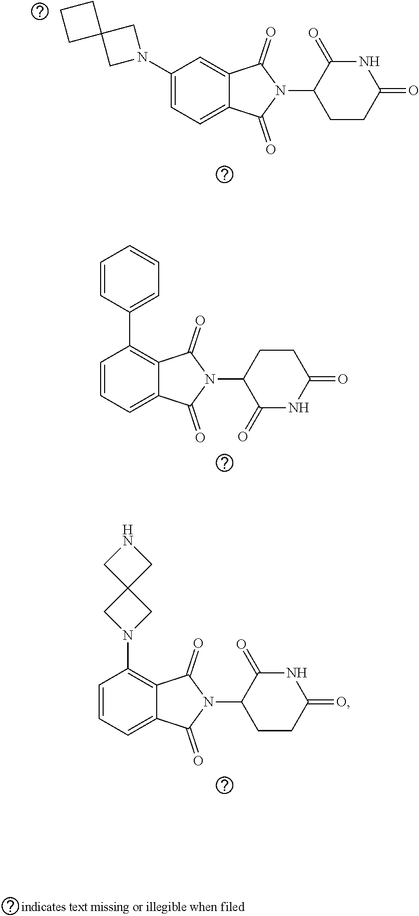 [SMILES: O=C1CCC(N2C(=O)c3ccc(N4CC5(CCC5)C4)cc3C2=O)C(=O)N1 ; O=C1CCC(N2C(=O)c3cccc(-c4ccccc4)c3C2=O)C(=O)N1 ; O=C1CCC(N2C(=O)c3cccc(N4CC5(CNC5)C4)c3C2=O)C(=O)N1]
and wherein the cell comprises one or more zine fingers selected from ZFN276 AA524-576, ZFN653 AA556-578, ZFN787 AA 178-200, ZFN517 AA452-474, ZFP91_400_422, E4F1 AA220-242, ZFN654 AA25-47, IKZF3_146_168, ZNF582 AA395-417, and combinations thereof.

72. The method of claim 65, wherein the molecule is selected from 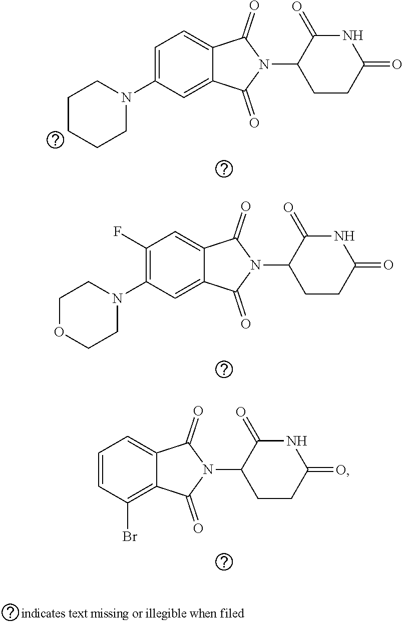 [SMILES: O=C1CCC(N2C(=O)c3ccc(N4CCCCC4)cc3C2=O)C(=O)N1 ; O=C1CCC(N2C(=O)c3cc(F)c(N4CCOCC4)cc3C2=O)C(=O)N1 ; O=C1CCC(N2C(=O)c3cccc(Br)c3C2=O)C(=O)N1]
and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, and combinations thereof.

73. The method of claim 65, wherein the molecule is selected from 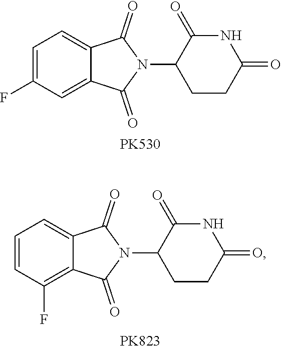
and wherein the cell comprises one or more zinc fingers selected from ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, IKZF3_146_168, ZNF582 AA395-417, ZKSC5_430_452, IKZF3 AA146-168 Q147E, SALL4 ZF2, and combinations thereof.

74. The method of claim 65, wherein the molecule is selected from 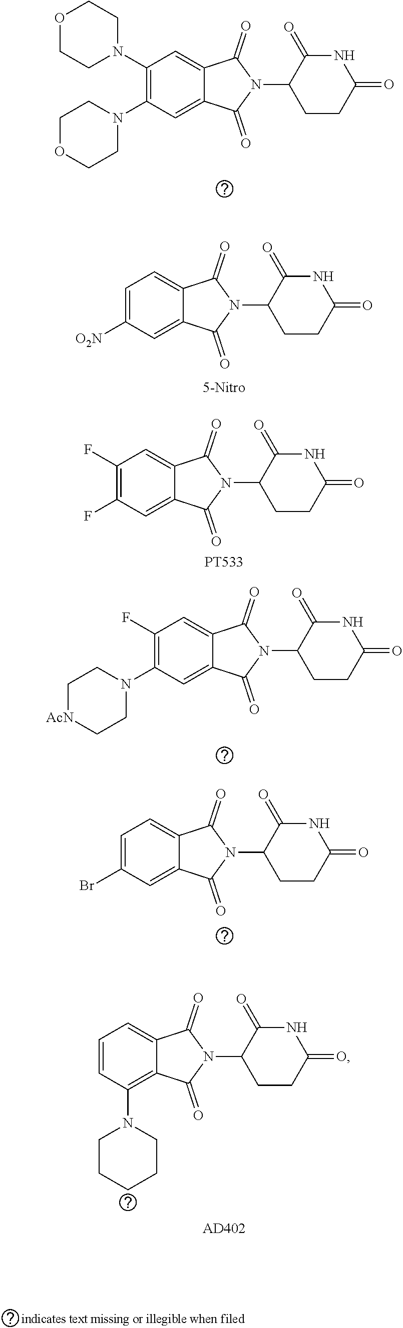
and wherein the cell comprises one or more zine finger ZFN653 AA556-578, ZFN827 AA374-396, ZFN787 AA 178-200, ZFN517 AA452-474, IKZF3_146-168, ZNF582 AA395-417, IKZF3 AA146-168 Q147E, SALL4 ZF2, and combinations thereof.

75. The method of claim 65, wherein the molecule is according to the formula 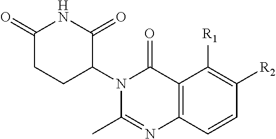
wherein R₁ is selected from —H and nitro groups, and
wherein R₂ is selected from —H and halogen groups, and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400-422, E4F1 AA220-242, PATZ1_383_405, ZFN654 AA25-47, IKZF3_146_168, ZNF582 AA395-417, ZKSC5_430_452, IKZF3 AA146-168 Q147E, SALL4 ZF2, and combinations thereof.

76. The method of claim 65, wherein the molecule is selected from 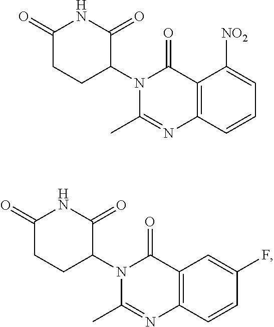 [SMILES: Cc1nc2cccc([N+](=O)[O-])c2c(=O)n1C1CCC(=O)NC1=O ; Cc1nc2ccc(F)cc2c(=O)n1C1CCC(=O)NC1=O]
and wherein the cell comprises one or more zinc fingers selected from ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, IKZF3 AA146-168 Q147E, and combinations thereof.

77. A method of inducing degradation of a variant protein in a cell, comprising exposing a cell transfected with variant protein comprising one or more FK506 binding protein (FKBP) domains, with a composition according to the formula:
A-(L)ₙ-B,
a pharmaceutically acceptable salt thereof, or any pharmaceutical combination of compositions according to the formula: A-(L)ₙ-B and/or pharmaceutically acceptable salts thereof,
wherein L is a linker,
wherein n is between 0 and 12,
wherein A is ligand that binds to one of the FKBP domains,
wherein B is a molecule according to any one of claims 1-64, and
wherein B is conjugated to A or (L)ₙ via R₁ or R₂.

78. The method of claim 77, wherein (L)ₙ-B comprises an alkyl, an alkyne, a glycol ether, a polyglycol ether, a heterocyclic, a heteroaryl, or an aryl group.

79. The method of claim 77 or 78, wherein (L)ₙ-B comprises a C₄₋₈ alkyl group.

80. The method of any one of claims 77-79, wherein (L)ₙ-B comprises a group selected from

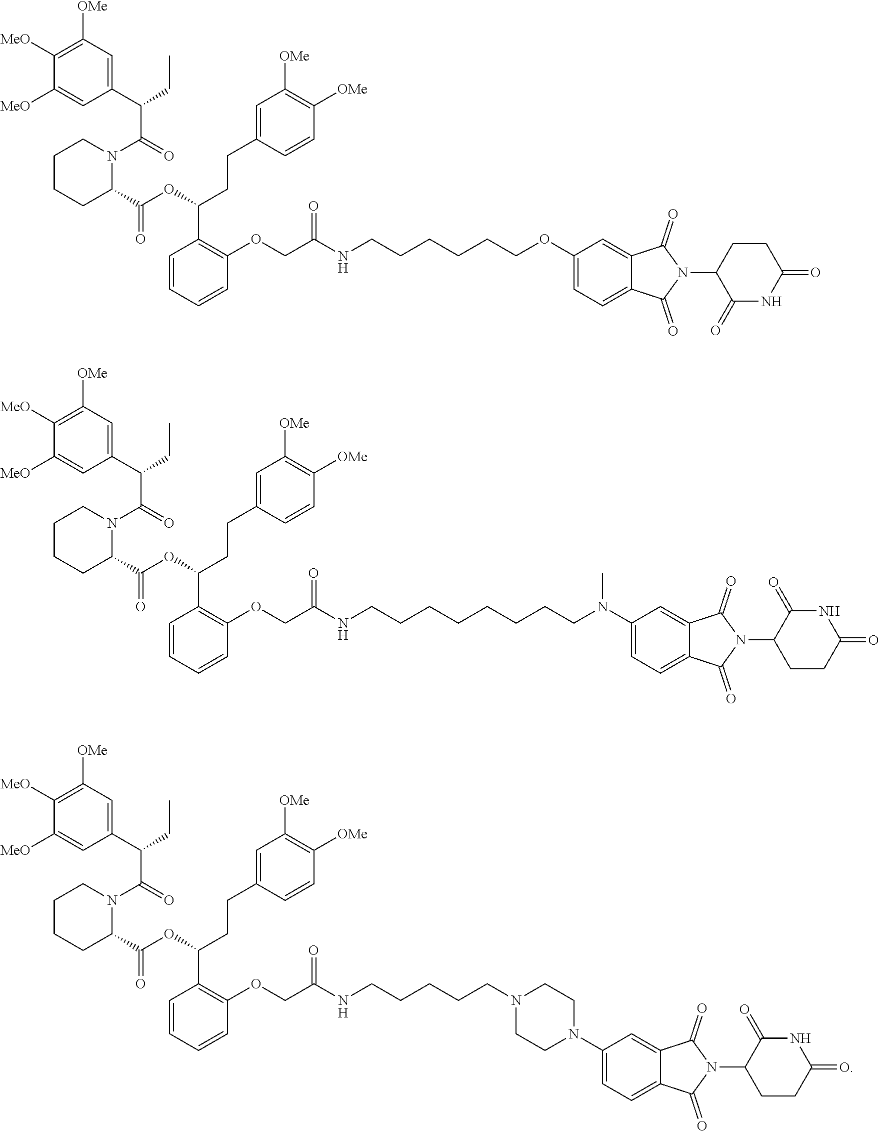 [SMILES: CC[C@H](C(=O)N1CCCC[C@H]1C(=O)O[C@H](CCc1ccc(OC)c(OC)c1)c1ccccc1OCC(=O)NCCCCCCOc1ccc2c(c1)C(=O)N(C1CCC(=O)NC1=O)C2=O)c1cc(OC)c(OC)c(OC)c1 ; CC[C@H](C(=O)N1CCCC[C@H]1C(=O)O[C@H](CCc1ccc(OC)c(OC)c1)c1ccccc1OCC(=O)NCCCCCCCCN(C)c1ccc2c(c1)C(=O)N(C1CCC(=O)NC1=O)C2=O)c1cc(OC)c(OC)c(OC)c1 ; CC[C@H](C(=O)N1CCCC[C@H]1C(=O)O[C@H](CCc1ccc(OC)c(OC)c1)c1ccccc1OCC(=O)NCCCCCN1CCN(c2ccc3c(c2)C(=O)N(C2CCC(=O)NC2=O)C3=O)CC1)c1cc(OC)c(OC)c(OC)c1]

81. The method of any one of claims 77-80, wherein (L)ₙ-B is selected from

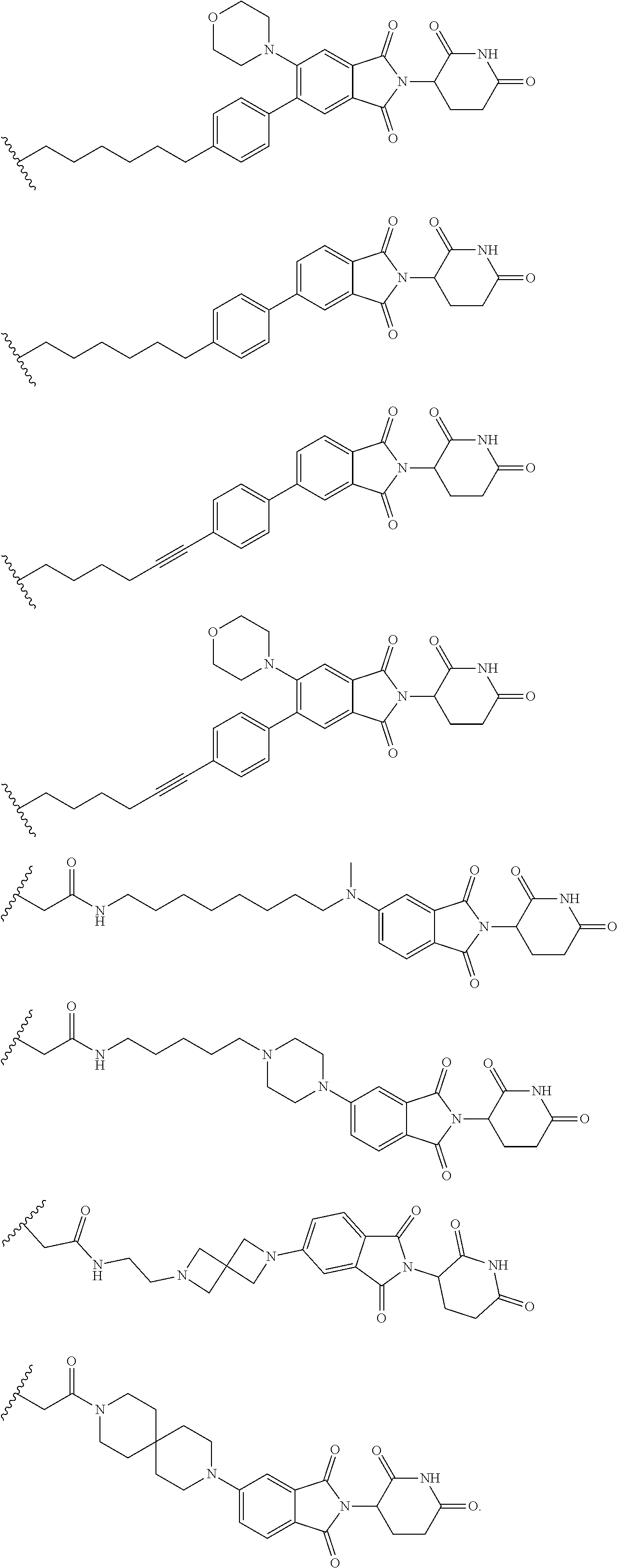

82. The method of any one of claims 77-79, wherein R₁ or R₂ is according to R₄, and wherein R₄ is an ether group according to the formula:
—NH—C(O)—CH₂—O— or —O—.

83. The method of any one of claims 77-79, and 82, wherein (L)ₙ-B is

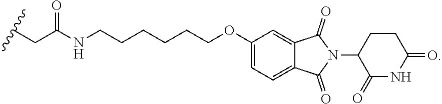 [SMILES: *CC(=O)NCCCCCCOc1ccc2c(c1)C(=O)N(C1CCC(=O)NC1=O)C2=O]

84. A method of inducing degradation of a target amine in a cell, comprising:
exposing a cell comprising a target amine with a composition according to the formula:
A-(L)ₙ-B,
a pharmaceutically acceptable salt thereof, or any pharmaceutical combination of compositions according to the formula: A-(L)ₙ-B and/or pharmaceutically acceptable salts thereof,
wherein L is a linker and wherein n is between 0 and 12,
wherein A is a ligand selective for the target amine,
wherein B is a molecule of any one of claims 1-64, and
wherein B is conjugated to A or (L)ₙ via R₁ or R₂.

85. The method of claim 77, wherein (L)ₙ-B comprises an alkyl, an alkyne, a glycol ether, a polyglycol ether, a heterocyclic, a heteroaryl, or an aryl group.

86. The method of claim 77 or 85, wherein (L)ₙ-B comprises a C₄₋₈ alkyl group.

87. The method of any one of claims 77-86, wherein (L)ₙ-B comprises a group selected from

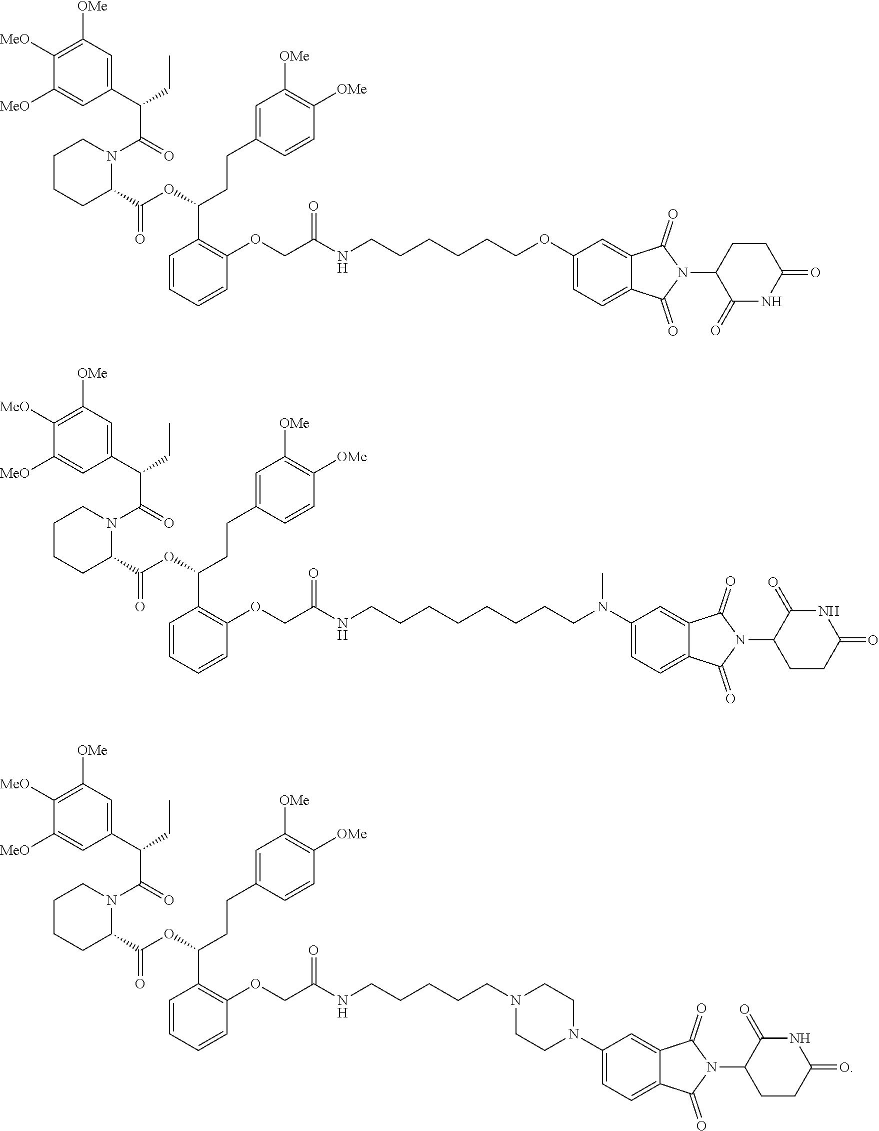 [SMILES: CC[C@H](C(=O)N1CCCC[C@H]1C(=O)O[C@H](CCc1ccc(OC)c(OC)c1)c1ccccc1OCC(=O)NCCCCCCOc1ccc2c(c1)C(=O)N(C1CCC(=O)NC1=O)C2=O)c1cc(OC)c(OC)c(OC)c1 ; CC[C@H](C(=O)N1CCCC[C@H]1C(=O)O[C@H](CCc1ccc(OC)c(OC)c1)c1ccccc1OCC(=O)NCCCCCCCCN(C)c1ccc2c(c1)C(=O)N(C1CCC(=O)NC1=O)C2=O)c1cc(OC)c(OC)c(OC)c1 ; CC[C@H](C(=O)N1CCCC[C@H]1C(=O)O[C@H](CCc1ccc(OC)c(OC)c1)c1ccccc1OCC(=O)NCCCCCN1CCN(c2ccc3c(c2)C(=O)N(C2CCC(=O)NC2=O)C3=O)CC1)c1cc(OC)c(OC)c(OC)c1]

88. The method of any one of claims 77-86, wherein (L)ₙ-B is selected from

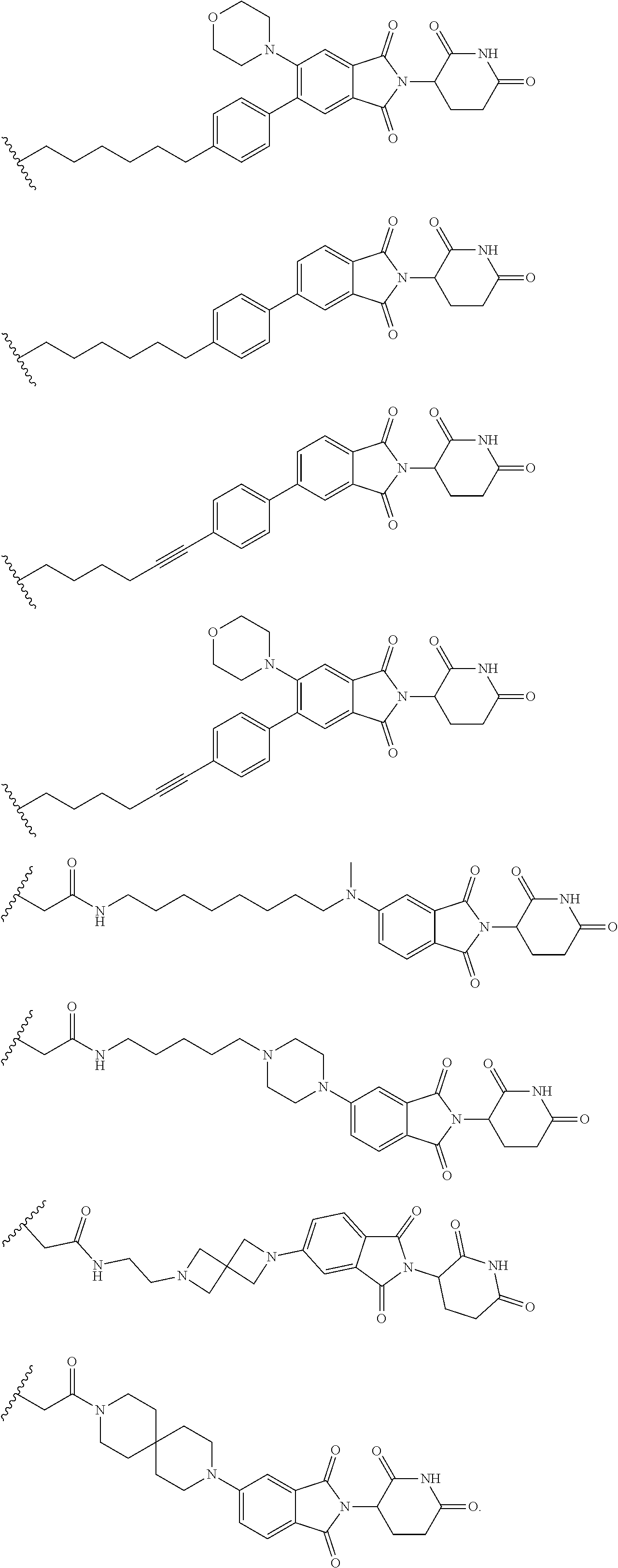

89. The method of any one of claims 77-86, wherein R₁ or R₂ is according to R₄, and wherein R₄ is an ether group according to the formula:
—NH—C(O)—CH₂—O— or —O—.

90. The method of any one of claims 77-86, and 89 wherein (L)ₙ-B is

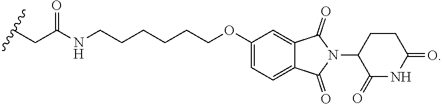 [SMILES: *CC(=O)NCCCCCCOc1ccc2c(c1)C(=O)N(C1CCC(=O)NC1=O)C2=O]

91. The method of any one of claims 77-90, wherein the target amine is a programmable nuclease, and wherein the cell is transfected with the programmable nuclease prior to the exposing step.

92. The method of any one of claims 77-91, wherein the programmable nuclease is selected from a CRISPR-Cas protein, a Zinc finger nuclease, a TALEN or a meganuclease.

93. The method of any one of claims 77-92, wherein R₁ or R₂ is according to R₄, and wherein R₄ is an ether group according to the formula —NH—C(O)—CH₂—O—, and wherein the cell comprises one or more zinc fingers selected from ZFN91-IKFZ3, ZFN276 AA524-576, ZFN653 AA556-578, ZFN827 AA374-396, ZFN787 AA 178-200, ZFN517 AA452-474, ZFP91_400_422, E4F1 AA220-242, PATZ1_383_405, ZFN654 AA25-47, IKZF3_146_168, ZNF582 AA395-417, ZKSC5_430_452, and combinations thereof.

94. The method of any one of claims 77-93, wherein R₁ or R₂ is according to R₄, and wherein R₄ is an ether group according to the formula —O—, and wherein the cell comprises one or more zinc fingers selected from ZFN787 AA 178-200, IKZF3_146_168, ZKSC5_430_452, and combinations thereof.
